(19) United States
(12) Patent Application Publication
(10) Pub. No.: US 2024/0101647 A1
(43) Pub. Date: Mar. 28, 2024

(54) SARBECOVIRUS BINDERS

(71) Applicants: VIB VZW, Gent (BE); UNIVERSITEIT GENT, Gent (BE); VRIJE UNIVERSITEIT BRUSSEL, Brussel (BE)

(72) Inventors: Xavier SAELENS, Ieper (BE); Dorien DE VLIEGER, Lotenhulle (BE); Iebe ROSSEY, Gent (BE); Sieglinde DE CAE, Lennik (BE); Koen SEDEYN, Heusden (BE); Loes VAN SCHIE, Gent (BE); Hannah EECKHAUT, Oosterzele (BE); Daria FIJALKOWSKA, Evergem (BE); Catelijne STORTELERS, Gent (BE); Bert SCHEPENS, Gent (BE); Nico CALLEWAERT, Nevele (BE); Han REMAUT, Roosbeek (BE); Wim NERINCKX, Astene (BE); Kenny ROOSE, Gent (BE); Dirk REITER, Brussel (BE)

(21) Appl. No.: 18/275,986

(22) PCT Filed: Feb. 7, 2022
(86) PCT No.: PCT/EP2022/052919

§ 371 Date: Aug. 4, 2023

(30) Foreign Application Priority Data
Feb. 5, 2021 (WO) PCT/EP2021/052885
Apr. 2, 2021 (EP) 21166835.5
May 12, 2021 (EP) 21173680.6

(51) Int. Cl. C07K 16/10; A61P 31/14

(52) U.S. Cl. CPC C07K 16/1003; A61P 31/14; A61K 2039/505

(57) ABSTRACT

Agents that bind to sarbecoviruses of multiple clades, neutralizing sarbecovirus infection, in particular neutralizing SARS-CoV-1 and SARS-CoV-2 infection. The agents bind to a unique epitope of the sarbecovirus ACE2-receptor binding domain (RBD) but do not inhibit binding of ACE2 with the RBD. Applications and uses of these agents are further disclosed.

### FIELD OF THE INVENTION

The invention relates to agents binding to sarbecoviruses of multiple clades and potently neutralizing sarbecovirus infection, in particular neutralizing SARS-CoV-1 and SARS-CoV-2 infection, including neutralizing a SARS-CoV-2 variant infection. The agents bind to a unique epitope of the sarbecovirus ACE2-receptor binding domain (RBD) but do not inhibit binding of ACE2 with the RBD. Application and uses of these agents are further part of this invention.

### INTRODUCTION TO THE INVENTION

Severe acute respiratory syndrome Coronavirus 2 (SARS-CoV-2) is the causative agent of COVID-19, a disease that has rapidly spread world-wide with devastating consequences. SARS-CoV-2 infections can be asymptomatic and mostly present with mild to moderately severe symptoms. However, in approximately 10% of patients, COVID-19 progresses to a more severe stage that is characterized by dyspnoea and hypoxemia, which may progress further to acute respiratory distress requiring often long-term intensive care and causing death in a proportion of patients. “Long-COVID” furthermore refers to long-term effects of COVID-19 infection, even when no SARS-CoV-2 virus can be detected anymore. Most likely, the ongoing inflammation triggered by the innate recognition of the SARS-CoV-2 virus, and possibly also by immune complexes with antibodies from an ineffective immune response, contributes to severe disease progression.

The approximately 30.000 nucleotide genome of the novel coronavirus (CoV) causing COVID-19 (2019-nCoV or WUHAN-Corona or SARS-CoV-2 virus) was elucidated in record time (see http://virological.org/t/novel-2019-coronavirus-genome/319 (accessed on 19 Jan. 2020).

Similar to the severe acute respiratory syndrome virus (SARS) caused by SARS-CoV-1, SARS-CoV-2 uses the angiotensin converting enzyme 2 (ACE2) as a receptor for entry into human cells. SARS-CoV-2 binds ACE2 with a higher affinity than SARS-CoV-1.

Prophylactic vaccines (active immunotherapy, vaccine-induced in vivo generation of neutralizing antibodies) is expected to become a cornerstone in controlling the pandemic. US and EU regulatory bodies have e.g. meanwhile approved RNA-based vaccines for treatment of COVID-19. Drawbacks of these vaccines are storage at very low temperatures (−70° C. or −20° C.). Other prophylactic vaccines based on e.g. engineered adenoviruses are underway which can be stored under more suitable circumstances. Protection offered by prophylactic vaccines may be insufficient. Indeed, immunity against coronaviruses can be short-lived, and especially elderly tend to be protected less efficiently upon vaccination. On the other hand, the emergence of new SARS-CoV-2 variants escaping from a previous immune response (whether by natural infection or by prophylactic vaccine) may hamper protection (e.g. Weisblum et al. 2020, eLife 2020; 9:e61312). Hence, therapeutic options to suppress or even prevent (further) viral replication in the lower airways will likely find an important place in rescuing patients (elderly or other) that have contracted or re-contracted COVID-19. Such therapeutic options for patients already suffering from SARS-CoV-2 infection remain, however, very limited.

A particular type of therapeutic approach potentially relies on neutralizing antibodies, i.e. on passive antibody therapy/immunotherapy (egress of immunoglobulin from the systemic circulation into the broncho-alveolar space is augmented due to inflammation in the lower airways, systemic administration of a neutralizing antibody is thus feasible). Rujas et al. 2020 (doi: https://doi.org/10.1101/2020.10.15.341636) provide a good overview of antibodies binding to the spike protein (S) of SARS-CoV-2 for which entries are available in the Protein Data Bank (PDB) or Electron Microscopy Data Bank (EMDB), and provide some new antibodies, some of which (antibodies 46 and 52) with a binding site shifting somewhat away from the receptor binding motif and potentially destabilizing the spike protein. Cross-reactivity of antibodies to the S-domain of SARS-CoV to SARS-CoV-2 is described by Bates et al. 2021 (Cell Rep 34:108737). Single domain antibody/nanobody-format neutralizers of both SARS-CoV-1 and -2 have been reported such as VHH72 by Wrapp et al. 2020 (Cell 184:1004-1015).

Multiple other single domain antibodies such as nanobodies capable of neutralizing SARS-CoV-2 have been described. For instance: Xiang et al. 2020 (Science 370:1479-1484) disclose 4 groups of nanobodies, each group binding to different epitopes, of which 2 groups are capable of competing with human ACE-2 for binding with the RBD (epitopes I and II), and of which 2 groups are not competing with ACE-2 for binding the RBD and which are capable of binding with trimeric spike protein only when 2 or 3 of the RBDs are in the up-conformation (epitopes III and IV)— of these, Nb20 and Nb21 binding to epitope I were later reported to loose neutralization potency when the E484K mutation is present in the spike protein, and Nb34 and Nb95 (binding to epitopes III and IV, resp.) were assigned as “class II Nbs”, most importantly, Nb34 and Nb95 were also reported as capable of blocking ACE2 binding at low nM concentrations (Sun et al. 2021, BioRxiv https://doi.org/10.1101/2021.03.09.434592); Sun et al. 2021, (BioRxiv https://doi.org/10.1101/2021.03.09.434592) report further nanobodies Nb17 and Nb36; Schoof et al. 2020 (Science 370:1473-1479) disclose a nanobody disrupting spike protein-ACE2 interaction and binding to the spike protein in inactive conformation; Huo et al. 2020 (Nat Struct Mol Biol 27:846-854) and Hanke et al. 2020 (Nat Comm 11:4420) disclose further nanobodies capable of blocking RBD-ACE2 interaction; Wu et al. 2020 (Cell Host Microbe 27:891) describe five groups of nanobodies, with group D neutralizing and group E moderately neutralizing, groups D and E allegedly not competing for binding between RBD and ACE2, and group D targeting a cryptic epitope on the spike trimeric interface and competing with antibody CR3022 (the latter a non-neutralizing monoclonal antibody)—group A antibodies were competing with ACE2 for binding the RBD but were not efficiently neutralizing; and Dong et al. 2020 (Emerging Microbes & Infections 9: 034-1036) describe nanobodies capable of blocking RBD-ACE2 interaction. Wu et al. 2021 (BioRxiv doi: https://doi.org/10.1101/2021.02.08.429275) reported a series of SARS-CoV-2 neutralizing nanobodies the effect of which on RBD-ACE-2 interaction is not known, but otherwise defined by CDR sequences; these authors focus on the fact that a bispecific nanobody format increases potency in the setting of intranasal administration.

Many variants of SARS-CoV-2 virus have been identified (26844 single mutations in 203346 hCoV-19 genomes, see https://users.math.msu.edu/users/weig/SARS-CoV-2 Mutation Tracker.html; at least 28 different amino acid variations in the receptor binding domain (RBD), see https://covidcg.org/?tab=location; accessed on 12 Feb. 2021), some of which appearing to be more infectious than the original SARS-CoV-2 strain, and not all prophylactic vaccines may offer protection against such variants. The monoclonal antibodies casirivimab and imdevimab (Regeneron) and bamlanivimab (Lilly), have received emergency use authorization from US FDA. SARS-CoV-2 variants B.1.351 (South Africa; includes variants in the RBD K417N, E484K, N501Y) and B.1.1.248 (Brazil; includes variants in the RBD K417T, E484K, and N501Y) were very recently reported to be partially resistant to casirivimab and to be fully resistant to bamlanivimab (Hoffmann et al. 2021, doi: https://doi.org/10.1101/2021.02.11.430787), amply demonstrating the need for additional therapeutic options.

### SUMMARY OF THE INVENTION

The invention relates in one aspect to sarbecovirus binding agents characterized in that these are binding to the sarbecovirus spike protein Receptor Binding Domain (SPRBD), are allowing binding of Angiotensin-Converting Enzyme 2 (ACE2) to SPRBD when themselves bound to SPRBD, are at least neutralizing SARS-CoV-2 and SARS-CoV-1, and, in certain embodiments, are binding to: at least one of the amino acids Thr393 (or alternatively Ser393 in some sarbecoviruses), Asn394 (or alternatively Ser394 in some sarbecoviruses), Val395, or Tyr396 of the SARS-CoV-2 spike protein as defined in SEQ ID NO:30 and at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses), Arg466, or Arg357 (or alternatively Lys357 in some sarbecoviruses) of the SARS-CoV-2 spike protein as defined in SEQ ID NO:30. In other embodiments, these binding agents are binding to at least one, or in increasing order of preference at least two, at least three, or at least four, of the amino acids Asn394 (or alternatively Ser394 in some sarbecoviruses), Tyr396, Phe464, Ser514, Glu516, and Arg355 of the SARS-CoV-2 spike protein as defined in SEQ ID NO:30; and optionally are further binding to amino acid Arg357 (or alternatively Lys357 in some sarbecoviruses) and/or Lys462 (or alternatively Arg462 in some sarbecoviruses) and/or Glu465 (or alternatively Gly465 in some sarbecoviruses) and/or Arg466 and/or Leu518.

A further aspect relates to a multivalent or multispecific sarbecovirus binding agent, wherein one or more of the above-described sarbecovirus binding agents are fused directly or via a linker, preferably fused via an Fc domain.

In a further aspect, the invention relates to isolated nucleic acids encoding a sarbecovirus binding agents comprising an immunoglobulin single variable domain or functional part thereof as described herein; as well as to recombinant vectors comprising such nucleic acid.

The invention likewise relates to pharmaceutical compositions comprising an above-described sarbecovirus binding agent, multivalent or multispecific sarbecovirus binding agent, isolated nucleic acid and/or a recombinant vector.

The invention likewise relates to an above-described sarbecovirus binding agent, multivalent or multispecific sarbecovirus binding agent, isolated nucleic acid and/or a recombinant vector and to pharmaceutical compositions comprising such sarbecovirus binding agent, multivalent or multispecific sarbecovirus binding agent, isolated nucleic acid and/or a recombinant vector, for use as a medicament, for use in the treatment of a sarbecovirus infection, or for use in passive immunisation of a subject. In particular in case of use in passive immunisation, the subject may be having a sarbecovirus infection, may not be having a sarbecovirus infection.

The invention likewise relates to an above-described sarbecovirus binding agent and/or multivalent or multispecific sarbecovirus binding agent for use in diagnosing a sarbecovirus infection.

The invention likewise relates to an above-described sarbecovirus binding agent, multivalent or multispecific sarbecovirus binding agent, isolated nucleic acid and/or a recombinant vector, for use in the manufacture of a diagnostic kit.

In any of the above, the sarbecovirus binding agent in particular may be SARS-CoV-1 or SARS-CoV-2.

### DESCRIPTION OF THE FIGURES

The drawings described are only schematic and are non-limiting. In the drawings, the size of some of the elements may be exaggerated and not drawn on scale for illustrative purposes.

FIG. 1. Identification of periplasmic extracts that contain VHHs that bind the SARS-CoV-2 RBD without competing with VHH72 for binding. (A) Binding of VHHs to monovalent RBD-SD1-monohuFc that was either directly coated to an ELISA plate (x-axis) or captured by VHH72-Fc that was coated on an ELISA plate (y-axis). The dot plot shows for every PE, the OD (450 nm) values of both ELISA analyses. The dotted lines represent 2 x the mean OD (450 nm) value obtained for 4 PBS samples. The individual PE samples are shown as grey diamonds, except from the PE samples that contain VHHs (PE_VHH3.42, PE_VHH3.117, PE_VHH3.92, PE_VHH3.94, and PE_VHH3.180) that belong to the VHH3.42 family. (B) Alignment of the VHHs of the VHH3.42 family with amino acid residue numbering according to Kabat numbering. CDR1, 2 and 3 are indicated by the boxed sequences.

FIG. 2. Periplasmic extracts containing VHH of the VHH3.42 family bind the SARS-CoV-2 spike and neutralize SARS-CoV-2 and SARS-CoV1 spike VSV pseudotypes. (A) Binding of serial dilutions of PE_VHH3.117 and PE_VHH3.42 to the SARS-CoV-2 spike protein as tested by ELISA. PE_VHH50 (containing a previously isolated VHH that is related to VHH72) and PE_VHH3.96 (a VHH that did not display binding in the PE-ELISA screen) were respectively used as positive and negative control. (B) VHHs of the VHH3.42 (PE3_42=PE of VHH3-42 etc.) family neutralize VSV-AG viruses pseudotyped with SARS-CoV-2 spike. VSV-AG pseudotyped with SARS-CoV-2 spike was mixed with equal volumes of 8-, 40- or 200-fold diluted PE. After 30 minutes incubation at 37° C. these mixtures were used to infect Vero E6 cells grown at sub-confluency in 96-well plates. Sixteen hours after infection the luciferase activity was measured. PBS, VHH72 (VHH72_h1_S56A at 1 mg/ml), VHH50 (1 mg/ml) were used as controls. The graph shows the luciferase values (cps) for each PE or purified VHH at its indicated final dilution. (C) VHHs of the VHH3.42 family neutralize VSV-AG viruses pseudotyped with SARS-CoV-1 spike. VSV-AG pseudotyped with SARS-CoV-1 spike that contain a luciferase and GFP expression cassette was mixed with equal volumes of 100-, or 1000-fold diluted PE to obtain a final dilution of 1/200 (“200”) or 1/2000 (“2000”), respectively. After 30 minutes incubation at 37° C., these mixtures were used to infect Vero E6 cells grown at sub-confluency in 96-well plates. Sixteen hours after infection the luciferase activity was measured. PBS, PE_VHH3.12 (“PE3_12”; a VHH that did not display binding in the screen PE-ELISA shown in FIG. 1), VHH72 (VHH72_h1_S56A at 1 mg/ml), VHH50 (1 mg/ml) or non-infected (NI) cells were used as controls. The graph shows the luciferase values (cps) for each PE extract or purified VHH at its indicated final dilution.

FIG. 3. SDS PAGE analysis of the purified VHHs. SDS-PAGE followed by Coomassie staining of the indicated purified VHHs produced by Pichia pastoris (A) or WK6 E. coli cells (B).

FIG. 4. VHH3.42 and VHH3.117 bind the SARS-CoV-2 RBD and spike protein and the SARS-CoV-1 spike protein. Binding of purified VHH3.42 and VHH3.117 to the RBD of SARS-CoV-2 (SARS-CoV-2 RBD-muFc) (A), to the spike protein of SARS-CoV-2 (B), and to the spike protein of SARS-CoV-1 (C). VHH72 and a control VHH targeting GFP (ctrl VHH) were respectively used as positive and negative control. Binding to BSA was tested as control, and not of the tested VHHs bound to BSA (not shown).

FIG. 5. Kinetics of VHH3.117 binding to RBD. (A) Comparison of the off rates of VHH3.117 (“VHH3_117”), VHH3.42 (“VHH3_042”) and VHH72_h1_S56A (“VHH72”) as measured by BLI at a single concentration (200 nM) to monomeric human Fc-fused SARS-CoV-2_RBD-SD1 immobilized on anti-human IgG Fc capture (AHC) biosensors (ForteBio). Each graph shows one of the duplicate measurements. (B) Binding kinetics of VHH3.117 to monomeric human Fc-fused SARS-CoV-2_RBD-SD1 immobilized on anti-human IgG Fc capture (AHC) biosensors (ForteBio), in replicate, at concentrations of 100 to 3.13 nM (2-fold dilution series). (C) Binding kinetics of VHH3.89 to monomeric human Fc-fused SARS-CoV-2_RBD-SD1 immobilized on anti-human IgG Fc capture (AHC) biosensors (ForteBio), in replicate, at concentrations of 50 to 3.13 nM (2-fold dilution series).

FIG. 6. VHH3.42 and VHH3.117 do not compete with VHH72 for the binding of RBD. (A) VHH3.42 and VHH3.117 can bind to monomeric SARS-CoV-2 RBD captured by VHH72-Fc. The graph shows the average (n=2+variation) binding (OD at 450 nm) of the VHHs and an irrelevant GFP binding VHH (GBP) at 0.5 μg/mIto RBD that was captured by coated VHH72-Fc. PBS and VHH72_h1_S56A (“VHH72”) at 10 μg/ml were included as reference. (B) In this BLI competition experiment, VHH72-Fc was loaded on anti-human Fc biosensor tip and subsequently dipped into a solution containing mouse IgG2a Fc-fused SARS-CoV-2-RBD-SD1 (Sino Biological) until saturation was achieved. Next, the tips were dipped into a solution containing VHH72_h1_S56A (“VHH72”), VHH3.42 (“VHH3_42”), VHH3.117 (“VHH3_117”) or no VHH (“buffer”). VHHs that compete with VHH72 for the binding of RBD (such as VHH72 itself) displace the captured RBD-muFc from the VHH72-Fc coated tips and will hence lower the BLI signal over time. VHH3.42 and VHH3.172 bind to VHH72-Fc captured RBD, resulting in an increased BLI signal. The graph shows the BLI signal over time starting from the moment the tips were dipped in the solution containing the indicated VHHs.

FIG. 7. VHH3.42, VHH3.117 and VHH3.92 neutralize VSV-G pseudotyped with the SARS-CoV-2 spike protein. (A) Neutralization of SARS-CoV-2 pseudotyped VSV (VSV-G spike SARS-CoV-2) by purified VHH3.42 (“VHH3,42”), VHH3.117 (“VHH3,117”) and VHH3.72_h1_S56A (“VHH72”). The graph shows the GFP fluorescence intensity of triplicate dilutions series (n=3±SEM), each normalized to the lowest and highest GFP fluorescence intensity value of that dilution series. (B) Neutralization of SARS-CoV-2 pseudotyped VSV (VSV-DG spike SARS-CoV-2) by VHH3.92 and VHH3.117. The graph shows the GFP fluorescence intensity of triplicate dilutions series (n=4±SEM), each normalized to the lowest and highest GFP fluorescence intensity value of that dilution series.

FIG. 8. VHH3.42 and VHH3.117 neutralize VSV-G pseudotyped with the SARS-CoV-1 spike protein. Neutralization of SARS-CoV-1 spike pseudotyped VSV (VSV-G spike SARS-CoV-1) by VHH3.42, VHH3.117 and VHH72_h1_S56A (“VHH72”). The graphs show the mean (n=2±variation) GFP fluorescence intensity of duplicate dilutions (n=2±variation) each normalized to the lowest and highest GFP fluorescence intensity value of that dilution series.

FIG. 9. VHH3.42, VHH3.92 and VHH3.117 do not interfere with the binding of RBD to recombinant ACE2. The graph shows the AlphaLISA signal that is detected upon binding of biotinylated RBD to recombinant ACE2 in the presence of dilution series of VHH3.42, VHH3.42 and VHH3.117. A control VHH targeting an irrelevant protein was used as negative control (ctrl VHH). VHH72_h1_S56A (“VHH72”) and the related VHH3.115 that both prevent binding of RBD to ACE2 were used as positive controls.

FIG. 10. VHH3.42, VHH3.92 and VHH3.117 do not prevent binding of RBD to ACE-2. (A-C) VHH3.42, VHH3.92 and VHH3.117 do not prevent binding of RBD to Vero E6 cells. (A) RBD-Fc binding to a Vero E6 cell that endogenously expresses ACE2; flow cytometric analysis of binding of RBD (0.4 μg/ml) that was pre-incubated with VHH3.42 or VHH3.117 (each at 1 μg/ml) to Vero E6 cells. As controls Vero E6 cells not treated with RBD (noRBD) and Vero E6 cells stained with RBD-muFc that was pre-incubated with PBS or an irrelevant control GFP targeting VHH (ctrl VHH) were used. VHH72_h1_S56A was used as reference. The bars represent one single analysis per VHH. The controls, PBS and noRBD were tested in duplicate. Binding of RBD-muFc was detected by an AF647 conjugated anti-mouse IgG antibody. (B) Flow cytometric analysis of binding of RBD (0.4 ug/ml) that was pre-incubated with a dilution series of VHH3.92 or VHH3.117 to Vero E6 cells. As controls Vero E6 cells not treated with RBD (noRBD) and Vero E6 cells stained with RBD-muFc that was pre-incubated with PBS or an irrelevant control GFP targeting VHH (ctrl VHH) were used. VHH3.115 (a VHH related to VHH72) was used as reference. Binding of RBD-muFc was detected by an AF647 conjugated anti-mouse IgG antibody. The graph shows the % RBD-muFc positive Vero E6 cells (n=1). (C) VHH3.117 does not prevent binding of human ACE2 fused to a human Fc to yeast cells expressing the SARS-CoV-2 RBD at their surface. Histograms showing the binding of ACE2-Fc that was pre-incubated with VHH72 or VHH3.117 (at 10, 1, 0.1, 0.01 or 0 ug/ml). Binding of ACE2-Fc was detected using an AF594 conjugated anti-human IgG antibody.

FIG. 11. VHHs of the VHH3.42 family do not compete with CR3022, S309 and CB6 for binding to the SARS-CoV-2 RBD. (A) VHH3.177 does not compete with 5309 and CR3022 for the binding to RBD. The graphs show the binding (OD at 450 nm) of VHH72_hl_S56A (“VHH72”, top panel) or VHH3.117 (bottom panel) dilution series to RBD-SD1 fused to monovalent human Fc (RBD-SD1-monoFc) that was either directly coated on an ELISA plate or captured by coated 5309 and CR3022. RBD that was captured by palivizumab, an antibody directed against the RSV F protein was used as negative control. (B) VHH3.92 does not compete with CB6, 5309 and CR3022 for the binding to RBD. The graphs show the binding (OD at 450 nm) of VHH3.92 dilution series to RBD-SD1 fused to monovalent human Fc (RBD-SD1-monoFc) that was either directly coated on an ELISA plate or captured by coated CB6, VHH72-Fc 5309 and CR3022. RBD that was captured by coated palivizumab, an antibody directed against the RSV F protein, and by coated VHH3.117 were used as controls.

FIG. 12. VHHs of the VHH3.42 family bind an epitope that is distant from that of CR3022, S309 and CB6 and is conserved between SARS-CoV-2 and -1. (A) The three panels show the surface representation of the SARS-CoV-2 RBD alone (left), or complexed with CB6, CR3022 and 5309 (middle), or complexed with VHH72 (right). (B) Further shown is a surface representation of the SARS-CoV-2 RBD alone rotated along its long axis together with the same rotations of the SARS-CoV-2 RBD complexed with CB6, CR3022 and 5309. The SARS-CoV-2 RBD amino acids that are identical in SARS-CoV-1 are shown in light grey and the ones that are different in SARS-CoV-1 are shown in dark grey. The arrows indicate a site that is not occluded, neither by the shown antibodies, nor by ACE2 (not shown) and is conserved between SARS-CoV-1 and SARS-CoV-2. This site is presumed to harbor the binding sited of the VHHs identified herein.

FIG. 13. VHH3.42, VHH3.92 and VHH3.117 recognize the RBD of a diverse range of Sarbecoviruses. (A) Cladogram (UPGMA method) based on the RBD of SARS-CoV-1-related (clade 1a), SARS-CoV-2-related (cladelb) and clade 2 and clade 3 Bat SARS-related Sarbecoviruses. (B) Flowcytometric analysis of the binding of VHHs to Saccharomyces cerevisiae cells that display the RBD of the indicated Sarbecoviruses. The graphs show for the tested RBD variants the ratio of the MFI of AF647 conjugated anti-mouse IgG antibody used to detect VHHs bound to the cells that express RBD (FITC conjugated anti-myc tag antibody positive) over that of cells that do not express RBD (FITC conjugated anti-myc tag antibody negative). A VHH targeting GFP (GBP) was used as a negative control antibody and VHH72_h1_S56A was used as reference. All VHHs were tested at 10 ug/ml.

FIG. 14. VHH3.117 recognizes the RBD of a diverse range of clade 1, 2 and 3 Sarbecoviruses. (A) Flowcytometric analysis of the binding of VHH3.117 to the indicated RBDs at 100 (left bar per datapoint on the X-axis), 1 (middle bar per datapoint on the X-axis) and 0.01 ug/ml (right bar per datapoint on the X-axis). (B) PBS was used as negative control and VHH72_h1_S56A (“VHH72”) was used as reference. The graphs show for the indicated RBD variants the ratio of the MFI of AF647 conjugated anti-mouse IgG antibody used to detect VHHs bound the Saccharomyces cerevisiae cells that express RBD (FITC conjugated anti-myc tag antibody positive) over that of cells that do not express RBD (FITC conjugated anti-myc tag antibody negative).

FIG. 15. Outlining of the VHH3.117 epitope identified by deep mutational scanning. (A) Indication of the RBD amino acid positions for which changes can significantly affect the binding of VHH72_h1_S56A (“VHH72 escape”) and VHH3.117 (“VHH3.117 escape”) as identified by deep mutational scanning using 2 independent libraries. The SARS-CoV-2 RBD amino acid sequence is shown in the upper and lower line. In the upper line the amino acids positions at which mutations result in escape from VHH72_h1_S56A are underlined and in bold. In the lower line the amino acids positions at which mutations result in escape from VHH3.117 are underlined and in bold. (B) Top left panel: Surface representation of the SARS-CoV-2 RBD (light grey) with the amino acid positions for which a change, as identified by deep mutational scanning, is associated with reduced VHH3.117 binding are indicated in dark grey. Top right panel: cartoon representation of the SARS-CoV-2 RBD (light grey). The amino acid positions for which certain substitutions are associated with reduced VHH3.117 binding and that are surface exposed are indicated in dark red and shown as sticks in the cartoon representation. Bottom left and right panels: amino acid positions at which substitutions that are associated with escape from VHH3.117 binding but are not exposed to the surface are indicated. The bottom left cartoon shows the C336-C361 and C391-0525 disulfide bonds. The bottom right panel illustrates that the aromatic side chains of Y365 and F392 are oriented inwards into the RBD core. (C) indication of the RBD amino acid positions for which changes can significantly affect the binding of VHH3.117 as identified by deep mutational scanning and represented in a surface representation rotated along its long or short axis as indicated.

FIG. 16. The location of the identified VHH3.117 epitope is in line with the ability of VHH3.117 to bind RBD that is bound by S309, CR3022 and CB6 and with its ability to cross-neutralize SARS-CoV-2 and SARS-CoV-1 viruses. (A) Left panel: surface representation of the SARS-CoV-2 RBD (light grey) in complex with 5309 and CR3022 Fabs (dark grey). Residues that are part of the VHH3.117 binding site are indicated in black in the RBD. Right panel: surface representation of the SARS-CoV-2 RBD with the amino acids that are identical in SARS-CoV-2 and SARS-CoV-1 colored in black, indicating that the binding site of VHH3.117 is conserved between SARS-CoV-2 and SARS-CoV-1. (B) The VHH3.117 binding site is conserved among clade 1, 2 and 3 Sarbecoviruses. Shown is alignment of the amino acid sequences of the RBDs of the Sarbecoviruses that were tested for VHH3.117 binding. The amino acid positions at which substitutions are associated with escape from VHH3.117 binding and that are surface exposed are indicated in bold. The amino acid positions at which substitutions that are associated with escape from VHH3.117 binding but are not surface exposed near the VHH3.117 binding site are underlined and in bold. For each tested Sarbecovirus RBD, the amino acids that are within the VHH3.117 binding site but are not identical to the amino acid at the respective position in the SARS-CoV-2 spike protein are indicated in bold. The numbers on top of the alignment indicate the positions of the amino acids in the SARS-CoV-2 spike protein. (C) The VHH3.117 binding site is highly conserved among the SARS-CoV-2 RBD sequences in the GISAID database. Surface representations of the SARS-CoV-2 RBD (white) showing conservation. The white to black gradient represents the most to the least conserved positions. Amino acids that are substituted in emerging variants of concern (K417, L452, E484 and N501) or in variants of interest (S477), as well as in N439 are pointed out by arrows. The amino acid sequence of SARS-CoV-2 RBD (spike protein amino acid positions 333-516 of Wuhan-Hu-1 isolate) is shown with all missense mutations, detected at least once in 440,769 SARS-CoV-2 genomes analyzed (available in GISAID on Feb. 12, 2021), depicted above each residue. Variants are ordered vertically at each position, according to frequency represented by the number of observed cases. Amino acids that are substituted in emerging variants of concern (K417, L452, E484 and N501) or in variants of interest (S477) are indicated by asterisk. The N439 position that is frequently substituted is also indicated. The amino acids for which substitutions were associated with loss of binding of VHH3.117 as determined by deep mutational scanning are indicated in boxes. (D) The VHH3.117 epitope is not accessible on intact spike proteins. The VHH3.117 binding site is not accessible on the RBD in down- or in up-conformation. Shown is the SARS-CoV-2 spike trimer (PDB: 6VSB, white) with 1 RBD in up-conformation and 2 RBDs in down conformation. The VHH3.117 binding region is marked in dark grey and indicated with one arrow that points to the RBD in the up position and another arrow that points to one of the RBDs in the down position. Inset: the VHH3.117 binding site on the RBD in up conformation is partially occluded by an NTD of an adjacent spike protomer.

FIG. 17. Surface representation of the SARS-CoV-2 RBD with indication of bound antibodies CB6 and mAb52. The VHH3.117 binding region in the RBD is indicated in light grey and by an arrow.

FIG. 18. Surface representation of the SARS-CoV-2 RBD with indication of the epitopes of nanobodies nb34 and nb95 (Xiang et al. 2020, Science 370:1479-1484; Sun et al. 2021, BioRxiv https://doi.org/10.1101/2021.03.09.434592), as well as of VHH3.117. The epitope regions are marked by asterisks.

FIG. 19. Dose-dependent inhibition of VHH72 binding to SARS-CoV-2 RBD by VHHs from different families.

Competition Alphascreen with avi-tagged biotinylated SARS-CoV-2 RBD (0.5 nM final) and Flag-tagged VHH72 h1 S56A (0.6 nM). VHHs belonging to the same (super) family are indicated in boxes.

FIG. 20. Dose-dependent inhibition of ACE-2 binding to SARS-CoV-2 RBD by VHHs from different families.

Competition Alphascreen with avi-tagged biotinylated SARS-CoV-2 RBD (1 nM final) and human ACE-2-mFc (0.2 nM). VHHs belonging to the same (super) family are indicated in boxes.

FIG. 21. VHH3.89 does not compete with VHH72, S309 or CB6 but does compete with VHH3.117 for binding to the SARS-CoV-2 RBD. (A) Binding of VHH3.89 to RBD pre-bound by well-characterized antibodies. The graphs show the average binding (OD at 450 nm) and variation (n=2) of dilution series of VHH3.92 that is related to VHH3.117 (left panel) or VHH3.89 (right panel) to RBD-SD1 fused to monovalent human Fc (RBD-SD1-monoFc) that was either directly coated on an ELISA plate or captured by coated 5309, CB6, D72-53 and VHH3.117 (without HA-tag). RBD that was captured by palivizumab (Synagis), an antibody directed against the RSV F protein was used as negative control. Binding of HA-tagged VHH3.92 and VHH3.89 was detected by an anti-HA tag antibody. (B) Surface representation of the SARS-CoV-2 RBD captured by 5309, CB6 and VHH72 shown as meshes. The black and white coloring of the RBD surface respectively indicate amino acids that are different or identical between SARS-CoV-1 and 2. (C) VHH3.117 binds to a concave site at the side of the RBD. The black coloring on the RBD surface representation indicates the amino acid positions at which substitutions are associated with reduced binding of VHH3.117 as determined by deep mutational scanning based on yeast surface display of RBD mutants.

FIG. 22. VHH3.89 does not prevent binding of RBD to ACE-2. Flow cytometric analysis of binding of RBD-muFc (0.4 ug/ml) that was pre-incubated with a dilution series of VHH3.89 or VHH3.117 to Vero E6 cells. Vero E6 cells not treated with RBD (noRBD) and Vero E6 cells stained with RBD-muFc that was pre-incubated with PBS or an irrelevant control GFP targeting VHH (ctrl VHH) were used as controls. VHH3.115, an VHH related to VHH72 and known to block the binding of RBD to ACE2, was used as control. Binding of RBD-muFc was detected by an AF647 conjugated anti-mouse IgG antibody. The graph shows the binding (n=1) of RBD-muFc (MFI of AF647) to Vero E6 cells.

FIG. 23. VHH3.89 neutralizes VSV-AG pseudotyped with the SARS-CoV-2 or SARS-CoV-1 spikes. (A) VHH3.89, neutralizes VSV-delG pseudotyped with the SARS-CoV-2 spikes. Neutralization of SARS-CoV-2 pseudotyped VSV (VSV-AG spike SARS-CoV-2) by purified VHH3.89, VHH3.117 and VHH3.92 and VHH3.83. The graph shows the GFP fluorescence intensity of quadruplicate dilutions series (n=4±SEM), each normalized to a non-infected and infected PBS treated sample included in each dilution series. The GFP binding VHH, GBP, was used as negative control (B) VHH3.89 neutralizes VSV-delG pseudotyped with the SARS-CoV-1 spike protein. Neutralization of SARS-CoV-1 pseudotyped VSV (VSV-ΔG spike SARS-CoV-2) by crude E. coli periplasmic extracts containing VHH3.89, VHH3.117, VHH3.92 or VHH3.83. The graph shows the GFP fluorescence intensity normalized to a non-infected sample and infected PBS treated sample. A periplasmic extract that did not contain an SARS-CoV-2 spike protein binding VHH (PE control) was used as negative control.

FIG. 24. VHH3.89 recognizes the RBD of a diverse range of sarbecoviruses. (A) Cladogram (UPGMA method) based on the RBD of SARS-CoV-1-related (clade 1a), SARS-CoV-2-related (cladelb) and clade 2 and clade 3 Bat SARS-related Sarbecoviruses. The arrows indicate the viruses of which the RBD was included in the binding analysis (B) Surface representation of the SARS-CoV-2 RBD displaying the degree of amino acid conservation among the tested sarbecoviruses as colored from red (most conserved) to blue (least conserved). Conservation analysis and visualization was done by Scop3D (Vermeire et al, 2015 Proteomics, 15(8):1448-52) and PyMol (DeLano, 2002). (C) Flow cytometric analysis of the binding of dilution series of VHH3.117 and VHH3.89 to Saccharomyces cerevisiae cells that display the RBD of the indicated Sarbecoviruses at their surface. The graphs show for the tested RBD variants the ratio of the MFI of AF647 conjugated anti-mouse IgG antibody used to detect VHHs bound to the cells that express RBD (FITC conjugated anti-myc tag antibody positive) over that of cells that do not express RBD (FITC conjugated anti-myc tag antibody negative). (D) VHH3.89 efficiently binds to the RBD of all clade 1 and 2 sarbecoviruses in a yeast cell ELISA. The graphs show the binding (OD at 450 nm) of dilution series of VHH3.89 and VHH3.117 to coated yeast cells expressing the RBD of the indicated sarbecoviruses at their surface.

FIG. 25. Humanization variants of VHH3.117 (A) and VHH3.89 (B). CDRs are indicated according to AbM annotation, and sequential numbering of the amino acid sequence is provided. In A, the X is any amino acid, preferably each independently Leu, Ile, Ala, or Val.

FIG. 26. Monovalent VHH3.117 and VHH3.89 potently neutralize various SARS-CoV-2 variants. Dilution series of the indicated antibodies or monovalent VHHs were incubated with VSVdeIG viral particles pseudotyped with the spike protein containing the RBD mutations of the original Wuhan (WT) (A), alpha (B), alpha+E484K (C), beta (D), beta+P348L (E), kappa (F), delta (G) and epsilon (H) SARS-CoV-2 variants and subsequently allowed to infect Vero E6 cells. The graph shows the GFP fluorescence intensity of dilutions series (N=3±SD for VHH3.117 and N=1 for VHH3.89, 5309, CB6 and palivizumab), each normalized to the highest GFP fluorescence intensity value of that dilution series and that of infected mock treated cells.

FIG. 27. VHH3.117-Fc and VHH3.89-Fc recognize the RBD of clade 1, clade 2 and clade 3 sarbecoviruses. The graphs show the binding (OD at 450 nm) of dilution series of VHH3.117-Fc (A), VHH3.89-Fc (B) and palivizumab (C) to coated yeast cells expressing the RBD of the indicated sarbecoviruses at their surface. The top panels show the binding to yeast cells displaying the RBD of clade 1 sarbecoviruses whereas the bottom panels show the binding of yeast cells displaying the RBD of the indicated clade 2 sarbecoviruses and the BM48-31 clade 3 sarbecovirus. Yeast cells not expressing any RBD (empty) were used as negative controls. For each VHH-Fc and palivizumab the binding curves of these yeast cells are shown in both the left and right panel as reference.

FIG. 28. VHH3.117-Fc binds to recombinant stabilized Spike proteins of SARS-CoV-2 WT and the omicron variant. ELISA analysis of the binding of palivizumab, 5309 and VHH3.117 to recombinant HexaPro stabilized spike protein (Spike-6P) of the Wuhan SARS-CoV-2 virus (A), recombinant HexaPro stabilized spike protein (Spike-6P) of the Wuhan SARS-CoV-2 BA.1 omicron variant (B) and BSA (C). The graphs show the OD at 450 for the indicated antibodies (N=2+SD for VHH3.117-Fc and N=1 for palivizumab and S309).

FIG. 29. Binding kinetics of VHH-Fc constructs to RBD and Spike protein of SARS CoV-2 WT and the omicron variant as measured by BLI. (A) Binding kinetics of VHH3.117-Fc to monovalent SARS-CoV-2_RBD-His immobilized on anti-human IgG Fc capture (AHC) biosensors (ForteBio) at concentrations of 100 to 6.25 nM (2-fold dilution series). Full grey lines represent double reference-subtracted data and dashed lines the fit to a global 1:1 binding model. (B) Binding kinetics of VHH72-S56A-Fc to monovalent SARS-CoV-2 BA.1/Omicron_RBD-His immobilized on anti-human IgG Fc capture (AHC) biosensors (FortéBio) at concentrations of 100 to 6.25 nM (2-fold dilution series). Full grey lines represent double reference-subtracted data and dashed lines the fit to a global 1:1 binding model. A representative experiment of three distinct BLI analyses is shown. Kinetics parameters are averages of triplicate experiments. (C) Binding kinetics of VHH3.89-Fc to monovalent SARS-CoV-2 BA.1/Omicron_RBD-His immobilized on anti-human IgG Fc capture (AHC) biosensors (FortéBio) at concentrations of 100 to 6.25 nM (2-fold dilution series). Full grey lines represent double reference-subtracted data and dashed lines the fit to a global 1:1 binding model. A representative experiment of three distinct BLI analyses is shown. Kinetics parameters are averages of triplicate experiments. (D) Binding kinetics of VHH3.117-Fc to monovalent SARS-CoV-2 BA.1/Omicron_RBD-His immobilized on anti-human IgG Fc capture (AHC) biosensors (FortéBio) at concentrations of 100 to 6.25 nM (2-fold dilution series). Full grey lines represent double reference-subtracted data and dashed lines the fit to a global 1:1 binding model. A representative experiment of three distinct BLI analyses is shown. Kinetics parameters are averages of triplicate experiments. (E) Binding kinetics of VHH3.89-Fc and VHH3.117-Fc to SARS-CoV-2 WT Spike-6P immobilized on anti-human IgG Fc capture (AHC) biosensors (FortéBio) at a single concentration (200 nM). A representative experiment of three distinct duplicate BLI analyses is shown. A binding model could not be fit for the 2:3 (bivalent VHH-Fc immobilized, trimeric analyte) interactions. (F) Binding kinetics of VHH3.89-Fc and VHH3.117-Fc to monovalent SARS-CoV-2 BA.1/Omicron Spike-6P immobilized on anti-human IgG Fc capture (AHC) biosensors (FortéBio) at a single concentration (200 nM). A representative experiment of three distinct Duplicate BLI analyses is shown. A binding model could not be fit for the 2:3 (bivalent VHH-Fc immobilized, trimeric analyte) interactions. The difference in signal observed for WT Spike-6P (E) and Omicron Spike-6P is likely caused by variation in methods used for spike-concentration (WT produced/quantified in-house, Omicron by Acro Biosystems).

FIG. 30. VHH3.117-Fc and VHH3.92-Fc neutralize VSV virus pseudotyped with the SARS-CoV-2 spike protein. Dilution series of VHH3.117-Fc and VHH3.92-Fc were incubated with VSVdeIG viral particles pseudotyped with the SARS-CoV-2 spike protein and subsequently allowed to infect Vero E6 cells. The graph shows the mean GFP fluorescence intensity of VHH-Fc dilutions series (N=3±SD) each normalized to the GFP fluorescence intensity value of non-infected and infected untreated control cells that were included in each dilution series.

FIG. 31. VHH3.117-Fc neutralizes the SARS-CoV-2 delta and gamma variants. (A) VHH3.117-Fc and VHH3.92-Fc neutralize VSVdeIG virus particles pseudotyped with the spike protein of WT SARS-CoV-2 (upper panel) or the with a spike protein containing the RBD mutations present in the delta variant (lower panel). The graphs show the mean GFP fluorescence intensity of VHH-Fc dilutions series (N=3±SEM) each normalized to the GFP fluorescence intensity value of non-infected and infected untreated control cells that were included in each dilution series. (B) VHH3.117-Fc neutralizes VSVdeIG virus particles pseudotyped with the spike protein of WT SARS-CoV-2 (upper panel) or the with a spike protein containing the RBD mutations present in the gamma variant (lower panel). The graphs show the mean GFP fluorescence intensity of VHH-Fc dilutions series (N=2±SD for VHH3.117-Fc and CB6 and N=1 for palivizumab) each normalized to the GFP fluorescence intensity value of non-infected control cells that were included in each dilution series and that of the cells treated with the lowest concentration.

FIG. 32. VHH3.117-Fc can neutralize the SARS-CoV-2 omicron BA.1 variant. Dilution series of VHH3.117-Fc, 5309 and palivizumab were incubated with VSVdeIG viral particles pseudotyped with the SARS-CoV-2 614G spike protein variant (A) or with the SARS-CoV-2 omicron BA.1 variant spike protein (B) and subsequently allowed to infect Vero E6 cells. The graph shows the mean GFP fluorescence intensity of VHH-Fc dilutions series (N=2±SD) each normalized to the GFP fluorescence intensity value of non-infected and infected untreated control cells that were included in each dilution series.

FIG. 33. VHH3.117-Fc can neutralize SARS-CoV-1. Dilution series of VHH3.117-Fc and 5309 were incubated with VSVdeIG viral particles pseudotyped with the SARS-CoV-2 spike protein (A) or with the SARS-CoV-1 spike protein (B) and subsequently allowed to infect Vero E6 cells. The graph shows the mean GFP fluorescence intensity of VHH-Fc dilutions series (N=2±SD for VSVdeIG-Spike SARS-CoV-2 and N=3±SD for VSVdeIG-Spike SARS-CoV-1) each normalized to the GFP fluorescence intensity value of non-infected and infected untreated control cells that were included in each dilution series.

FIG. 34. VHH3.117-Fc neutralizes VSVdeIG virus particles pseudotyped with SARS-CoV-2 spike on Vero E6 cells that stably express human TMPRSS2. Dilution series of VHH3.117-Fc were incubated with VSVdeIG viral particles pseudotyped with the SARS-CoV-2 spike protein and subsequently allowed to infect Vero E6 cells or Vero E6 TMPRSS2 cells. The graphs show the mean GFP fluorescence intensity of VHH-Fc dilutions series (N=3±SEM) each normalized to the GFP fluorescence intensity value of non-infected and infected untreated control cells that were included in each dilution series.

FIG. 35. VHH3.117-Fc is able to neutralize replication-competent VSV virus containing the SARS-CoV-2 Spike protein. Dilution series of VHH3.117, VHH3.89 or VHH3.117-Fc were incubated with replication-competent VSV S1-1a WT VSV virus described by Koenig et al. (Koenig et al. (2021) Science 371:eabe6230) and allowed to infect Vero E6 for two days. The graphs show the mean GFP fluorescence intensity of VHH-Fc dilutions series (N=3±SEM for VHH3.117 and VHH3.89 and N=2±SD for VHH3.117-Fc) each normalized to the GFP fluorescence intensity value of non-infected and infected untreated control cells that were included in each dilution series.

FIG. 36. VHH3.117 and VHH3.89-Fc induce premature shedding of the spike S1 subunit. (A) VH H72-Fc and VHH3.117 induce 51 shedding from cells expressing the SARS-CoV-2 spike protein. (B) VHH3.89-Fc induces 51 shedding from cells expressing the SARS-CoV-2 spike protein. Anti-S1 Western blot analysis is shown of the growth medium and cell lysates of Raji cells expressing the SARS-CoV-2 spike protein (Raji Spike) or not (Raji) incubated for 30 minutes with the indicated VHH constructs or antibodies. The lower an upper triangle at the right side of the blots indicate respectively the 51 spike subunit generated after furin mediated cleavage of the spike protein and cellular uncleaved spike proteins.

FIG. 37. Identification of the VHH3.89 family member VHH3.183 that can neutralize SARS-CoV-2 via binding to the RBD of the SARS-CoV-2 spike protein. (A) The VHHs present in periplasmic extracts (PE) of E coli cells expressing VHH3.89 (PE_89) and VHH3.183 (PE_183) bind the SARS-CoV-2 spike protein and RBD. The graph shows the binding (OD at 450 nm) of PE_12, PE_89 and PE_183 to BSA, RBD and spike protein as tested by ELISA. (B) The VHHs present in periplasmic extracts of E coli cells expressing VHH3.89 (PE_89) and VHH3.183 (PE_183) are able to neutralize VSVdeIG-spike pseudovirus. The graph shows the luciferase signal of cell infected with luciferase-GFP expressing VSVdeIG-spike pseudovirus that was pre-incubated with 16, 80 and 400-fold diluted PE_12, PE_89 and PE_183. (C) Alignment of the VHH3.89 and VHH3.183 amino acids sequences (D) SDS-PAGE followed by coomassie staining of the indicated purified VHHs produced WK6 E. coli cells. (E) Purified VHH3.183 can neutralize VSVdeIG virus particles pseudotyped with SARS-CoV-2 spikes. Dilution series of VHH3.183 and VHH3.89 were incubated with VSVdeIG viral particles pseudotyped with the SARS-CoV-2 spike protein and subsequently allowed to infect Vero E6 cells. The graph shows the GFP fluorescence intensity of VHH dilutions series each normalized to the GFP fluorescence intensity value of non-infected and infected untreated control cells that were included in each dilution series. (F) Off-rates of monovalent VHH3.89 and VHH3.183 as measured by BLI at a single concentration (200 nM) binding to monomeric human Fc-fused SARS-CoV-2_RBD-SD1 immobilized on anti-human IgG Fc capture (AHC) biosensors (FortéBio). Full black (VHH3.89) and grey (VHH3.183) lines represent double reference-subtracted data and dashed lines the fit of triplicate data to a global 1:1 binding model.

FIG. 38. Determination of SARS-CoV-2 RBD amino acid positions that can lose binding to VHH3.117 and VHH3.89 when mutated, by deep mutational scanning. Deep mutational scanning signal (expressed as % escape) obtained with VHH3.117 (A) or VHH3.89 (B) plotted over the entire length of the SARS-CoV-2 RBD (amino acid positions indicated on the ‘site’ axis). (C-D) The amino acid sequence of SARS-CoV-2 RBD (spike protein amino acid positions 336-525 of Wuhan-Hu-1 isolate) is shown and the amino acids for which substitutions were associated with loss of binding of VHH3.117 (C) or VHH3.83 (D) as determined by deep mutational scanning are indicated in boxes.

FIG. 39. Binding mode of VHH3.89 and VHH3.117 to the RBD of the SARS-CoV-2 (SC2) spike protein. Left, middle and right column show the SC2 RBD (left column), and its complexes with VHH3.89 (middle column) or VHH3.117 (right column), shown in frontal (upper row), and a 90 degree rotated view to the right (middle row) or left (lower row). Complexes of the SARS-CoV-2 spike protein in complex with the VHH were determined by cryoEM (see FIG. 40), and are here shown as solvent accessible surface, colored light gray (SC2 RBD), dark gray (VHH3.89) or middle gray (VHH3.117). On the SC2 RBD surface, the residues identified as escape mutations for VHH3.89 and/or VHH3.117 binding as identified by deep mutational scanning (FIG. 38) are shown in stick representation, labelled and highlighted in dark gray; residues proposed by the cryo-EM experiment as forming a minimal common core (or ‘epitope core’; comprising residues R355, N394, Y396, Y464, 5514 and E516) for the binding of VHH3.89 and VHH3.117 family member binders are shown in stick representation, colored black, labeled and highlighted by a box. The epitope core forms a continuous surface area encompassing approximately 300 Å 2.

FIG. 40. Cryo-EM reconstructions of VHH3.89 and VHH3.117 bound to the SARS-CoV-2 spike protein. Electron potential maps of the SARS-CoV-2 spike protein (SC2) in complex with VHH3.117 (upper; 3 Å resolution) or VHH3.89 (lower; 3.1 Å resolution), shown in side (left) and top (middle) view. Shown to the right are the refined cryo-EM structures of the SC2— VHH complexes shown in surface representation and with the receptor binding domain and N-terminal domain of the three SC2 protomers labelled RBD1-3 and NTD1-3. In the SC2-VHH3.117 complex the RBD domain in each of the protomers is in conformationally similar up position and bound by a single VHH3.117 each. In the SC2— VHH3.89 complex all three RBD domains are in up position but in different angles relative to the SC2 core. Two VHH3.89 copies are bound, one to the RBD of SC2 protomer 1 (labelled RBD-1), and a second to the RBD of SC2 protomer 2 (RBD-2). RBD-3 is poorly defined in the cryo-EM maps, indicative of a large conformational flexibility. Based on this experiment, VHH3.117 and VHH3.89 are proposed to bind a largely common epitope comprising residues R355, N394, Y396, Y464, 5514 and E516, and which are shielded in the RBD down conformation of the apo SC2 protein.

FIG. 41. VHH3.89 and VHH3.117 target a largely overlapping epitope on the SARS-CoV-2 spike protein. Structure of the SARS-CoV-2 RBD (residues 330-530) shown as solvent accessible surface, and as frontal view relative to the VHH3.89 and VHH3.117 epitopes. On the SC2 RBD surface, the residues identified as escape mutations for VHH3.89 and/or VHH3.117 binding by deep mutational scanning (FIG. 38) are shown in stick representation, labelled and highlighted in dark gray; residues here proposed by the cryo-EM experiment as forming a minimal common core (or ‘epitope core’; comprising residues R355, N394, Y396, Y464, 5514 and E516) for the binding of VHH3.89 and VHH3.117 family member binders are shown in stick representation, colored black, labeled and highlighted by a box. The epitope core forms a continuous surface area encompassing approximately 300 Å ². Binding of VHH3.89 to the epitope core of SC2 RBD results in the burying of approximately 290 Å ² surface with a calculated Gibbs free energy of −2.3 kcal/mol (as determined by PDBePISA).

FIG. 42. VHH3.117 and VHH3.89 amino acid sequence and illustration of the different CDR annotations as used herein. CDR annotations according to MacCallum, AbM, Chothia, Kabat and IMGT in grey labeled boxes corresponding to the sequences of VHH3.117 and VHH3.89.

FIG. 43. Detailed view of the binding interface between VHH3.89 and SARS-CoV-2 RBD, as observed in the cryoEM structure provided in FIG. 39. Core epitope residues of the VHH3.89 are indicated in thick stick representation, and are labelled accordingly and pointed at through arrows. The residues of VHH3.89 that make the contacts with these core epitope residues are also labelled accordingly and pointed at through arrows. Measurements of the distance between VHH3.89 amino acid side chain atoms and SARS-CoV-2 RBD amino acid side chain atoms were done in PyMOL, and the measured contacts are indicated with dotted lines, and the measured distance is indicated, in Angstrom. All of these contacts are below 4 Angstrom. Views are provided of the interface from two different angles, in order to better visualize the set of measurements.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim.

The work leading to the present invention identified binding agents which specifically interact with an epitope on the Receptor binding domain (RBD) present in the spike protein of the sarbecoviruses such as the SARS-CoV-1 virus and the SARS-Cov-2 Corona virus. Binding between the agent and the spike protein results in a neutralization of the infection capacity of the sarbecovirus without inhibiting binding of the RBD with ACE-2. The binding agents as described herein induce 51 shedding and consequently premature spike triggering and, without wishing to be bound by any theory, may as such not allowing the sarbecovirus to complete the infection or entry process into the host cell. In characterizing the epitope, it was found that the current binding agents interact with RBD amino acids that are very conserved within the RBD of sarbecoviruses of multiple clades which indicates that the epitope is stable and not subject of frequent mutational changes. Such sarbecovirus-neutralizing agents are in view of the multiple emerging SARS-CoV-2 variants, some of these being more infectious and/or causing more severe disease symptoms (including in younger people) and/or escaping some of the existing vaccines and/or diagnostic tests, necessary tools to be added to the overall still limited number of SARS-CoV-2 treatment options currently available. The binding agents identified herein as well as their applications are described in more detail hereinafter. But at first, some more background on sarbecoviruses is provided.

Sarbecoviruses/Coronaviridae

The Coronaviridae family has its name from the large spike protein molecules that are present on the virus surface and give the virions a crown-like shape. The Coronoviridae family comprises four genera: Alphacoronavirus, Betacoronavirus, Gammacoronavirus, and Deltacoronavirus. Coronaviruses represent a diverse family of large enveloped positive-stranded RNA viruses that infect a wide range of animals, a wide variety of vertebrate species, and humans. The spike (S) proteins of coronaviruses are essential for host receptor-binding and subsequent fusion of the viral and host cell membrane, effectively resulting in the release of the viral nucleocapsids in the host cell cytoplasm (Letko et al. 2020, Nat Microbiol 5:562-569). Four coronaviruses, presumably from a zoonotic origin, are endemic in humans: HCoV-NL63 and HCoV-229E (α-coronaviruses) and HCoV-0C43 and HCoV-HKU1 ((3-coronaviruses). In addition, 3 episodes of severe respiratory disease caused by (3-coronaviruses have occurred since 2000. In 2002, severe acute respiratory syndrome virus (SARS), caused by SARS-CoV-1, emerged from a zoonotic origin (bats via civet cats as an intermediate species) and disappeared in 2004 (Drosten et al. 2003, N Engl J Med 348:1967-1976). Over 8000 SARS cases were reported with a mortality rate of approximately 10%. In 2012, Middle East respiratory syndrome (MERS) emerged in the Arabian Peninsula. MERS is caused by MERS-CoV, has been confirmed in over 2500 cases and has a case fatality rate of 34% (de Groot et al. 2013, N Engl J Virol 87:7790-7792). Starting at the end of 2019, the third zoonotic human coronavirus emerged with cases of severe acquired pneumonia were reported in the city of Wuhan (China) being caused by a new (3-coronavirus, now known as SARS-CoV-2, given its genetic relationship with SARS-CoV-1 (Chen et al. 2020, Lancet doi:10.1016/50140-6736(20)30211-7). Similar to severe acute respiratory syndrome coronavirus (SARS-CoV) and Middle East respiratory syndrome coronavirus (MERS-CoV) infections, patients exhibited symptoms of viral pneumonia including fever, difficult breathing, and bilateral lung infiltration in the most severe cases (Gralinski et al. 2020, Viruses 12:135). Severe acute respiratory syndrome Coronavirus 2 (SARS-CoV-2) is the causative agent of COVID-19 (Zhu et al. 2020, N Engl J Med 382:727-733). SARS-CoV-2 infections can be asymptomatic or present with mild to moderately severe symptoms. However, in approximately 10% of patients, COVID-19 progresses to a more severe stage that is characterized by dyspnoea and hypoxemia, which may progress further to acute respiratory distress requiring often long-term intensive care and causing death in a proportion of patients. “Long-COVID” furthermore refers to long-term effects of COVID-19 infection, even when no SARS-CoV-2 virus can be detected anymore. Most likely, the ongoing inflammation triggered by the innate recognition of the SARS-CoV-2 virus, and possibly also by immune complexes with antibodies from an ineffective immune response (Shrock et al. 2020, Science 370(6520): eabd4250), contributes to severe disease progression.

The first available genome sequence placed the novel human pathogen SARS-CoV-2 in the Sarbecovirus subgenus of Coronaviridae, the same subgenus as the SARS virus. Although SARS-CoV-2 belongs to the same genus Betacoronavirus as SARS-CoV (lineage B) and MERS-CoV (lineage C), genomic analysis revealed greater similarity between SARS-CoV-2 and SARS-CoV, supporting its classification as a member of lineage B (from the International Committee on Taxonomy of Viruses). Among other betacoronaviruses, this virus is characterized by a unique combination of polybasic cleavage sites, a distinctive feature known to increase pathogenicity and transmissibility. A bat sarbecovirus, Bat CoV RaTG13, sampled from a Rhinolophus affinis horseshoe bat was reported to cluster with SARS-CoV-2 in almost all genomic regions with approximately 96% genome sequence identity (and over 93% similarity in the receptor binding domain (RBD) of the Spike protein); another mammalian species may have acted as intermediate host. One of the suspected intermediate hosts, the Malayan pangolin, harbours coronaviruses showing high similarity to SARS-CoV-2 in the receptor-binding domain, which contains mutations believed to promote binding to the angiotensin-converting enzyme 2 (ACE2) receptor and demonstrates a 97% amino acid sequence similarity. SARS-CoV-1 and -2 both use angiotensin converting enzyme 2 (ACE2) as a receptor on human cells. SARS-CoV-2 binds ACE2 with a higher affinity than SARS-CoV-1 (Wrapp et al. 2020, Science 367, 1260-1263). SARS-CoV-2 differentiates from SARS-CoV and several SARS-related coronaviruses (SARSr-CoVs) as outlined in e.g. Abdelrahman et al. 2020 (Front Immunol 11: 552909).

Vaccines and passive antibody immunotherapy are being developed for prophylactic prevention and therapeutic intervention, respectively, in tackling the COVID-19 pandemic. The application of passive antibody immunotherapy with neutralizing molecules, to prevent or suppress viral replication in the lower airways, as therapeutic intervention in COVID-19 patients seems supported by patient data. Indeed, the early development of sufficient titers of neutralizing antibodies by the patient correlates with avoidance of progression to severe disease (Lucas et al. 2020, medRxiv doi:10.1101/2020.12.18.20248331), and early administration of recombinant neutralizing antibodies or those present in high-titer convalescent plasma can avert severe disease (Weinreich et al. 2020, N Engl J Med doi:10.1056/NEJMoa2035002; Chen et al. 2020, N Engl J Med doi:10.1056/NEJMoa2029849; Libster et al. 2021, N Engl J Med doi:10.1056/NEJMoa2033700). In relation to passive immunotherapy, classical antibodies usually comprise an IgG Fc moiety which has the advantage of long half-life imparted by the FcRn-mediated recycling into circulation of such antibodies (Pyzik et al. 2019, Front Immunol 10:1540). It is currently not clear of such classical antibodies would exacerbate inflammatory disease in COVID-19. It may, however, be prudent to engineer out effector functions from the antibody Fc domain, e.g. by introducing IgG Fc-LALA mutations or LALAPG mutations (Wines et al. 2000, J Immunol 164:5313-5318; Schlothauer et al. 2016, Protein Eng Des Sel 29:457-466).

Syrian hamsters (Mesocricetus auratus) have been proposed as a small animal model to study SARS-CoV-induced pathogenicity and the involvement of the immune response in aggravating lung disease. Their superiority as pre-clinical model is currently of interest to rationalize and assess the therapeutic benefit of new antivirals or immune modulators for the treatment of COVID-19 patients. SARS-CoV-2 contains as structural proteins the spike (S) protein, the envelope (E) protein, the membrane (M) protein, and the nucleocapsid (N) protein. Furthermore, sixteen nonstructural proteins (nspl-16) have been discerned, and being involved in replication and modifying the host defense. The Nsp12 protein corresponds to a RNA-dependent RNA polymerase (RdRp).

Of specific interest in the current invention is the spike or S protein which is a transmembrane glycoprotein forming homotrimers protruding from the viral surface and giving the virus a crown-like look. The spike protein has two subunits: 51 and S2. The 51 subunit comprises an N-terminal domain (NTD), a receptor binding domain (RBD)— as indicated above, the RBD is binding to human ACE-2— and subdomains 1 and 2 (SD1, SD2). The S2 subunit is involved in fusing the membranes of viruses and host cells, and comprises multiple domains: an ST protease cleavage site (cleavage by a host protease required for fusion), a fusion peptide (FP), a heptad repeat 1 (HR1) domain, a central helix (CH) domain, a connector domain (CD), a heptad repeat 2 (HR2) domain, a transmembrane (TM) domain, and a cytoplasmic tail (CT) domain (Wang et al. 2020, Front Cell Infect Microbiol 10:587269). In the prefusion conformation, 51 and S2, cleaved at the S1-S2 furin cleavage site during biosynthesis, remain non-covalently bound to each other—this is different from SARS-CoV in which 51 and S2 remains uncleaved. In the closed state of the S protein (PDB: 6VXX), the 3 RBD domains in the trimer do not protrude from the trimer whereas in the open state (PDB:6VYB), or “up” conformation, one of the RBD does protrude from the trimer. The S-trimer ectodomain with triangular cross-section has a length of approximately 160-Angstrom wherein the 51 domain adopts a V-shaped form. Sixteen of the 22 N-linked glycosylation sites per protomer appear glycosylated (Walls et al. 2020, Cell 180:281-292).

The RBD domain (amino acids 438-506 of the S1 domain) contains a core beta-sheet region formed by antiparallel strands. Between two of the antiparallel strands is inserted the receptor binding motif (RBM) forming an extended structure (formed by 2 short beta-strands, 2 alpha-helices and loops) containing most of the residues binding to ACE2 (Lan et al. 2020, Nature 581:215-220).

The Sars-Cov-2 Spike protein sequence can be found under/corresponds with or to Genbank Accession: QHQ82464, version QHQ82464.1; and is also defined herein as SARS-CoV-2 surface glycoprotein, and as SEQ ID NO:30. Herein, the SARS-CoV-2 Spike protein RBD domain region (also defined as Spike receptor binding domain; pfam09408) corresponds with/to amino acids 330-583 of SEQ ID NO:30 and as depicted hereafter (SEQ ID NO:32); or alternatively corresponds with/to amino acids 330-518 of SEQ ID NO:30 and as depicted hereafter (SEQ ID NO:33):

| [SEQ ID NO: 32] | |
|---|---|
| 330 | p nitnlcpfge vfnatrfasv yawnrkrisn |
| 361 | cvadysvlyn sasfstfkcy gvsptkindl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tle |
| or | |
| [SEQ ID NO: 33] | |
| 330 | p nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfell |

The Sars-Cov-1 Spike protein sequence can be found under/corresponds with or to GenBank accession NP_828851.1; and is also defined herein as SARS-CoV-1 E2 glycoprotein precursor, and as SEQ ID NO:31. Herein, the SARS-CoV-1 Spike protein RBD domain region corresponds with/to amino acid residues 318-569 of SEQ ID NO:31, which is the region corresponding with/to the Spike receptor binding domain of SARS-CoV-2 as depicted hereafter (SEQ ID NO:34); or alternatively corresponds with/to amino acids 320-502 of SEQ ID NO:31 and as depicted hereafter (SEQ ID NO:35):

| [SEQ ID NO: 34] | |
|---|---|
| 318 | nit nlcpfgevin atkfpsvyaw erkkisncva dysvlynstf |
| 361 | fstfkcygvs atklndlcfs nvyadsfvvk gddvrqiapg qtgviadyny klpddfmgcv |
| 421 | lawntrnida tstgnynyky rylrhgklrp ferdisnvpf spdgkpctpp alncywplnd |
| 481 | ygfytttgig yqpyrvvvls fellnapatv cgpklstdli knqcvnfnfn gltgtgvltp |
| 541 | sskrfqpfqq fgrdvsdftd svrdpktse |
| or | |
| [SEQ ID NO: 35] | |
| 320 | t nlcpfgevfn atkfpsvyaw erkkisncva dysvlynstf |
| 361 | fstfkcygvs atklndlcfs nvyadsfvvk gddvrqiapg qtgviadyny klpddfmgcv |
| 421 | lawntrnida tstgnynyky rylrhgklrp ferdisnvpf spdgkpctpp alncywplnd |
| 481 | ygfytttgig yqpyrvvvls fe |

“Angiotensin converting enzyme 2”, “ACE2”, or “ACE-2” as used herein interchangeably refers to mammalian protein belonging to the family of dipeptidyl carboxydipeptidases, and sometimes classified as EC:3.4.17.23. The genomic location of the human ACE2 gene is on chrX:15,561,033-15,602,158 (GRCh38/hg38; minus strand), or alternatively on chrX:15,579,156-15,620,271(GRCh37/hg19; minus strand). ACE2 acts as a receptor for at least human coronaviruses SARS-CoV and SARS-CoV-2, and NL63/HCoV-NL63 (also known as New Haven coronavirus). UniProtKB identifier of human ACE2 protein: Q9BYF1. Isoform 1 (identifier: Q9BYF1-1) has been chosen as the canonical′ sequence. Reference DNA sequence of the human ACE2 gene in GenBank: NC_000023.11. Reference mRNA sequences of human ACE2 in GenBank NM_001371415.1 and N M_021804.3.

Binding Agents/Sarbecovirus Binding Agents

The binding agents or sarbecovirus binding agents (can be used interchangeably) according to the current invention can in one aspect be described functionally by any individual function/embodiment or by any combination of any number of the individual functions/embodiments described hereafter and given an arbitrary number “n” between brackets “(n)”. The numerical order of these individual functions is random and not imposing any preference on an individual function; similarly, this random numerical order is not imposing any preference on any combination of two or more of the individual functions. Any such combination is furthermore not to be considered as arbitrary as the binding agents or sarbecovirus binding agents herein exert each of these individual functions.

As such the binding agents are agents (1) capable of neutralizing, inhibiting, blocking or suppressing sarbecoviruses, in particular (2) capable of neutralizing, inhibiting, blocking or suppressing infection with sarbecoviruses or the infective capacity of sarbecoviruses and/or (3) capable of neutralizing, inhibiting, blocking or suppressing replication of sarbecoviruses. For instance, interaction (binding, specific binding) between a binding agent as identified herein and the sarbecovirus spike protein results in a neutralization of the infection capacity or infective capacity of the sarbecovirus, such as determined in any assay as described herein or as known in the art.

Another function of the binding agents described herein is that these agents are (4) capable of binding or of specifically binding to a spike protein of sarbecoviruses. In particular, these agents are (5) capable of binding or of specifically binding to the RBD domain or motif, or to part of RBD domain or motif, in a sarbecovirus spike protein, in particular in the spike protein of many different sarbecoviruses, more in particular to a highly conserved epitope in RBD domain or motif, or to part of RBD domain or motif, in sarbecovirus spike proteins. Furthermore, in particular these agents are (6) capable of binding or of specifically binding to a partially open conformation of the spike protein of a sarbecovirus; alternatively, these agents are (7) not capable of binding to the closed conformation of the spike protein of a sarbecovirus, or, further alternatively, are (8) not capable of binding to the fully open conformation of the spike protein of a sarbecovirus. Furthermore, in particular these agents are (9) capable of binding or of specifically binding to a spike protein of a sarbecovirus at a site on an RBD domain that is partially in the open conformation, i.e. in a conformation wherein the N-terminal domain of the spike protein is not hindering binding of the binding agent to an RBD domain of a sarbecovirus. At present it is not fully clear how the binding agents according to the current invention are neutralizing, inhibiting, blocking or suppressing sarbecovirus infection. The binding agents of the current invention are (77) capable of inducing S1 shedding. Consequently, the binding agents are capable of inducing premature spike triggering and may as such not allowing the sarbecovirus to complete the infection or entry process into the host cell. Without wishing to be bound by any theory, interaction (binding, specific binding) of these binding agents to an RBD may result in a destabilization of the spike trimer and consequently promote S1 shedding and premature spike triggering. Alternatively, and again without being bound to any theory, interaction (binding) of these binding agents to an RBD may lock or freeze the spike protein in a conformation not allowing the sarbecovirus to complete the infection or entry process into the host cell. Alternatively, and again without being bound to any theory, interaction (binding, specific binding) of these binding agents to an RBD may lead to a destabilization of the spike protein in turn not allowing the sarbecovirus to complete the infection or entry process into the host cell. Independent of their mechanism of action, the binding agents according to the invention are neutralizing sarbecovirus infection efficiently/efficaciously.

A further function of the binding agents described herein is that these agents are (10) not blocking or not preventing binding, thus allowing binding, of a sarbecovirus RBD with ACE2 when the binding agents are themselves bound to the sarbecovirus RBD (alternatively, the binding agent itself can bind to a sarbecovirus RBD to which ACE2 is bound), or are (11) not competing with ACE2 for binding a sarbecovirus RBD (thus allowing binding of ACE2 and the sarbecovirus RBD when the binding agents are themselves bound to the sarbecovirus RBD; (alternatively, the binding agent itself can bind to a sarbecovirus RBD to which ACE2 is bound)), or are (12) not competing with a sarbecovirus RBD for binding with ACE2 (thus allowing binding of the sarbecovirus RBD and ACE2 when the binding agents are themselves bound to the sarbecovirus RBD; (alternatively, the binding agent itself can bind to a sarbecovirus RBD to which ACE2 is bound)). The binding agents are thus capable of neutralizing sarbecovirus, specifically SARS-CoV virus infection, through a modus operandi different from blocking ACE2 binding to the RBD.

A further functional characteristic of the binding agents described herein is that these agents are (13) not competing with the known immunoglobulin CR3022 (ter Meulen et al. 2006, PLoS Med 3:e237; Tian et al. 2020, Emerging Microbes & Infections 9:382-385), and/or are (14) not competing with the known immunoglobulin VHH72 (Wrapp et al. 2020, Cell 184:1004-105), and/or are (15) not competing with the known immunoglobulin CB6 (Shi et al. 2020, Nature 584:120-124), and/or are (16) not competing with the known immunoglobulin 5309 (Pinto et al. 2020, Nature 583:290-295), all for binding or for specifically binding to the spike protein (or RBD domain therein) of sarbecoviruses—this indicates that the binding agents described herein are characterized by a different spike protein/RBD binding pattern compared to the spike protein/RBD binding pattern of any of the immunoglobulins CR3022, VHH72, CB6, or 5309. Alternatively, these binding agents allow binding of CR3022, VHH72, CB6 or 5309 to the sarbecovirus RBD or spike protein when these binding agents are themselves bound to the sarbecovirus RBD. Alternatively, the binding agent itself can bind to a sarbecovirus RBD to which CR3022, VHH72, CB6 or 5309 is bound.

A further functional characteristic of the binding agents described herein is that these agents (17) bind or specifically bind to an epitope in the spike protein or RBD of a sarbecovirus different from the epitope as bound by immunoglobulin mAb52 or Fab52 (Rujas et al. 2020, Biorxiv 2020.10.15.341636v1); and/or (18) bind or specifically bind to an epitope in the spike protein or RBD of a sarbecovirus different from the epitope as bound by immunoglobulin nb34 (Xiang et al. 2020, Science 370:1479-1484); and/or (19) bind or specifically bind to an epitope in the spike protein or RBD of a sarbecovirus different from the epitope as bound by immunoglobulin nb95 (Xiang et al. 2020, Science 370:1479-1484); and/or (20) bind or specifically bind to an epitope in the spike protein or RBD of a sarbecovirus different from the epitope as bound by immunoglobulins n3088 and/or n3130 (Wu et al. 2020, Cell Host Microbe 27:891-898); and/or (21) bind or specifically bind to an epitope in the spike protein or RBD of a sarbecovirus different from the epitope as bound by immunoglobulins n3086 and/or n3113 (Wu et al. 2020, Cell Host Microbe 27:891-898).

A further functional characteristic of the binding agents described herein is that these agents (22) bind or specifically bind to a conserved epitope in the spike protein or RBD of many sarbecoviruses. In particular, the epitope is conserved between different clades of sarbecoviruses. In particular, the epitope is conserved between clade 1.A, cladel.B, clade 2, and clade 3 sarbecoviruses.

A further functional characteristic of the binding agents described herein is that these agents (23) neutralize SARS-CoV-2 and/or SARS-CoV-1 in a pseudotype virus neutralization assay with an IC₅₀ of 10 μg/mL or less, such as with an IC₅₀ of 5 μg/mL or less, such as with an IC₅₀ of 2.5 μg/mL or less, or such as with an IC₅₀ of 1 μg/mL or less. In particular, the pseudotype virus neutralization assay is based on pseudotyped VSV-delG virus containing the spike protein of SARS-CoV-2 or SARS-CoV-1 (see Table 2). Yet a further functional characteristic of the binding agents as described herein is that these agents (78) neutralize SARS-CoV-2 variants, as defined further herein, in a pseudotype virus neutralization assay with an IC₅₀ of 10 μg/mL or less, such as with an IC₅₀ of 5 μg/mL or less, such as with an IC₅₀ of 2.5 μg/mL or less, or such as with an IC₅₀ of 1 μg/mL or less. In particular, the pseudotype virus neutralization assay is based on pseudotyped VSV-delG virus containing the spike protein of SARS-CoV-2 containing the RBD mutations that are associated with the SARS-CoV-2 variant or the spike protein of the SARS-CoV-2 variant. In particular, the binding agents as described herein may neutralize a SARS-CoV-2 variant at position N439, K417, 5477, L452, T478, E484, P384, N501 and/or D614 (relative to the SARS-CoV-2 spike amino acid sequence as defined in SEQ ID NO:30). More particularly, the binding agents as described herein may neutralize one or more, preferably all, of a SARS-CoV-2 variant selected from the group consisting of a SARS-CoV-2 variant comprising a mutation at position N501 such as a N501Y variant (e.g. SARS-CoV-2 alpha variant); a SARS-CoV-2 variant comprising a mutation at positions N501 and E484 such as a N501Y and E484K variant (e.g. SARS-CoV-2 alpha+E484K variant); a SARS-CoV-2 variant comprising a mutation at positions K417, E484 and N501 such as a K417N, E484K and N501Y variant (e.g. SARS-CoV-2 beta variant); a SARS-CoV-2 variant comprising a mutation at positions P384, K417, E484 and N501 such as a P384L, K417N, E484K and N501Y variant (e.g. SARS-CoV-2 beta+P384L variant); a SARS-CoV-2 variant comprising a mutation at positions L452 and E484 such as a L452R and E484Q variant (e.g. SARS-CoV-2 kappa variant); a SARS-CoV-2 variant comprising a mutation at positions L452 and T478 such as a L452R and T478K variant (e.g. SARS-CoV-2 delta variant); a SARS-CoV-2 variant comprising a mutation at position L452 such as a L452R variant (e.g. SARS-CoV-2 epsilon variant); a SARS-CoV-2 variant comprising a mutation at position K417 such as a K417T variant (e.g. SARS-CoV-2 gamma variant) and a SARS-CoV-2 variant comprising a mutation at position D614 such as a D614G variant (e.g. SARS-CoV-2 omicron variant or SARS-CoV-2 BA.1 variant). Even more particularly, the binding agents as described herein are further characterized in that they (79) neutralize SARS-CoV-2 alpha variant, (80) neutralize SARS-CoV-2 alpha+E484K variant, (81) neutralize SARS-CoV-2 beta variant, (82) neutralize SARS-CoV-2 beta+P384L variant, (83) neutralize SARS-CoV-2 kappa variant, (84) neutralize SARS-CoV-2 delta variant, (85) neutralize SARS-CoV-2 epsilon variant, (86) neutralize SARS-CoV-2 gamma variant and/or (87) neutralize SARS-CoV-2 omicron variant or SARS-CoV-2 BA.1 variant, in a pseudotype virus neutralization assay with an IC₅₀ of 10 μg/mL or less, such as with an IC₅₀ of 5 μg/mL or less, such as with an IC₅₀ of 2.5 μg/mL or less, or such as with an IC₅₀ of 1 μg/mL or less. In certain embodiments, binding agents are disclosed which are (88) binding or specifically binding to the SARS-CoV-2 Spike protein (SEQ ID NO:30), or binding or specifically binding to the RBD of the binding to the SARS-CoV-2 Spike protein (SEQ ID NO: 32 or 33). In particular, the agents are (89) binding or specifically binding such that any part of the agent comes within 4 Angstrom of at least one of the amino acids Asn394 (or alternatively Ser394 in some sarbecoviruses), or Tyr396; and/or in particular, these agents are (90) binding or specifically binding such that any part of the agent comes within 4 Angstrom of amino acid Phe464 (or alternatively Tyr464 in some sarbecoviruses); and/or in particular, these agents are (91) binding or specifically binding such that any part of the agent comes within 4 Angstrom to at least one of the amino acids Ser514 or Glu516; and/or in particular, these agents are (92) binding or specifically binding such that any part of the agent comes within 4 Angstrom to amino acid Arg355. In certain embodiments, the agents are (93) binding or specifically binding such that any part of the agent comes within 4 Angstrom of at least one of the amino acids Asn394 (or alternatively Ser394 in some sarbecoviruses), Tyr396, Phe464, Ser514, Glu516, and Arg355. In certain embodiments, the agents are (94) binding or specifically binding such that parts of the agent come within 4 Angstrom of at least two of the amino acids Asn394 (or alternatively Ser394 in some sarbecoviruses), Tyr396, Phe464, Ser514, Glu516, and Arg355. In certain embodiments, the agents are (95) binding or specifically binding such that parts of the agent come within 4 Angstrom of at least three of the amino acids Asn394 (or alternatively Ser394 in some sarbecoviruses), Tyr396, Phe464, Ser514, Glu516, and Arg355. In certain embodiments, the agents are (95) binding or specifically binding such that parts of the agent come within 4 Angstrom of at least four of the amino acids Asn394 (or alternatively Ser394 in some sarbecoviruses), Tyr396, Phe464, Ser514, Glu516, and Arg355. In certain embodiments, the agents are (96) binding or specifically binding such that parts of the agent come within 4 Angstrom of at least five of the amino acids Asn394 (or alternatively Ser394 in some sarbecoviruses), Tyr396, Phe464, Ser514, Glu516, and Arg355. In certain embodiments, the agents are (97) binding or specifically binding such that parts of the agent come within 4 Angstrom of all six of the amino acids Asn394 (or alternatively Ser394 in some sarbecoviruses), Tyr396, Phe464, Ser514, Glu516, and Arg355.

In certain embodiments, the agents are (98) binding or specifically binding to at least one of the amino acids Asn394 (or alternatively Ser394 in some sarbecoviruses), or Tyr396; and/or in particular, these agents are (99) binding or specifically binding to Phe464 (or alternatively Tyr464 in some sarbecoviruses); and/or in particular, these agents are (100) binding or specifically binding to at least one of the amino acids Ser514 or Glu516; and/or in particular, these agents are (101) binding or specifically binding to Arg355. In certain embodiments, the agents are (102) binding or specifically binding to at least one of the amino acids Asn394 (or alternatively Ser394 in some sarbecoviruses), Tyr396, Phe464, Ser514, Glu516, and Arg355. In certain embodiments, the agents are (103) binding or specifically binding to at least two of the amino acids Asn394 (or alternatively Ser394 in some sarbecoviruses), Tyr396, Phe464, Ser514, Glu516, and Arg355. In certain embodiments, the agents are (104) binding or specifically binding to at least three of the amino acids Asn394 (or alternatively Ser394 in some sarbecoviruses), Tyr396, Phe464, Ser514, Glu516, and Arg355. In certain embodiments, the agents are (105) binding or specifically binding to at least four of the amino acids Asn394 (or alternatively Ser394 in some sarbecoviruses), Tyr396, Phe464, Ser514, Glu516, and Arg355. In certain embodiments, the agents are (106) binding or specifically binding to at least five of the amino acids Asn394 (or alternatively Ser394 in some sarbecoviruses), Tyr396, Phe464, Ser514, Glu516, and Arg355. In certain embodiments, the agents are (107) binding or specifically binding to all six of the amino acids Asn394 (or alternatively Ser394 in some sarbecoviruses), Tyr396, Phe464, Ser514, Glu516, and Arg355. In certain embodiments, the agents are (108) binding or specifically binding such that parts of the agent come within 4 Angstrom of at least Tyr396, Ser514, and Glu516. In certain embodiments, the agents are (109) binding or specifically binding to at least Tyr396, Ser514, and Glu516. In certain embodiments, the agents are (110) binding or specifically binding such that parts of the agent come within 4 Angstrom of at least Asn394 (or alternatively Ser394 in some sarbecoviruses), Tyr396, Ser514, and Glu516. In certain embodiments, the agents are (111) binding or specifically binding to at least Asn394 (or alternatively Ser394 in some sarbecoviruses), Tyr396, Ser514, and Glu516. In certain embodiments, the agents are (112) binding or specifically binding such that parts of the agent come within 4 Angstrom of at least Asn394 (or alternatively Ser394 in some sarbecoviruses), Tyr396, Phe464, Ser514, and Glu516. In certain embodiments, the agents are (113) binding or specifically binding to at least Asn394 (or alternatively Ser394 in some sarbecoviruses), Tyr396, Phe464, Ser514, and Glu516.

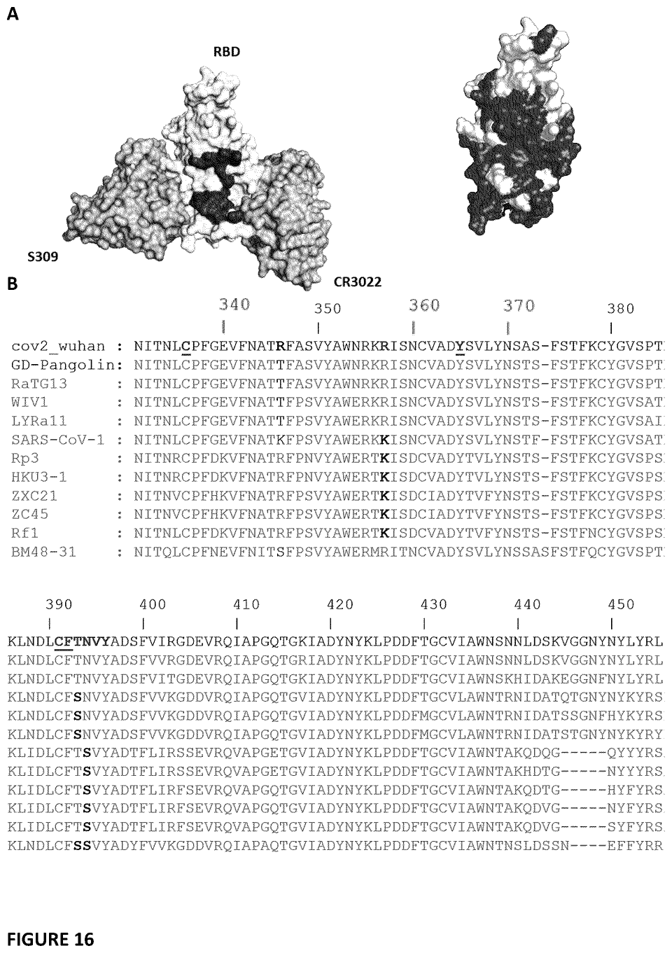

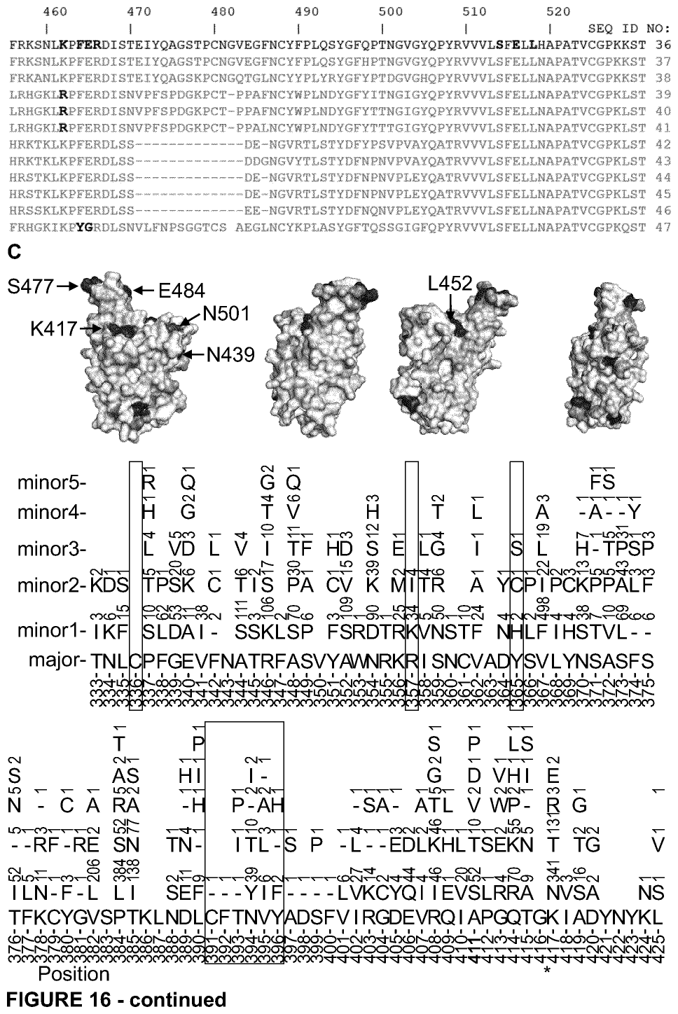

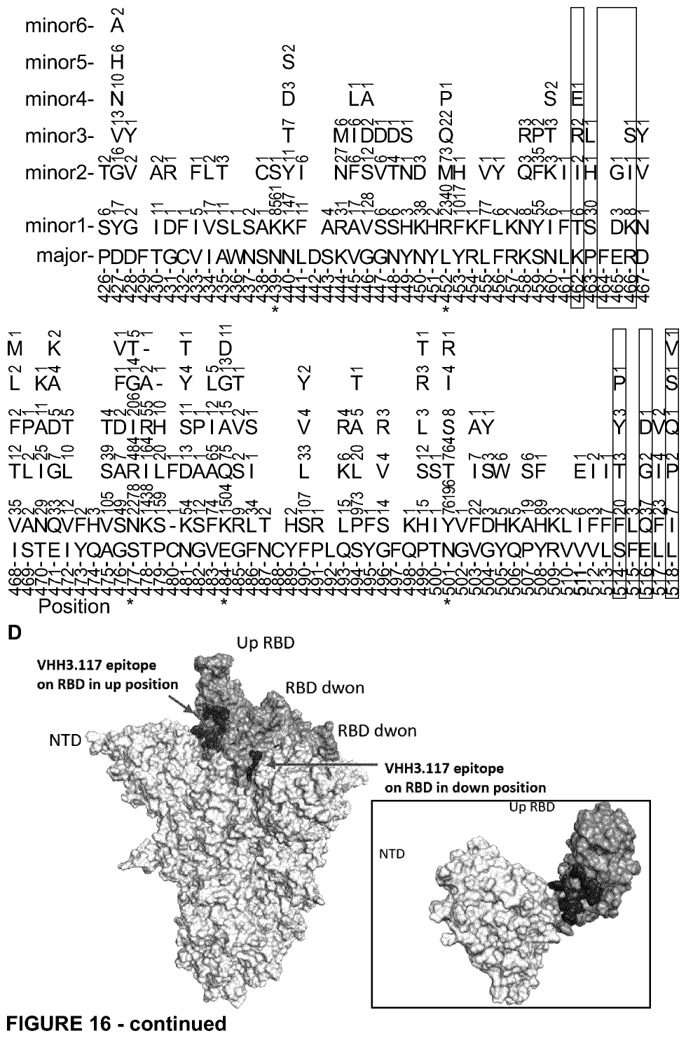

Optionally, any of the foregoing agents are (114) further binding or specifically binding to amino acid Arg357 (or alternatively Lys357 in some sarbecoviruses) and/or Lys462 (or alternatively Arg462 in some sarbecoviruses) and/or Glu465 (or alternatively Gly465 in some sarbecoviruses) and/or Arg466 and/or Leu518, such as (115) further binding or specifically binding to at least two, or in increasing order of preference at least three or all four of amino acid Arg357 (or alternatively Lys357 in some sarbecoviruses) and/or Lys462 (or alternatively Arg462 in some sarbecoviruses) and/or Glu465 (or alternatively Gly465 in some sarbecoviruses) and/or Arg466 and/or Leu518. Optionally, any of the foregoing agents are (116) binding or specifically binding to a sarbecovirus spike protein wherein Cys336 (conserved between sarbecovirus clades) is forming an intramolecular disulfide bridge and/or are (117) binding or specifically binding to a sarbecovirus Spike protein wherein Cys391 (conserved between sarbecovirus clades) is forming an intramolecular disulfide bridge; in particular, (118) Cys336 may be forming an intramolecular disulfide bridge with Cys361 (conserved between sarbecovirus clades) and/or (119) Cys391 may be forming an intramolecular disulfide bridge with Cys525 (conserved between sarbecovirus clades). Optionally, these agents are (120) binding or specifically binding to a sarbecovirus Spike protein wherein amino acid 365 is a tyrosine (Tyr365; conserved between sarbecovirus clades) and/or are (121) binding or specifically binding to a sarbecovirus Spike protein wherein amino acid 392 is a phenylalanine (Phe392; conserved between sarbecovirus clades) and/or are (122) binding or specifically binding to a sarbecovirus Spike protein wherein amino acid 393 is a threonine (Thr393; or alternatively Ser393 in some sarbecoviruses), and/or are (123) binding or specifically binding to a sarbecovirus Spike protein wherein amino acid 395 is a valine (Val395; or alternatively Ser393 in some sarbecoviruses) and/or are (124) binding or specifically binding to a sarbecovirus Spike protein wherein amino acid 518 is a leucine (Leu518). The amino acids and amino acid numbering referred to hereinabove is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B).

In certain embodiments, binding agents are disclosed which are (125) binding or specifically binding to the SARS-CoV-2 Spike protein (SEQ ID NO:30), or binding or specifically binding to the RBD of the binding to the SARS-CoV-2 Spike protein (SEQ ID NO: 32 or 33). In particular, the agents are (126) binding or specifically binding whereby a binding interface is generated (for example, as determined by PDBePISA) that covers at least 25%, at least 33%, at least 50%, or at least 75% of the RBD surface area circumferentially defined by R355, N394, Y396, F464, 5514 and E516. The RBD surface area that is contacted can be calculated to optionally include the intervening surface area that is sterically between these residues.

The above listed functional characteristics of the binding agents according to the invention can in general be determined by methodology as e.g. employed in the Examples described herein, or as described in some of the hereinabove cited and other publications. Determination of the sarbecovirus spike protein epitope or sarbecovirus RBD domain epitope can be performed by means of e.g. binding competition experiments (such as outlined in the Examples herein or in many of the hereinabove cited publications), or e.g. by mutational analysis (such as outlined in the Examples herein), or e.g. by any means of determining interaction at the 3D-level, including in silico modeling (such as outlined herein). In one specific embodiment, some of the functional characteristics of a binding agent or sarbecovirus binding agent as described hereinabove are combined such as to characterizing such agent, e.g. to be binding to the sarbecovirus spike protein Receptor Binding Domain (SPRBD), not to be blocking binding of Angiotensin-Converting Enzyme 2 (ACE2) to SPRBD, to be at least neutralizing SARS-CoV-2 and SARS-CoV-1, in particular at least neutralizing SARS-CoV-2 and SARS-CoV-2 variants as described herein and SARS-CoV-1, and not to be competing with antibody CR3022 for binding with SPRBD. Such agent may further be characterized by neutralizing SARS-CoV-2 and/or SARS-CoV-2 variants and/or SARS-CoV-1 in a pseudotype virus neutralization assay with an IC₅₀ of 10 μg/mL or lower; and/or by not competing with antibodies VHH72, 5309, and CB6; and/or by inducing 51 shedding.

A further functional characteristic of the binding agents described herein is that these agents are (24) binding or specifically binding to the SARS-CoV-2 Spike protein (SEQ ID NO:30), or binding or specifically binding to the RBD of the binding to the SARS-CoV-2 Spike protein (SEQ ID NO: 32 or 33). In particular, these agents are (25) binding or specifically binding to at least one of the amino acids Thr393 (or alternatively Ser393 in some sarbecoviruses), Asn394 (or alternatively Ser394 in some sarbecoviruses), Val395, or Tyr396; and/or in particular, these agents are (26) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and/or in particular, these agents are (27) binding or specifically binding to at least one of the amino acids Ser514, Glu516, or Leu518; and/or in particular, these agents are (28) binding or specifically binding to amino acid Arg357 (or alternatively Lys357 in some sarbecoviruses). In particular, these agents are (29) binding or specifically binding to at least 3, to at least 4, to at least 5, to at least 6, to at least 7, to at least 8, to at least 9, to at least 10, to at least 11, or to all of the amino acids listed in (25) to (28). Optionally, these agents are (30) binding or specifically binding to a sarbecovirus spike protein wherein Cys336 (conserved between sarbecovirus clades, see FIG. 16B) is forming an intramolecular disulfide bridge and/or are (31) binding or specifically binding to a sarbecovirus Spike protein wherein Cys391 (conserved between sarbecovirus clades, see FIG. 16B) is forming an intramolecular disulfide bridge; in particular, (32) Cys336 may be forming an intramolecular disulfide bridge with Cys361 (conserved between sarbecovirus clades, see FIG. 16B) and/or (33) Cys391 may be forming an intramolecular disulfide bridge with Cys525 (conserved between sarbecovirus clades, see FIG. 16B). Optionally, these agents are (34) binding or specifically binding to a sarbecovirus Spike protein wherein amino acid 365 is a tyrosine (Tyr365; conserved between sarbecovirus clades, see FIG. 16B) and/or are (35) binding or specifically binding to a sarbecovirus Spike protein wherein amino acid 392 is a phenylalanine (Phe392; conserved between sarbecovirus clades, see FIG. 16B). The amino acids and amino acid numbering referred to hereinabove is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B).

In multiple further individual embodiments, the binding agents identified herein are:
(36) binding or specifically binding to at least one of the amino acids Thr393 (or alternatively Ser393 in some sarbecoviruses), Asn394 (or alternatively Ser394 in some sarbecoviruses), Val395, or Tyr396; and (further) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; or are
(37) binding or specifically binding to at least one of the amino acids Thr393 (or alternatively Ser393 in some sarbecoviruses), Asn394 (or alternatively Ser394 in some sarbecoviruses), Val395, or Tyr396; and (further) binding or specifically binding to at least one of the amino acids Ser514, Glu516, or Leu518; or are
(38) binding or specifically binding to at least one of the amino acids Thr393 (or alternatively Ser393 in some sarbecoviruses), Asn394 (or alternatively Ser394 in some sarbecoviruses), Val395, or Tyr396; and (further) binding or specifically binding to amino acid Arg357; or are
(39) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and (further) binding or specifically binding to at least one of the amino acids Ser514, Glu516, or Leu518; or are
(40) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and (further) binding or specifically binding to amino acid Arg357; or are
(41) binding or specifically binding to at least one of the amino acids Ser514, Glu516, or Leu518; and (further) binding or specifically binding to amino acid Arg357; or are
(42) binding or specifically binding to at least one of the amino acids Thr393 (or alternatively Ser393 in some sarbecoviruses), Asn394 (or alternatively Ser394 in some sarbecoviruses), Val395, or Tyr396; and (further) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and (further) binding or specifically binding to at least one of the amino acids Ser514, Glu516, or Leu518; or are
(43) binding or specifically binding to at least one of the amino acids Thr393 (or alternatively Ser393 in some sarbecoviruses), Asn394 (or alternatively Ser394 in some sarbecoviruses), Val395, or Tyr396; and (further) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and (further) binding or specifically binding to amino acid Arg357; or are
(44) binding or specifically binding to at least one of the amino acids Thr393 (or alternatively Ser393 in some sarbecoviruses), Asn394 (or alternatively Ser394 in some sarbecoviruses), Val395, or Tyr396; and (further) binding or specifically binding to at least one of the amino acids Ser514, Glu516, or Leu518; and (further) binding or specifically binding to amino acid Arg357; or are
(45) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and (further) binding or specifically binding to at least one of the amino acids Ser514, Glu516, or Leu518; and (further) binding or specifically binding to amino acid Arg357; or are
(46) binding or specifically binding to at least one of the amino acids Thr393 (or alternatively Ser393 in some sarbecoviruses), Asn394 (or alternatively Ser394 in some sarbecoviruses), Val395, or Tyr396; and (further) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and (further) binding or specifically binding to at least one of the amino acids Ser514, Glu516, or Leu518; and (further) binding or specifically binding to amino acid Arg357; or are
(47) binding or specifically binding to amino acids Thr393 (or alternatively Ser393 in some sarbecoviruses), Asn394 (or alternatively Ser394 in some sarbecoviruses), Val395, Tyr396, Lys462 (or alternatively Arg462 in some sarbecoviruses), Phe464 (or alternatively Tyr464 in some sarbecoviruses), Glu465 (or alternatively Gly465 in some sarbecoviruses), Arg466, Ser514, Glu516, or Leu518 and Arg357.

The amino acids and amino acid numbering referred to hereinabove is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B).

The binding or specific binding to at least one of the amino acids Thr393 (or alternatively Ser393 in some sarbecoviruses), Asn394 (or alternatively Ser394 in some sarbecoviruses), Val395, or Tyr396 is further explained in (48) to (58) hereafter. In particular, these agents are (25) binding or specifically binding to at least one of the amino acids Thr393 (or alternatively Ser393 in some sarbecoviruses), Asn394 (or alternatively Ser394 in some sarbecoviruses), Val395, or Tyr396;
such as (48) binding or specifically binding to at least amino acids Thr393 (or alternatively Ser393 in some sarbecoviruses) and Asn394 (or alternatively Ser394 in some sarbecoviruses);
such as (49) binding or specifically binding to at least amino acids Thr393 (or alternatively Ser393 in some sarbecoviruses) and Val395;
such as (50) binding or specifically binding to at least amino acids Thr393 (or alternatively Ser393 in some sarbecoviruses) and Tyr396;
such as (51) binding or specifically binding to at least amino acids Asn394 (or alternatively Ser394 in some sarbecoviruses) and Val395;
such as (52) binding or specifically binding to at least amino acids Asn394 (or alternatively Ser394 in some sarbecoviruses) and Tyr396;
such as (53) binding or specifically binding to at least amino acids Val395 and Tyr396;
such as (54) binding or specifically binding to at least amino acids Thr393 (or alternatively Ser393 in some sarbecoviruses), Asn394 (or alternatively Ser394 in some sarbecoviruses) and Val395;
such as (55) binding or specifically binding to at least amino acids Thr393 (or alternatively Ser393 in some sarbecoviruses), Asn394 (or alternatively Ser394 in some sarbecoviruses) and Tyr396;
such as (56) binding or specifically binding to at least amino acids Thr393 (or alternatively Ser393 in some sarbecoviruses), Val395 and Tyr396;
such as (57) binding or specifically binding to at least amino acids Asn394 (or alternatively Ser394 in some sarbecoviruses), Val395 and Tyr396; or such as (58) binding or specifically binding to at least amino acids Thr393 (or alternatively Ser393 in some sarbecoviruses), Asn394 (or alternatively Ser394 in some sarbecoviruses), Val395 and Tyr396;
the amino acids and amino acid numbering referred to hereinabove is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B).

The binding or specific binding to at least one of the amino acids Lys462, Phe464, Glu465 or Arg466 is further explained in (59) to (69) hereafter. In particular, these agents are (26) binding or specifically binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466;
such as (59) binding or specifically binding to at least amino acids Lys462 (or alternatively Arg462 in some sarbecoviruses) and Phe464 (or alternatively Tyr464 in some sarbecoviruses);
such as (60) binding or specifically binding to at least amino acids Lys462 (or alternatively Arg462 in some sarbecoviruses) and Glu465 (or alternatively Gly465 in some sarbecoviruses);
such as (61) binding or specifically binding to at least amino acids Lys462 (or alternatively Arg462 in some sarbecoviruses) and Arg466;
such as (62) binding or specifically binding to at least amino acids Phe464 (or alternatively Tyr464 in some sarbecoviruses) and Glu465 (or alternatively Gly465 in some sarbecoviruses);
such as (63) binding or specifically binding to at least amino acids Phe464 (or alternatively Tyr464 in some sarbecoviruses) and Arg466;
such as (64) binding or specifically binding to at least amino acids Glu465 (or alternatively Gly465 in some sarbecoviruses) and Arg466;
such as (65) binding or specifically binding to at least amino acids Lys462 (or alternatively Arg462 in some sarbecoviruses), Phe464 (or alternatively Tyr464 in some sarbecoviruses) and Glu465;
such as (66) binding or specifically binding to at least amino acids Lys462 (or alternatively Arg462 in some sarbecoviruses), Phe464 (or alternatively Tyr464 in some sarbecoviruses) and Arg466;
such as (67) binding or specifically binding to at least amino acids Lys462 (or alternatively Arg462 in some sarbecoviruses), Glu465 (or alternatively Gly465 in some sarbecoviruses) and Arg466;
such as (68) binding or specifically binding to at least amino acids Phe464 (or alternatively Tyr464 in some sarbecoviruses), Glu465 (or alternatively Gly465 in some sarbecoviruses) and Arg466; or such as (69) binding or specifically binding to at least amino acids Lys462 (or alternatively Arg462 in some sarbecoviruses), Phe464 (or alternatively Tyr464 in some sarbecoviruses), Glu465 (or alternatively Gly465 in some sarbecoviruses) and Arg466;
the amino acids and amino acid numbering referred to hereinabove is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B).

The binding or specific binding to at least one of the amino acids Ser514, Glu516, or Leu518 is further explained in (70) to (73) hereafter. In particular, these agents are (27) binding or specifically binding to at least one of the amino acids Ser514, Glu516, or Leu518;
such as (70) binding or specifically binding to at least amino acids Ser514 and Glu516;
such as (71) binding or specifically binding to at least amino acids Ser514 and Leu518;
such as (72) binding or specifically binding to at least amino acids Glu516 and Leu518; or such as (73) binding or specifically binding to at least amino acids Ser514, Glu516, and Leu518;
the amino acids and amino acid numbering referred to hereinabove is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B).

In one particular embodiment, the sarbecovirus binding agent may be defined/may be characterized in that the agent is binding to the sarbecovirus spike protein Receptor Binding Domain (SPRBD), is allowing binding of Angiotensin-Converting Enzyme 2 (ACE2) to SPRBD when the sarbecovirus binding agent itself is bound to SPRBD, is at least neutralizing SARS-CoV-2 and SARS-CoV-1, in particular at least neutralizing SARS-CoV-2, SARS-CoV-2 variants as described herein and SARS-CoV-1, and is binding to at least one of the amino acids Thr393 (or alternatively Ser393 in some sarbecoviruses), Asn394 (or alternatively Ser394 in some sarbecoviruses), Val395, or Tyr396 of the SARS-CoV-2 spike protein as defined in SEQ ID NO:30. Such agent may further be characterized by inducing 51 shedding.

Interaction of a binding agent or partner as described herein to a sarbecovirus spike protein or RBD domain therein can be derived from structural models. In particular, it can be described in terms of intermolecular distances between an atom of the binding partner (e.g. an amino acid or an amino acid side chain or an amino acid hydrogen) and an atom of the sarbecovirus spike protein or RBD domain therein (e.g. an amino acid or an amino acid side chain or an amino acid hydrogen). Algorithms exist by which binding free energy of complexes are estimated, such as FastContact (Champ et al. 2007, Nucleic Acids Res 35:W556-W560). In the FastContact algorithm, the range of desolvation interaction can be adapted, e.g. 6 Angstrom (potential going down to zero between 5 and 7 Ångström) or 9 Ångström (potential going down to zero between 8 and 10 Angstrom); electrostatic and van der Waals energy are other components used by the FastContact algorithm.

Thus, (74) interaction of a binding agent or partner as described herein to a sarbecovirus spike protein or RBD domain therein can be derived from structural models by defining an interaction between an atom of the binding partner and an atom of the sarbecovirus spike protein or RBD domain therein (as described hereinabove) as a true interaction if the distance between the two atoms is e.g. between 1 Ångström (Å) and 10 Å, between 1 Å and 9 Å, between 1 Å and 8 Å, between 1 Å and 7 Å, between 1 Å and 6 Å, between 1 Å and 5 Å, between 1 Å and 4 Å, between 1 Å and 3 Å, between 1 Å and 2 Å, and depending on the resolution at which the structure has been resolved. Alternatively, residues of the sarbecovirus spike protein or RBD domain therein are in ‘in contact’ with residues of the binding agent or partner, and such ‘contact’ can be defined herein as (intermolecular) contacts between residues with a distance of 4 Å or less, of 5 Å or less, of 6 Å or less, of 7 Å or less, of 8 Å or less, of 9 Å or less, or of 10 Å or less.

In particular, the (75) binding agent or partner is or comprises one or more complementary determining regions (CDRs) of an immunoglobulin single variable domain (ISVD) as described herein, or comprises one or more ISVDs as described herein, and binds to a part of the sarbecovirus spike protein or RBD domain as described in detail hereinabove (the epitope of the ISVDs). As such, amino acids (or parts thereof) of the herein described ISVDs contact or interact with sarbecovirus spike protein/RBD domain amino acids (or parts thereof) wherein the contacting or interaction distance is between 1 Ångström (A) and 10 Å, between 1 Å and 9 Å, between 1 Å and 8 Å, between 1 Å and 7 Å, between 1 Å and 6 Å, between 1 Å and 5 Å, between 1 Å and 4 Å, between 1 Å and 3 Å, between 1 Å and 2 Å; or is 4 Å or less, 5 Å or less, 6 Å or less, 7 Å or less, 8 Å or less, 9 Å or less, or 10 Å or less, wherein the lower limit of distance is defined by the resolution of the determined structure.

In particular, (76) parts of the binding agents or partners (such as amino acids (or parts thereof) of the herein described CDRs and/or ISVDs), are contacting or interacting with a distance of between 1 Ångström (A) and 10 Å, between 1 Å and 9 Å, between 1 Å and 8 Å, between 1 Å and 7 Å, between 1 Å and 6 Å, between 1 Å and 5 Å, between 1 Å and 4 Å, between 1 Å and 3 Å, between 1 Å and 2 Å; or of 4 Å or less, 5 Å or less, 6 Å or less, 7 Å or less, 8 Å or less, 9 Å or less, or 10 Å or less:
with at least one of the amino acids Thr393 (or alternatively Ser393 in some sarbecoviruses), Asn394 (or alternatively Ser394 in some sarbecoviruses), Val395, or Tyr396; and/or with at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and/or with at least one of the amino acids Ser514, Glu516, or Leu518; and/or with amino acid Arg357 (or alternatively Lys357 in some sarbecoviruses). The amino acids and amino acid numbering referred to hereinabove is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B); or
with at least one of the amino acids Asn394 (or alternatively Ser394 in some sarbecoviruses), Tyr396, Phe464, Ser514, Glu516, and Arg355 of the SARS-CoV-2 spike protein as defined in SEQ ID NO:30; optionally further with amino acid Arg357 (or alternatively Lys357 in some sarbecoviruses) and/or Lys462 (or alternatively Arg462 in some sarbecoviruses) and/or Glu465 (or alternatively Gly465 in some sarbecoviruses) and/or Arg466 and/or Leu518.

The binding agents according to the current invention are in another aspect structurally defined as polypeptidic binding agents (i.e. binding agents comprising a peptidic, polypeptidic or proteic moiety, or binding agents comprising a peptide, polypeptide, protein or protein domain) or polypeptide binding agents (i.e. binding agents being peptides, polypeptides or proteins). More in particular, the binding agents according to the current invention can be structurally defined as polypeptidic or polypeptide binding agents comprising a complementarity determining region (CDR) as comprised in any of the immunoglobulin single variable domains (ISVDs) defined hereinafter. More in particular, the binding agents according to the current invention can in one embodiment be structurally defined as polypeptidic or polypeptide binding agents comprising at least CDR3 as comprised in an immunoglobulin single variable domains (ISVDs) as defined hereinafter. In another embodiment, the binding agents according to the current invention can be structurally defined as polypeptidic or polypeptide binding agents comprising at least two of CDR1, CDR2 and CDR3 (e.g. CDR1 and CDR3, CDR2 and CDR3, CDR1 and CDR2), or all three of CDR1, CDR2 and CDR3, as comprised in an immunoglobulin single variable domains (ISVDs) as defined hereinafter. More in particular such CDRs are comprised in any of VH H3.117 (defined by/set forth in SEQ ID NO:1), VHH3.92 (defined by/set forth in SEQ ID NO:2), VHH3.94 (defined by/set forth in SEQ ID NO:3), VHH3.42 (defined by/set forth in SEQ ID NO:4), or VHH3.180 (defined by/set forth in SEQ ID NO:5) as depicted hereafter:

VHH3.117:
(SEQ ID NO: 1)
QVQLQESGGGLVQPGGSLRLSCAASGKAVSISDMGWYRQPPGKQRELVAT
ITKTGSTNYADSAQGRFTISRDNTKSAVYLEMKSLKPEDTAVYYCNAWLP
YGMGPDYYGMELWGKGTQVTVSS
VHH3.92:
(SEQ ID NO: 2)
QVQLQESGGGLVQPGGSLRLSCAASGKAVSISDMGWYRQPPGKQRELVAT
ITKTGNTNYADSAQGRETISRDNAKSAVYLEMASLKPEDTAVYYCNAWLP
YGMGPDYYGMELWGKGTQVTVSS
VHH3.94:
(SEQ ID NO: 3)
QVQLQESGGGLVQPGGSLRLSCAASGKAVSISDMGWYRQPPGKQRELVAT
ITKSGSTNYANSAQGRETISRDNAKSAVYLEMNSLKPEDTAVYYCNAWLP
YGMGPDYYGMELWGEGTQVTVSS
VHH3.42:
(SEQ ID NO: 4)
QVQLQESGGGLVQPGGSLRLSCAASGSAVSINDMGWYRQPPGKQRELVAT
ITKTGSTNYADSVKGRETISRDNAKNAVYLEMNSLKPEDTATYYCNAWLP
YGMGPDYYGMELWGKGTQVTVSS
VHH3.180:
(SEQ ID NO: 5)
QVQLQESGGGSVQAGRSLTLNCAASGKAVSISDMGWYRQPPGKQRELVAT
ITKTGSTNYADSAQGRFTISRDNAKSAVYLEMNSLKPEDTAVYYCNAWLL
YGMGPDYYGMELWGEGTQVTVSS

In other embodiments, such CDRs may be comprised in any of VHH3.89 (defined by/set forth in SEQ ID NO:53), VHH3_183 (defined by/set forth in SEQ ID NO:54) or VHH3C_80 (defined by/set forth in SEQ ID NO:55) as depicted hereafter:

VHH3.89:
(SEQ ID NO: 53)
QVQLQESGGGLVQPGGSLRLSCAASGETLDYYAIGWFREVPGKEREGLSR
IDSSDGSTYYADSVKGRFTISRDNTKNIVYLQMNNLKPEDTAVYYCATDP
IIQGRNWYWTGWGQGTQVTVSS
VHH3 183:
(SEQ ID NO: 54)
QVQLQESGGGLVQPGGSLRLSCAASGLDYYAIGWFRQAPGKEREGLSRIE
SSDGSTYYADSVKGRETISRDNTKNTVYLQMNSLKPEDTAVYYCATDPII
QGSSWYWTSWGQGTQVTVSS
VHH3C 80:
(SEQ ID NO: 55)
QVQLQESGGGSVQPGESLRLSCVGSGHTLDDYDVGWFRQAPGKEREVLSR
IDSSDGSTYYADSVKGRFTISRDNTKNIVYLQMNMLKPEDTAAYYCATDP
IIRGHNWYWTGWSQSTHITVSS

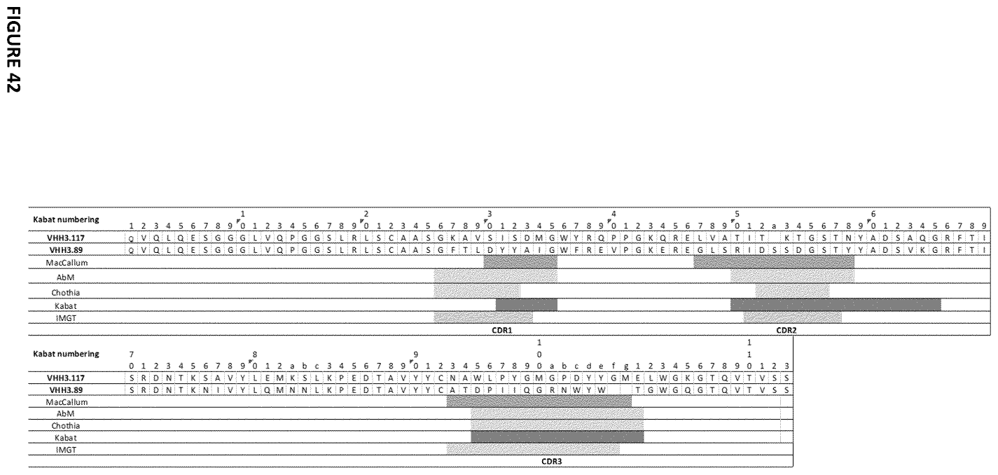

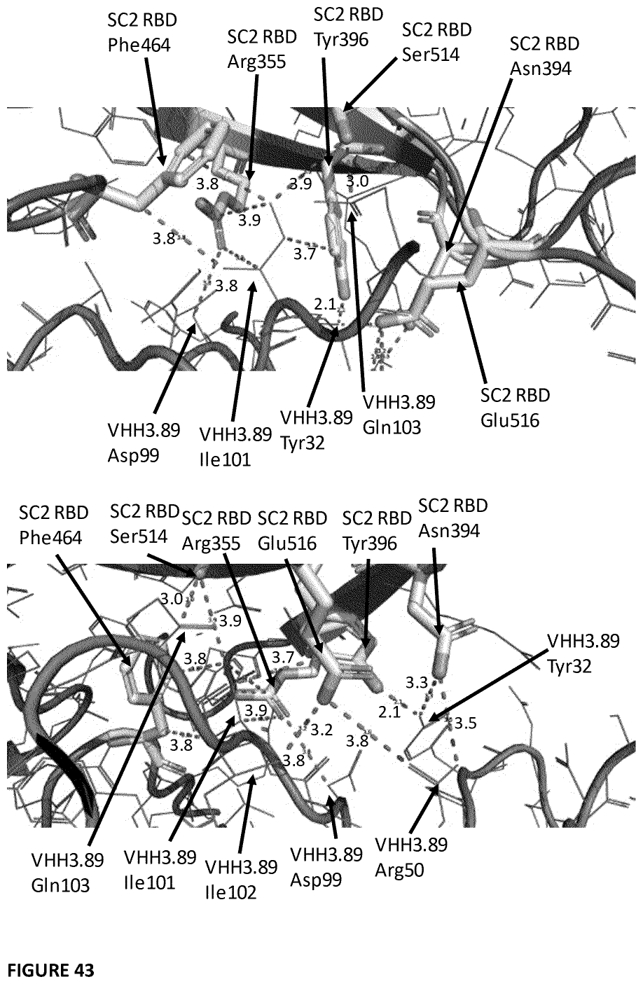

As outlined and defined herein (see definitions and FIG. 42), many systems or methods (Kabat, MacCallum, IMGT, AbM, Chothia,) exist for numbering amino acids in immunoglobulin protein sequences, including for delineation of CDRs and framework regions (FRs) in these protein sequences. These systems or methods are known to a skilled artisan who thus can apply these systems or methods on any immunoglobulin protein sequences without undue burden. A binding agent or sarbecovirus binding agent as described herein may thus e.g. be characterized in that it is comprising the complementarity determining regions (CDRs) present in any of SEQ ID NOs: 1 to 5 or 53 to 55, wherein the CDRs are are annotated according to Kabat, MacCallum, IMGT, AbM, or Chothia (as illustrated for VHH3.117 and VHH3.89 in FIG. 42).

Solely as non-limiting example, the CDRs comprised in any of VHH3.117, VHH3.92, VHH3.94, VHH3.42, or VHH3.180 were determined according to Kabat or according to the Kabat system or method. By employing the Kabat methodology as example, CDRs comprised in the ISVDs of the invention can, in embodiments, be defined as:
CDR1: IXDMG, wherein X (Xaa) at position 2 is S (Ser, serine) or N (Asn, asparagine)(SEQ ID NO:6). More in particular, CDR1 can be defined as ISDMG (SEQ ID NO:9; comprised in VHH3.117, VHH3.92, VHH3.94 and VHH3.180) or INDMG (SEQ ID NO:10; comprised in VHH3.42);
CDR2: TITKXGXTNYAXSXXG, wherein X (Xaa) at position 5 is T (Thr, threonine) or S (Ser, serine), X (Xaa) at position 7 is S (Ser, serine) or N (Asn, asparagine), X (Xaa) at position 12 is D (Asp, aspartic acid) or N (Asn, asparagine), X (Xaa) at position 14 is A (Ala, alanine) or V (Val, valine), and X (Xaa) at position 15 is Q (Gln, glutamine) or K (Lys, lysine) (SEQ ID NO:7). More in particular, CDR2 can be defined as TITKTGSTNYADSAQG (SEQ ID NO:11; comprised in VHH3.117 and VHH3.180), TITKTGNTNYADSAQG (SEQ ID NO:12; comprised in VHH3.92), TITKSGSTNYANSAQG (SEQ ID NO:13; comprised in VHH3.94), or TITKTGSTNYADSVKG (SEQ ID NO:14; comprised in VHH3.42);
CDR3: WLXYGMGPDYYGME, wherein X (Xaa) at position 3 is P (Pro, proline) or L (Leu, leucine) (SEQ ID NO:8). More in particular, CDR3 can be defined as WLPYGMGPDYYGME (SEQ ID NO:15; comprised in VHH3.117, VHH3.92, VHH3.94 and VHH3.42), or WLLYGMGPDYYGME (SEQ ID NO:16; comprised in VHH3.180).

More in particular, polypeptidic or polypeptide binding agents of the current invention can be defined as comprising one of following sets of three complementarity determining regions (CDRs), wherein the CDRs are defined according to Kabat:
CDR1 defined by/set forth in SEQ ID NO:6, CDR2 defined by/set forth in SEQ ID NO:7, and CDR3 defined by/set forth in SEQ ID NO:8; or
CDR1 defined by/set forth in SEQ ID NO:9, CDR2 defined by/set forth in SEQ ID NO:11, and CDR3 defined by/set forth in SEQ ID NO:15; or
CDR1 defined by/set forth in SEQ ID NO:9, CDR2 defined by/set forth in SEQ ID NO:12, and CDR3 defined by/set forth in SEQ ID NO:15; or
CDR1 defined by/set forth in SEQ ID NO:9, CDR2 defined by/set forth in SEQ ID NO:13, and CDR3 defined by/set forth in SEQ ID NO:15; or
CDR1 defined by/set forth in SEQ ID NO:10, CDR2 defined by/set forth in SEQ ID NO:14, and CDR3 defined by/set forth in SEQ ID NO:15; or
CDR1 defined by/set forth in SEQ ID NO:9, CDR2 defined by/set forth in SEQ ID NO:11, and CDR3 defined by/set forth in SEQ ID NO:16.

Solely as further non-limiting example, the CDRs comprised in any of VHH3.89, VHH3_183, or VHH3C_80, were determined according to Kabat or according to the Kabat system or method. By employing the Kabat methodology as example, CDRs comprised in the ISVDs of the invention can, in alternative embodiments, be defined as:
CDR1: XYXXG, wherein X (Xaa) at position 1 is D or Y; X (Xaa) at position 3 is D or A, and X (Xaa) at position 4 is V or I (SEQ ID NO: 76). More in particular, CDR1 can be defined as YYAIG (SEQ ID NO: 69; comprised in VHH3.89 and VHH3_183) or DYDVG (SEQ ID NO:70; comprised in VHH3C_80);
CDR2: RIXSSDGSTYYADSVKG, wherein X (Xaa) at position 3 is D or E (SEQ ID NO:77). More in particular, CDR2 can be defined as RIDSSDGSTYYADSVKG (SEQ ID NO:71; comprised in VHH3.89 and VHH3C_80), RIESSDGSTYYADSVKG (SEQ ID NO:72; comprised in VHH3_183);
CDR3: DPIIXGXXWYWT, wherein X (Xaa) at position 5 is R or Q, X (Xaa) at position 7 is R, S or H, and wherein X(Xaa) at position 8 is N or S (SEQ ID NO:78). More in particular, CDR3 can be defined as DPIIQGRNWYWT (SEQ ID NO:73; comprised in VHH3.89), or DPIIQGSSWYWT (SEQ ID NO:74, comprised in VHH3_183), or DPIIRGHNWYWT (SEQ ID NO:75, comprised in VHH3C_80).

More in particular, polypeptidic or polypeptide binding agents of the current invention can be defined as comprising one of following sets of three complementarity determining regions (CDRs), wherein the CDRs are defined according to Kabat:
CDR1 defined by/set forth in SEQ ID NO:76, CDR2 defined by/set forth in SEQ ID NO:77, and CDR3 defined by/set forth in SEQ ID NO:78; or
CDR1 defined by/set forth in SEQ ID NO:69, CDR2 defined by/set forth in SEQ ID NO: 71, and CDR3 defined by/set forth in SEQ ID NO: 73 (corresponding to the CDRs as present in VHH3.89); or
CDR1 defined by/set forth in SEQ ID NO: 69, CDR2 defined by/set forth in SEQ ID NO: 72, and CDR3 defined by/set forth in SEQ ID NO: 74 (corresponding to the CDRs as present in VHH3_183); or
CDR1 defined by/set forth in SEQ ID NO: 70, CDR2 defined by/set forth in SEQ ID NO: 71, and CDR3 defined by/set forth in SEQ ID NO: 75 (corresponding to the CDRs as present in VHH3C_80.

In a further aspect, the polypeptidic or polypeptide binding agents according to the current invention can be comprising one or more framework regions (FRs) as comprised in any of the ISVDs defined hereinabove. More in particular, such binding agents may be comprising an FR1, FR2, FR3, of FR4 region as comprised in any of the ISVDs defined hereinabove. More in particular, such binding agents may be comprising an FR1 and FR2 region, an FR1 and FR3 region, an FR1 and FR4 regions, an FR2 and FR3 region, an FR2 and FR4 region, an FR3 and FR4 region, an FR1, FR2 and FR3 region, an FR1, FR2 and FR4 region, an FR2, FR3 and FR4, or an FR1, FR3 and FR4 region as comprised in any of the ISVDs defined hereinabove. In one embodiment, such binding agents are comprising an FR1 region or an FR4 region or an FR2 and FR3 region as comprised in any of the ISVDs defined hereinabove.

As outlined and defined hereinabove, many systems or methods (Kabat, MacCallum, IMGT, AbM, or Chothia) exist for numbering amino acids in immunoglobulin protein sequences, including for delineation of FRs in these protein sequences. These systems or methods are known to a skilled artisan who thus can apply these systems or methods on any immunoglobulin protein sequences without undue burden.

Solely as non-limiting example, the FRs comprised in any of VHH3.117, VHH3.92, VHH3.94, VHH3.42, or VHH3.180 were determined according to Kabat or according to the Kabat system or method. By employing the Kabat methodology as example, FRs comprised in the ISVDs of the invention can, in embodiments, be defined as:
FR1: QVQLQESGGGXVQXGXSLXLXCAASGXAVS, wherein X(Xaa) at position 11 is L (Leu, leucine) or S (Ser, serine), X(Xaa) at position 14 is P (Pro, proline) or A (Ala, alanine), X(Xaa) at position 16 is G (Gly, glycine) or R (Arg, arginine), X(Xaa) at position 19 is R (Arg, arginine) or T (Thr, threonine), X(Xaa) at position 21 is S (Ser, serine) or N (Asn, asparagine), and X(Xaa) at position 27 is K (Lys, lysine) or S (Ser, serine) (SEQ ID NO:17). More in particular, FR1 can be defined as QVQLQESGGGLVQPGGSLRLSCAASGKAVS (SEQ ID NO:21, comprised in VHH3.117, VHH3.92, and VHH3.94), QVQLQESGGGLVQPGGSLRLSCAASGSAVS (SEQ ID NO:22, comprised in VHH3.42), or QVQLQESGGGSVQAGRSLTLNCAASGKAVS (SEQ ID NO:23, comprised in VHH3.180);
FR2: WYRQPPGKQRELVA (SEQ ID NO:18, comprised in VHH3.117, VHH3.92, VHH3.94, VHH3.42 and VHH3.180);
FR3: RFTISRDNXKXAVYLEMXSLKPEDTAXYYCNA, wherein X(Xaa) at position 9 is T (Thr, threonine) or A (Ala, alanine), X(Xaa) at position 11 is S (Ser, serine) or N (Asn, asparagine), X(Xaa) at position 18 is K (Lys, lysine), A (Ala, alanine) or N (Asn, asparagine), and X(Xaa) at position 27 is V (Val, valine) or T (Thr, threonine) (SEQ ID NO:19). More in particular, FR3 can be defined as RFTISRDNTKSAVYLEMKSLKPEDTAVYYCNA (SEQ ID NO:24, comprised in VHH3.117), RFTISRDNAKSAVYLEMASLKPEDTAVYYCNA (SEQ ID NO:25, comprised in VHH3.92), RFTISRDNAKSAVYLEMNSLKPEDTAVYYCNA (SEQ ID NO:26, comprised in VHH3.94 and VHH3.180), or RFTISRDNAKNAVYLEMNSLKPEDTATYYCNA (SEQ ID NO:27, comprised in VHH3.42);
FR4: LWGXGTQVTVSS, wherein X(Xaa) at position 4 is K (Lys, lysine) or E (Glu, glutamine) (SEQ ID NO:20).

More in particular, FR4 can be defined as LWGKGTQVTVSS (SEQ ID NO:28, comprised in VHH3.117, VHH3.92 and VHH3.42) or LWGEGTQVTVSS (SEQ ID NO:29, comprised in VHH3.94 and VHH3.180).

More in particular, polypeptidic or polypeptide binding agents of the current invention can be defined as comprising a set of framework regions FR1, FR2, FR3 and FR4 that together have an amino acid sequence that is at least 90%, at least 95% or at least 97% identical to a combination of the amino acid sequence of an FR1 selected from the sequences defined by SEQ ID NO: 21 to 23, the amino acid sequence of an FR2 defined by SEQ ID NO:18, the amino acid sequence of an FR3 selected from the sequences defined by SEQ ID NO: 24 to 27, and the amino acid sequence of an FR4 selected from the sequences defined by SEQ ID NO: 28 or 29. This is to be understood such as that in the 4 individual amino acids alignments of FR sequence pairs (i.e. variant FR1 with one of SEQ ID NO: 21 to 23; variant FR2 with SEQ ID NO:18; variant FR3 with one of SEQ ID NO: 24 to 27; and variant FR4 with one of SEQ ID NO: 28 or 29) all together at least 90%, at least 95% or at least 97% of the amino acids is identical.

More in particular, polypeptidic or polypeptide binding agents of the current invention can be defined as comprising one of following sets of framework regions (FRs), wherein the FRs are defined according to Kabat:
FR1 defined by/set forth in SEQ ID NO:17, FR2 defined by/set forth in SEQ ID NO:18, FR3 defined by/set forth in SEQ ID NO:19, and FR4 defined by/set forth in SEQ ID NO:20; or
FR1 defined by/set forth in SEQ ID NO:21, FR2 defined by/set forth in SEQ ID NO:18, FR3 defined by/set forth in SEQ ID NO:24, and FR4 defined by/set forth in SEQ ID NO:28; or
FR1 defined by/set forth in SEQ ID NO:21, FR2 defined by/set forth in SEQ ID NO:18, FR3 defined by/set forth in SEQ ID NO:25, and FR4 defined by/set forth in SEQ ID NO:28; or
FR1 defined by/set forth in SEQ ID NO:21, FR2 defined by/set forth in SEQ ID NO:18, FR3 defined by/set forth in SEQ ID NO:26, and FR4 defined by/set forth in SEQ ID NO:28; or
FR1 defined by/set forth in SEQ ID NO:22, FR2 defined by/set forth in SEQ ID NO:18, FR3 defined by/set forth in SEQ ID NO:27, and FR4 defined by/set forth in SEQ ID NO:28; or
FR1 defined by/set forth in SEQ ID NO:23, FR2 defined by/set forth in SEQ ID NO:18, FR3 defined by/set forth in SEQ ID NO:26, and FR4 defined by/set forth in SEQ ID NO:29.

Solely as a further non-limiting example, the FRs comprised in any of VHH3.89, VHH3_183 and VHH3C_80 were determined according to Kabat or according to the Kabat system or method. By employing the Kabat methodology as example, FRs comprised in the ISVDs of the invention can, in alternative embodiments, be defined as:
FR1: QVQLQESGGGXVQPGXSLRLSCXXSGXTLD, wherein X(Xaa) at position 11 is S or L; X(Xaa) at position 16 is E or G; X(Xaa) at position 23 is A or V; X(Xaa) at position 24 is G or A; X(Xaa) at position 27 is H, or F (SEQ ID NO:82) which more in particular can be defined as QVQLQESGGGLVQPGGSLRLSCAASGFTLD (SEQ ID NO:79, comprised in VHH3.89), or QVQLQESGGGSVQPGESLRLSCVGSGHTLD (SEQ ID NO:81, comprised in VHH3C_80). Alternatively, FR1 is presented by QVQLQESGGGLVQPGGSLRLSCAASGLD (SEQ ID NO:80, comprised in VHH3.183);
FR2: WFRXXPGKEREXLS (SEQ ID NO:86), wherein X(Xaa) at position 4 is Q or E; X(Xaa) at position 5 is A or V; X(Xaa) at position 12 is G or V. More in particular, FR2 can be defined as WFREVPGKEREGLS (SEQ ID NO: 83 as comprised in VHH3.89), or as WFRQAPGKEREGLS (SEQ ID NO: 84 as comprised in VHH3_183), or as WFRQAPGKEREVLS (SEQ ID NO: 85 as comprised in VHH3C_80).
FR3: RFTISRDNTKNXVYLQMNXLKPEDTAXYYCAT, wherein X(Xaa) at position 12 is I or T; X(Xaa) at position 19 is M, N or S; X(Xaa) at position 27 is V or A (SEQ ID NO:90). More in particular, FR3 can be defined as RFTISRDNTKNIVYLQMNNLKPEDTAVYYCAT (SEQ ID NO: 87, as comprised in VHH3.89), RFTISRDNTKNTVYLQMNSLKPEDTAVYYCAT (SEQ ID NO: 88, as comprised in VHH3_183), or RFTISRDNTKNIVYLQM NM LKPEDTAAYYCAT (SEQ ID NO: 89 as comprised in VHH3C_80);
FR4: XWXQXTXXTVSS, wherein X(Xaa) at position 1 is S or G; X(Xaa) at position 3 and 5 is G or 5; X(Xaa) at position 7 is Q or H; X(Xaa) at position 8 is V or I (SEQ ID NO:94). More in particular, FR4 can be defined as GWGQGTQVTVSS (SEQ ID NO:91, comprised in VHH3.89) or SWGQGTQVTVSS (SEQ ID NO:92, comprised in VHH3_183), or GWSQSTHITVSS (SEQ ID NO:93 as comprised in VHH3C_80).

More in particular, polypeptidic or polypeptide binding agents of the current invention can be defined as comprising a set of framework regions FR1, FR2, FR3 and FR4 that together have an amino acid sequence that is at least 90%, at least 95% or at least 97% identical to a combination of the amino acid sequence of an FR1 selected from the sequences defined by SEQ ID NO: 79-82, the amino acid sequence of an FR2 selected from the sequences defined by SEQ ID NO:83-86, the amino acid sequence of an FR3 selected from the sequences defined by SEQ ID NO: 87-90, and the amino acid sequence of an FR4 selected from the sequences defined by SEQ ID NO: 91-94. This is to be understood such as that in the 4 individual amino acids alignments of FR sequence pairs (i.e. variant FR1 with one of SEQ ID NO: 79-82; variant FR2 with one of SEQ ID NO:83-86; variant FR3 with one of SEQ ID NO: 87-90; and variant FR4 with one of SEQ ID NO: 91-94) all together at least 90%, at least 95% or at least 97% of the amino acids is identical.

More in particular, polypeptidic or polypeptide binding agents of the current invention can be defined as comprising one of following sets of framework regions (FRs), wherein the FRs are defined according to Kabat:
FR1 defined by/set forth in SEQ ID NO:79, FR2 defined by/set forth in SEQ ID NO:83, FR3 defined by/set forth in SEQ ID NO:87, and FR4 defined by/set forth in SEQ ID NO:91; or
FR1 defined by/set forth in SEQ ID NO:80, FR2 defined by/set forth in SEQ ID NO:84, FR3 defined by/set forth in SEQ ID NO:88, and FR4 defined by/set forth in SEQ ID NO:92; or
FR1 defined by/set forth in SEQ ID NO:81, FR2 defined by/set forth in SEQ ID NO:85, FR3 defined by/set forth in SEQ ID NO:89, and FR4 defined by/set forth in SEQ ID NO:93.

In one particular embodiment, the polypeptidic or polypeptide binding agents of the current invention can be defined as full ISVDs, i.e., as defined by or set forth in any of SEQ ID NOs: 1, 2, 3, 4 or 5; or as polypeptidic or polypeptide binding agents comprising any of the ISVDs as defined by or set forth in any of SEQ ID NOs: 1, 2, 3, 4 or 5. In another particular embodiment, the polypeptidic or polypeptide binding agents of the current invention can be defined as full ISVDs, i.e., as defined by or set forth in any of SEQ ID NOs: 53, 54 or 55; or as polypeptidic or polypeptide binding agents comprising any of the ISVDs as defined by or set forth in any of SEQ ID NOs: 53, 54 or 55.

In a further embodiment, said polypeptidic or polypeptide binding agents binding agents are comprising one or more ISVDs individually defined by or set forth in any of SEQ ID NOs: 1, 2, 3, 4 or 5, or comprising one or more ISVDs selected from the group of SEQ ID NO: 1 to 5. In a further embodiment, said polypeptidic or polypeptide binding agents binding agents are comprising one or more ISVDs individually defined by or set forth in any of SEQ ID NOs: 53, 54 or 55, or comprising one or more ISVDs selected from the group of SEQ ID NO: 53, 54 or 55.

In a further embodiment, said polypeptidic or polypeptide binding agents binding agents are comprising one or more amino acid sequences with at least 90% identity to an amino acid sequence selected from the group of SEQ ID NO: 1 to 5, or with at least 95% identity to an amino acid sequence selected from the group of SEQ ID NO: 1 to 5. In particular, such non-identity or variability, is limited to non-identity or variability in FR amino acid residues. In particular, such non-identity or variability may be introduced to obtain a humanized variant of an ISVD defined by or set forth in any of SEQ ID NOs: 1, 2, 3, 4 or 5, such as a humanized variant for example but not limited to any one of an ISVD defined by SEQ ID NO:57-61. In particular, such humanized variant is a functional orthologue of the original ISVD, wherein the functional features are one or more of the functional features (1) to (126) outlined extensively hereinabove.

In a further embodiment, said polypeptidic or polypeptide binding agents binding agents are comprising one or more amino acid sequences with at least 90% identity to an amino acid sequence selected from the group of SEQ ID NO: 53, 54 or 55, or with at least 95% identity to an amino acid sequence selected from the group of SEQ ID NO: 53, 54 or 55, in particular, such non-identity or variability, is limited to non-identity or variability in FR amino acid residues. In particular, such non-identity or variability may be introduced to obtain a humanized variant of an ISVD defined by or set forth in any of SEQ ID NOs: 53, 54 or 55, such as a humanized variant for example but not limited to SEQ ID NO:56. In particular, such humanized variant is a functional orthologue of the original ISVD, wherein the functional features are one or more of the functional features (1) to (126) outlined extensively hereinabove.

Another embodiment relates to said polypeptidic or polypeptide binding agents that are comprising one or more ISVDs (or variants or humanized forms thereof as described herein) wherein the at least one or more ISVD (or variant or humanized form thereof as described herein) is bound or fused to an Fc domain, wherein with Fc domain is meant the fragment crystallizable region (Fc region) of an antibody, which is the tail region known to interact with cell surface receptors called Fc receptors and some proteins of the complement system. Said Fc domain is composed of two identical protein fragments, derived from the second and third constant domains of the antibody's two heavy chains. All conventional antibodies comprise an Fc domain, hence, the Fc domain fusion may comprise an Fc domain derived from or as a variant of the IgG, IgA and IgD antibody Fc regions, even more specifically an IgG1, IgG2 or IgG4. The hinge region of IgG2, may be replaced by the hinge of human IgG1 to generate ISVD fusion constructs, and vice versa. Additional linkers that are used to fuse a herein identified ISVD to the IgG1 and IgG2 Fc domains comprise (G₄S)₂₋₃. In addition, Fc variants with known half-live extension may be used such as the M257Y/S259T/T261E (also known as YTE) or the LS variant (M428L combined with N434S). These mutations increase the binding of the Fc domain of a conventional antibody to the neonatal receptor (FcRn).

In a particular further embodiment, the polypeptidic or polypeptide binding agents of the invention are comprising one or more ISVDs (or variants or humanized forms thereof as described herein) are in a “multivalent” or “multispecific” form and are formed by bonding, chemically or by recombinant DNA techniques, together two or more identical or variant monovalent ISVDs (or variants or humanized forms thereof as described herein). Said multivalent forms may be formed by connecting the building block directly or via a linker, or through fusing the with an Fc domain encoding sequence. Non-limiting examples of multivalent constructs include “bivalent” constructs, “trivalent” constructs, “tetravalent” constructs, and so on. The ISVDs (or variants or humanized forms thereof as described herein comprised within a multivalent construct may be identical or different. In another particular embodiment, the ISVDs (or variants or humanized forms thereof as described herein) of the invention are in a “multi-specific” form and are formed by bonding together two or more ISVDs, of which at least one with a different specificity. Non-limiting examples of multi-specific constructs include “bi-specific” constructs, “tri-specific” constructs, “tetra-specific” constructs, and so on. To illustrate this further, any multivalent or multi-specific (as defined herein) ISVD of the invention may be directed against two or more different antigens, for example against the Corona RBD and one as a half-life extension against Serum Albumin or SpA. Multivalent or multi-specific ISVDs of the invention may also have (or be engineered and/or selected for) increased avidity and/or improved selectivity for the desired Corona RBD interaction, and/or for any other desired property or combination of desired properties that may be obtained by the use of such multivalent or multi-specific immunoglobulin single variable domains. In another embodiment, the invention provides a polypeptidic or polypeptide binding agent comprising any of the ISVDs (or variants or humanized forms thereof as described herein) according to the invention, either in a monovalent, multivalent or multi-specific form. Thus, monovalent, multivalent or multi-specific polypeptidic or polypeptide binding agents comprising a herein described ISVD (or variant or humanized form thereof as described herein) or part thereof are included here as non-limiting examples.

Particularly, a single ISVD (or variant or humanized form thereof) as described herein may be fused at its C-terminus to an IgG Fc domain, such as a construct as defined in any of SEQ ID NO:63 to 65, resulting in a sarbecovirus binding agents of bivalent format wherein two of said ISVDs (or variants or humanized forms thereof as described herein), form a heavy chain only-antibody-type molecule through disulfide bridges in the hinge region of the IgG Fc part. Said humanized forms thereof, include but are not limited to the IgG humanization variants known in the art, such as C-terminal deletion of Lysine, alteration or truncation in the hinge region, LALA or LALAPG mutations as described herein, among other substitutions in the IgG sequence.

Other binding agents according to the invention are any compounds or molecules binding to the same epitope as bound by any of the ISVDs defined by or set forth in any of SEQ ID NOs: 1 to 5 or SEQ ID NO:53 to 55, or any compounds or molecules competing with an ISVD defined by an amino acid sequence selected from the group of SEQ ID NO: 1 to 5 or SEQ ID NO:53 to 55 for binding to a sarbecovirus spike protein or part thereof (as described hereinabove). With “competing” is meant that the binding of ISVD defined by an amino acid sequence selected from the group of SEQ ID NO: 1 to 5 or SEQ ID NO:53 to 55 to a sarbecovirus spike protein or part thereof, in particular to the SARS-CoV-2 RBD as depicted in SEQ ID NO:32 or SEQ ID NO:33 or to the SARS-CoV-1 RBD as depicted in SEQ ID NO:34 or SEQ ID NO:35, is reduced with at least 30%, or at least 50%, or preferably at least 80% in strength in the presence of said competing binding agent. More specifically, said competing binding agent specifically binds to an epitope on a sarbecovirus spike protein comprising at least one of the amino acids Thr393 (or alternatively Ser393 in some sarbecoviruses), Asn394 (or alternatively Ser394 in some sarbecoviruses), Val395, or Tyr396; and/or with at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and/or with at least one of the amino acids Ser514, Glu516, or Leu518; and/or with amino acid Arg357 (or alternatively Lys357 in some sarbecoviruses); wherein the amino acids and amino acid numbering referred to is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B). In particular, such other binding agents ideally retain one or more of the functional features (1) to (126) outlined extensively hereinabove.

As such, the invention in one aspect relates to methods of screening for compounds (compounds of interest) binding to a sarbecovirus spike protein, in particular to a sarbecovirus RBD domain in a sarbecovirus spike protein, and competing with an ISVD or functional part thereof as described herein for binding to a sarbecovirus spike protein, in particular to a sarbecovirus RBD domain in a sarbecovirus spike protein. Such methods in general comprise one or more of the following steps:
providing a compound or pool of compounds;
contacting the compound or pool of compounds with a sarbecovirus RBD domain in the absence of an ISVD or functional part thereof as described herein;
contacting the compound or pool of compounds with a sarbecovirus RBD domain in the presence of an ISVD or functional part thereof as described herein;
measuring, assessing, determining, assaying whether the compound or pool of compounds is capable of reducing the amount of ISVD or functional part thereof bound to the sarbecovirus RBD; or measuring, assessing, determining, assaying whether the ISVD or functional part thereof is capable of reducing the amount of compound or pool of compounds bound to the sarbecovirus RBD;
identifying a compound as competitor of the ISVD or functional part thereof for binding to the sarbecovirus RBD when the amount of ISVD or functional part thereof bound to the sarbecovirus RBD is reduced in the presence of the compound; or identifying a pool of compounds to comprise one or more compounds as competitor of the ISVD or functional part thereof for binding to the sarbecovirus RBD when the amount of ISVD or functional part thereof bound to the sarbecovirus RBD is reduced in the presence of the compound; or identifying a compound as competitor of the ISVD or functional part thereof for binding to the sarbecovirus RBD when the amount of compound bound to the sarbecovirus RBD is reduced in the presence of the ISVD or functional part thereof; or identifying a pool of compounds to comprise one or more compounds as competitor of the ISVD or functional part thereof for binding to the sarbecovirus RBD when the amount of compound or pool of compounds bound to the sarbecovirus RBD is reduced in the presence of the ISVD or functional part thereof.

In yet another aspect, the invention provides nucleic acid molecules such as isolated nucleic acids, (isolated) chimeric gene constructs, expression cassettes, recombinant vectors (such as expression or cloning vectors) comprising a nucleotide sequence, such a a coding sequence, that is encoding the polypeptide portion of a polypeptidic or polypeptide sarbecovirus binding agent as identified herein.

One further aspect of the invention provides for a host cell comprising a polypeptidic or polypeptide sarbecovirus binding agent or part thereof, such as an ISVD or part thereof, as described herein. The host cell may therefore comprise the nucleic acid molecule encoding said polypeptide binding agent. Host cells can be either prokaryotic or eukaryotic. The host cell may also be a recombinant host cell, which involves a cell which has been genetically modified to contain an isolated DNA molecule, nucleic acid molecule encoding the polypeptide binding agent of the invention. Representative host cells that may be used to produce said ISVDs, but are not limited to, bacterial cells, yeast cells, plant cells and animal cells. Bacterial host cells suitable for production of the binding agents of the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. Animal host cells suitable for use with the invention include insect cells and mammalian cells (most particularly derived from Chinese hamster (e.g. CHO), and human cell lines, such as HeLa). Exemplary insect cell lines include, but are not limited to, Sf9 cells, baculovirus-insect cell systems (e.g. review Jarvis, Virology Volume 310, Issue 1, 25 May 2003, Pages 1-7). Alternatively, the host cells may also be transgenic animals or plants.

A further aspect of the invention relates to medicaments or pharmaceutical compositions comprising a binding agent (or sarbecovirus binding agent), and/or nucleic acid encoding it, and/or a recombinant vector comprising the nucleic acid, as described herein. In particular, a pharmaceutical composition is a pharmaceutically acceptable composition; such compositions are in a particular embodiment further comprising a (pharmaceutically) suitable or acceptable carrier, diluent, stabilizer, etc.

A further aspect of the invention relates to a binding agent, nucleic acid encoding it as described herein, or to a pharmaceutical composition comprising a binding agent, nucleic acid encoding it, and/or a recombinant vector comprising such nucleic acid, as described herein, for use as a medicine or medicament. Alternatively, use of a binding agent or nucleic acid encoding it as described herein, or use of a pharmaceutical composition comprising a binding agent, nucleic acid encoding it, and/or a recombinant vector comprising such nucleic acid, as described herein, in the manufacture of a medicine or medicament is envisaged. In particular, the binding agent or nucleic acid encoding it as described herein, or the medicament or pharmaceutical composition comprising a binding agent, nucleic acid encoding it, and/or a recombinant vector comprising such nucleic acid, as described herein, is for use in passive immunisation, for use in treating a subject with a sarbecovirus infection, for use in preventing infection of a subject with a sarbecovirus, or for use in protecting a subject from infection with a sarbecovirus. When for use in passive immunisation, the subject may have an infection with a sarbecovirus (therapeutic passive immunisation) or may not have an infection with a sarbecovirus (prophylactic passive immunisation).

A further aspect of the invention relates to methods for treating a subject suffering from/having/that has contracted an infection with a sarbecovirus, the methods comprising administering a binding agent or nucleic acid encoding it as described herein to the subject, or comprising administering a medicament or pharmaceutical composition comprising a binding agent or nucleic acid encoding it as described herein to the subject.

A further aspect of the invention relates to methods for protecting a subject from infection with a sarbecovirus or for preventing infection of a subject with a sarbecovirus, the methods comprising administering a binding agent or nucleic acid encoding it as described herein to the subject prior to infection, or comprising administering a medicament or pharmaceutical composition comprising a binding agent or nucleic acid encoding it as described herein to the subject prior to infection.

In particular, in the above medical aspects, the sarbecovirus is a coronavirus, more in particular a zoonotic coronavirus, even more in particular SARS-CoV-2 or SARS-CoV-1, even more in particular SARS-CoV-2 variants such as variants at position N439, K417, 5477, L452, T478, E484, P384, N501 and/or D614 (relative to the SARS-CoV-2 spike amino acid sequence as defined in SEQ ID NO:30), more particularly a variant at position N501 such as a N501Y variant (e.g. SARS-CoV-2 alpha variant), a variant at position N501 and E484 such as a N501Y and E484K variant (e.g. SARS-CoV-2 alpha+E484K variant), a variant at position K417, E484 and N501 such as a K417N, E484K and N501Y variant (e.g. SARS-CoV-2 beta variant), a variant at position P384, K417, E484 and N501 such as a P384L, K417N, E484K and N501Y variant (e.g. SARS-CoV-2 beta+P384L variant), a variant at position L452 and E484 such as a L452R and E484Q variant (e.g. SARS-CoV-2 kappa variant), a variant at position L452 and T478 such as a L452R and T478K variant (e.g. SARS-CoV-2 delta variant), a variant at position L452 such as a L452R variant (e.g. SARS-CoV-2 epsilon variant), a variant at position K417 such as a K417T variant (e.g. SARS-CoV-2 gamma variant) or a variant at position D614 such as a D614G variant (e.g. SARS-CoV-2 omicron variant or SARS-CoV-2 BA.1 variant). In particular, treatment is referring to passive immunisation of a subject having contracted a sarbecovirus infection. In particular, prevention of infection with a sarbecovirus is useful in case of e.g. epidemic or pandemic conditions during which subjects known to be most vulnerable to develop severe disease symptoms can be prophylactically treated (preventive or prophylactic immunisation) with a binding agent or nucleic acid encoding it as described herein such as to prevent infection overall, or such as to prevent development or occurrence of severe disease symptoms. In order to achieve the preventive or prophylactic effect, the binding agent or nucleic acid encoding it as described herein may need to be administered to a subject multiple times, such as with an interval of 1 week or 2 weeks; the interval being dictated by the pharmacokinetic behaviour or characteristics (half-time) of the binding agent or nucleic acid. Further in particular, the subject is a mammal susceptible to infection with the sarbecovirus, such as a human subject that is susceptible to infection with SARS-CoV-2 such as a SARS-CoV-2 variant, or SARS-CoV-1.

Furthermore, in particular to the above medical aspects, a nucleic acid encoding a binding agent as described herein can be used in e.g. gene therapy setting or RNA vaccination setting.

A further specific embodiment relates to prophylactic treatment, in which a single dose of a binding agent as described herein is administered and wherein the single dose is in the range of 0.5 mg/kg to 25 mg/kg. Alternatively, a therapeutic treatment with a binding agent is envisaged wherein a single dose in the range of 0.5 mg/kg to 25 mg/kg is envisaged. In both prophylactic and therapeutic settings, multiple doses may need to be administered, and the time interval between two subsequent doses being determined by the half-life of the binding agent in the subject's circulation.

Furthermore in particular to the above medical aspects, the binding agent, nucleic acid or pharmaceutical composition may be administered to a subject via intravenous injection, subcutaneous injection, or intranasally, or, alternatively via inhalation or pulmonary delivery.

Furthermore, in particular to the above medical aspects, a therapeutically effective amount of e binding agent, nucleic acid or pharmaceutical composition is administered to a subject in need thereof; the administration of such therapeutically effective amount leading to inhibiting or preventing infection with a sarbecovirus, and/or leading to curing infection with a sarbecovirus.

A further aspect of the invention relates to a binding agent as described herein for use in diagnosing a sarbecovirus infection, for use as a diagnostic agent, or for use in the manufacture of a diagnostic agent or diagnostic kit, such as an in vitro diagnostic agent or kit. Alternatively, use of a binding agent as described herein in the manufacture of a diagnostic agent/in vitro diagnostic agent is envisaged. In particular, the binding agent as described herein is for use in detecting the presence (or absence) of a sarbecovirus in a sample, such as a sample obtained from a subject, such as from a subject suspected to be infected with a sarbecovirus infection. A nucleic acid encoding a binding agent or sarbecovirus binding agent as described herein, or a recombinant vector comprising such nucleic acid can likewise be used in or be for use in the manufacture of a diagnostic agent or diagnostic kit, such as an in vitro diagnostic agent or kit.

A further aspect of the invention relates to methods for detecting a sarbecovirus in a sample, such as a sample obtained from a subject, such as from a subject suspected to be infected with a sarbecovirus infection. Such methods usually comprise the steps of obtaining a sample, contacting the sample with a binding agent as described herein, and detecting, determining, assessing, assaying, identifying or measuring binding of the binding agent with a sarbecovirus.

In particular, in the above diagnostic aspects, the sarbecovirus is a coronavirus, more in particular a zoonotic coronavirus, even more in particular SARS-CoV-2 such as a SARS-CoV-2 variant or SARS-CoV-1. Further in particular, the subject is a mammal susceptible to infection with the sarbecovirus, such as a human subject that is susceptible to infection with SARS-CoV-2 such as a SARS-CoV-2 variant or SARS-CoV-1.

Further in particular, in the above diagnostic aspects, the binding agent as described herein is comprising a detectable moiety fused to it, bound to it, coupled to it, linked to it, complexed to it, or chelated to it. A “detectable moiety” in general refers to a moiety that emits a signal or is capable of emitting a signal upon adequate stimulation, or to a moiety that is capable of being detected through binding or interaction with a further molecule (e.g. a tag, such as an affinity tag, that is specifically recognized by a labelled antibody), or is detectable by any means (preferably by a non-invasive means, if detection is in vivo/inside the human body). Furthermore, the detectable moiety may allow for computerized composition of an image, as such the detectable moiety may be called an imaging agent. Detectable moieties include fluorescence emitters, phosphorescence emitters, positron emitters, radioemitters, etc., but are not limited to emitters as such moieties also include enzymes (capable of measurably converting a substrate) and molecular tags. Examples of radioemitters/radiolabels include ⁶⁸Ga, ^{110m}In, ¹⁸F, ⁴⁵Ti, ⁴⁴Sc, ⁴⁷Sc, ⁶¹Cu, ⁶⁰Cu, ⁶²Cu, ⁶⁶Ga, ⁶⁴Cu, ⁵⁵Ca, ⁷²As, ⁸⁶Y, ⁹⁰Y, ⁸⁹Zr, ¹²⁵I, ⁷⁴Br, ⁷⁵Br, ⁷⁶Br, ⁷⁷Br, ⁷⁸Br, ¹¹¹In, ^{114m}In, ¹¹⁴In, ^{99m}Tc, ¹¹C, ³²Cl, ³³Cl, ³⁴Cl, ¹²³I, ¹²⁴I, ¹³¹I, ¹⁸⁶Re, ¹⁸⁸Re, ¹⁷⁷Lu, ⁹⁹Tc, ²¹²Bi, ²¹³Bi, ²¹²Pb, ²²⁵Ac, ¹⁵³Sm, and ⁶⁷Ga. Fluorescence emitters include cyanine dyes (e.g. Cy5, Cy5.5, Cy7, Cy7.5), FITC, TRITC, coumarin, indolenine-based dyes, benzoindolenine-based dyes, phenoxazines, BODIPY dyes, rhodamines, Si-rhodamines, Alexa dyes, and derivatives of any thereof. Affinity tags, such as chitin binding protein (CBP), maltose binding protein (MBP), glutathione-S-transferase (GST), poly(His) (e.g., 6×His or His6), biotin or streptavidin, such as Strep-Tag®, Strep-tag II® and Twin-Strep-Tag®; solubilizing tags, such as thioredoxin (TRX), poly(NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase).

Binding agents as describe herein and comprising a detectable moiety may for example be used for in vitro, in vivo or in situ assays (including immunoassays known per se such as ELISA, RIA, EIA and other “sandwich assays”, etc.) as well as in vivo imaging purposes, depending on the choice of the specific label. A specific embodiment discloses the use of the binding agent, optionally in a labelled form, for detection of a virus or Spike protein of said virus, wherein said virus is selected from the group of clade 1a, 1b, 2 and/or clade 3 bat SARS-related sarbecoviruses, such as SARS-Cov-2, GD-Pangolin, RaTG13, WIV1, LYRa11, RsSHC014, Rs7327, SARS-CoV-1, Rs4231, Rs4084, Rp3, HKU3-1, or BM48-31 viruses. In another alternative aspect of the invention, any of the binding agents described herein, optionally with a label, or any of the nucleic acid molecules encoding said agent, or any of the compositions, or vectors as described herein may as well be used as a diagnostic, or in detection of a corona virus, as described herein. Diagnostic methods are known to the skilled person and may involve biological samples from a subject. Also in vitro methods may be in scope for detection of viral protein or particles using the binding agents as described herein. Finally, the binding agents as described herein, optionally labelled, may also be suitable for use in in vivo imaging.

A further aspect of the invention relates to kits comprising a binding agent or nucleic acid encoding it as described herein, or a pharmaceutical composition comprising a binding agent or nucleic acid encoding it as described herein.

Such kits comprise pharmaceutical kits or medicament kits which are comprising a container or vial (any suitable container or vial, such as a pharmaceutically acceptable container or vial) comprising an amount of binding agent or nucleic acid encoding it as described herein, and further comprising e.g. a kit insert such as a medical leaflet or package leaflet comprising information on e.g. intended indications (prophylactic or therapeutic treatment of sarbecovirus infection) and potential side-effects. Pharmaceutical kits or medicament kits may further comprise e.g. a syringe for administering the binding agent or nucleic acid encoding it as described herein to a subject.

Such kits comprise diagnostic kits comprising a container or vial (any suitable container or vial, such as a pharmaceutically acceptable container or vial) comprising an amount of binding agent as described herein, such as a binding agent comprising a detectable moiety. Such diagnostic kits may further comprise e.g. one or more reagents to detect the detectable moiety and/or e.g. instructions on how to use said binding agent for detection of a sarbecovirus in a sample.

Crystal Complexes

Another aspect of the invention relates to a complex comprising a sarbecovirus RBD and a binding agent as described herein. In a one embodiment, said complex is of a crystalline form. The crystalline allows to use the atomic details of the interactions in said complex as a molecular template to design molecules that will recapitulate the key features of interfaces of the binding agent as described herein with the sarbecovirus RBD domain. In the light of recent developments in computational docking and in pharmacophore building, the isolation of small compounds that can mimic protein-protein interface is becoming a realistic strategy.

Another embodiment relates to a computer-assisted method and/or in silico method of identifying, designing or screening for a binding agent as described herein, in particular for a binding agent with one or more of the functional features selected from the group consisting of (1) to (126) as described extensively hereinabove, wherein said methods are comprising one or more steps of:
i. introducing into a suitable computer program the parameters defining the three-dimensional (3D) structure comprising the binding site of an ISVD defined by/set forth in an amino acid sequence selected from SEQ ID NOs: 1 to 5 or SEQ ID NO: 53 to 55, or comprising the binding site of a functional fragment of such ISVD;
ii. generation, creating or modelling (in the same or other suitable computer program as used in i.) or importing (in the same or other suitable computer program as used in i.) a 3D structure of a test compound; in particular such test compound is a compound suspected to bind to the 3D structure introduced in i.;
iii. (computationally) superimposing (or computer-assisted superimposement of) the 3D structure introduced in i., and the 3D structure of the test compound generated, created, modelled or imported in ii.; in particular the superimposing process is repetitive such as until the energetically most favourable fit between the two three-dimensional structures is obtained; and
iv. (computationally) assessing, determining, evaluating (or computer-assisted assessment, determination, evaluation of) whether said test compound model fits spatially and chemically into the 3D binding site (as introduced in i.); in particular this step may comprise comparison of the fit with the spatial and chemical interaction of the 3D binding site with an ISVD or functional part thereof as described herein.

In particular, said test compound is selected from the group consisting of (1) peptides such as soluble peptides, including Ig-tailed fusion peptides and members of random peptide libraries and combinatorial chemistry-derived molecular libraries made of D- and/or L-configuration amino acids; (2) phosphopeptides (e.g. members of random and partially degenerate, directed phosphopeptide libraries, (3) immunoglobulin variable domains or antibodies (e.g., polyclonal, monoclonal, humanized, anti-idiotypic, chimeric, and single chain antibodies, nanobodies, intrabodies, affibodies, as well as Fab, (Fab)₂, Fab expression library and epitope-binding fragments of antibodies); (4) non-immunoglobulin binding proteins such as but not restricted to avimers, DARPins, alphabodies, affitins, nanofitins, anticalins, monobodies and lipocalins; (5) nucleic acid-based aptamers; (6) small organic and inorganic molecules; and (7) polypeptidic compounds such as bicyclic peptides (also known as Bicycles®).

Said binding site as described herein is also referred to herein as the epitope of the invention. Moreover, the epitope here refers to specific residues in the RBD of a sarbecovirus Spike protein, i.e. an epitope on a sarbecovirus spike protein comprising at least one of the amino acids Thr393 (or alternatively Ser393 in some sarbecoviruses), Asn394 (or alternatively Ser394 in some sarbecoviruses), Val395, or Tyr396; and/or with at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and/or with at least one of the amino acids Ser514, Glu516, or Leu518; and/or with amino acid Arg357 (or alternatively Lys357 in some sarbecoviruses); wherein the amino acids and amino acid numbering referred to is relative to/corresponding to the SARS-CoV-2 Spike protein as defined in SEQ ID NO:30; corresponding amino acids in spike proteins or RBD domains of other sarbecoviruses can be easily determined by aligning multiple amino acid sequences, e.g. as depicted in FIG. 16B). In particular, such other binding agents ideally retain one or more of the functional features (1) to (126) outlined extensively hereinabove. In particular, the spatial and chemical fitting, such as determined computationally, is determined based on the contact points of the test compound with the 3D binding site (as introduced in i.); such contact points are residues in that are in ‘in contact’ with each other. In particular, such contact distances are outlined in functional features (74) to (76) hereinabove.

Rational Drug Design

Using a variety of known modelling techniques, the crystal structures described hereinabove can be used to produce 3D-models for evaluating the interaction of (test) compounds with a sarbecovirus, in particular with a sarbecovirus RBD; or for evaluating the design of novel compounds mimicking the interaction of an ISVD or functional part thereof as described herein with a sarbecovirus RBD. As used herein, the term “modelling” includes the quantitative and qualitative analysis of molecular structure and/or function based on atomic structural information and interaction models. The term “modelling” includes conventional numeric-based molecular dynamic and energy minimisation models, interactive computer graphic models, modified molecular mechanics models, distance geometry and other structure-based constraint models. Molecular modelling techniques can be applied to the atomic coordinates of a sarbecovirus RBD, such as of the SARS-CoV-2 RBD domain, to derive a range of 3D models and to investigate the structure of binding sites, such as the binding sites with chemical entities. These techniques may also be used to screen for or design small and large chemical entities which are capable of binding the SARS-CoV-2 RBD domain, or with the ISVDs or functional parts thereof as disclosed herein, that may modulate the neutralization of sarbecovirus (infection). Such a screen may employ a solid 3D screening system or a computational screening system. Such modelling methods are to design or select chemical entities that possess stereochemical complementary to identified binding sites or pockets in the RBD domain. By “stereochemical complementarity” it is meant that a compound of interest makes a sufficient number of energetically favourable contacts with the RBD domain as to have a net reduction of free energy on binding to the RBD domain. By “stereochemical similarity” it is meant that the compound of interest makes about the same number of energetically favourable contacts with the RBD domain set out by a determined set of coordinates. Stereochemical complementarity is characteristic of a molecule that matches intra-site surface residues lining the groove of the receptor site as enumerated by the set of determined coordinates. By “match” is in this context meant that the identified portions interact with the surface residues, for example, via hydrogen bonding or by non-covalent Van der Waals and Coulomb interactions (with surface or residue) which promote dissolvation of the molecule within the site, in such a way that retention of the molecule at the binding site is favoured energetically. It is preferred that the stereochemical complementarity is such that the compound has a Ka for the binding site of less than 10⁻⁴ M, more preferably less than 10⁻⁹ M and more preferably 10⁻⁶ M. In a most particular embodiment, the Ka value is less than 10⁻⁸ M and more particularly less than 10⁻⁹ M.

A number of methods may be used to identify chemical entities possessing stereochemical complementarity to the structure or substructures of the RBD binding domain. For instance, the process may begin by visual inspection of a selected binding site in the RBD domain on the computer screen based on the set of determined coordinates generated from the machine-readable storage medium. Alternatively, selected fragments or chemical entities may then be positioned in a variety of orientations, or docked, within the selected binding site. Modelling software is well known and available in the art. This modelling step may be followed by energy minimization with standard available molecular mechanics force fields. Once suitable chemical entities or fragments have been selected, they can be assembled into a single compound. In one embodiment, assembly may proceed by visual inspection of the relationship of the fragments to each other on the three-dimensional image displayed on a computer screen in relation to the atomic coordinates of selected binding site or binding pocket in the RBD binding site. This can be followed by manual model building, typically using available software or in a computer-assisted manner. Alternatively, fragments may be joined to additional atoms using standard chemical geometry. The above-described evaluation process for chemical entities may be performed in a similar fashion for chemical compounds.

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.), Chemical Abstracts Service (Columbus, Ohio), the Available Chemical Directory (Symyx Technologies, Inc.), the Derwent World Drug Index (WDI), BioByteMasterFile, the National Cancer Institute database (NCI), Medchem Database (BioByte Corp.), ZINC docking database (University of California, Sterling and Irwin, J. Chem. Inf. Model, 2015), and the Maybridge catalogue. Once an entity or compound of interest has been designed or selected by the above methods, the efficiency with which that entity or compound may bind to the RBD domain or binding site can be tested and optimised by computational evaluation. An effective sarbecovirus RBD binding compound must preferably demonstrate a relatively small difference in energy between its bound and free states (i.e. a small deformation energy of binding). Thus, the most efficient RBD binding compound should preferably be designed with a deformation energy of binding of not greater than about 10 kcal/mole, particularly, not greater than 7 kcal/mole. RBD binding compounds may interact with, for instance but not limited to, the RBD domain in more than one conformation that are similar in overall binding energy. In those cases, the deformation energy of binding is taken to be the difference between the energy of the free compound and the average energy of the conformations observed when the compound binds to the protein. Further, a compound designed or selected as binding to the RBD domain may be further computationally optimised so that in its bound state it would preferably lack repulsive electrostatic interaction with the target protein.

Once a sarbecovirus RBD domain binding compound has been optimally selected or designed, as described above, substitutions may then be made in some of its atoms or side groups to improve or modify its binding properties. Generally, initial substitutions are conservative, i.e. the replacement group will have approximately the same size, shape, hydrophobicity and charge as the original group. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. It should, of course, be understood that components known in the art to alter conformation should be avoided. Such substituted chemical compounds may then be analysed for efficiency of fit to the RBD domain by the same computer methods described above.

Specific computer software is available in the art to evaluate compound deformation energy and electrostatic interaction. The screening/design methods may be implemented in hardware or software, or a combination of both. However, preferably, the methods are implemented in computer programs executing or running on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design. Each program is preferably implemented in a high-level procedural or object-oriented programming language to communicate with a computer system. However, the programs can be implemented in assembly or machine language, if desired. In any case, the language may be compiled or interpreted language. Each such computer program is preferably stored on a storage medium or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

Compounds, Test Compounds, Compounds of Interest

The term “compound” or “test compound” or “candidate compound” or “drug candidate compound” or “compound of interest” or “other binding agent” as used herein describes any molecule, different from the ISVDs (or ISVD-comprising compounds) or functional parts thereof as described herein, and either naturally occurring or synthetic that may be tested in an assay, such as a screening assay or drug discovery assay, or specifically in the method for identifying a compound capable of binding and neutralizing a sarbecovirus (infection) as described herein. As such, these compounds comprise organic and inorganic compounds. The compounds may be small molecules, chemicals, peptides, antibodies or active antibody fragments (see further).

Compounds of the present invention include both those designed or identified using an in silico screening method and those using wet-lab screening methods such as described above. Such compounds capable of binding and neutralizing a sarbecovirus may be produced using a screening method based on use of the atomic coordinates corresponding to the 3D structure of a complex of a sarbecovirus RBD with an ISVD or functional fragment thereof as presented herein. The candidate compounds and/or compounds identified or designed using a method of the present invention may be any suitable compound, synthetic or naturally occurring. In one embodiment, a synthetic compound selected or designed by the methods of the invention preferably has a molecular weight equal to or less than about 5000, 4000, 3000, 2000, 1000 or more preferably less than about 500 daltons. In another embodiment, such synthetic compound is a polypeptide, protein or peptide, or is a polypeptidic compound (comprising in part a polypeptide, protein or peptide). A compound of the present invention is preferably soluble under physiological conditions. Such compounds can comprise functional groups necessary for structural interaction with proteins, particularly hydrogen bonding, and typically include at least an amine, carbonyl, hydroxyl or carboxyl group, preferably at least two of the functional chemical groups. The compound may comprise cyclic or heterocyclic structures and/or aromatic or polyaromatic structures substituted with one or more functional groups. Compounds can also comprise biomolecules including peptides, saccharides, fatty acids, steroids, purines, pyrimidines, derivatives, structural analogues, or combinations thereof. Compounds may include, for example: (1) peptides such as soluble peptides, including Ig-tailed fusion peptides and members of random peptide libraries and combinatorial chemistry-derived molecular libraries made of D- and/or L-configuration amino acids; (2) phosphopeptides (e.g. members of random and partially degenerate, directed phosphopeptide libraries, (3) immunoglobulin variable domains or antibodies (e.g., polyclonal, monoclonal, humanized, anti-idiotypic, chimeric, and single chain antibodies, nanobodies, intrabodies, affibodies, as well as Fab, (Fab)₂, Fab expression library and epitope-binding fragments of antibodies); (4) non-immunoglobulin binding proteins such as but not restricted to avimers, DARPins, alphabodies, affitins, nanofitins, anticalins, monobodies and lipocalins; (5) nucleic acid-based aptamers; (6) small organic and inorganic molecules; and (7) polypeptidic compounds such as bicyclic peptides (also known as Bicycles®).

Synthetic compound libraries are commercially available from, for example, Maybridge Chemical Co. (Tintagel, Cornwall, UK), AMRI (Budapest, Hungary) and Chem Div (San Diego, Calif.), Specs (Delft, The Netherlands), ZINC15 (Univ. of California). In addition, numerous means are available for random and directed synthesis of a wide variety of organic compounds and biomolecules, including expression of randomized oligonucleotides. Alternatively, libraries of natural compounds in the form of bacterial, fungal, plant and animal extracts can be readily produced. In addition, natural or synthetic compound libraries and compounds can be readily modified through conventional chemical, physical and biochemical means and may be used to produce combinatorial libraries. In addition, numerous methods of producing combinatorial libraries are known in the art, including those involving biological libraries; spatially addressable parallel solid phase or solution phase libraries; synthetic library methods requiring deconvolution; the “one-bead one-compound” library method; and synthetic library methods using affinity chromatography selection. Compounds also include those that may be synthesized from leads generated by fragment-based drug design, wherein the binding of such chemical fragments is assessed by soaking or co-crystallizing such screen fragments into crystals provided by the invention and then subjecting these to an X-ray beam and obtaining diffraction data. Difference Fourier techniques are readily applied by those skilled in the art to determine the location within e.g. the sarbecovirus RBD structure at which these fragments bind, and such fragments can then be assembled by synthetic chemistry into larger compounds with increased affinity for the sarbecovirus RBD. Further, compounds identified or designed using the methods of the invention can be a peptide or a mimetic thereof. The isolated peptides or mimetics of the invention may be conformationally constrained molecules or alternatively molecules which are not conformationally constrained such as, for example, non-constrained peptide sequences. The term “conformationally constrained molecules” means conformationally constrained peptides and conformationally constrained peptide analogues and derivatives. In addition, the amino acids may be replaced with a variety of uncoded or modified amino acids such as the corresponding D-amino acid or N-methyl amino acid. Other modifications include substitution of hydroxyl, thiol, amino and carboxyl functional groups with chemically similar groups. With regard to peptides and mimetics thereof, still other examples of other unnatural amino acids or chemical amino acid analogues/derivatives can be introduced as a substitution or addition. Also, a peptidomimetic may be used. A peptidomimetic is a molecule that mimics the biological activity of a peptide but is no longer peptidic in chemical nature. By strict definition, a peptidomimetic is a molecule that no longer contains any peptide bonds (that is, amide bonds between amino acids). However, the term peptide mimetic is sometimes used to describe molecules that are no longer completely peptidic in nature, such as pseudo-peptides, semi-peptides and peptoids. Whether completely or partially non-peptide, peptidomimetics for use in the invention, provide a spatial arrangement of reactive chemical moieties that closely resembles the three-dimensional arrangement of active groups in the peptide on which the peptidomimetic is based.

For instance, a peptide or peptidomimetic may be designed as to mimic the 3D structure of the epitope described herein; and could possibly serve as an immunogen or vaccine, serving as an artificial antigen to present the conformational epitope to the immune system of a subject. Alternatively, a screening method is disclosed which screens for artificial peptide antigen molecules that specifically bind the ISVDs of the invention, as to produce a novel vaccine comprising said peptide, optionally presented in a suitable scaffold structure (some of which included in the list of possible compounds hereinabove).

Typically, as a result of this similar active-site geometry, peptidomimetics has effects on biological systems which are similar to the biological activity of the peptide. There are sometimes advantages for using a mimetic of a given peptide rather than the peptide itself, because peptides commonly exhibit two undesirable properties: (1) poor bioavailability; and (2) short duration of action. Peptide mimetics offer an obvious route around these two major obstacles, since the molecules concerned are small enough to be both orally active and have a long duration of action. There are also considerable cost savings and improved patient compliance associated with peptide mimetics, since they can be administered orally compared with parenteral administration for peptides. Furthermore, peptide mimetics are generally cheaper to produce than peptides. Naturally, those skilled in the art will recognize that the design of a peptidomimetic may require slight structural alteration or adjustment of a chemical structure designed or identified using the methods of the invention.

Pharmaceutical Compositions

A further aspect provides for a pharmaceutical composition comprising said binding agent or nucleic acid molecule, or recombinant vector as provided herein, optionally comprising a carrier, diluent, adjuvant, or excipient. A “carrier”, or “adjuvant”, in particular a “pharmaceutically acceptable carrier” or “pharmaceutically acceptable adjuvant” is any suitable carrier or adjuvant which, by themselves, do not induce the production of antibodies harmful to the individual receiving the composition nor do they elicit protection. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Preferably, a pharmaceutically acceptable carrier or adjuvant enhances the immune response elicited by an antigen. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. The term “excipient”, as used herein, is intended to include all substances which may be present in a pharmaceutical composition and which are not active ingredients but may contribute to e.g. long-term stability, or therapeutic enhancement on the active ingredient (such as by facilitating drug absorption, reducing viscosity, or enhancing solubility). Excipients include salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, such as in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles. A pharmaceutically effective amount of polypeptides, or conjugates of the invention and a pharmaceutically acceptable carrier is preferably that amount which produces a result or exerts an influence on the particular condition being treated. For therapy, the pharmaceutical composition of the invention can be administered to any patient in accordance with standard techniques. The administration can be by any appropriate mode, including oral, parenteral, topical, nasal, ophthalmic, intrathecal, intra-cerebroventricular, sublingual, rectal, vaginal, and the like. Still other techniques of formulation as nanotechnology and aerosol and inhalant are also within the scope of this invention. The dosage and frequency of administration will depend on the age, sex and condition of the patient, concurrent administration of other drugs, counter-indications and other parameters to be taken into account by the clinician. The pharmaceutical composition of this invention can be lyophilized for storage and reconstituted in a suitable carrier prior to use. When prepared as lyophilization or liquid, physiologically acceptable carrier, excipient, stabilizer need to be added into the pharmaceutical composition of the invention (Remington's Pharmaceutical Sciences 22nd edition, Ed. Allen, Loyd V, Jr. (2012). The dosage and concentration of the carrier, excipient and stabilizer should be safe to the subject (human, mice and other mammals), including buffers such as phosphate, citrate, and other organic acid; antioxidant such as vitamin C, small polypeptide, protein such as serum albumin, gelatin or immunoglobulin; hydrophilic polymer such as PVP, amino acid such as amino acetate, glutamate, asparagine, arginine, lysine; glycose, disaccharide, and other carbohydrate such as glucose, mannose or dextrin, chelate agent such as EDTA, sugar alcohols such as mannitol, sorbitol; counter-ions such as Na+, and/or surfactant such as TWEEN™, PLURONICS™ or PEG and the like. The preparation containing pharmaceutical composition of this invention should be sterilized before injection. This procedure can be done using sterile filtration membranes before or after lyophilization and reconstitution. The pharmaceutical composition can be packaged in a container or vial with sterile access port, such as an i.v. solution bottle with a rubber stopper—the pharmaceutical composition can be present as liquid, or the container or vial is filled with a liquid pharmaceutical composition that is subsequently lyophilized or dried; or can be packaged in a pre-filled syringe.

When referring to sarbecovirus hereinabove, in one embodiment SARS-CoV-1 or SARS-CoV-2 is meant.

The present invention is in particular captured by aspects and embodiments including any one or any combination of one or more aspects and embodiments as set forth in the below numbered statements:
(1) A sarbecovirus binding agent characterized in that the agent is binding to the sarbecovirus spike protein Receptor Binding Domain (SPRBD), is allowing binding of Angiotensin-Converting Enzyme 2 (ACE2) to SPRBD when the sarbecovirus binding agent itself is bound to SPRBD, is at least neutralizing SARS-CoV-2 and SARS-CoV-1, and is binding to at least one of the amino acids Thr393 (or alternatively Ser393 in some sarbecoviruses), Asn394 (or alternatively Ser394 in some sarbecoviruses), Val395, or Tyr396 of the SARS-CoV-2 spike protein as defined in SEQ ID NO:30.
(2) The sarbecovirus binding agent according to (1) which is neutralizing SARS-CoV-2 and/or SARS-CoV-1 in a pseudotype virus neutralization assay with an IC₅₀ of 10 μg/mL or less.
(3) The sarbecovirus binding agent according to (1) which is further allowing binding of antibodies VHH72, 5309, or CB6 to SPRBD when the sarbecovirus binding agent itself is bound to SPRBD.
(4) The sarbecovirus binding agent according to any one of (1) to (3) which is further binding to at least one of the amino acids Ser514, Glu516, or Leu518 of the SARS-CoV-2 spike protein as defined in SEQ ID NO:30.
(5) The sarbecovirus binding agent according to any one of (1) to (4) which is further binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses), Arg466 or Arg357 (or alternatively Lys357 in some sarbecoviruses) of the SARS-CoV-2 spike protein as defined in SEQ ID NO:30.
(6) The sarbecovirus binding agent according to any of (1) to (5) which is comprising an immunoglobulin single variable domain or functional part thereof.
(7) The sarbecovirus binding agent according to any of (1) to (6) characterized in that it is comprising the complementarity determining regions (CDRs) present in any of SEQ ID NOs: 1 to 5, wherein the CDRs are annotated according to Kabat, MacCallum, IMGT, AbM, aHo, Chothia, Gelfand, or Honegger.
(8) The sarbecovirus binding agent according to (7) wherein CDR1 is defined by SEQ ID NO:6, CDR2 defined by SEQ ID NO:7, and CDR3 defined by SEQ ID NO:8, wherein the annotations are according to Kabat.
(9) The sarbecovirus binding agent according to (8) wherein CDR1 is selected from the sequences defined by SEQ ID NO: 9 or 10, CDR2 is selected from the sequences defined by SEQ ID NO: 11 to 14, and CDR3 is selected from the sequences defined by SEQ ID NO:15 or 16.
(10) The sarbecovirus binding agent according to any of (7) to (9) further comprising:
a framework region 1 (FR1) defined by SEQ ID NO:17, an FR2 defined by SEQ ID NO:18, an FR3 defined by SEQ ID NO:19, and an FR4 defined by SEQ ID NO:20; or
an FR1 selected from the sequences defined by SEQ ID NO: 21 to 23, an FR2 defined by SEQ ID NO:18, an FR3 selected from the sequences defined by SEQ ID NO: 24 to 27, and an FR4 selected from the sequences defined by SEQ ID NO: 28 or 29; or
FR1, FR2, FR3 and FR4 regions that together have an amino acid sequence that is at least 90% amino acid identical to a combination of an FR1 selected from the sequences defined by SEQ ID NO: 21 to 23, an FR2 defined by SEQ ID NO:18, an FR3 selected from the sequences defined by SEQ ID NO: 24 to 27, and an FR4 selected from the sequences defined by SEQ ID NO: 28 or 29.
(11) The sarbecovirus binding agent according to any one of (7) to (10) which is comprising or consisting of an immunoglobulin single variable domain (ISVD) defined by any of SEQ ID NOs: 1 to 5, or defined by any amino acid sequence that is at least 90% amino acid identical to any of SEQ ID NOs: 1 to 5, wherein the non-identical amino acids are located in one or more FRs.
(12) An isolated nucleic acid encoding a sarbecovirus binding agent according to any one of (6) to (11).
(13) A recombinant vector comprising the nucleic acid according to (12).
(14) A pharmaceutical composition comprising a sarbecovirus binding agent according to any one of (1) to (11), an isolated nucleic acid according to (12) and/or a recombinant vector according to (13).
(15) The sarbecovirus binding agent according to any one of (1) to (11), the isolated nucleic acid according to (12), the recombinant vector according to (13), or the pharmaceutical composition according to (14) for use as a medicament.
(16) The sarbecovirus binding agent according to any one of (1) to (11), the isolated nucleic acid according to (12), the recombinant vector according to (13), or the pharmaceutical composition according to (14) for use in the treatment of a sarbecovirus infection.
(17) The sarbecovirus binding agent according to any one of (1) to (11), the isolated nucleic acid according to (12), the recombinant vector according to (13), or the pharmaceutical composition according to (14) for use in passive immunisation of a subject.
(18) The sarbecovirus binding agent, the isolated nucleic acid, the recombinant vector, or the pharmaceutical composition for use according (17) wherein the subject is having a sarbecovirus infection, or wherein the subject is not having a sarbecovirus infection.
(19) The sarbecovirus binding agent according to any one of (1) to (11) for use in diagnosing a sarbecovirus infection.
(20) The sarbecovirus binding agent according to any one of (1) to (11), the isolated nucleic acid according to (12), or recombinant vector according to (13) for use in the manufacture of a diagnostic kit.
(21) The sarbecovirus binding agent according any of the preceding claims wherein the sarbecovirus is SARS-CoV-1 or SARS-CoV-2.
(1′) A sarbecovirus binding agent characterized in that the agent is binding to the sarbecovirus spike protein Receptor Binding Domain (SPRBD), is allowing binding of Angiotensin-Converting Enzyme 2 (ACE2) to SPRBD when the sarbecovirus binding agent itself is bound to SPRBD, is at least neutralizing SARS-CoV-2 and SARS-CoV-1, and is binding to:
   at least one of the amino acids Thr393 (or alternatively Ser393 in some sarbecoviruses), Asn394 (or alternatively Ser394 in some sarbecoviruses), Val395, or Tyr396 of the SARS-CoV-2 spike protein as defined in SEQ ID NO:30; and
   at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses), Arg466, or Arg357 (or alternatively Lys357 in some sarbecoviruses) of the SARS-CoV-2 spike protein as defined in SEQ ID NO:30.
(2′) The sarbecovirus binding agent according to (1′) which is binding to at least amino acids Asn394 (or alternatively Ser394 in some sarbecoviruses) and Tyr396.
(3′) The sarbecovirus binding agent according to (1′) or (2′) which is binding to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses), or Arg466 of the SARS-CoV-2 spike protein as defined in SEQ ID NO:30.
(4′) The sarbecovirus binding agent according to any one (1′) to (3′) which is further binding to at least one of the amino acids Ser514, Glu516, or Leu518 of the SARS-CoV-2 spike protein as defined in SEQ ID NO:30.
(5′) The sarbecovirus binding agent according to (4′) which is binding to at least amino acids Ser514 and Glu516.
(6′) The sarbecovirus binding agent according to any one of (1′) to (5′) which is further binding to the amino acid Arg355 of the SARS-CoV-2 spike protein as defined in SEQ ID NO:30.
(7′) A sarbecovirus binding agent characterized in that the agent is binding to the sarbecovirus spike protein Receptor Binding Domain (SPRBD), is allowing binding of Angiotensin-Converting Enzyme 2 (ACE2) to SPRBD when the sarbecovirus binding agent itself is bound to SPRBD, is at least neutralizing SARS-CoV-2 and SARS-CoV-1, and is binding to at least one, or in increasing order of preference at least two, at least three, or at least four, of the amino acids Asn394 (or alternatively Ser394 in some sarbecoviruses), Tyr396, Phe464, Ser514, Glu516, and Arg355 of the SARS-CoV-2 spike protein as defined in SEQ ID NO:30;
optionally is further binding to amino acid Arg357 (or alternatively Lys357 in some sarbecoviruses) and/or Lys462 (or alternatively Arg462 in some sarbecoviruses) and/or Glu465 (or alternatively Gly465 in some sarbecoviruses) and/or Arg466 and/or Leu518.
(8′) The sarbecovirus binding agent according to any one of (1′) to (7′), which is neutralizing a SARS-CoV-2 variant comprising a mutation at position N439, K417, 5477, L452, T478, E484, P384, N501 and/or D614 of the SARS-CoV-2 spike protein as defined in SEQ ID NO:30.
(9′) The sarbecovirus binding agent according to any one of (1′) to (8′) which is neutralizing SARS-CoV-2 and/or a SARS-CoV-2 variant and/or SARS-CoV-1 in a pseudotype virus neutralization assay with an IC₅₀ of 10 μg/mL or less.
(10′) The sarbecovirus binding agent according to any one of (1′) to (9′), which is inducing 51 shedding.
(11′) The sarbecovirus binding agent according to any one of (1′) to (10′) which is further allowing binding of antibodies VHH72, 5309, or CB6 to SPRBD when the sarbecovirus binding agent itself is bound to SPRBD.
(12′) The sarbecovirus binding agent according to any of the preceding claims which is comprising an immunoglobulin single variable domain or functional part thereof.
(13′) The sarbecovirus binding agent according to any of the preceding claims characterized in that it is comprising the complementarity determining regions (CDRs) present in any of SEQ ID NOs: 1 to 5 or SEQ ID NO: 53-55, wherein the CDRs are annotated according to Kabat, MacCallum, IMGT, AbM, or Chothia.
(14′) The sarbecovirus binding agent according to (13′) wherein CDR1 is defined by SEQ ID NO:6, CDR2 defined by SEQ ID NO:7, and CDR3 defined by SEQ ID NO:8, wherein the annotations are according to Kabat.
(15′) The sarbecovirus binding agent according to (14′) wherein CDR1 is selected from the sequences defined by SEQ ID NO: 9 or 10, CDR2 is selected from the sequences defined by SEQ ID NO: 11 to 14, and CDR3 is selected from the sequences defined by SEQ ID NO:15 or 16.
(16′) The sarbecovirus binding agent according to any of (13′) to (15′) further comprising:
   a framework region 1 (FR1) defined by SEQ ID NO:17, an FR2 defined by SEQ ID NO:18, an FR3 defined by SEQ ID NO:19, and an FR4 defined by SEQ ID NO:20; or
   an FR1 selected from the sequences defined by SEQ ID NO: 21 to 23, an FR2 defined by SEQ ID NO:18, an FR3 selected from the sequences defined by SEQ ID NO: 24 to 27, and an FR4 selected from the sequences defined by SEQ ID NO: 28 or 29; or
   FR1, FR2, FR3 and FR4 regions that together have an amino acid sequence that is at least 90% amino acid identical to a combination of an FR1 selected from the sequences defined by SEQ ID NO: 21 to 23, an FR2 defined by SEQ ID NO:18, an FR3 selected from the sequences defined by SEQ ID NO: 24 to 27, and an FR4 selected from the sequences defined by SEQ ID NO: 28 or 29.
(17′) The sarbecovirus binding agent according to any one of (13′) to (16′) which is comprising or consisting of an immunoglobulin single variable domain (ISVD) defined by any of SEQ ID NOs: 1 to 5, or defined by any amino acid sequence that is at least 90% amino acid identical to any of SEQ ID NOs: 1 to 5, wherein the non-identical amino acids are located in one or more FRs.
(18′) The sarbecovirus binding agent according to (13′) wherein CDR1 is defined by SEQ ID NO:76, CDR2 defined by SEQ ID NO:77, and CDR3 defined by SEQ ID NO:78, wherein the annotations are according to Kabat.
(19′) The sarbecovirus binding agent according to (18′) wherein CDR1 is selected from the sequences defined by SEQ ID NO: 69 or 70, CDR2 is selected from the sequences defined by SEQ ID NO: 71 or 82, and CDR3 is selected from the sequences defined by SEQ ID NO:73 to 75.
(20′) The sarbecovirus binding agent according to (18′) or (19′) further comprising:
   a framework region 1 (FR1) defined by SEQ ID NO:82, an FR2 defined by SEQ ID NO:86, an FR3 defined by SEQ ID NO:90, and an FR4 defined by SEQ ID NO:94; or
   an FR1 selected from the sequences defined by SEQ ID NO: 79 to 81, an FR2 defined by SEQ ID NO:83 to 85, an FR3 selected from the sequences defined by SEQ ID NO: 87 to 89, and an FR4 selected from the sequences defined by SEQ ID NO: 91 to 93; or
   FR1, FR2, FR3 and FR4 regions that together have an amino acid sequence that is at least 90% amino acid identical to a combination of an FR1 selected from the sequences defined by SEQ ID NO: 19 to 81, an FR2 defined by SEQ ID NO:83 to 85, an FR3 selected from the sequences defined by SEQ ID NO: 87 to 89, and an FR4 selected from the sequences defined by SEQ ID NO: 91 to 93.
(21′) The sarbecovirus binding agent according to any one of (18′) to (20′) which is comprising or consisting of an immunoglobulin single variable domain (ISVD) defined by any of SEQ ID NOs: 53 to 55, or defined by any amino acid sequence that is at least 90% amino acid identical to any of SEQ ID NOs: 53 to 55, wherein the non-identical amino acids are located in one or more FRs.
(22′) A multivalent or multispecific sarbecovirus binding agent, wherein one or more of the binding agents according to any one of (1′) to (21′) are fused directly or via a linker, preferably fused via an Fc domain.
(23′) An isolated nucleic acid encoding a sarbecovirus binding agent according to any one (12′) to (21′).
(24′) A recombinant vector comprising the nucleic acid according tp (23′).
(25′) A pharmaceutical composition comprising a sarbecovirus binding agent according to any one of (1′) to (21′), a multivalent or multispecific sarbecovirus binding agent according to (22′), an isolated nucleic acid according to (23′) and/or a recombinant vector according to (24′).
(26′) The sarbecovirus binding agent according to any one of (1′) to (21′), the multivalent or multispecific sarbecovirus binding agent according to (22′), the isolated nucleic acid according to (23′), the recombinant vector according to (24′), or the pharmaceutical composition according to (25′) for use as a medicament.
(27′) The sarbecovirus binding agent according to any one of (1′) to (21′), the multivalent or multispecific sarbecovirus binding agent according to (22′), the isolated nucleic acid according to (23′), the recombinant vector according to (24′), or the pharmaceutical composition according to (25′) for use in the treatment of a sarbecovirus infection.
(28′) The sarbecovirus binding agent according to any one of (1′) to (21′), the multivalent or multispecific sarbecovirus binding agent according to (22′), the isolated nucleic acid according (23′), the recombinant vector according to (24′), or the pharmaceutical composition according to (25′) for use in passive immunisation of a subject.
(29′) The sarbecovirus binding agent, the isolated nucleic acid, the recombinant vector, or the pharmaceutical composition for use according to (28′) wherein the subject is having a sarbecovirus infection, or wherein the subject is not having a sarbecovirus infection.
(30′) The sarbecovirus binding agent according to any one of (1′) to (21′) or the multivalent or multispecific sarbecovirus binding agent according to (22′) for use in diagnosing a sarbecovirus infection.
(31′) The sarbecovirus binding agent according to any one of (1′) to (21′), the multivalent or multispecific sarbecovirus binding agent according to (22′), the isolated nucleic acid according to (23′), or recombinant vector according to (24′), for use in the manufacture of a diagnostic kit.
(32′) The sarbecovirus binding agent according any of the preceding claims wherein the sarbecovirus is SARS-CoV-1 or SARS-CoV-2.

### Definitions

The following terms or definitions are provided solely to aid in the understanding of the invention.

Where an indefinite or definite article is used when referring to a singular noun e.g. “a” or “an”, “the”, this includes a plural of that noun unless something else is specifically stated.

Where the term “comprising” is used herein, it does not exclude other elements or steps. The term comprising thus encompasses but is broader than the term “consisting”, or “consisting of” which is limiting. For example, “comprising A” can mean consisting of A, consisting of A and B, consisting of A, B, C, etc.; whereas “comprising A and B” can mean consisting of A and B, consisting of A, B, C, etc. Furthermore, the terms first, second, third and the like are used herein for distinguishing between similar elements and not necessarily for describing a sequential or chronological order. It is to be understood that the terms so used are interchangeable under appropriate circumstances and that the embodiments of the invention as described herein are capable of operation in other sequences than described or illustrated herein.

Unless specifically defined, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology, John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

“Nucleic acid(s)” or “nucleic acid molecule(s)” as used herein refers to a polymeric form of nucleotides of any length, either ribonucleotides or deoxyribonucleotides; the sequential linear arrangement of the nucleotides together resulting in/forming the “nucleotide sequence”, “DNA sequence”, or “RNA sequence”. This term refers only to the primary structure of the molecule. Thus, this term includes double- and single-stranded DNA, and RNA. It also includes known types of modifications, for example, methylation, “caps”, and substitution of one or more of the naturally occurring nucleotides with an analog. Modifications to nucleic acids can be introduced at one or more levels: phosphate linkage modification (e.g. introduction of one or more of phosphodiester, phosphoramidate or phosphorothioate bonds), sugar modification (e.g. introduction of one or more of LNA (locked nucleic acids), 2′-O-methyl, 2′-O-methoxy-ethyl, 2′-fluoro, S-constrained ethyl or tricyclo-DNA and/or non-ribose modifications (e.g. introduction of one or more of phosphorodiamidate morpholinos or peptide nucleic acids).

By “nucleic acid construct” it is meant a nucleic acid molecule that has been constructed in order to comprise one or more functional units not found together in nature, thus having a nucleotide sequence not found in nature (non-native nucleotide sequence). Examples include circular, linear, double-stranded, extrachromosomal DNA molecules (plasmids), cosmids (plasmids containing COS sequences from lambda phage), viral genomes comprising non-native nucleic acid sequences, and the like.

A “coding sequence” is a nucleotide sequence that can be transcribed into mRNA and/or translated into a polypeptide when placed under the control of appropriate (gene) regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

With a “chimeric gene” or “chimeric construct” or “chimeric gene construct” is interchangeably meant a recombinant nucleic acid sequence in which a (gene) promoter or regulatory nucleic acid sequence is operably or operatively linked to, or associated with, a nucleic acid sequence of interest that codes for an RNA (e.g. a coding sequence, an shRNA, etc.), such that the regulatory nucleic acid sequence is able to regulate transcription or expression of the nucleic acid of interest. The operable or operative linkage in a chimeric gene between the regulatory nucleic acid sequence and the nucleic acid sequence of interest is not found in nature.

An “expression cassette” comprises any nucleic acid construct capable of directing the expression of a gene/coding sequence of interest, which is operably linked to a (gene) promoter. Expression cassettes are generally DNA constructs preferably including (5′ to 3′ in the direction of transcription): a (gene) promoter region, a polynucleotide sequence of interest with a transcription initiation region, and a termination sequence including a stop signal for RNA polymerase and a polyadenylation signal; all these elements being operably or operatively linked meaning that all of these regions should be capable of operating (being expressed) in a cell, such as prokaryotic (e.g. bacterial) or eukaryotic (e.g. mammalian, yeast, insect, fungal, plant, algal) cells, when transformed into that cell. The promoter region comprising the transcription initiation region, which preferably includes the RNA polymerase binding site, and the polyadenylation signal may be native to the cell to be transformed, may be derived from an alternative source, or may be synthetic, as long as it is functional in the cell. Such expression cassettes can be constructed in e.g. a “vector” or “expression vector” (linear or circular nucleic acids, plasmids, cosmids, viral vectors, phagemids, etc.).

The term “vector”, “vector construct”, “expression vector”, “recombinant vector” or “gene transfer vector”, as used herein, is intended to refer to a nucleic acid molecule capable of carrying another nucleic acid molecule to which it has been linked. More particular, said vector may include any vector known to the skilled person, including any suitable type, but not limited to, for instance, plasmid vectors, cosmid vectors, phage vectors, such as lambda phage, viral vectors, even more particular a lentiviral, adenoviral, AAV or baculoviral vectors, or artificial chromosome vectors such as bacterial artificial chromosomes (BAC), yeast artificial chromosomes (YAC), or P1 artificial chromosomes (PAC). Said vectors may include a cloning or expression vector, as well as a delivery vehicle such as a viral, lentiviral or adenoviral vector. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments. The construction of expression vectors for use in transfecting cells is also well known in the art, and thus can be accomplished via standard techniques (see, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clif ton, N.J.), and the Ambion 1998 Catalog (Ambion, Austin, Tex.).

Nucleic acids, vectors, etc. encoding a binding agent as described herein can be employed in a therapeutic setting. Such nucleic acid, vector, etc. can be administered through gene therapy or RNA vaccination. “Gene therapy” as used herein refers to therapy performed by the administration to a subject of an expressed or expressible nucleic acid. For such applications, the nucleic acid molecule or vector as described herein allow for production of the binding agent within a cell. A large set of methods for gene therapy are available in the art and include, for instance (adeno-associated) virus mediated gene silencing, or virus mediated gene therapy (e.g. US 20040023390; Mendell et al 2017, N Eng J Med 377:1713-1722). A plethora of delivery methods are well known to those of skill in the art and include but are not limited to viral delivery systems, microinjection of DNA plasmids, biolistics of naked nucleic acids, use of a liposome or an artificial exosome, administration of the nucleic acid or vector formulated in a nanoparticle or lipid or lipid-comprising particle. In vivo delivery by administration to an individual patient occurs typically by systemic administration (e.g., intravenous, intraperitoneal infusion or brain injection; e.g. Mendell et al 2017, N Eng J Med 377:1713-1722). An “RNA vaccine” or “messenger RNA vaccine” or “mRNA vaccine” relies on RNA, mRNA or synthetic (m)RNA encoding the antigen (or antigens) of interest. Administration of an RNA vaccine or vaccination with an RNA vaccine results in in vivo production of the antigen (or antigens) of interest by cells of the subject to which the RNA vaccine is administered. The subject's immune system subsequently can mount an immune response to this antigen(s).

The terms “protein”, “polypeptide”, and “peptide” are interchangeably used herein to refer to a polymer of amino acid residues and to variants and synthetic analogues of the same; the sequential linear arrangement of the amino acids together resulting in/forming the “amino acid sequence” or “protein sequence”. A “peptide” may also be referred to as a partial amino acid sequence derived from its original protein, for instance after enzymatic (e.g. tryptic) digestion. These terms apply to naturally-occurring amino acid polymers as well as to amino acid polymers in which one or more amino acid residues is a synthetic non-naturally occurring amino acid, such as a chemical analogue of a corresponding naturally occurring amino acid. Also included are proteins comprising one or more posttranslational modifications such as covalent addition of functional groups or proteins (such as glycosylation, phosphorylation, acetylation, ubiquitination, methylation, lipidation and nitrosylation) or such as proteolytic processing. Based on the amino acid sequence and the modifications, the atomic or molecular mass or weight of a polypeptide is expressed in (kilo)dalton (kDa). A further modification of proteins includes addition of a tag, such as a His-tag or sortag. By sortagging (sortase-mediated transpeptidation; Popp et al. 2007, Nat Chem Biol 3:707-708) for instance, a multi-arm PEG nanobody neutralizing SARS-CoV2 was constructed (Moliner-Morro et al. 2020, Biomolecules 10:1661).

A “protein domain” is a distinct functional and/or structural unit in or part of a protein. Usually, a protein domain is responsible for a particular function or interaction, contributing to the overall (biological) role of a protein. Domains may exist in a variety of biological contexts, where similar domains can be found in different proteins with similar or different functions. Protein domains can have a rigid 3D-structure if confined by e.g. a number of intramolecular cysteines (e.g. cysteine-knot proteins) or can, depending on e.g. presence or absence of a bound ligand or e.g. presence or absence of a posttranslational modification, assume different 3D-conformations, or can have a less defined, more fluid 3D-structure.

Amino acids are presented herein by their 3- or 1-lettercode nomenclature as defined and provided also in the IUPAC-IUB Joint Commission on Biochemical Nomenclature (Nomenclature and Symbolism for Amino Acids and Peptides. Eur. J. Biochem. 138: 9-37 (1984)); as follows: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

By “isolated” or “purified” is meant material that is substantially or essentially free from components that normally accompany it in its native state. For example, an “isolated polypeptide” or “purified polypeptide” refers to a polypeptide which has been isolated or purified by any suitable means from a mixture of molecules comprising the to be isolated or to be purified polypeptide of interest. An isolated or purified polypeptide of interest can for instance be an immunoglobulin, antibody or nanobody, and the mixture can be a mixture or molecules as present in a cell producing the immunoglobulin, antibody or nanobody, and/or the culture medium into which the immunoglobulin, antibody or nanobody is secreted into (likely together with other molecules secreted by the cell). An isolated protein or peptide can be generated by chemical protein synthesis, by recombinant production or by purification from a complex sample. A similar explanation applies to “isolated nucleic acids” or “isolated nucleic acid molecules”.

The term “fused to”, as used herein, and interchangeably used herein as “connected to”, “conjugated to”, “ligated to” refers in one aspect to “genetic fusion”, e.g., by recombinant DNA technology, as well as to “chemical and/or enzymatic conjugation” resulting in a stable covalent link between two nucleic acid molecules. The same applies for the term “inserted in”, wherein a fragment of one nucleic acid may be inserted in a second nucleic acid molecule by fusing or ligating the two sequences genetically, enzymatically or chemically. Peptides or polypeptides can likewise be fused or connected to one another, such as via peptide bonds or via linking one peptide to a side chain of an amino acid in a second peptide.

The term “wild-type” or “native” refers to a gene or gene product isolated from a naturally occurring source. A wild-type gene is that which is most frequently observed in a population and is thus arbitrarily designed the “normal” or “wild-type” form of the gene or gene product. In contrast, the term “modified”, “mutant”, “engineered” or “variant” refers to a gene or gene product that displays modifications (such as a substitution, mutation or variation) in sequence, post-translational modifications and/or modification of biological or functional properties (i.e., altered characteristics) when compared to the wild-type gene or gene product. It is noted that naturally occurring mutants or variants can be isolated; these are identified by the fact that they have altered characteristics when compared to the wild-type gene or gene product. The altered characteristics can solely reside at the sequence level, or can additionally confer altered biological and/or functional properties to the mutants or variants compared to the wild-type gene or gene product. It is understood that conservative amino acid substitutions can be introduced in a protein or polypeptide whereby such substitutions have no essential or substantial effect on the protein's activity. A “homologue”, or “homologues” of a protein of interest encompass(es) proteins having amino acid substitutions, deletions and/or insertions relative to an unmodified (e.g. native, wild-type) protein of interest and having essentially or substantially similar biological and functional activity as the unmodified protein from which it is/they are derived.

A “percentage (of) sequence identity” is calculated by comparing two optimally aligned (amino acid or nucleic acid) sequences over the window of comparison, determining the number of positions at which the identical amino acid or nucleotide residue occurs in both sequences to yield the number of matched positions, dividing the number of matched positions by the total number of positions in the window of comparison (i.e., the window size), and multiplying the result by 100 to yield the percentage of (amino acid or nucleic acid) sequence identity.

The term “molecular complex” or “complex” refers to a molecule associated with at least one other molecule, which may e.g. be another protein or a chemical entity. The term “associated with” refers to a condition of proximity between (parts or portions of) two entities of a molecular complex. The association maybe non-covalent—wherein the juxtaposition is energetically favored by hydrogen bonding or van der Waals or electrostatic interactions—or it may be covalent. The term “chemical entity” refers to chemical compounds, complexes of at least two chemical compounds, and fragments of such compounds or complexes. The chemical entity may be, for example, a ligand, a substrate, a phosphate, a nucleotide, an agonist, antagonist, inhibitor, antibody, a single domain antibody, drug, peptide, peptidomimetic, protein or compound.

As used herein, the term “crystal” means a structure (such as a three-dimensional (3D) solid aggregate) in which the plane faces intersect at definite angles and in which there is a regular structure (such as an internal structure) of the constituent chemical species. The term “crystal” refers in particular to a solid physical crystal form such as an experimentally prepared crystal. The term “co-crystal” as used herein refers to a structure that consist of two or more components that form a unique crystalline structure having unique properties, wherein the components may be atoms, ions or molecules. In the context of current application, a co-crystal comprising an RBD domain of a Corona virus S protein and a herein described binding agent/immunoglobulin single variant domain (ISVD) is equivalent to a crystal of the RBD domain in complex with the herein described binding agent/ISVD. The term “crystallization solution” refers to a solution which promotes crystallization comprising at least one agent such as a buffer, one or more salts, a precipitating agent, one or more detergents, sugars or organic compounds, lanthanide ions, a poly-ionic compound, a stabilizer, or combinations of two or more of such agents.

The terms “suitable conditions” refers to the environmental factors, such as temperature, movement, other components, and/or “buffer condition(s)” among others, wherein “buffer conditions” refer specifically to the composition of the solution in which the molecules are present. A composition includes buffered solutions and/or solutes such as pH buffering substances, water, saline, physiological salt solutions, glycerol, preservatives, etc. for which a person skilled in the art is aware of the suitability to obtain optimal assay performance. Suitable conditions as used herein could also refer to suitable binding conditions, for instance when Nbs are aimed to bind a RBD. Suitable conditions as used herein could also refer to suitable crystallization or cryo-EM conditions, which may alternatively mean suitable conditions wherein the aimed structural analysis is expected. Suitable conditions may further relate to buffer conditions in which thermal stability assays can be performed.

The term “binding pocket” or “binding site” refers to a region of a molecule or molecular complex, that, as a result of its shape and charge, associates with (see above) another chemical entity, compound, protein, peptide, antibody, single domain antibody or ISVD. For antibody-related molecules, the term “epitope” or “conformational epitope” is also used interchangeably herein and refers to the binding pocket or binding site of the protein to which an immunoglobulin (or part thereof), antibody or ISVD is binding. The term “pocket” includes, but is not limited to cleft, channel or site. The RBD domain of a Corona virus comprises binding pockets or binding sites for e.g. ACE-2 and for many different neutralizing and non-neutralizing antibodies or nanobodies. The term “part of a binding pocket/site” refers to less than all of the amino acid residues that define the binding pocket, binding site or epitope. For example, the atomic coordinates of residues that constitute part of a binding pocket may be specific for defining the chemical environment of the binding pocket, or useful in designing fragments of a molecule that may interact with those residues. For example, the portion of residues may be key residues that themselves are (directly) involved in ligand binding; or may be residues that define a three-dimensional compartment of the binding pocket in order for the ligand to bind to the key residues and not necessarily directly involved in ligand binding. The residues, such as amino acids, may be contiguous or non-contiguous in a primary sequence, such as amino acid sequence.

“Binding” means any interaction, be it direct or indirect. A direct interaction implies a contact (e.g. physical or chemical) between two binding partners. An indirect interaction means any interaction whereby the interaction partners interact in a complex of more than two molecules. An interaction can be completely indirect (e.g. two molecules are part of the same complex with the help of one or more bridging molecules but don't bind in the absence of the bridging molecule(s)). An interaction may be partly direct or partly indirect: there is still a direct contact between two interaction partners, but such contact is e.g. not stable, and is stabilized by the interaction with one or more additional molecules.

“Specificity of binding” or “binding specificity” or “specifically binding” refers to the situation in which a molecule A is, at a certain concentration (e.g. sufficient to inhibit or neutralize a protein or process of interest) binding to a target of interest (e.g. protein) with higher affinity (e.g. at least 2-fold, 5-fold, or at least 10-fold higher affinity, e.g. at least 20-, 50- or 100-fold or more higher affinity) than the affinity with which it is possibly (if at all) binding to other targets (targets not of interest). Specific binding does not mean exclusive binding. However, specific binding does mean that a binder has a certain increased affinity or preference for one or a few of its targets. Exclusivity of binding refers to the situation in which a binder is binding only to the target of interest.

The term “affinity”, as used herein, generally refers to the degree to which one molecule (e.g. ligand, chemical, protein or peptide) binds to another molecule (e.g. (target) protein or peptide) so as to shift the equilibrium of single molecule monomers towards a complex formed by (specific)(non-covalent) binding of the two molecules. Non-covalent interaction or binding between 2 or more binding partners may involve interactions such as van der Waals interaction, hydrogen bonding, and salt bridges.

A “binding agent” relates to a molecule that is capable of binding to at least one other molecule, wherein said binding is preferably a specific binding, such as on a defined binding site, pocket or epitope. The binding agent may be of any nature or type and is not dependent on its origin. The binding agent may be chemically synthesized, naturally occurring, recombinantly produced (and optionally purified), as well as designed and synthetically produced (and optionally purified). Said binding agent may hence be a small molecule, a chemical, a peptide, a polypeptide, an antibody, or any derivative of any thereof, such as a peptidomimetic, an antibody mimetic, an active fragment, a chemical derivative, among others. A functional fragment of a binding agent or a functional part of a binding agent refers to a fragment or part of that binding agent that is functionally equivalent to that binding agent. In particular, such functional fragment or part of a binding agent as described herein ideally retains one or more of the functional features (1) to (126) of that binding agent as outlined extensively hereinabove. Well-known functional fragments of antibodies, for example, are Fab-fragments, scFv-fragments, etc.

An “epitope”, as used herein, refers to an antigenic determinant of a polypeptide, constituting a binding site or binding pocket on a target molecule, such as a Corona virus RBD domain, more particularly a 2019-nCoV RBD domain. An epitope could comprise 3 amino acids in a spatial conformation (linear or conformational), which is unique to the epitope. Generally, an epitope consists of at least 4, 5, 6, 7 amino acids, and more usually, consists of at least 8, 9, or 10 amino acids.

A “linear epitope” is an epitopes that is linear in nature, or that can be mimicked by linear (poly)peptides, indicating that a stretch of (continuous) amino acids as contained in a protein or polypeptide is forming the epitope. A common way to identify linear epitopes is peptide scanning wherein the protein or polypeptide of interest and known to contain an epitope for a binding agent is divided in a set of overlapping peptides (usually chemically synthesized) which all are tested for binding with the binding agent. From the peptide(s) out of the set of overlapping peptides that bind with the binding agent, the location of the epitope can be derived. If none of the peptide(s) out of the set of overlapping peptides is binding with the binding agent, then the epitope is likely not to be a linear epitope but to be a conformational epitope which cannot be mimicked by simple linear peptides.

A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state, such as in a linear peptide). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., a-helix, (3-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as phosphorylation, glycosylation, ubiquitination, nitrosylation, methylation, acetylation, lipidation, ligand binding, sulf(on)ation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein, or the spatial conformation of amino acids in a protein, may be determined by either functional assay for activity or binding to another molecule or by means of physical methods such as X-ray crystallography, (multi-dimensional) nuclear magnetic resonance (NMR), spin labeling, or cryo-EM among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “antibody” refers to an immunoglobulin (Ig) molecule or a molecule comprising an immunoglobulin (Ig) domain, which specifically binds with an antigen. “Antibodies” can further be intact immunoglobulins derived from natural sources or from recombinant sources and can be immunoreactive portions of intact immunoglobulins. The term “active antibody fragment” refers to a portion of any antibody or antibody-like structure that by itself has high affinity for an antigenic determinant, or epitope, and contains one or more CDRs accounting for such specificity. Non-limiting examples include immunoglobulin domains, Fab, F(ab)′2, scFv, heavy-light chain dimers, immunoglobulin single variable domains, Nanobodies (or VHH antibodies), domain antibodies, and single chain structures, such as a complete light chain or complete heavy chain.

The term “antibody fragment” and “active antibody fragment” as used herein refer to a protein comprising an immunoglobulin domain or an antigen binding domain capable of specifically binding a spike protein, or to an RBD domain present in the spike protein of a sarbecovirus, such as the SARS-CoV-2 virus. Antibodies are typically tetramers of immunoglobulin molecules. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs), and in particular the CDRs therein, even more particular CDR3 therein, that confer specificity to an antibody for the antigen by carrying the antigen or epitope-binding site. Typically, in conventional immunoglobulins, a heavy chain variable domain (VH) and a light chain variable domain (VL) interact to form an antigen binding site. In this case, the complementarity determining regions (CDRs) of both VH and VL contribute (although not necessarily evenly) to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An “immunoglobulin single variable domain” (or “ISVD”) as used herein, refers to a protein with an amino acid sequence comprising 4 Framework regions (FR) and 3 complementary determining regions (CDR) according to the format of FR1-CDR1-FR2-CDR2-FR3-CDR3-FR4.

An “immunoglobulin domain” of this invention refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” (or an amino acid sequence that is suitable for use as a dAb) or a Nanobody (as defined herein, and including but not limited to a VHH); other single variable domains, or any suitable fragment of any one thereof. In particular, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al. 1993, Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VH Hs and Nanobody, reference is made to the review article by Muyldermans 2001 (Rev Mol Biotechnol 74: 277-302), as well as to the following patent applications, which are mentioned as general background art: WO 94/04678, WO 95/04079, WO 96/34103, WO 94/25591, WO 99/37681, WO 00/40968, WO 00/43507, WO 00/65057, WO 01/40310, WO 01/44301, EP 1134231, WO 02/48193, WO 97/49805, WO 01/21817, WO 03/035694, WO 03/054016, WO 03/055527, WO 03/050531, WO 01/90190, WO 03/025020 (=EP 1433793), WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references, Nanobody (in particular VHH sequences and partially humanized Nanobody) can in particular be characterized by the presence of one or more “hallmark residues” in one or more of the framework sequences. For numbering of the amino acid residues of any IVD different numbering schemes can be applied. For example, numbering can be performed according to the AHo numbering scheme for all heavy (VH) and light chain variable domains (VL) given by Honegger & Pluckthun 2001 (J Mol Biol 309:657-70), as applied to VHH domains from camelids. Alternative methods for numbering the amino acid residues of VH domains, which can also be applied in an analogous manner to VHH domains, are known in the art. For example, the delineation of the FR and CDR sequences can be done by using the Kabat numbering system as applied to VHH domains from camelids by Riechmann & Muyldermans 1999 (J Immunol Methods 231:25-38). It should be noted that—as is well known in the art for V H domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

The determination of the CDR regions in an antibody/immunoglobulin sequence generally depends on the algorithm/methodology applied: Kabat (Kabat et al. 1991; 5^{th} edition, NIH publication 91-3242), Chothia (Chothia & Lesk 1987, Mol Biol 196:901-17), IMGT (ImMunoGeneTics information system)-numbering schemes; see, e.g. http://www.bioinf.org.Uk/abs/index.html #kabatnum and http://www.imgt.org/IMGTScientificChart/Numbering/IMGTnumbering.html; LeFranc 2014, Frontiers in Immunology 5: 1-22). Determination of CDR regions may also be done according to other methods, such as the designation based on contact analysis and binding site topography as described in MacCallum et al. 1996 (J Mol Biol 262:732-745). Or alternatively the annotation of CDRs may be done according to AbM (AbM is Oxford Molecular Ltd.'s antibody modelling package as described on http://www.bioinf.org.uk/abs/index.html). Applying different methods to the same antibody/immunoglobulin sequence may give rise to different CDR amino acid sequences wherein the differences may reside in CDR sequence length and/or—delineation within the antibody/immunoglobulin/IVD sequence. The CDRs of the ISVD binding agents as described herein can therefore be described as the CDR sequences as present in the single variable domain antibodies characterized herein. Alternatively, these CDRs can be described as the CDR sequences present in the single variable domain antibodies (as described herein) as determined or delineated according to a well-known methodology such as according to the Kabat-, Chothia-, aHo, MacCallum et al. 1996, AbM-, or IMGT, numbering scheme or -method.

VHHs or Nbs are often classified in different families according to amino acid sequences, or even in superfamilies, as to cluster the clonally related sequences derived from the same progenitor during B cell maturation (Deschaght et al. 2017, Front Immunol 8:420). This classification is often based on the CDR sequence of the Nbs, and wherein for instance each Nb (or VHH) family is defined as a cluster of (clonally) related sequences with a sequence identity threshold of the CDR3 region. Within a single VHH family defined herein, the CDR3 sequence is thus identical or very similar in amino acid composition, preferably with at least 80% identity, or at least 85% identity, or at least 90% identity in the CDR3 sequence, resulting in Nbs of the same family binding to the same binding site, and having the same effect such as functional effect.

Immunoglobulin single variable domains such as Domain antibodies and Nanobody® (including VHH domains) can be subjected to humanization, i.e. to increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized. Humanized immunoglobulin single variable domains, in particular Nanobody®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the parental (non-humanized) VHH, such as the antigen-binding capacity. Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, and to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles and used for humanisation construct design. A humanisation technique applied to Camelidae VHHs may also be performed by a method comprising the replacement of specific amino acids, either alone or in combination. Said replacements may be selected based on what is known from literature, from known humanization efforts, as well as from human consensus sequences compared to the natural VHH sequences, or from the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden or need of humanization.

Suitable mutations, in particular substitutions, can be introduced during humanization to generate a polypeptide with reduced binding to pre-existing antibodies (reference is made for example to WO 2012/175741 and WO2015/173325), for example at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation. In some cases, at least one of the typical Camelidae hallmark residues with hydrophilic characteristics at position 37, 44, 45 and/or 47 is replaced (see Table A-03 of WO2008/020079). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 10 103, 104, 108 and/or 111 (see Tables A-05-A08 of WO2008/020079; all numbering according to the Kabat-methodology). Humanization typically only concerns substitutions in the FR and not in the CDRs, as this could/would impact binding affinity to the target and/or potency.

As used herein, a “therapeutically active agent” means any molecule that has or may have a therapeutic effect (i.e. curative or prophylactic effect) in the context of treatment of a disease (as described further herein). Preferably, a therapeutically active agent is a disease-modifying agent, which can be a cytotoxic agent, such as a toxin, or a cytotoxic drug, or an enzyme capable of converting a prodrug into a cytotoxic drug, or a radionuclide, or a cytotoxic cell, or which can be a non-cytotoxic agent. Even more preferably, a therapeutically active agent has a curative effect on the disease. The binding agent or the composition, or pharmaceutical composition of the invention may act as a therapeutically active agent, when beneficial in treating patients infected with corona virus infections, such as SARS Corona virus or patients suffering from COVID-19. The binding agent may include an agent comprising a variant of the sarbecovirus-binding ISVDs as described herein, preferably an improved variant binding to the same binding region of the RBD, and more preferably a humanized variant thereof, and may contain or be coupled to additional functional groups, advantageous when administrated to a subject. Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, PA (1980). Such functional groups may for example be linked directly (for example covalently) to the ISVD or active antibody fragment, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). For example, for this purpose, PEG may be attached to a cysteine residue that naturally occurs in a immunoglobulin single variable domain of the invention, a immunoglobulin single variable domain of the invention may be modified so as to suitably introduce one or more cysteine residues for attachment of PEG, or an amino acid sequence comprising one or more cysteine residues for attachment of PEG may be fused to the N- and/or C-terminus of an ISVD or active antibody fragment of the invention, all using techniques of protein engineering known per se to the skilled person. Another, usually less preferred modification comprises N-linked or O-linked glycosylation, usually as part of co-translational and/or post-translational modification, depending on the host cell used for expressing the antibody or active antibody fragment. Another technique for increasing the half-life of a binding domain may comprise the engineering into bifunctional or bispecific domains (for example, one ISVD or active antibody fragment against the target RBD of Corona virus and one against a serum protein such as albumin or Surfactant Protein A (SpA)—which is a surface protein abundantly present in the lungs aiding in prolonging half-life)) or into fusions of antibody fragments, in particular immunoglobulin single variable domains, with peptides (for example, a peptide against a serum protein such as albumin). In yet another example, the variant ISVD of the invention can be fused to an immunoglobulin Fc domain such as an IgA Fc domain or an IgG Fc domain, such as for example IgG1, IgG2 or IgG4 Fc domains. Examples are further shown in the experimental section and are also depicted in the sequence listing.

The term “compound” or “test compound” or “candidate compound” or “drug candidate compound” as used herein describes any molecule, either naturally occurring or synthetic that is designed, identified, screened for, or generated and may be tested in an assay, such as a screening assay or drug discovery assay, or specifically in the method for identifying a compound capable of neutralizing Corona virus, specifically 2019-Corona virus infections. As such, these compounds comprise organic and inorganic compounds. For high-throughput purposes, test compound libraries may be used, such as combinatorial or randomized libraries that provide a sufficient range of diversity. Examples include, but are not limited to, natural compound libraries, allosteric compound libraries, peptide libraries, antibody fragment libraries, synthetic compound libraries, fragment-based libraries, phage-display libraries, and the like. Such compounds may also be referred to as binding agents; as referred to herein, these may be “small molecules”, which refers to a low molecular weight (e.g., <900 Da or <500 Da) organic compound. The compounds or binding agents also include chemicals, polynucleotides, lipids or hormone analogs that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies, antibody mimetics, antibody fragments or antibody conjugates.

As used herein, the terms “determining”, “measuring”, “assessing”, “identifying”, “screening”, and “assaying” are used interchangeably and include both quantitative and qualitative determinations. “Similar” as used herein, is interchangeable for alike, analogous, comparable, corresponding, and -like or alike, and is meant to have the same or common characteristics, and/or in a quantifiable manner to show comparable results i.e. with a variation of maximum 20%, 10%, more preferably 5%, or even more preferably 1%, or less.

The term “subject”, “individual” or “patient”, used interchangeably herein, relates to any organism such as a vertebrate, particularly any mammal, including both a human and other mammals, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” or “subject” herein. However, it will be understood that the aforementioned terms do not imply that symptoms are present.

The term “treatment” or “treating” or “treat” can be used interchangeably and are defined by a therapeutic intervention that slows, interrupts, arrests, controls, stops, reduces, inhibits, or reverts the progression or severity of a sign, symptom, disorder, condition, or disease, but does not necessarily involve a total elimination of all disease-related signs, symptoms, conditions, or disorders. Therapeutic treatment is thus designed to treat an illness or to improve a person's health, rather than to prevent an illness. Treatment may also refer to a prophylactic treatment which relates to a medication or a treatment designed and used to prevent a disease from occurring.

It is to be understood that although particular embodiments, specific configurations as well as materials and/or molecules, have been discussed herein for methods, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. Isolation of Neutralizing VHHs that do not Compete with VHH72 for the Binding of SARS-CoV-2 RBD

To obtain SARS-Cov-1 and SARS-CoV-2 cross reactive VHHs, a llama that was previously immunized with recombinant prefusion stabilized SARS-CoV-1 and MERS spike protein was additionally immunized 3 times with recombinant SARS-CoV-2 spike protein stabilized in its prefusion conformation (Wrapp et al. 2020, Cell 181:1436-1441; Wrapp et al. 2020, Science 367:1260-1263). After the immunization, peripheral blood lymphocytes were isolated from the llama and an immune VHH-displaying phagemid library was constructed. SARS-CoV-2 spike-specific VHHs were selected using different panning strategies using immobilized SARS-CoV-2 spike or RBD in the presence or absence of bivalent head-to-tail fused VHH72 (Wrapp et al. 2020, Cell 181:1436-1441). Periplasmic extracts (PEs) were prepared from individual phagemid clones obtained after the panning and the binding of the VHHs in these extracts to the SARS-CoV-2 spike and RBD-SD1-Fc was evaluated by ELISA. For the majority of tested PE VHH binding to RBD could be demonstrated. Remarkably, all VHHs that bind the spike protein also bind the RBD-SD1-Fc, illustrating that none of the selected spike-binding VHHs bind the spike at sites apart from the RBD-SD1. This yielded the VHHs as listed in Table 1.

TABLE 1
Overview of the bio-panning strategies used to isolate SARS-CoV-2
neutralizing VHHs. VHHs were isolated after 1 or 2 rounds of bio-
panning using the indicated antigens in the presence (yes) or absence
(no) of a bivalent head-to-tail fused VHH72 targeting the SARS-
CoV RBD core (Wrapp et al. 2020, Cell 181: 1436-1441).

| | VHH | Antigen used | Number of | VHH72 |
|---|---|---|---|---|
| Strategy | numbering | for panning | panning rounds | added |
| 1 | VHH3.42 | SARS-CoV-2 spike | 2 | no |
| 2 | VHH3.92 | SARS-CoV-2 spike | 2 | yes |
| 3 | VHH3.94 | SARS-CoV-2 spike | 2 | yes |
| 4 | VHH3.117 | SARS-CoV-2 RBD | 1 | yes |
| 5 | VHH3.180 | SARS-CoV-2 RBD | 1 | no |

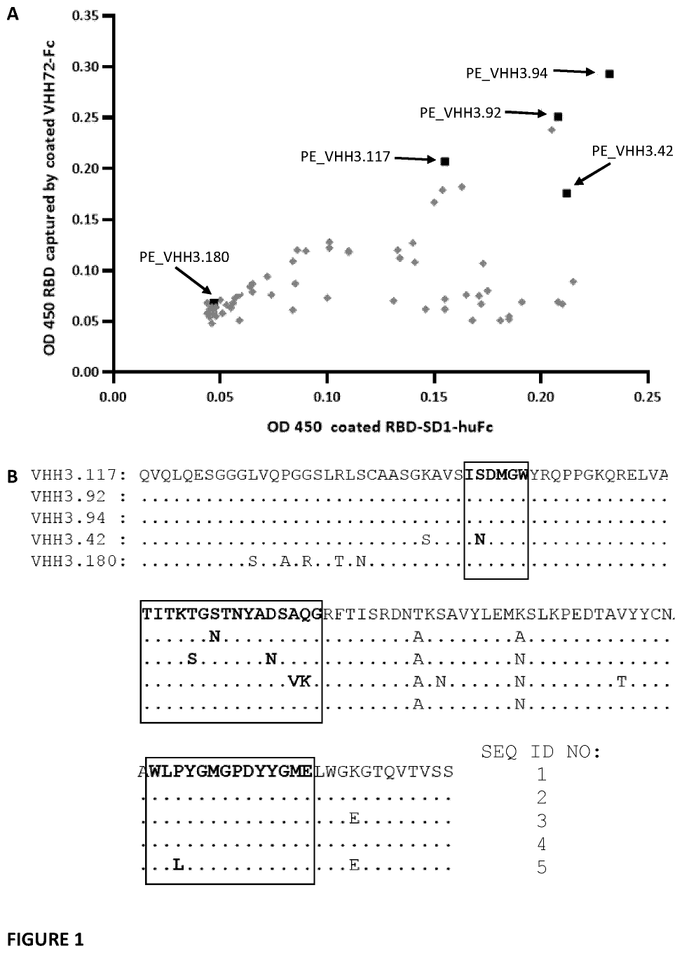

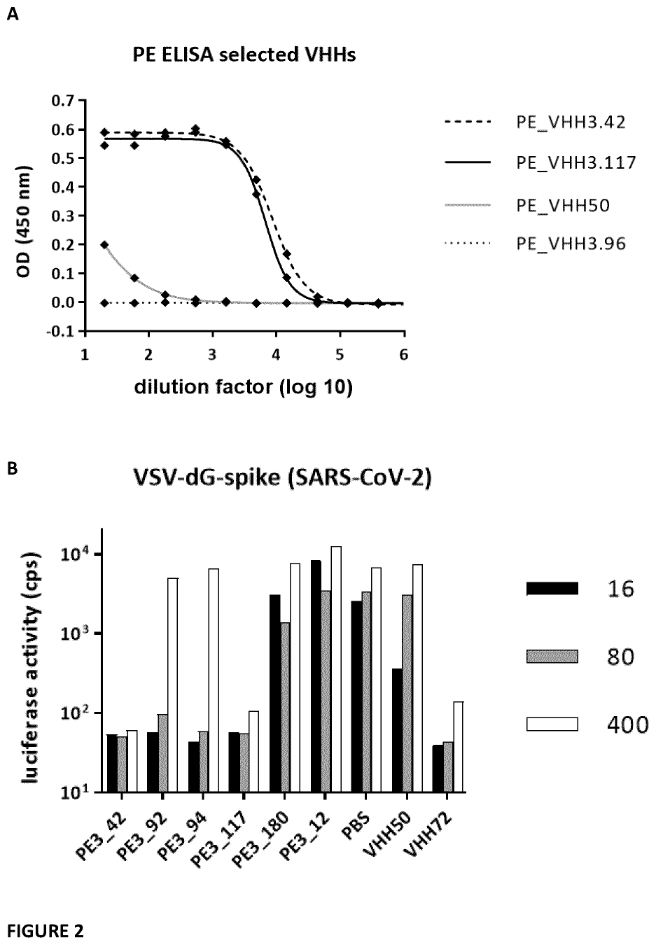

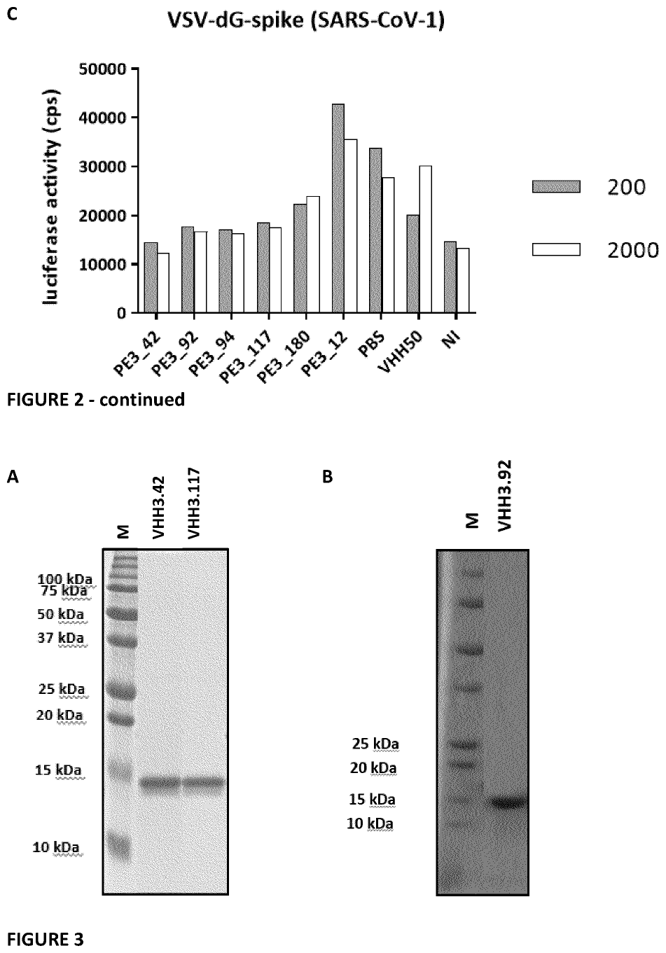

One strategy to overcome viral escape or to expand broadness of binding specificity is to combine two VHHs that target non-overlapping epitopes or do not compete for binding to a single RBD. To identify the VHHs that do not compete with VHH72 for binding to RBD, an ELISA was performed using either directly coated RBD or monovalent RBD captured by VHH72-Fc that was coated on beforehand to the wells of an ELISA plate. FIG. 1A illustrates that only few VHHs that potently bind to directly coated RBD can also bind to RBD captured by VHH72-Fc (defined by OD VHH3.x>2×OD control sample). Four (VHH3.42, VHH3.92, VHH3.94, and VHH3.117) out of the five VHHs that could most potently bind to VHH72-Fc captured monovalent RBD had highly similar amino acid sequences and belonged to the same VHH family (VHH3.42 family), the amino acid sequences of which are depicted in FIG. 1B; the amino acids sequence of a further family member, VHH3.180, is also depicted in FIG. 2B. The PEs containing VHH3.42 family members were further tested for binding to RBD (RBD-SD1-hu monoFc). FIG. 2A shows that PE extracts containing VHH3.117 (PE_117) and VHH3.42 (PE_42) contain VHH that can potently bind to SARS-CoV-2 RBD. Much lower binding was observed for a control PE extract containing a VHH related to VHH-72 (VHH50) or to a VHH (PE_96) for which no binding was observed in the initial PE-ELISA screen. To test if the VHH3.42 family members can neutralize SARS-CoV-1 and SARS-CoV-2 infections, different dilutions of the corresponding PEs were tested in a neutralization assays using pseudotyped VSV-delG containing the spike protein of SARS-CoV-1 or SARS-CoV-2. All VHH3.42 family members could neutralize pseudotyped VSV-delG containing the spike protein of SARS-CoV-1 (FIG. 2C). All VHH3.42 family members except for VHH3.180, could neutralize pseudotyped VSV-delG containing the spike protein of SARS-CoV-2 (FIG. 2B); VHH3.180 being an exception could, however, be due to the fact that periplasmic extracts (PEs) were tested. Again, VHH50, or a VHH (PE3_12) for which no binding was observed in the initial PE-ELISA screen were included as controls, as well as buffer (PBS) only.

### Example 2. Production and Purification of Selected VHHs

VHH3.42 and VHH3.117 were selected for production in Pichia pastoris and therefore re-cloned in a Pichia pastoris expression vector. The produced VH Hs contain a C-terminal GS linker followed by HA-His-TAG (TAG indicated an in frame stop codon) that was used for purification by Ni-NTA affinity chromatography. The purified VHHs were tested by SDS-PAGE and Coomassie staining (FIG. 3A). VHH3.42 and VHH3.117 migrated at the expected molecular weight of around 14.6 kDa. VHH3.92 was produced in the WK6 E. coli strain that (in contrast to the TG1 cells used for the bio-panning an PE extract preparation) do not suppress the in-frame TAG Amber stopcodon that is between the VHH-HA-HIS tag and the p3 phage protein. To this end the VHH coding pMEC vector present in the selected VHH3.93 phagmid clone was purified and used to transform WK6 cells. After production, the VHHs were extracted from the periplasm and purified by Ni-NTA affinity chromatography. SDS-PAGE analysis illustrated that the purified VHH3.92 (containing a C-terminal HA- and HIS-tag) migrated at the expected molecular weight of 15.5 kDa (FIG. 3B).

### Example 3. VHH3.42 and VHH3.117 Bind the SARS-CoV-2 and SARS-CoV-1 RBD and Spike Proteins at a Site that is Distant from the VHH72 Epitope

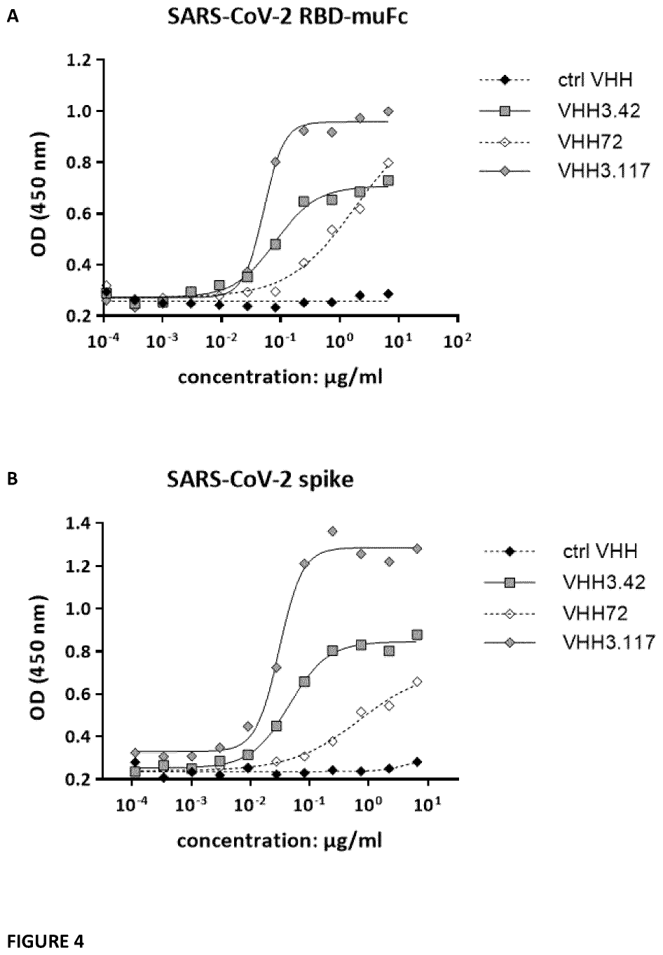

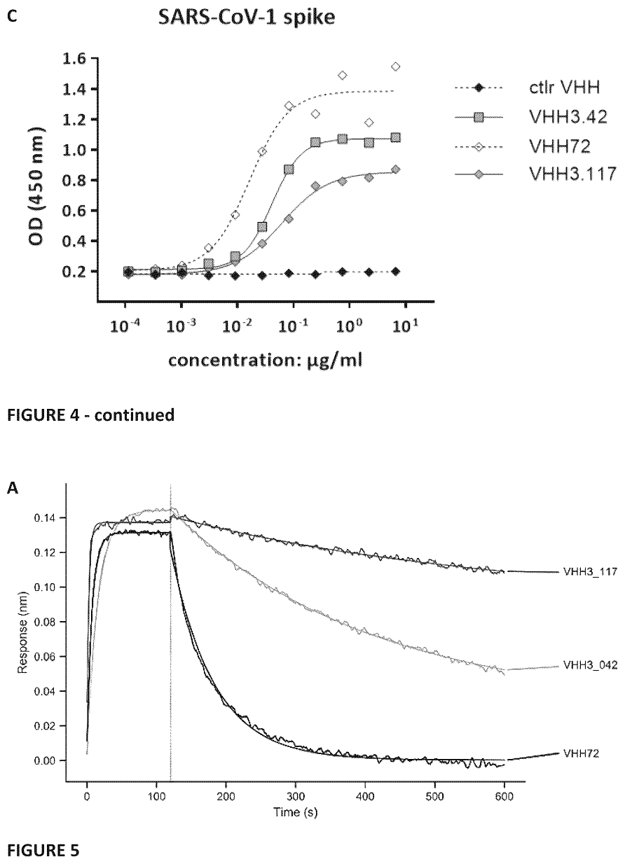

The binding of purified VHH3.42, VHH3.92 and VHH3.117 to the SARS-CoV-2 RBD and spike protein and the SARS-CoV-1 spike protein was tested by ELISA. FIGS. 4A and 4B illustrate that VHH3.42 and VHH3.117 bind the SARS-CoV-2 RBD and spike protein with higher affinity than VHH72 (VHH72_h1_S56A; humVHH_S56A in Schepens et al. 2021, BioRxiv doi.org/10.1101/2021.03.08.433449). In addition, for both the SARS-CoV-2 RBD and SARS-CoV-2 spike protein, VHH3.117 binds somewhat more efficiently than VHH3.42 (FIGS. 4A and 4B). Both VHH3.42 and VHH3.117 also bind the SARS-CoV-1 spike, with a comparable affinity as measured for the SARS-CoV-2 spike protein (FIG. 4C). As expected VHH72_h1_S56A (which was isolated after SARS-CoV-1 immunization) binds the SARS-CoV-1 spike with somewhat higher affinity than the SARS-CoV-2 spike (Wrapp et al. 2020, Cell 181:1436-1441).

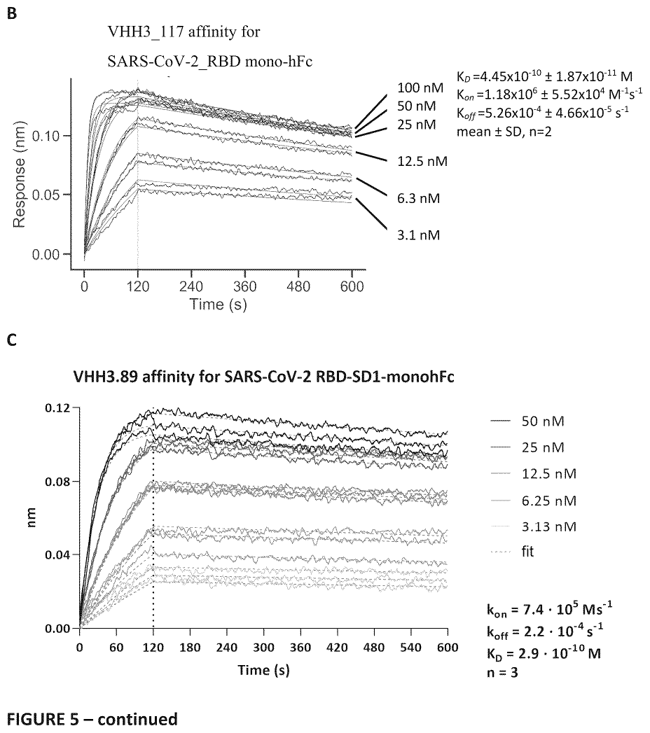

Binding of the VHHs to the RBD of SARS-CoV-2 was also tested by biolayer interferometry (BLI) in which monovalent SARS-CoV-2 RBD-human Fc was immobilized at 30 nM on an anti-human Fc biosensors (AMC FortéBio). This revealed that VHH3.42 and VHH3.117 bound RBD with a considerable slower off rate than VHH72 (FIG. 5A, each VHH at 200 nM). In line with the ELISA data, the off rate of VHH3.117 was somewhat slower than that of VHH3.42. For a 100 to 3.13 nM 2-fold dilution series of VHH3.117 and a 50 to 3.13 nM 2-fold dilution series of VHH3.89, the binding kinetics were determined using the same BLI setup. FIGS. 56 and 5C illustrate that VHH3.117 and VHH3.89 bind monomeric RBD with a K_{D} of 4.45 10⁻¹⁰ M and 2.92·10⁻¹⁰ M, respectively.

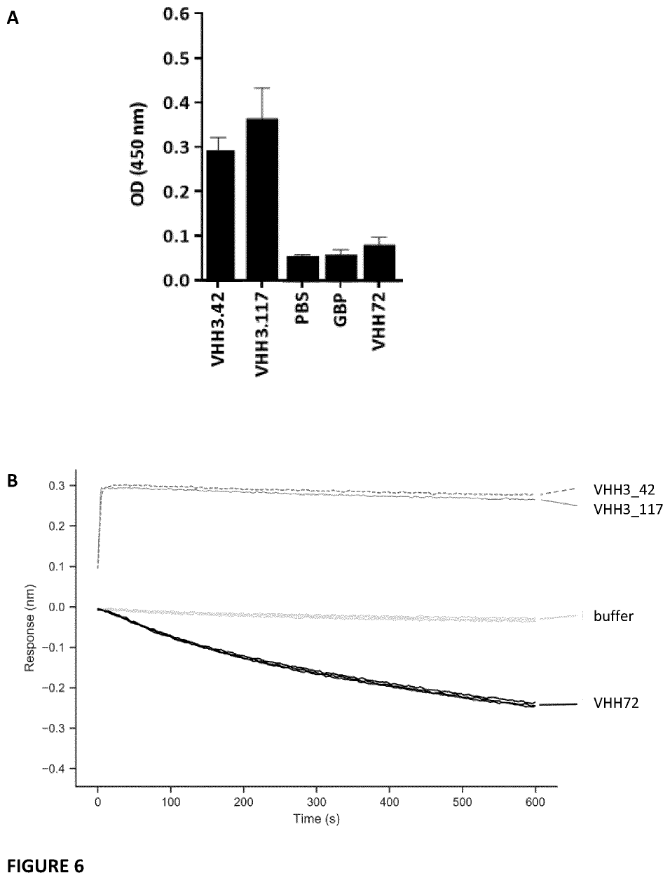

To test if VHH3.42 and VHH3.117 compete with VHH72 for binding to RBD, monomeric RBD (RBD-SD1-Avi (biotinylated Avi-tag) was captured on ELISA plates coated with VHH72-S56A-Fc (D72-23=humVHH_S56A/LALAPG-Fc; Schepens et al. 2021, BioRxiv doi.org/10.1101/2021.03.08.433449); this is a VHH72-human IgG1 Fc fusion in which VHH72 has a S56A substitution in CDR2 which increases its affinity for SARS-CoV-1 and -2 RBD) (FIG. 6A). VHH72 and several VHHs for which the PEs did display competition with VHH72 for the binding to the RBD were included as controls. In contrast to VHH72 and the control VHHs (not shown), VHH3.42 and VHH3.117 were able to bind monomeric RBD immobilized by VHH72-S56A-Fc (FIG. 6A). A similar competition experiment was performed by BLI in which VHH72-S56A-Fc was immobilized on anti-human Fc biosensors (AHC, FortéBio) and pretreated with RBD-muFc to allow binding of the latter to the immobilized VHH72-S56A-Fc. This biosensor was subsequently applied to a solution containing 1 μM of either VHH72-S56A-Fc, VHH3.42, VHH3.117 or only buffer. As expected, applying the biosensor probed with VHH72-huFc/RBD-muFc into a VHH72 containing solution reduced the BLI response signal, indicating the release of RBD-Fc from the biosensor. This confirms that VHH72 can compete with (displace) VHH72-S56A-Fc for the binding of RBD. In sharp contrast to this, applying a VHH72-huFc/RBD-muFc probed biosensor into a solution containing either VHH3.42 or VHH3.117 resulted in a clear enhancement of the BLI response signal (FIG. 6B). This illustrates that VHH3.117 and VHH3.42 can bind the RBD at a site that is distant from the VHH72 epitope.

### Example 4. VHH3.42, VHH3.117 Neutralize SARS-CoV-2 and SARS-CoV-1

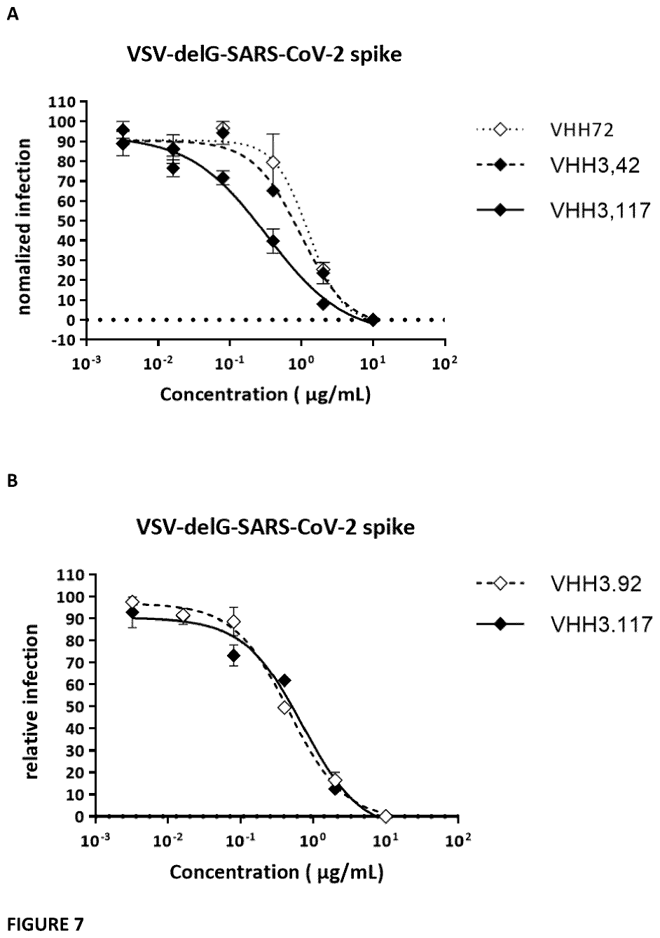

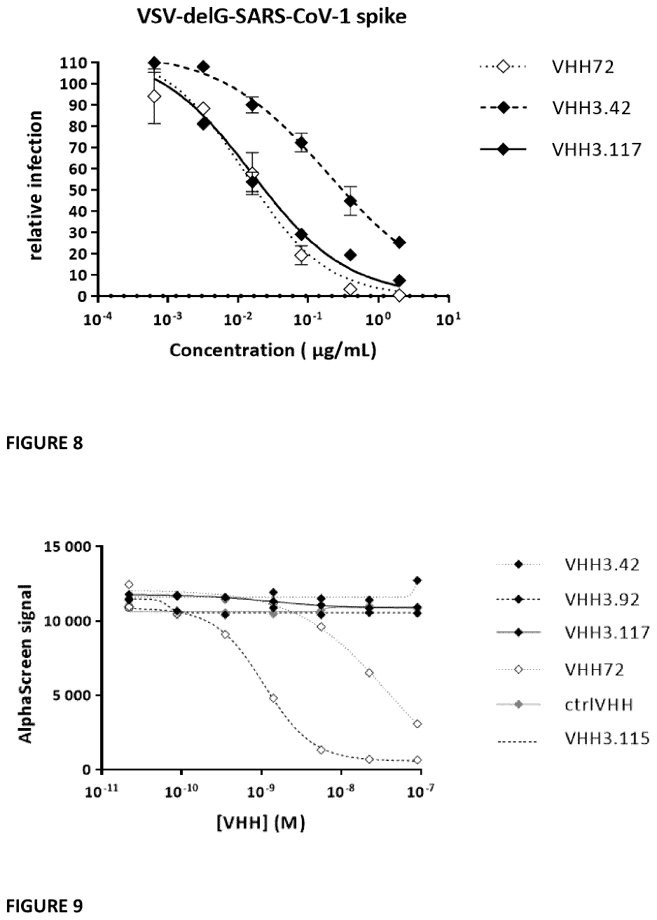

To test the neutralizing activity of purified VHH3.42, VHH3.117 and VHH3.92 we performed neutralization assays using pseudotyped VSV-delG containing the spike protein of SARS-CoV-2 or SARS-CoV-1. FIGS. 7A and 7B, and Table 2 illustrate that VHH3.42, VHH3.117 and VHH3.92 could neutralize pseudotyped VSV-delG containing the spike protein of SARS-CoV-2, and this about 6 times more efficiently than VHH72_h1_S56A. We also tested if VHH3.42 and VHH3.117 could also neutralize SARS-CoV-1. FIG. 8 and Table 2 illustrate that both VHH3.42 and VHH3.117 could potently neutralize VSV-deIG pseudotyped with SARS-CoV-1 spike. For both SARS-CoV-1 and SARS-CoV-2 the neutralizing activity of VHH3.117 was somewhat higher than that of VHH3.42.

TABLE 2
The IC50 values of independent neutralization
assays using pseudotyped VSV-delG containing
the spike protein of SARS-CoV-2 or SARS-CoV-1.

| | IC₅₀ Mean VSV-dG- | IC₅₀ Mean VSV-dG- |
|---|---|---|
| | spike SARS-CoV-2 | spike SARS-CoV-1 |
| VHH3.42 | 0.80 ± 0.76 ug/ml (n = 5) | 0.19 ug/ml (n = 1) |
| VHH3.117 | 0.20 ± 0.15 ug/ml (n = 6) | 0.016 ug/ml (n = 1) |
| VHH3.92 | 0.21 ± 0.13 ug/ml (n = 2) | NT |
| VHH72_h1_S56A | 1.14 ± 0.67 ug/ml (n = 5) | 0.013 ug/ml (n = 1) |

(NT = not tested)

### Example 5. VHH3.42, VHH3.117 and VH3.92 do not Prevent Binding of RBD to its Receptor, ACE2

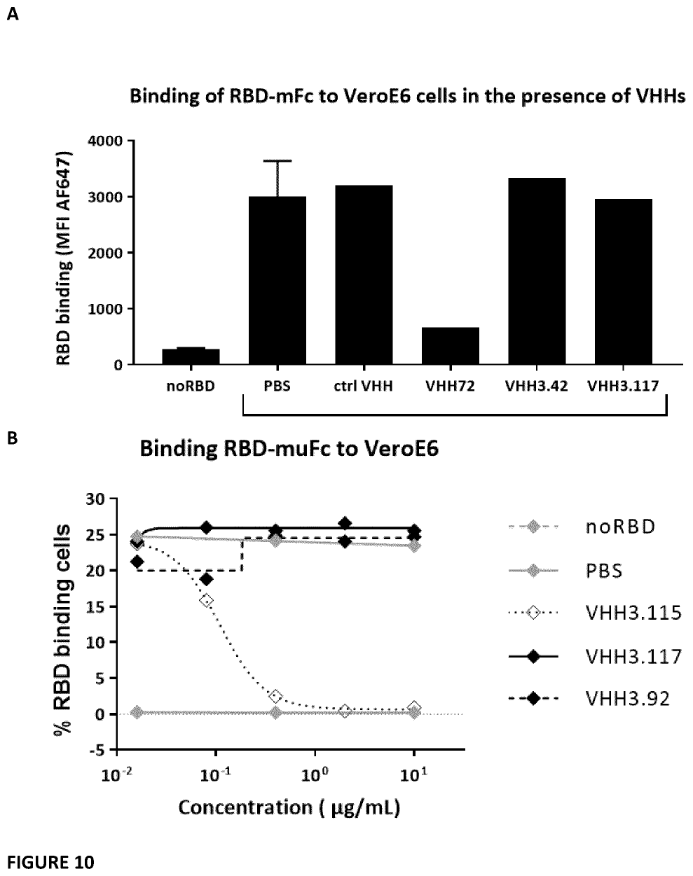

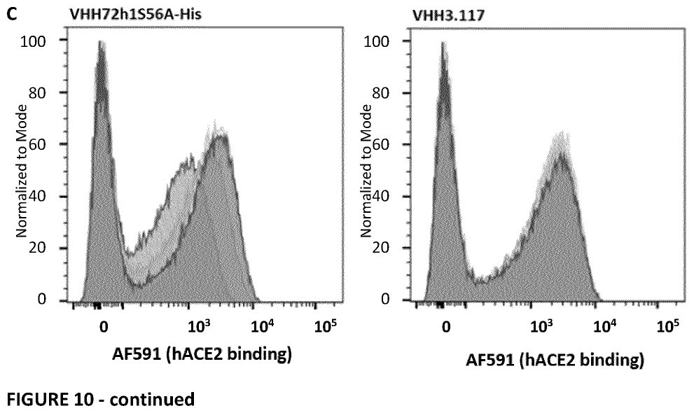

Most reported monoclonal antibodies and VHHs neutralize by preventing the binding of RBD to its receptor ACE2. Although VHH72 binds the RBD outside its receptor-binding motif (RBM) it prevents RBD from binding to ACE2 by steric hindrance (Wrapp et al. 2020, Cell 181:1436-1441). To investigate if the neutralizing VHHs identified herein are able to inhibit binding of RBD to ACE2, we investigated the impact of these VHHs on the interaction of recombinant RBD with recombinant ACE2 proteins by AlphaUSA. Serial dilutions of VHHs (final concentration ranging between 90 nM-0.04 nM) were made in assay buffer (PBS containing 0.5% BSA and 0.05% Tween-20), and mixed with SARS-CoV-2 RBD that was biotinylated through an Avi-tag (AcroBiosystems, Cat nr. SPD-C82E9) (final concentration 1 nM) in white low binding 384-well 88 microtiter plates (F-bottom, Greiner Cat nr 781904). Recombinant human ACE-2-Fc (final concentration 0.2 nM) was added to the mixture. After 1 hour incubation at room temperature, donor and acceptor beads were added to a final concentration of 20 μg/mL for each in a final volume of 0.025 mL. RBD was captured on streptavidin coated Alpha Donor beads (Perkin Elmer, Cat nr. 6760002). Human ACE-2-mFc protein (Sino Biological Cat nr. 10108-H05H) was captured on anti-mouse IgG (Fc specific) acceptor beads (Perkin Elmer, Cat nr. AL105C). The mixed beads were incubated for an additional 1 hour at room temperature in the dark. Interaction between beads was assessed after illumination at 680 nm and reading at 615 nm on an Ensight instrument. In contrast to VHH72 and the related VHH3.115, neither of the herein identified VHH3.42, VHH3.117 and VHH3.92 could interfere with the RBD/ACE2 interaction even at doses well above their respective neutralization IC₅₀ (54.8 nM, 13.7 nM and 13.55 nM) (FIG. 10).

To investigate if the herein identified VHHs are also unable to inhibit binding of RBD to ACE2 expressed at a cell surface, we determined binding of bivalent SARS-CoV-2 RBD, fused to a mouse Fc, to Vero cells (FIG. 9). FIGS. 10A and 10B illustrate that VHH3.42, VHH3.117 and VHH3.92 could not prevent the interaction of bivalent SARS-CoV-2 RBD with VeroE6 cells, even at concentrations well above their respective neutralization IC₅₀ (Table 2). This indicates that these VHHs neutralize SARS-CoV infections via an alternative mechanism that does not involve prevention of RBD mediated viral attachment to target cells.

Next, we tested if VHHs of the VHH3.42 family would also fail to interfere with the binding of recombinant ACE2 to cell-surface expressed RBD. Therefore, we investigated if VHH72 or VHH3.117 can prevent the binding of recombinant ACE2 fused to a mouse Fc to RBD expressed at the surface of yeast cells (FIG. 10C). As expected, VHH72 (VHH72_h1_S56A) could inhibit the binding of recombinant ACE2-Fc to yeast cells that express SARS-CoV-2 RBD at their cell surface. In contrast, VHH3.117 could not do so.

Taken together these data consistently demonstrate that herein identified VHHs cannot prevent binding of RBD to ACE2, i.e. the canonical sarbecovirus (such as SARS-CoV-1 and -2) receptor that is expressed at the surface of target cells. This indicates that these VHHs neutralize sarbecovirus infections via an alternative mechanism.

### Example 6. VHH3.42-Family Members Bind an Epitope that is Distant from that of VHH72, CB6, CR3022 and S309

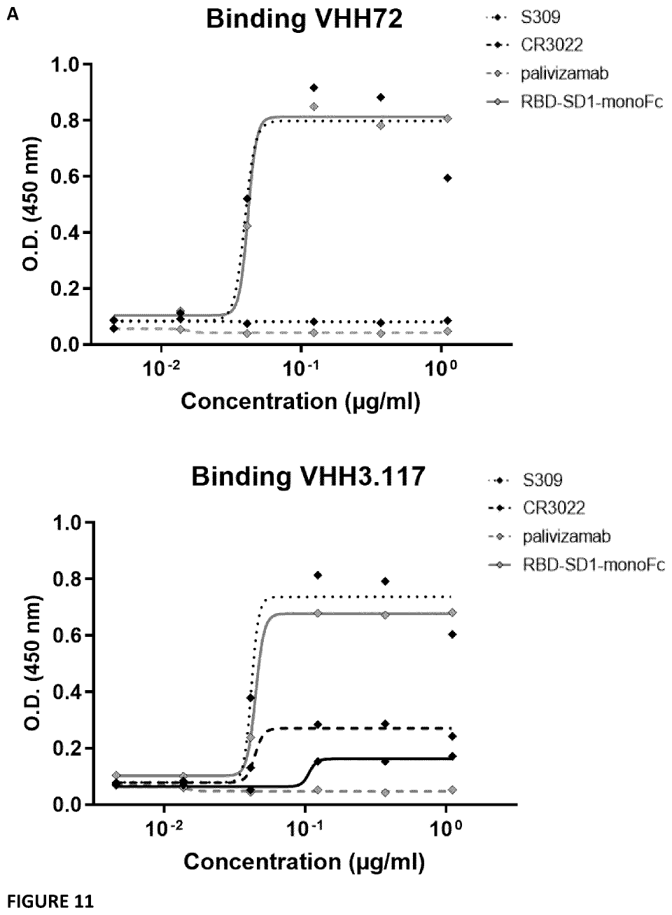

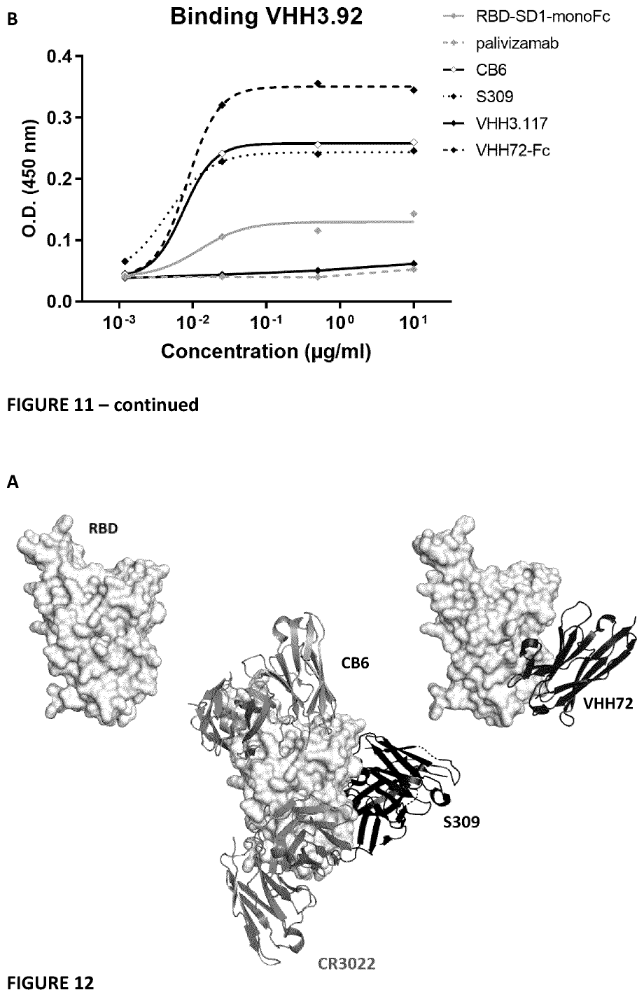

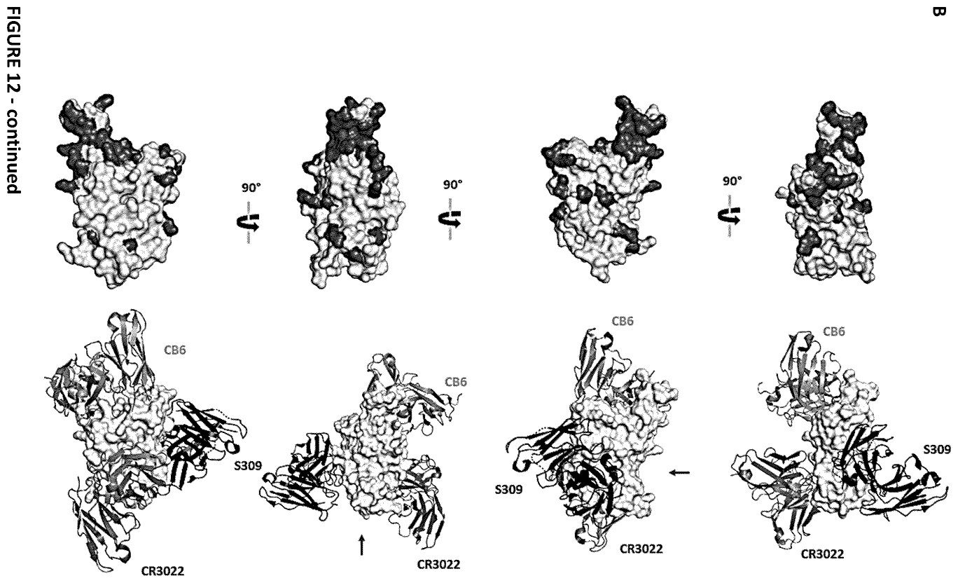

The observation that the herein identified VHHs family do not compete with VHH72 or ACE2 for RBD binding, illustrates that these VHHs bind to an epitope that is distant from VHH72 and from the RBM (receptor binding motif (sub)domain in the RBD). To further narrow down the epitope of these VHHs we tested the binding of VHH72 and VHH3.117 to monovalent RBD (RBD-SD1-monohuFc) that was immobilized by various antibodies that were coated in the wells of an ELISA plate. FIGS. 11A and 16A illustrates that binding of 5309 (binds RBD core at a site that is opposite to the VHH72 contact region), or CR3022 (binds an epitope that largely overlaps with that of VHH72 but extends to the lower side of the RBD) does not interfere with the binding of VHH3.117 (Pinto et al. 2020, Nature 583:290-295; Yuan et al. 2020, Science 369:1119-1123). As expected, binding of VHH72 was prevented by CR3022. In a separate experiment we investigated the binding of VHH3.92 to monovalent RBD that was immobilized on wells of an ELISA plate by coated CB6 (human monoclonal antibody that binds the RBM), 5309, VHH72-Fc or VHH3.117 (Shi et al. 2020, Nature 584:120-124). Binding of VHH3.92 to RBD was not affected by 5309 and VHH72-Fc but was abrogated by VHH3.117 (FIG. 11B). In addition, binding of VHH3.92 to the RBD was not affected by CB6 (FIG. 11B). Taking into account the ability of VHH3.117 and related VHHs to cross-bind and cross-neutralize SARS-CoV-2 and -1, these data strongly indicate that only few sites on the RBD can be recognized by these VHHs. Especially, the lateral side of the RBD opposite of the VHH72 and 5309 binding sites is conserved between SARS-CoV-1 and -2 and not occluded by the above described monoclonal antibodies. So, most likely, the binding site of VHH3.117 and related VHHs is located within this region (see FIG. 12).

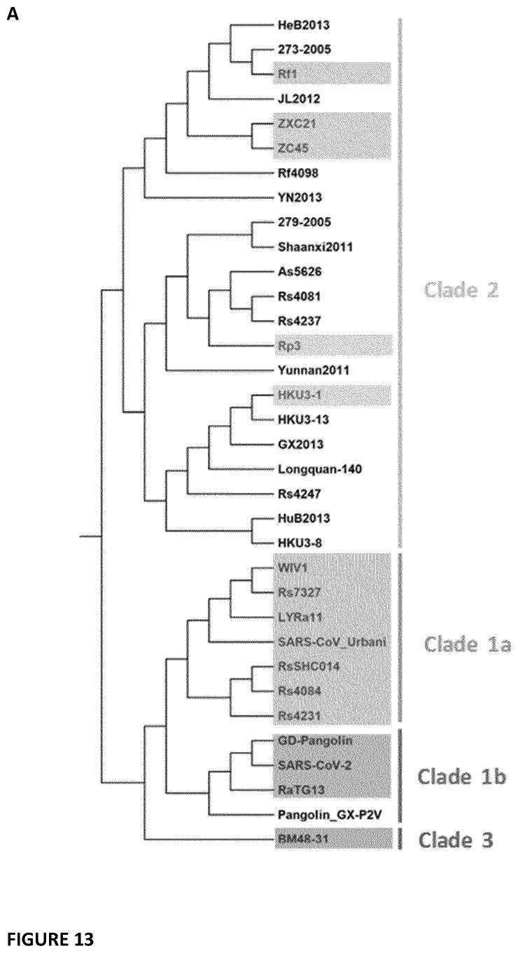

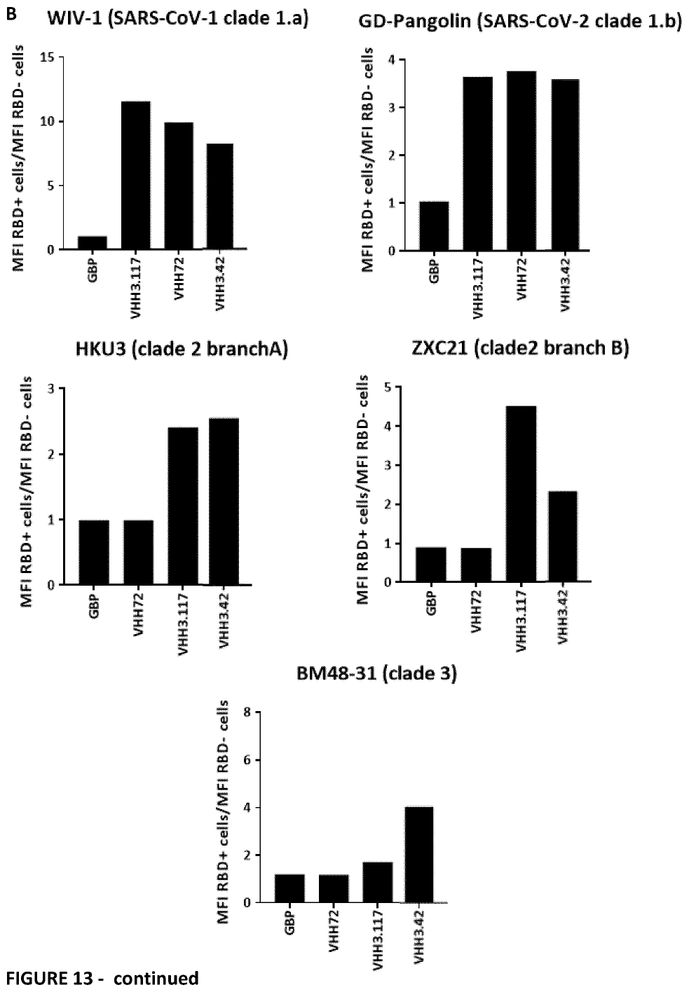

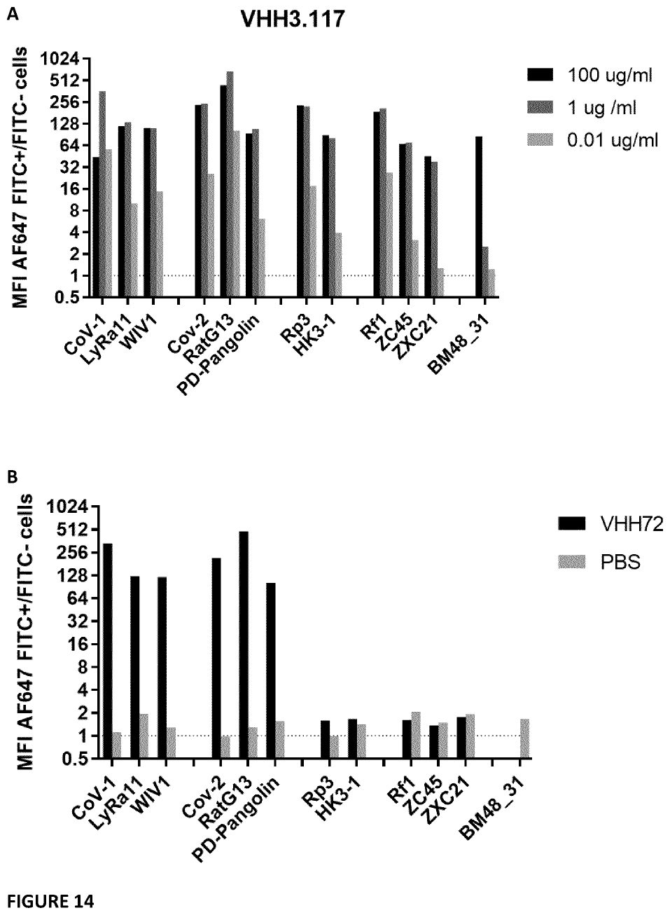

To further delineate the epitope of the herein identified VHH family and to define their potential for cross-reacting with other sarbecoviral RBDs, we investigated their binding to the RBD of various sarbecoviruses. To this end, binding of these VHHs to yeast cells expressing the RBD of representative clade 1.A (WIV1), cladel.B (GD-pangolin), clade 2 (HKU3 and ZCX21) and clade 3 (BM48-31) sarbecoviruses (FIG. 13A) was tested by flow cytometry. In line with the binding to the spike proteins of SARS-CoV-2 and -1 in ELISA, all tested VHHs (at 10 μg/ml), except for the GBP (GFP binding protein) control VHH, bind yeast cells expressing the RBD of clade 1.A (WIV1) and cladel.B (GD-pangolin) at their surface (FIG. 13B). In addition, VHH3.117, VHH3.42 and VHH3.92 are able to bind to the RBD of HKU3 and ZXC21, representing the two clade 2 branches. Moreover, VHH3.42, VHH3.92 and to a lesser extent VHH3.117 could also bind to the RBD of the clade 3 BM48-31 sarbecovirus (FIG. 13B). In a separate experiment, the binding of VHH3.117 to a broader range of clade 1, 2 and 3 sarbecoviruses was tested. FIG. 14A illustrates that VHH3.117 can bind to all tested RBD variants, and is binding to more RBD variants compared to VHH72 (FIG. 14B). These observations are in line with the hypothesis that VHH3.117 targets an RBD region that is highly conserved among the tested RBD variants.

### Example 7. Determination of the Binding Site of VHH3.117 on the RBD by Deep Mutational Scanning

To determine the binding site of the herein identified VHHs on the RBD we performed deep mutational scanning. VHH72 (VHH72_h1_S56A), for which a crystal structure in complex with the related SARS-CoV-1 RBD is available, was included as a reference (Wrapp et al. 2020, Cell 181:1436-1441; Schepens et al., doi.org/10.1101/2021.03.08.433449). We made use of a yeast-display platform consisting of 2 independently generated libraries of Saccharomyces cerevisiae cells, each expressing a particular single RBD variant labeled with a unique barcode and a myc-tag, developed as described by Starr et al. 2020 (Cell 182: 1295-1310). As such this approach allows deep-mutational scanning to pinpoint the involvement of any amino acid residue in the RBD for a given phenotype (in our case VHH3.117 binding). The 2 libraries of RBD variants were generated by PCR-based mutagenesis to generate a comprehensive collection of RBD variants in which each position has been substituted to all other amino acids. The RBD variants contain on average 2.7 amino acid substitutions. To retain only functional RBD variants the yeast RBD-display libraries were presorted by FACS based on their ability to bind recombinant ACE2 (data not shown). To identify yeast cells that express an RBD variant with reduced affinity for the tested VHHs in a sensitive manner we defined for each VHH a concentration at which binding was just below saturation. For each of the tested VHHs this concentration was first determined by staining yeast cells expressing wild type SARS-CoV-2 RBD with a dilution series of VHHs. Using this approach, we selected 400 ng/ml for VHH72_h1_S56A (VHH72) and 100 ng/ml for VHH3.117. This difference in concentration to reach a comparable “just below the saturation” concentration reflects the higher affinity for VHH3.117 for SARS-CoV-2 RBD compared with VHH72. To identify yeast cells expressing an RBD variant with reduced affinity for the tested VHH, the presorted library was stained with the VHH and anti-myc-tag antibody. RBD expressing cells that displayed low VHH staining were sorted, grown and used for next generation sequencing of their respective barcodes. To identify the RBD amino acids that are significantly involved in VHH binding, the substitutions that are enriched in the sorted population were determined as described by Greaney et al. 2021 (Cell Host Microbe 29:44-57).

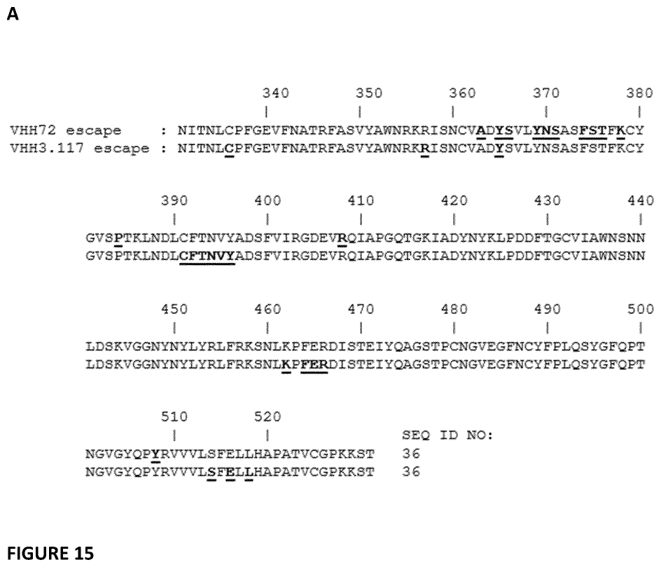

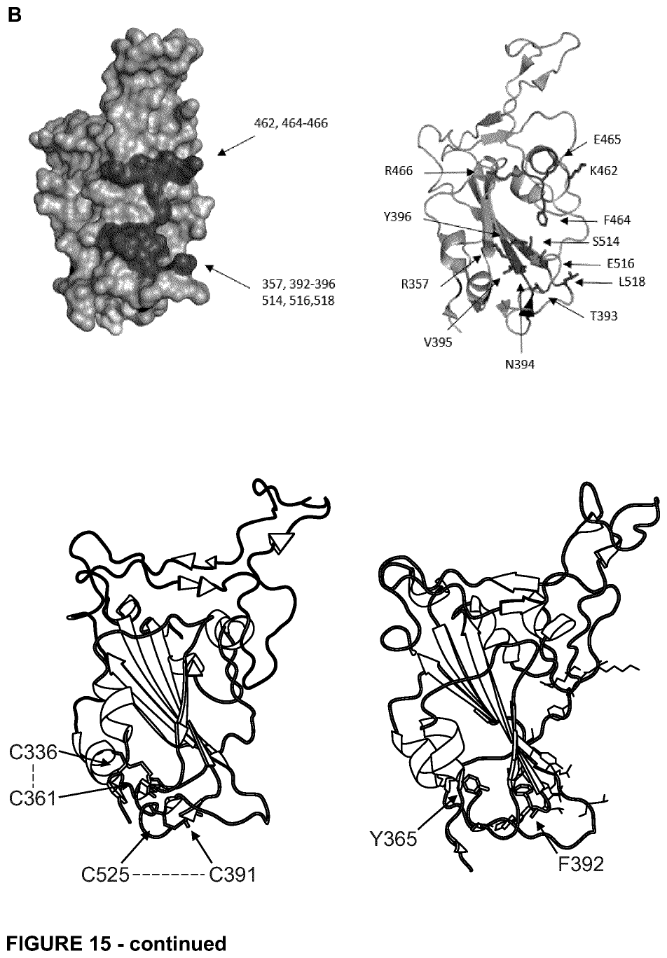

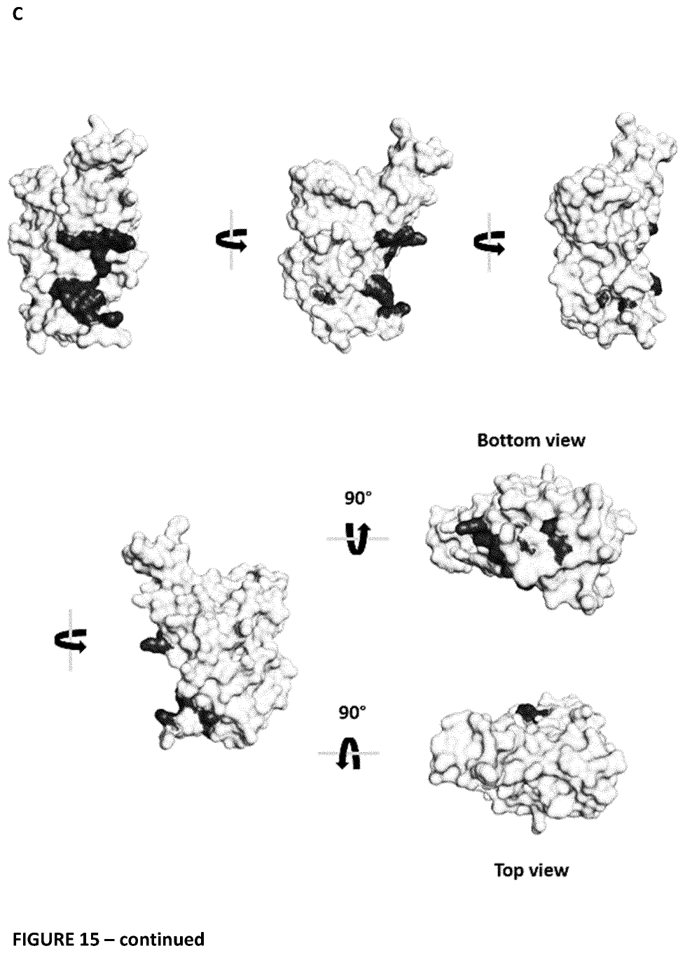

FIGS. 15A and 16C shows for the two tested VHH the overall profile of positions in the RBD for which substitutions result in reduced VHH binding. It is clear that VHH3.117 and VHH72_h1_S56A have very distinct RBD binding profiles. Escape profile analysis as established by Greaney et al. 2021 (supra), identified A363, Y365, 5366 Y369, N370, 5371, F374, 5375, T376, K378, P384, and Y508 as amino acid positions that are involved (based on the average of the two libraries) in binding of VHH72_h1_S56A. For VHH3.117, the escape profile analysis identified C336, R357, Y365, C391, F392, T393, N394, V395, Y396, K462, F464, E465, R466, 5514, E516 and L518 as important for RBD binding (FIGS. 15A and 15B). Except for C336, Y365, C391 and F392 all these amino acids cluster around a cleft at the side to the RBD that represents the likely VHH3.117 binding site based on the above described experiments. This binding site is also in agreement with the general preference of VHHs to bind clefts rather than protruding protein surfaces. C336 and C391 form disulfide bridges with respectively C361 and C525 that are likely very important for the overall stability of the RBD, explaining why these residues were identified by the deep mutational scanning (FIG. 15B). Y365 and F392 locate near the likely VHH3.117 binding surface and are oriented towards the inside of the RBD core (FIG. 15B). Hence, mutations at those positions can have an allosteric impact on the binding of VHH3.117. Deep mutational scanning revealed that Y365 is also important for VHH72 binding. Y365 is located in the RBD core at a site that is opposite of the VHH3.117 binding region. Likewise, Y365 does not locate at the RBD surface that is recognized by VHH72 but is oriented toward the inner RBD core between the VHH3.117 and VHH72 binding regions. This indicates that Y365 is important for the overall conformation of the RBD core. Importantly, the identified VHH3.117 binding site is in agreement with our findings that VHH3.117 does not compete with ACE2, 5309, VHH72, CR3022 and CB6 for the binding of RBD (illustrated for 5309 and CR3022 in FIG. 16A), in agreement with its ability to bind to the RBD of clade 1, 2 and 3 sarbecoviruses (amino acid conservation illustrated in FIG. 16B) and in agreement with its SARS-CoV-1 and -2 cross-neutralizing activity. Analysis of the amino acid variations among circulating SARS-CoV-2 viruses for which the genome sequence was submitted to GiSAID on the surface of the RBD revealed that the VHH3.117 binding region as identified by deep mutational scanning is highly conserved as illustrated by the projection of those variations on the RBD surface (FIG. 16C).

Binding of herein identified VHHs to the RBD does not interfere with binding of RBD to ACE2 at the surface of target cells. Consequently, these VHHs prevent infection via an alternative mechanism, for example by locking the SARS-CoV-2 spike in its inactive closed conformation as has been described for S309 and mNb6-tri (Pinto et al. 2020, Nature 583:290-295; Schoof et al. 2020, Science 370: 1473-1479). To get insight in the mechanism by which VHH3.117 related VHHs can neutralize SARS-CoV-1 we displayed the VHH3.117 binding site on a Spike timer with 1 RBD in up-conformation. This reveals that the VHH3.117 site is almost completely occluded on the RBDs that are in the down-conformation. Moreover, on RBDs in up-conformation the VHH3.117 binding site is largely shielded by the NTD of a second spike protomer (FIG. 16D). This demonstrates that VHH3.117 and related VHHs neutralize via mechanism that does not involve locking the RBD in its down-conformation but rather by interfering with the overall spike conformation and/or function.

### Example 8. Theoretical Interaction of ACE-2, SARS-CoV RBD, and mAb52

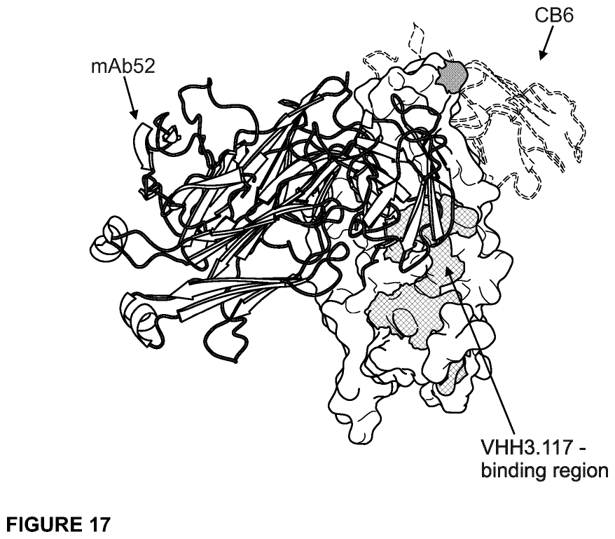

From FIG. 4A of Rujas et al. 2020 (Biorxiv 2020.10.15.341636v1), it appears that mAb52 is interfering with binding between ACE-2 and the RBD. That Figure indicates cross-competition for binding the SARS-CoV-2 RBD between antibodies 46 and 52 (defining “site 1”) on the one hand, and between antibodies 298, 82, 324, 236, and 80 (defining “site 2”) on the other hand. That same Figure furthermore indicates competition of the “site 1”-binding antibodies as well as of the “site 2”-binding antibodies with ACE-2 for binding the SARS-CoV-2 RBD. A similar conclusion can be drawn from Figure S5 of Rujas et al. 2020. Furthermore, to theoretically determine the contact points of antibody 52 (Rujas et al. 2020, Biorxiv 2020.10.15.341636v1) with SARS-CoV RBD and/or ACE-2, the available structures were 3D-modelled in silico. The resulting theoretical interactions are indicated in FIG. 17. Therefrom, it appears that mAb52 is unlikely to bind/neutralize the RBD of SARS-CoV-1 as 4 out of the 7 of the amino acids in SARS-CoV2 RBD that are important for binding to mAb52 are different in the RBD of SARS-CoV-1. Finally, mAb52 appears to bind to RBD amino acids 484 (variations known in South African, Brazilian and British SARS-CoV-2 strain) and 452 (variation known in emerging Californian SARS-CoV-2 strain). Interaction of mAb52 with RBD amino acids 484 and 452 was confirmed by Rujas et al. 2020 (supra).

### Example 9. VHH-117 and mAb52 Epitopes

As outlined in Example 7, the VHH3.117 epitope comprises one or more of the SARS-CoV-2 RBD amino acids Arg357, Thr393, Asn394, Val395, Tyr396, Lys462, Phe464, Glu465, Arg466, Ser514, Glu516 and/or Leu518 (with Cys336, Tyr 365, Cys391, Phe392 being important to keep the RBD in a conformation recognized by VHH-117). Overall, VHH3.117 does not bind to RBD amino acids known to be prone to variation in newly emerging SARS-CoV-2 strains (South African and Brazilian strains: variations in Lys417, Glu484, Asn501; Californian strain: variation in Leu452; British strain: variation in Glu484). This contrasts with the mAb52 epitope comprising one or more of the SARS-CoV-2 RBD amino acids Arg346, Tyr351, Ala352, Asn354, Arg355, Lys356, Arg357, Tyr449, Asn450, Leu452, Lys462, Glu465, Arg466, Asp467, Ile468, Ser469, Thr470, Glu471, Ile472, Asn481, Gly482, Val483, Glu484, Phe490, Leu492, and/or Gln493 (Rujas et al. 2020, Biorxiv 2020.10.15.341636v1). From both lists, it appears that the VHH3.117 epitope and mAb52 epitope are potentially overlapping only in one or more of the SARS-CoV-2 RBD amino acids Lys462, Glu465, and/or Arg466. The epitope of VHH3.117 is thus substantially different from the epitope of mAb52 both in location (limited potential overlap) and in potential function (VHH-117 likely to be able to neutralize the above-listed SARS-CoV-2 variants while this is questionable for mAb52; and VHH3.117 is not able to block ACE2 binding while mAb52 can).

### Example 10. VHH-117, Nb34, Nb95, Nb105, Nb17 and Nb36 Epitopes and Binding to Spike Protein

Xiang et al. 2020 (Science 370:1479-1484) disclose 2 groups are not competing with ACE-2 for binding the RBD and which are capable of binding with trimeric spike (S) protein only when 2 or 3 of the RBDs are in the up-conformation (epitopes III, represented by nanobody 34 or Nb34; and epitope IV, represented by nanobody 95 or Nb95). Later on, however, Nb34 and Nb95, as well as a further member Nb105, were reported as capable of blocking ACE2 binding at low nM concentrations, and Nb95 to largely loose its binding to RBD mutants E484K, Y453F and N439K (residues not part of the VHH3.17 epitope) (Sun et al. 2021, BioRxiv https://doi.org/10.1101/2021.03.09.434592). As shown in FIG. 18 herein, the locations of the epitopes of Nb34 and Nb95 as depicted in the 3D-structures of the SARS-CoV-2 RBD in Supplementary FIG. 12 of Xiang et al. 2020 were recapitulated, and compared to the epitope location of VHH3.117 on similar 3D-structures. This comparison clarifies that while overlaps exist between the Nb34 and VHH3.117 epitopes, and between the Nb95 and VHH3.117, these overlaps are only partial. This is further corroborated by the fact that Nb34 and Nb95 require 2 or 3 of the RBDs to be in the up-conformation in order to bind to the S protein (Xiang et al. 2020) while binding of VHH3.117 to the S protein is hindered by the N-terminal domain(s) when either one or more of the RBDs are in the up-conformation. The precise interaction between VHH3.117 and the RBD or Spike protein therefore is not yet fully understood although nevertheless resulting in SARS-virus neutralization.

Some characteristics of Nb17 and Nb36 have been determined by Sun et al. 2021 (BioRxiv https://doi.org/10.1101/2021.03.09.434592). In contrast to VHH3.117, nb17 is binding to the trimeric SARS-CoV-2 spike protein with all 3 RBDs in the up conformation. The epitopes of Nb17 and Nb36 were reported to be partially overlapping. For Nb17, the SARS-CoV-2 RBD amino acids (numbering relative to SARS-CoV-2 spike protein) reported to form the epitope are amino acids 345-356, 448-455, 466-472 and 482-484, with amino acids 468 and 470 being critical; for Nb36, these are amino acids 353-360 and 464-469. The VHH3.117 is only partially overlapping with the epitopes of any of these Nbs, and none of these nbs is contacting SARS-CoV-2 RBD amino acids 393-396, 514, 516 and 518.

### Example 11. VHH-117, Antibodies n3088/n3130 and n3086/n3113

Wu et al. 2020 (Cell Host Microbe 27:891-898) disclose group D antibodies n3088 and n3130, and group E antibodies n3086 and n3113, which do not compete with ACE-2 for binding to the SARS-CoV2 spike protein. Both groups of antibodies are only moderate potent in neutralizing SARS-CoV-2 pseudovirus infection, and reported IC₅₀ values are on the high end: 3.3 mg/mL for n3088; 3.7 mg/mL for n3130; 26.6 mg/mL for n3086; and 18.9 mg/mL for n3113. Although a different SARS-CoV-2 pseudovirus infection neutralization assay was used herein, all of VHH3.117, VHH3.42 and VHH3.92 neutralize SARS-CoV-2 infection with an IC₅₀ value below 1 ug/m L.

In contrast to VHH3.117, the group D antibodies of Wu et al. 2020 compete with antibody CR3022 (a human monoclonal antibody binding both to SARS-CoV-1 and SARS-CoV-2 RBD; ter Meulen et al. 2006, PLoS Med 3:e237; Tian et al. 2020, Emerging Microbes & Infections 9:382-385) for binding to the SARS-CoV2 spike protein, thus indicating binding of VHH-117 and group D antibodies to different epitopes. This is further corroborated by the fact that binding of group D antibodies to SARS-CoV2 spike protein is lost when RBD amino acids D428, F429 or E516 are substituted by an Alanine—the deep mutational scanning as performed for VHH3.117 did not implicate residues D428, F429 or E516 as being part of the VHH3.117 epitope on the SARS-CoV2 RBD.

Binding of group E antibodies to SARS-CoV2 spike protein is lost when the RBD comprises the amino acid substitutions N354D and D364Y, but not when the RBD comprises the amino acid substitution V367F—the deep mutational scanning as performed for VHH3.117 did not implicate residues N354, D364 or V367 as being part of the VHH-117 epitope on the SARS-CoV2 RBD. This indicates binding of VHH3.117 and group E antibodies to different epitopes.

Finally, the CDR3 sequences of antibodies n3088/n3130 and n3086/n3113 are provided by Wu et al. 2020 (Table S3 therein). A listing of the CDR3 sequence of the antibodies of the current invention (SEQ ID NO:8) and the CDR3 sequences of antibodies n3088/n3130 and n3086/n3113 is given below, from which can be concluded that there is overall low or no similarity between these CDR3 sequences.

SEQ ID NO: 8
WLXYGMGPDYYGME
n3088 group D
(SEQ ID NO: 48)
ARVREYYDILTGYSDYYGMDV
n3130 group D
(SEQ ID NO: 49)
ATRSPYGDYAFSY
n3086 group E
(SEQ ID NO: 50)
ARDENWGVDY
n3113 group E
(SEQ ID NO: 51)
VSNWASGSTGDY

### Example 12. Inhibition of VHH72 Binding to the RBD of the Spike Protein by AlphaLISA Immuneassay

The capacity of VHHs to compete with VHH72 for binding to SARS-CoV-2 RBD was assessed in a competition AlphaLISA (amplified luminescent proximity homogeneous assay).

Selected clones representing different VHH families were recloned for production in either Pichia pastoris or E. coli for further characterization as purified monovalent proteins. Monovalent VHHs contained a C-terminal His6 tag, or C-terminal HA-His6 tag, respectively. Purification was done using Ni-NTA affinity chromatography.

Serial dilutions of anti-SARS-CoV-2 VHHs and irrelevant control VHH (final concentration ranging between 90 nM-0.04 nM) were made in assay buffer (PBS containing 0.5% BSA and 0.05% Tween-20). VHHs were subsequently mixed with VHH72-hl (S65A)-Flag3-His6 (final concentration 0.6 nM) and SARS-CoV-2 RBD protein Avi-tag biotinylated (AcroBiosystems, Cat nr. SPD-C82E9) (final concentration 0.5 nM) in white low binding 384-well microtitre plates (F-bottom, Greiner Cat nr 781904). After an incubation for 1 hour at room temperature, donor and acceptor beads were added to a final concentration of 20 μg/mL for each in a final volume of 0,025 mL. Biotinylated RBD was captured on streptavidin coated Alpha Donor beads (Perkin Elmer, Cat nr. 6760002), and VHH72_h1(S56A)-Flag3-His6 was captured on anti-Flag AlphaLISA acceptor beads (Perkin Elmer, Cat nr. AL112C) in an incubation of 1 hour at room temperature in the dark. Binding of VHH72 and RBD captured on the beads leads to an energy transfer from one bead to the other, assessed after illumination at 680 nm and reading at 615 nm of on an Ensight instrument.

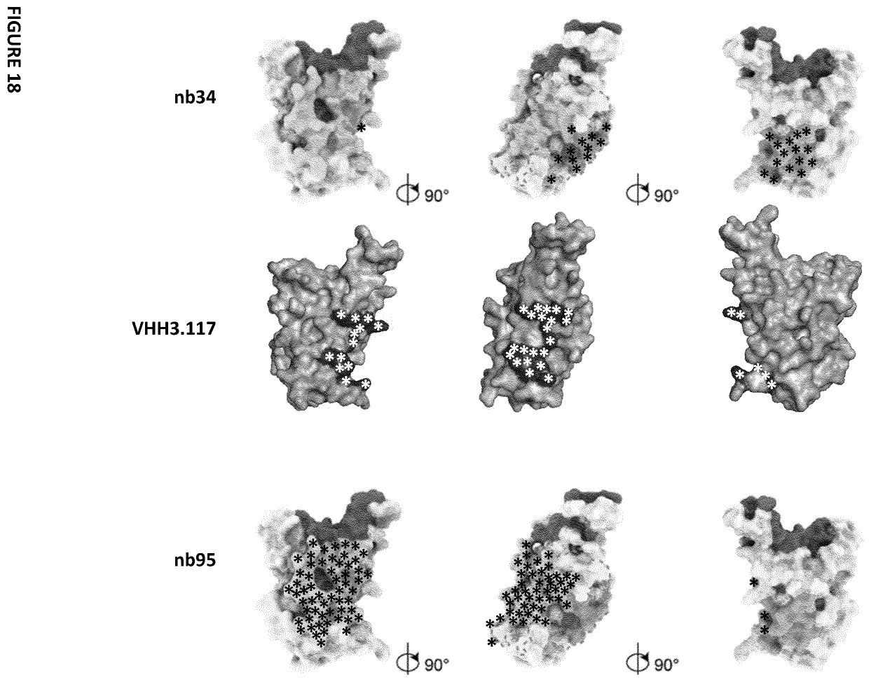

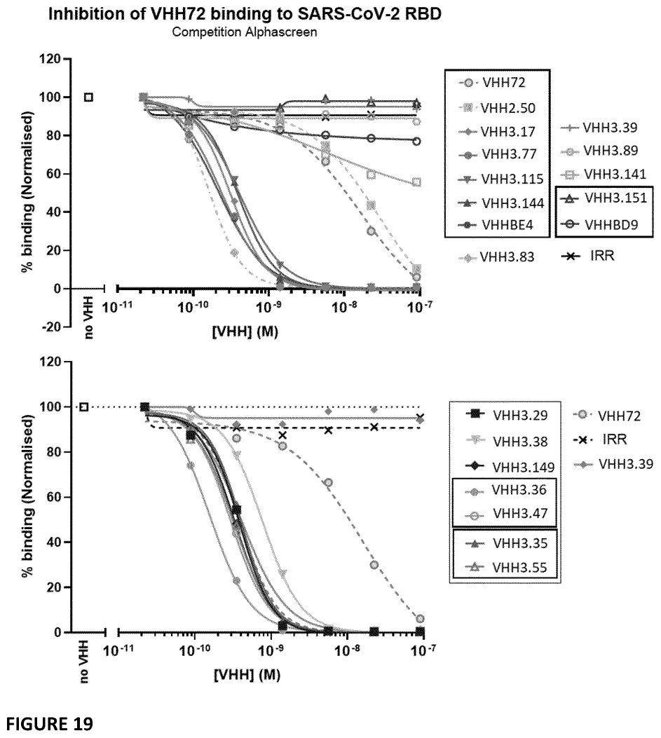

Results are shown in the FIG. 19. Results indicate that 7 VHHs (families F-36/55/29/38/149) that are part of a superfamily, and VHH3.83 (Family 83) fully block the interaction of VHH72 to the SARS-CoV-2 RBD protein, indicating they bind to at least overlapping or the same epitope as VHH72. A number of other VHH families, including VHH3.151, VHHBD9, VHH3.39, VHH3.89, and VHH3.141 are non-competitors of VHH72, indicating they bind a different epitope than VHH72.

### Example 13. Inhibition of the ACE-2/RBD Interaction by AlphaLISA Immunoassay

Dose-dependent inhibition of the interaction of SARS-CoV-2 RBD protein with the ACE-2 receptor was assessed in a competition AlphaLISA.

Selected clones representing different VHH families were recloned for production in either Pichia pastoris or E. coli for further characterization as purified monovalent proteins. Monovalent VHHs contained a C-terminal His6 tag, or C-terminal HA-His6 tag, respectively. Purification was done using Ni-NTA affinity chromatography.

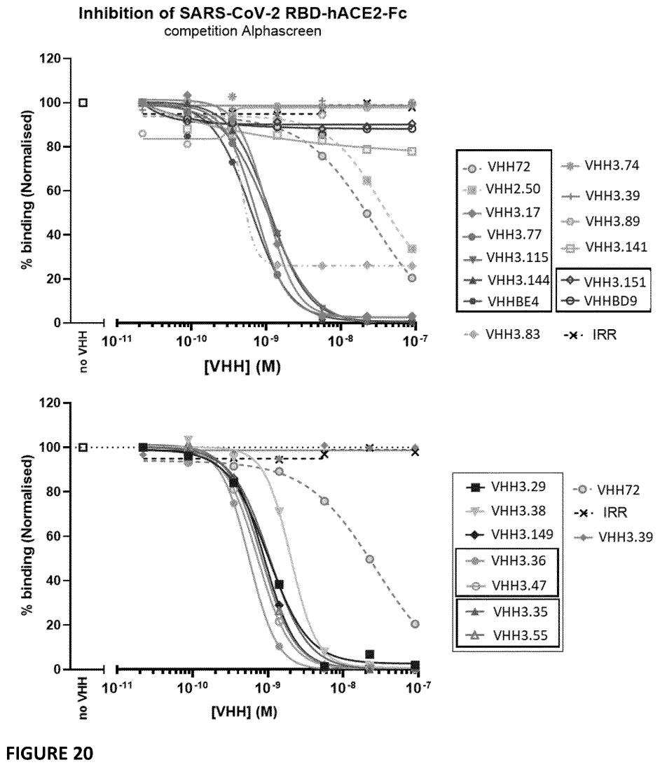

Serial dilutions of VHHs (final concentration ranging between 90 nM-0.04 nM) were made in assay buffer (PBS containing 0.5% BSA and 0.05% Tween-20), and mixed with SARS-CoV-2 RBD that was biotinylated through an Avi-tag (AcroBiosystems, Cat nr. SPD-C82E9) (final concentration 1 nM) in white low binding 384-well microtitre plates (F-bottom, Greiner Cat nr 781904). Recombinant human ACE-2-Fc (final concentration 0.2 nM) was added to the mixture. After an incubation for 1 hour at room temperature, donor and acceptor beads were added to a final concentration of 20 μg/mL for each in a final volume of 0.025 mL. RBD was captured on streptavidin coated Alpha Donor beads (Perkin Elmer, Cat nr. 6760002). Human ACE-2-mFc protein (Sino Biological Cat nr. 10108-H05H) was captured on anti-mouse IgG (Fc specific) acceptor beads (Perkin Elmer, Cat nr. AL105C) in an additional incubation of 1 hour at room temperature in the dark. Interaction between beads was assessed after illumination at 680 nm and reading at 615 nm of on an Ensight instrument. Results are shown in the FIG. 20. All VHHs that were competing with VHH72 also block the interaction of human ACE2 to the SARS-CoV-2 RBD protein.

In conclusion, the competition assay results confirm that purified VHHs from families F-83, 36, 55, 29, 38 and 149 bind to the same epitope as VHH72, and compete with ACE-2 binding similar to the VHH72 family members.

### Example 14. Identification of the VHH3.89 Family as Binding Agents for the VHH3.117 Epitope

VHH3.89 (SEQ ID NO:53) was identified as previously reported (PCT/EP2021/052885), and several additional family members of this Nb have been revealed herein, corresponding to VHH3_183, and VHH3C_80 (respectively depicted in SEQ ID NO:54 and 55).

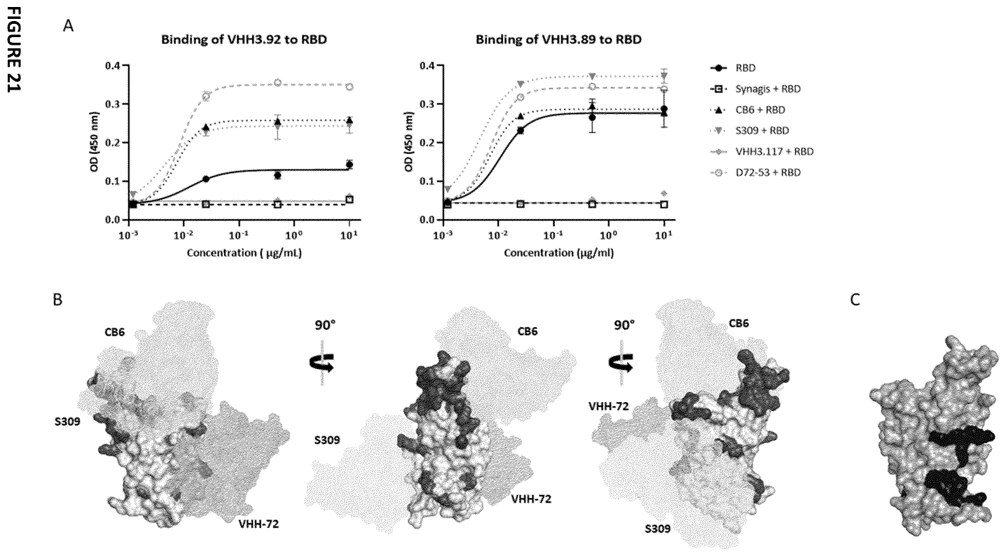

Previous analysis revealed that next to VHH3.117 also VHH3.89 does not compete with VHH72 for the binding of the SARS-CoV-2 RBD (see FIG. 19). To confirm this and to further characterize the binding site of VHH3.89 binding of this VHH to monovalent RBD that was either directly coated to ELISA plates or captured by coated monoclonal antibodies 5309, CB6 or by VHH3.117 or by VHH72-S56A fused to a human IgG1 Fc (D72-53=VHH72_h1_E1D_S56A-(G4S)2-hIgG1hinge_EPKSCdel-hIgG1_LALA_Kdel) was investigated (Pinto et al., Nature, 2020; Shi et al., Nature 2020). FIG. 21A demonstrates that VHH3.89 just like VHH3.92, a VHH that belongs to the family of VHH3.117, does not compete with 5309, CB6 and D72-53 but does compete with VHH3.117. This demonstrates that the binding site of VHH3.89 overlaps with that of VHH3.117 and VHH3.92 (FIG. 21).

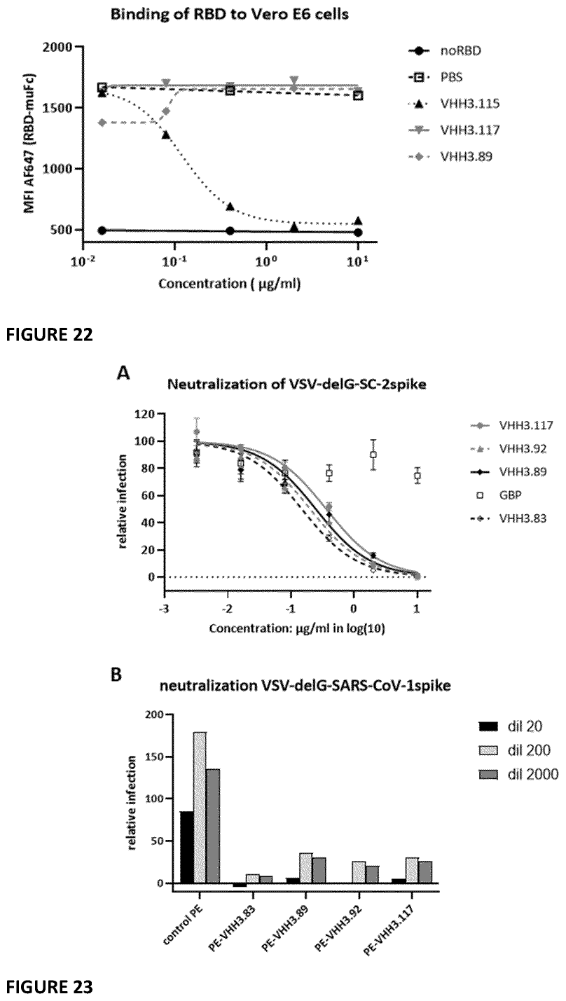

The binding site of VHH3.117 on the RBD is distant from the ACE2 binding region and consequently VHH3.117 and related VHHs fail to prevent binding of RBD to ACE2 (see Examples 5 and 7). Using Alpha LISA we previously demonstrated that also VHH3.89 does not interfere with the binding of the RBD to recombinant ACE2 in solution (see Example 13 and FIG. 20). To confirm that VHH3.89 can also not prevent the binding of SARS-CoV-2 RBD to the human receptor on the surface of target cells, we tested the binding of RBD-muFc that was pre-incubated with VHH3.89 to Vero E6 target cells. VHH3.117 and VHH3.115, which is related to VHH72 and known to prevent RBD from binding ACE2, were used as controls. FIG. 22 shows that just like VHH3.117, VHH3.89 cannot prevent the binding of RBD to ACE2 expressing Vero E6 cells at concentrations above its EC50 for neutralization of VSV-delG pseudotyped with the SARS-CoV-2 spikes (see below and FIG. 23).

To test if, similar to VHH3.117, VHH3.89 can neutralize SARS-CoV-2 without being able to block binding of RBD to ACE2, we investigated if VHH3.89 can neutralize SARS-CoV-2 spike pseudotyped VSV-delG. A GFP targeting VHH (GBP) was used as negative control, VHH3.117 and VHH3.92 were used as references and VHH3.83 that bind to the VHH72 epitope and does interfere with RBD binding to ACE2 was used as positive control (PCT/EP2021/052885). FIG. 23A illustrates that VHH3.89 neutralizes VSV-del G pseudotyped with SARS-CoV-2 spikes with an EC50 that is comparable to that of VHH3.117 and VHH3.92. In addition, PE extracts containing VHH3.89, VHH3.83, VHH3.117 or VHH3.92 were also able to neutralize SARS-CoV-1 spike pseudotyped VSV-delG (FIG. 23B). Taking into account the variation between the RBDs of SARS-CoV-2 and -1 this cross-neutralizing activity underscores that VHH3.117 and VHH3.92 bind highly similar epitopes (FIGS. 21 B and C).

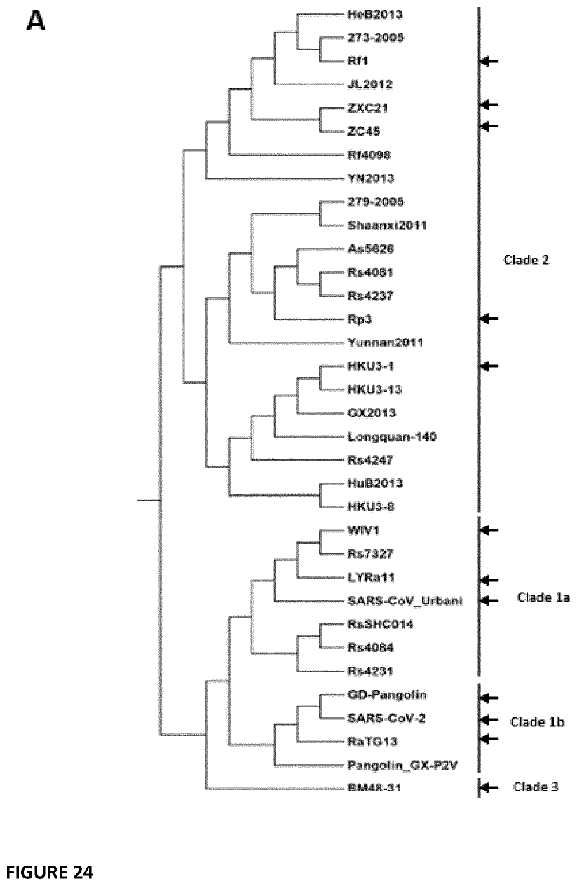

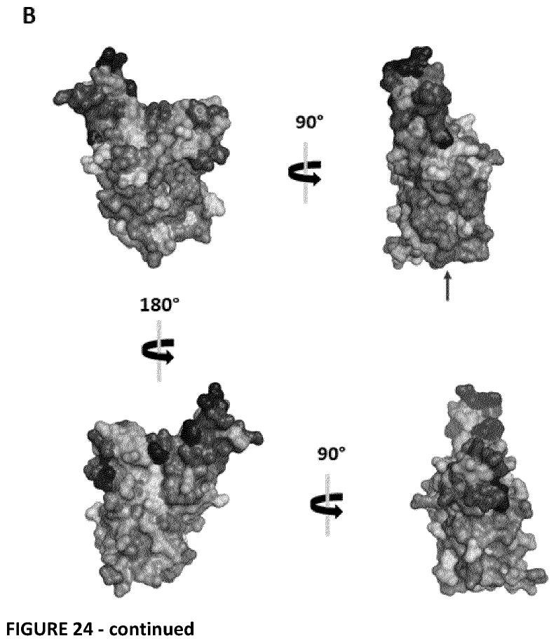

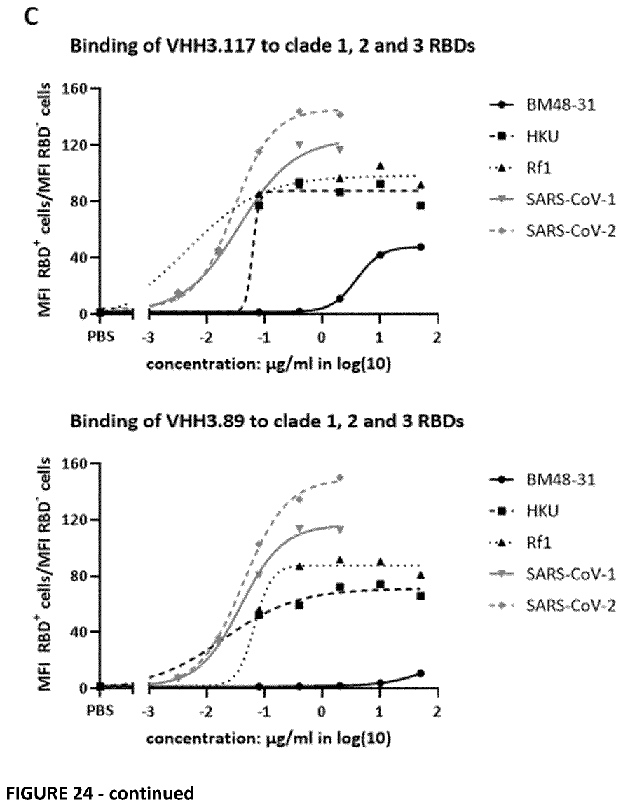

Previous analysis revealed that VHH3.117 can potently bind to the RBD of clade 1 and clade 2 sarbecoviruses and to the RBD of clade 3 BM48-31 sarbecovirus, although with reduced affinity (see Example 6, FIGS. 13 and 14). If VHH3.89 binds the RBD to a site that is highly similar to the binding site of VHH3.117, it should be able to bind the RBD of clade 1 and 2 and to lesser extent to the RBD of clade 3 sarbecoviruses. To test this, we investigated the binding of VHH3.89 to yeast cells expressing the RBD of SARS-CoV-2 (clade LB), SARS-CoV-1 (clade LA), HKU3 (clade 1), Rf1 (clade 3) and BM48-31 (clade 3) by flow cytometric analysis (FIG. 24A-C). Both VHH3.117 and VHH3.89 were able to potently bind the RBD of clade 1 and 2 sarbecoviruses and to a markedly lower extent to the RBD of the BM48-31 clade 3 virus. In addition, potent binding of both VHH3.117 and VHH3.89 was also observed for a more extended series of clade 1 and 2 viruses when tested by yeast cell ELISA (FIG. 24 D). Taking into account the few sites on the RBD that are conserved among clade 1, 2 and 3 sarbecoviruses, these results strongly argue that VHH3.89 recognizes an epitope that is highly similar to the VHH3.117 binding site.

### Example 15. Humanization of VHH3.117-Epitope Binding Agents

The skilled person is aware of the methodologies and techniques for humanization as known in the art, and has the knowledge at hand to try out a number of humanization substitutions. In particular, humanizations and reduction of chemical heterogeneity propensity of VHH sequences are based on alignment with the human immunoglobulin G heavy chain variable domain germline-3 (IGHy3) consensus sequence, or polymorphic variations thereof as described in L. Mitchell and L. J. Colwell (2018. Proteins 86: 697-706); this analysis is performed both by sequence comparison and by checking all residue positions in 3D structures of a typical camelid-VHH framework (e.g. the 3D-structure of VHH72; as is accessible in PDB entry 6WAQ). The camelid polar sequon at positions 43-47 (e.g. KEREG (SEQ ID NO:67), sequential numbering) is preserved (in classical heavy chain/light chain-antibodies this is KGLEW (SEQ ID NO:68) and comprises the heavy chain/light chain interaction zone). The framework and CDRs are analysed for possible problematic residues/sequons (e.g. NXT glycan sequon, methionine, asparagine deamidation, aspartate isomerisation, potential furin cleavage sites) and are corrected when deemed necessary and possible without majorly affecting the binding affinity of the VHH. The preferred positions and residues for humanization of camelid VHH sequences has been described herein above.

We further provide insights and constructs to make humanized variants of the binders described herein.

For VHH3.117-epitope binding agents, such as VHH3.117, a humanized version may constitute a variant with substitutions Q1D, QSV, K83R, and Q108L (according to Kabat numbering).

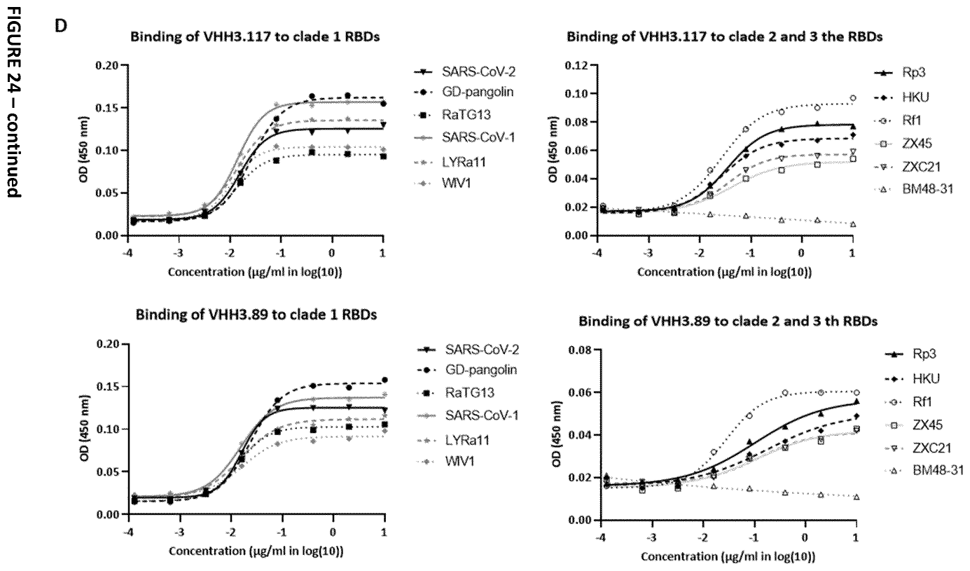

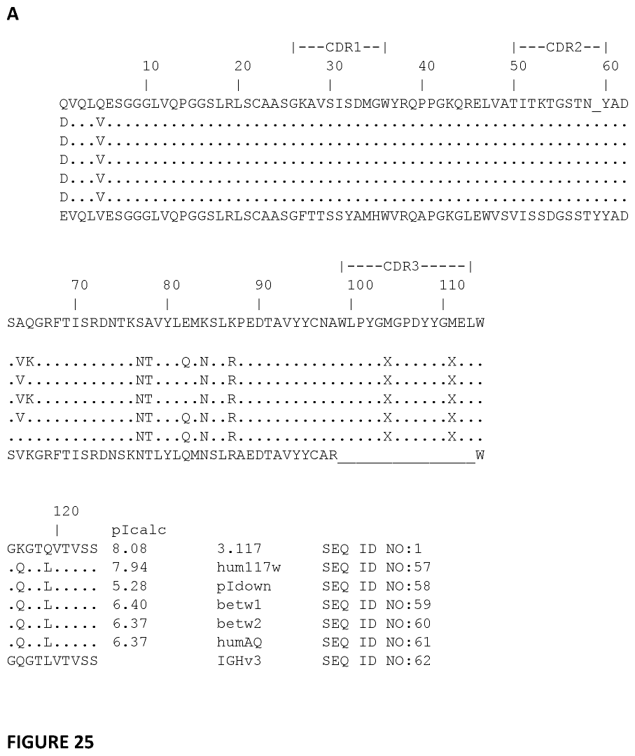

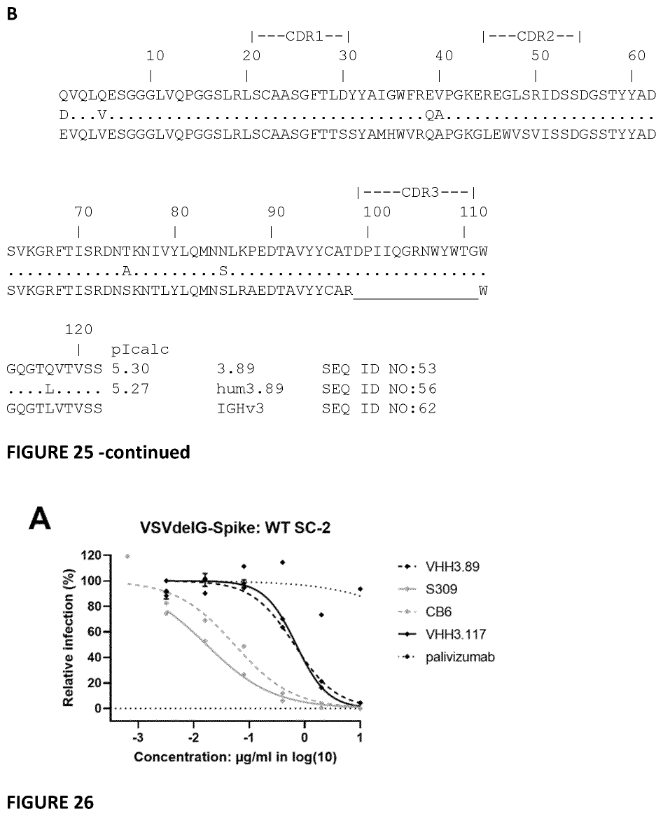

As shown in FIG. 25A, the following substitutions are proposed for humanization of VHH3.117 (using sequential numbering as presented in the alignment shown in FIG. 25A):

(1) Framework 1: humanize Q1 to E, or substitute Q1 to D (in order to eliminate possibility for N-term pyro-glutamate formation), humanize Q5 to V.

(2) Framework 3: humanize 64-65 AQ to VK, 77-78 SA to NT, E82 to O K84 to N, K87 to R.

(3) CDR3: contains two methionine residues that are potentially sensitive to oxidation. Versions of the VHH3.117 can be made in which either or both methionine residues are mutated to alanine to investigate whether binding of the VHH3.117 to its antigen (SARS-CoV-2 receptor-binding domain, SARS-CoV-2 spike or orthologs of these proteins from related viruses) is influenced by these mutations. Subsequently or alternatively, either or both residues can be mutated to preferably another hydrophobic acid, most preferably isoleucine or leucine, and the resulting protein variants can be investigated for binding of the resulting variant of VHH117 to its antigen. ‘X’ in FIG. 25A stands for any other amino acid, preferably each independently Leu, Ile, Ala, or Val.

(4) End-framework: humanize K116 to O Q119 to L.

The binding of the adapted humVHH3.117 protein variants (most preferably incorporating all of the mutations set forth above, with both methionine residues substituted to isoleucine) is then assessed to its antigen (SARS-CoV-2 receptor-binding domain, SARS-CoV-2 spike or orthologs of these proteins from related viruses) in comparison to the native VHH3.117 protein.

It will be clear to the person skilled in the art that in other embodiments, proteins variants containing only a subset of the above mutations can be made and assessed for antigen binding.

Examples of such variants containing only a subset of the above mutations are shown in FIG. 25A. In one of these examples, the isoelectric point of the molecule is taken into account as an additional design parameter, and the E82 is retained (E occasionally occurs in that position also in human IGVH sequences) to retain a negatively charged residue that is predicted to lower the isoelectric point of the adapted VHH117 sequence ‘betw1’ (E82 is human-allowed), in which the two Met residues in CDR3 can, for instance, be mutated to Ile or Leu.

Alternatively, a number of humanized variants are envisaged for characterization of VHH3.117, with the five most prominent candidate residues for humanization substitutions at locations (according to Kabat numbering): Q1, to substitute with D as to avoid pyroglutamate, though the N-terminal substitution may affect the binding properties of VHH3.117 since this is closely located near the epitope region. So a further in-depth analysis of such a variant as to confirm binding potential may be required. Additionally, Q5 replacement with V, K84 replacement with N, K87 with R and Q108 with L are envisaged herein.

Specifically for the original llama-based sequence of VHH3.117 (SEQ ID NO:1) there may be a requirement for its developability to substitute two methionine residues in CDR3 for obtaining a proper humanized variant. Care should however be taken not to loose or affect its binding capacity, so a sequential substitution approach is recommended.

Furthermore, additional residues may require substitutions for obtaining proper humanized variants, including the Proline at position 39 in framework 2, for instance by an Alanine, the A-Q at position 64-65, and the S-A at positions 77-78, as well as the E82 in framework 3, for instance to be replaced with VK, NT or NA, and Q, resp), and the K on position 108 with Q (according to Kabat numbering).

In addition to humanization of VHH3.117, similar substitutions may be envisaged in the family members including VHH3.92, 3.94, 3.42 and 3.180 (as presented in SEQ ID Nos:2-5).

Specifically the framework residues may be substituted with residues that are known to be more ‘human-like’, while the CDR residues are preferably maintained. Specifically, in the case of humanization of VHH3.117 family members, the CDR sequences as provided in SEQ ID NO: 6 for CDR1, SEQ ID NO:7 for CDR2 and SEQ ID NO:8 for CDR3 should remain as provided herein and the humanized variant solely differs in substitutions in the framework residues, preferably one or more of the FR residue positions as listed herein for the particular VHH, and with at least 90% identity of the humanized FR1, 2, 3 or 4, as compared to the original FR1, 2, 3 or 4 sequence.

The VHH3.89 family as described in Example 13 herein may as well be taken in consideration for humanization, similar to the humanization substitutions as typically considered in the art.

In particular, as shown in FIG. 25B, the following substitutions (using sequential numbering as presented in SEQ ID NO:53) are proposed for humanization of VHH3.89 (SEQ ID NO:53) to humanized VHH3.89 variant (SEQ ID NO:56):

(1) Framework 1: humanize Q1 to E, or substitute Q1 to D (in order to eliminate possibility for N-term pyro-glutamate formation), Q5 to V.

(2) Framework 2: humanize 39-40 EV to QA.

(3) Framework 3: humanize T75 to A, and N85 to S.

(4) End-framework: humanize Q117 to L.

The binding of the adapted humVHH3.89 protein is then assessed to its antigen (SARS-CoV-2 receptor-binding domain, SARS-CoV-2 spike or orthologs of these proteins from related viruses) in comparison to the native VHH3.89 protein.

It will be clear to the person skilled in the art that in other embodiments, proteins variants containing only a subset of the above mutations can be made and assessed for antigen binding.

Alternatively, a humanized variant constituting a ‘chimeric’ VHH based on the different family members of the VHH3.89 family may be considered, as to combine the original sequence of CDRs and FRs closest to the human-like sequences. For instance, combine CDR1 of VHH3.89 with the FRs of VHH3.83, which has a double deletion in CDR1 as compared to the other family members.

The expression and purification of said proposed humanized variants can be done according to the methods disclosed herein for cloning, expression and production, and as known to the skilled person. The analysis for selection of the most suitable humanized variants includes (but is not limited to) verification of the specific binding capacity of the humanized VHH as compared to the original VHH for binding to the RBD, for its affinity and for its neutralization potential.

### Example 16. Monovalent VHH3.117 and VHH3.89 Potently Neutralize SARS-CoV-2 Variants

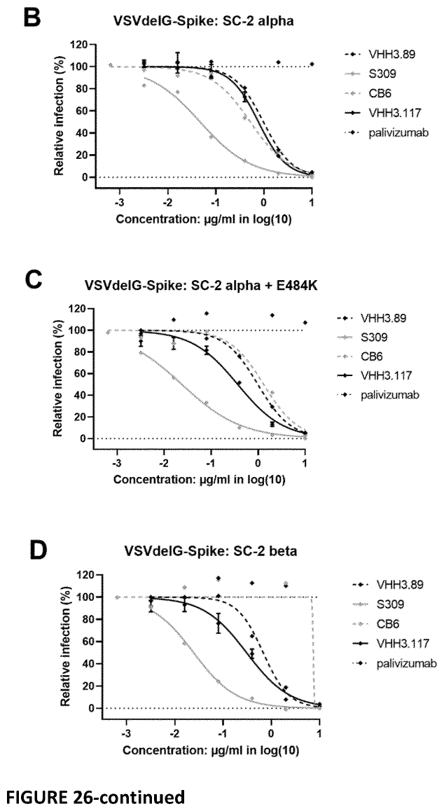

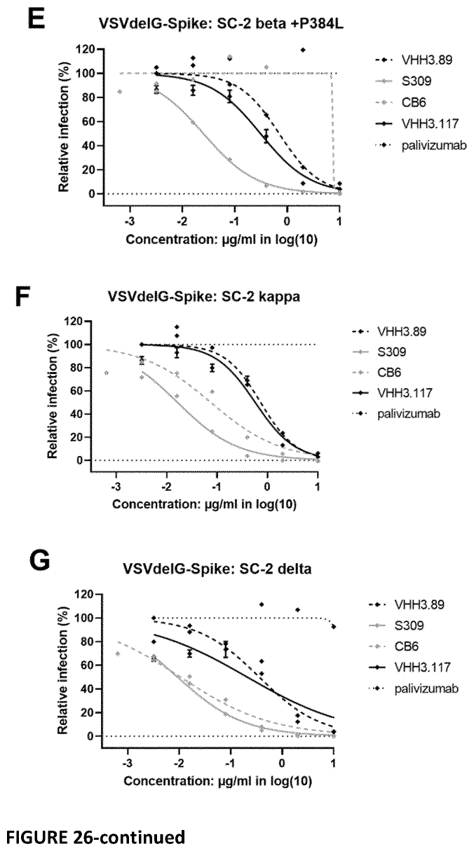

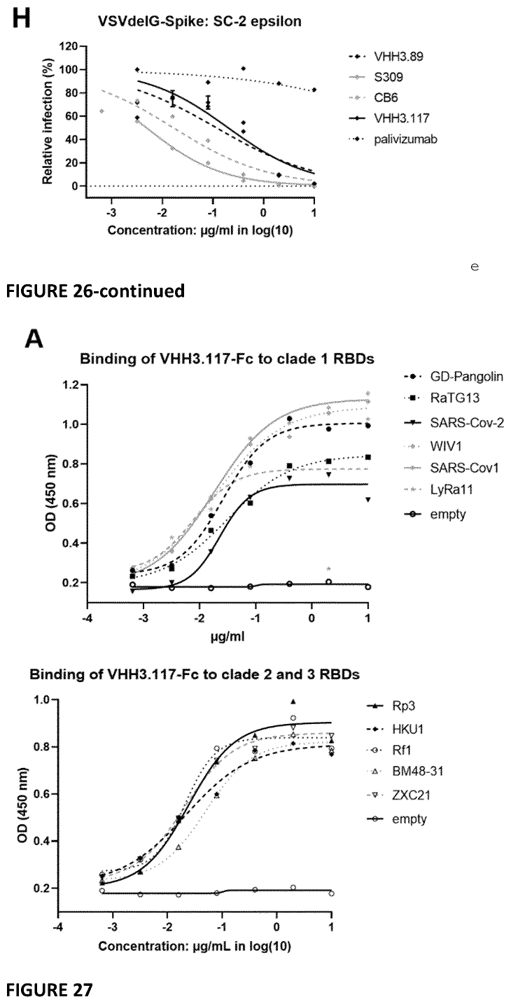

To test if VHH3.117 and VHH3.89 can neutralize SARS-CoV-2 variants of concern and variants of interest, pseudotyped VSV-delG viruses decorated with SARS-CoV-2 spikes containing the RBD mutations that are associated with those variants were generated. For the following variants the mutations in the RBD are: N501Y for the alpha variant, N501Y+E484K for the alpha+E484K variant, K417N+E484K+N501Y for the beta variant, K417N+E484K+N501Y+P384L for the beta+P384L variant, L452R+E484Q for the kappa variant, L452R+T478K for the delta variant and L452R for the epsilon variant. The neutralizing activity of VHH3.117 and VHH3.89 for the original WT SARS-CoV-2, the alpha variant, the alpha+E484K variant, the beta variant, the beta+P384L variant, the kappa variant, the delta variant and the epsilon variant was tested in a pseudovirus neutralization assay using the above described pseudotyped VSV viruses. The well described neutralizing monoclonal antibodies 5309 and CB6 and the RSV specific mononclonal antibody palivizumab, were used as controls. FIG. 26 illustrates that monovalent VHH3.117 and VHH3.89 and 5309 retain strong neutralizing activity against all tested variant viruses, whereas CB6 was not effective against the beta and beta+P384L variants.

### Example 17. Production and Purification of VHH3.117-Fc, VHH3.89-Fc and VHH3.92-Fc

The coding sequence of VHH3.117-Fc, VHH3.89-Fc, VHH3.92-Fc and VHH72-Fc were synthesized as gBlocks and cloned into an expression vector for protein production in mammalian cells. The plasmids were transiently transfected in in ExpiCHO-S™ cells for protein production. Secreted VHH-Fc proteins were purified from the growth medium by protein A affinity chromatography using a MAbSelect SuRe column. The mass and quality of the purified VHH117-Fc and VHH89-Fc were analyzed by intact and peptide mass spectrometry. For the intact protein mass spectrometry analysis, the protein was first reduced, then separated with reversed phase liquid chromatography, and finally analyzed with an Orbitrap mass spectrometer; for the peptide mass spectrometry analysis, the protein was reduced, alkylated and cleaved with trypsin, after which peptides were separated on a C18 column and online measured with an Orbitrap mass spectrometer. Peptide mapping resulted in sequence coverage of 81.9% for VHH117-Fc and 80.4% for VHH89-Fc, which was expected after tryptic digest (data not shown). Together, intact MS and peptide mapping confirmed the molecular structure of the proteins. The predominant, experimental mass of the intact protein matches with the theoretical mass of the protein, still having 2 intermolecular disulfide bonds and carrying an A2G0F N-glycosylation. Minor glycosylation types were found with intact MS and peptide mapping, for example the Man5 species (Fidata not shown). For VHH3.92-Fc no MS analysis was performed but Coomassie staining after SDS-PAGE analysis confirmed that VHH3.92-Fc is successfully purified, is intact and runs at the expected size (data not shown).

Amino acid sequences of VHH3.117-Fc, VHH3.89-Fc, VHH3.92-Fc and VHH72-Fc are as depicted hereafter:

VHH3.117-Fc:
(SEQ ID NO: 64)
DVQLQESGGGLVQPGGSLRLSCAASGKAVSISDMGWYRQPPGKQRELVAT
ITKTGSTNYADSAQGRFTISRDNTKSAVYLEMKSLKPEDTAVYYCNAWLP
YGMGPDYYGMELWGKGTQVTVSSGGGGSGGGGSDKTHTCPPCPAPEAAGG
PSVFLFPPKPKDTLMISRTPEVTCVVVDVSHEDPEVKENWYVDGVEVHNA
KTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVESCSVMHEALHNHYT
QKSLSLSPG
VHH3.89-Fc:
(SEQ ID NO: 65)
DVQLQESGGGLVQPGGSLRLSCAASGFTLDYYAIGWFREVPGKEREGLSR
IDSSDGSTYYADSVKGRETISRDNTKNIVYLQMNNLKPEDTAVYYCATDP
IIQGRNWYWTGWGQGTQVTVSSGGGGSGGGGSDKTHTCPPCPAPEAAGGP
SVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQ
KSLSLSPG
VHH3.92-Fc:
(SEQ ID NO: 63)
DVQLQESGGGLVQPGGSLRLSCAASGKAVSISDMGWYRQPPGKQRELVAT
ITKTGNTNYADSAQGRETISRDNAKSAVYLEMASLKPEDTAVYYCNAWLP
YGMGPDYYGMELWGKGTQVTVSSGGGGSGGGGSDKTHTCPPCPAPEAAGG
PSVELFPPKPKDTLMISRTPEVTCVVVDVSHEDPEVKENWYVDGVEVHNA
KTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYT
QKSLSLSPG
VHH72-Fc
(SEQ ID NO: 66)
DVQLVESGGGLVQPGGSLRLSCAASGRTFSEYAMGWFRQAPGKEREFVAT
ISWSGGATYYTDSVKGRFTISRDNAKNTVYLQMNSLRPEDTAVYYCAAAG
LGTVVSEWDYDYDYWGQGTLVTVSSGGGGSGGGGSDKTHTCPPCPAPEAA
GGPSVELFPPKPKDTLMISRTPEVTCVVVDVSHEDPEVKENWYVDGVEVH
NAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNH
YTQKSLSLSPG.

### Example 18. VHH3.117-Fc and VHH3.89-Fc Recognize the RBD of Clade 1, Clade 2 and Clade 3 Sarbecoviruses

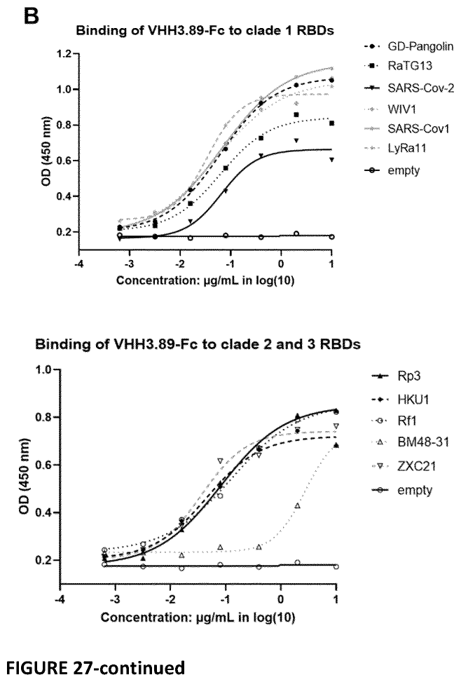

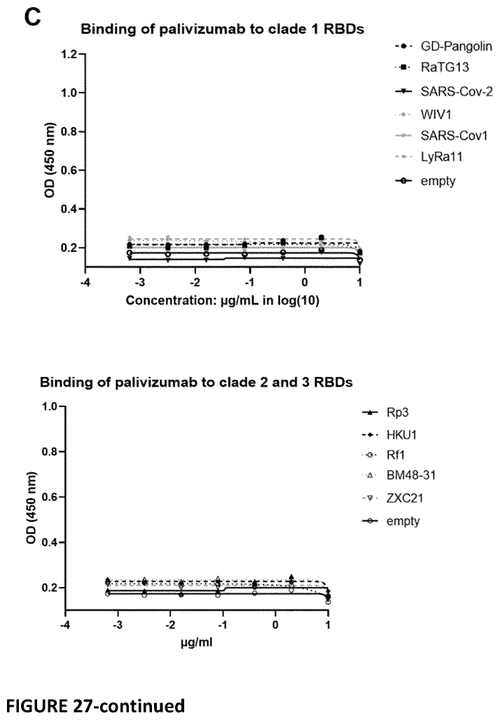

Previously we demonstrated that monovalent VHH3.117 and VHH3.89 could readily bind to the RBD of clade 1 and clade 2 sarbecoviruses but not to that of the clade 3 BM48-31 sarbecovirus (FIG. 24). To test the binding of VHH3.117 and VHH3.89 Fc fusions (VHH3.117-Fc and VHH3.89-Fc) to the RBD of sarbecoviruses we performed ELISA based on coated yeast cells expressing the RBD of diverse sarbecoviruses. FIG. 27 shows that in contrast to their monovalent counterparts VHH3.117-Fc and VHH3.89-Fc could next to clade 1 and clade 2 RBD also bind to yeast cells displaying the RBD of the BM48-31 clade 3 sarbecovirus. No binding was observed to yeast cells not displaying any RBD. These data demonstrate that VHH3.117-Fc and VHH3.89-Fc have pan-sarbecovirus specificity.

### Example 19. VHH3.177-Fc and VHH3.89-Fc Bind to RBD and Spike Protein of SARS-CoV-2 WT and the Omicron Variant

To be able to neutralize sarbecoviruses RBD-specific VHH-Fc constructs must bind to the RBD within the spike protein. Therefore, we tested the binding of VHH3.117-Fc to the spike protein of SARS-CoV-2 by ELISA using in house made recombinant stabilized Spike-HexaPro (Spike-6P) protein. This protein was produced using the SARS-CoV-2 S HexaPro expression plasmid obtained from addgene (addgene plasmid #154754, Hsieh et al. (2020) Science 369(6510):1501-1505).

The recently emerged SARS-CoV-2 omicron variant harbors multiple mutations within the RBD that enable escape from many described RBD-specific neutralizing antibodies (Liu et al. (2021) Nature). Binding of VHH3.117-Fc to the spike of the SARS-CoV-2 omicron variant was tested by ELISA using recombinant stabilized SARS-CoV-2 BA.1 Spike-HexaPro protein (Acro Biosystems, SPN-052 Hz) in ELISA.

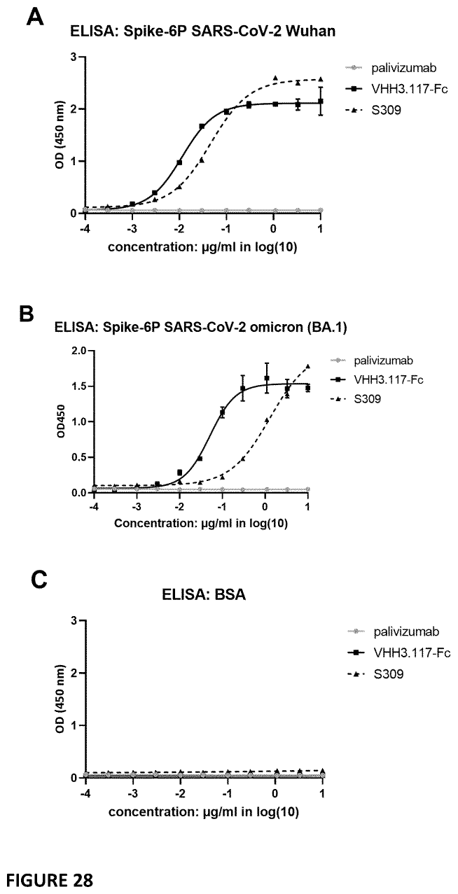

Both 5309 and VHH3.117 can bind to the spike proteins of both the original (Wuhan) and omicron SARS-CoV-2 variants (FIG. 28).

Binding of the VHH-Fc constructs to the RBD of SARS-CoV-2 original (Wuhan) and omicron variants was also tested by biolayer interferometry (BLI).

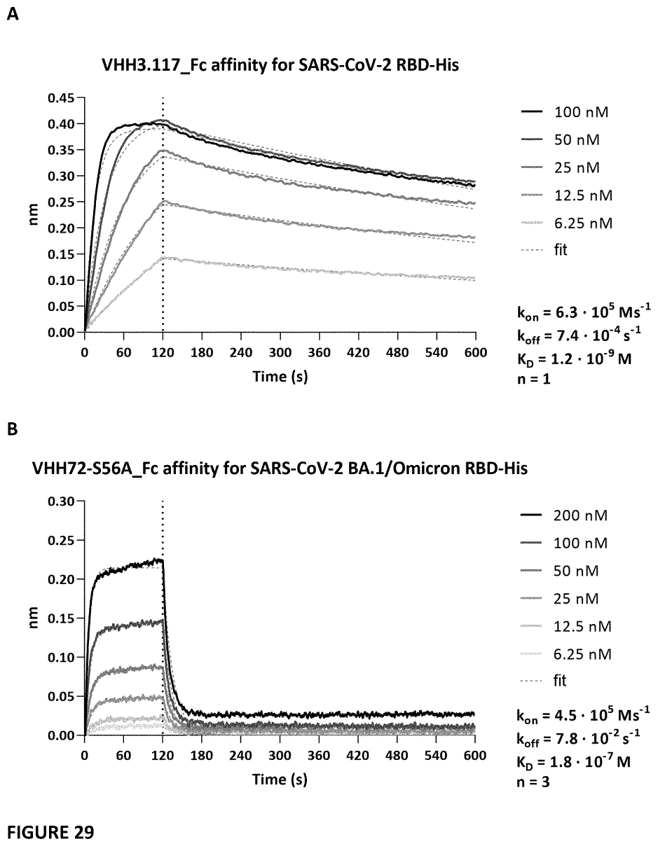

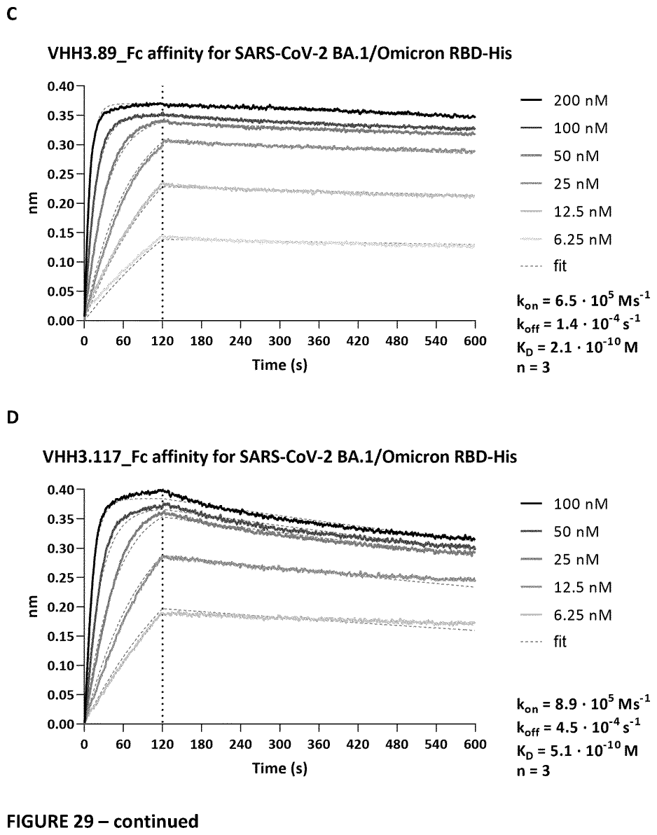

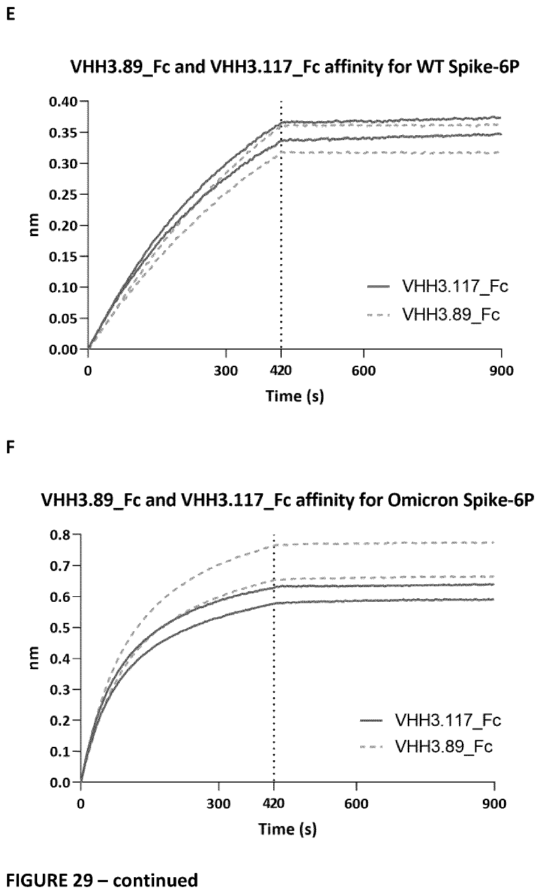

VHH3.117-Fc or VHH3.89-Fc was immobilized on anti-human IgG Fc capture (AHC) biosensors (Sartorius) via the Fc as to present the VHH to the surface. Association (120 s) and dissociation (480 s) of two-fold dilution series of His-tagged monovalent SARS-CoV-2 RBD (FIG. 29A) or His-tagged monovalent SARS-CoV-2 BA.1/Omicron RBD-His (FIG. 29 C,D) in kinetics buffer were measured. Between analyses of binding kinetics, biosensors were regenerated by three times 20 s exposure to regeneration buffer (10 mM glycine pH 1.7). Data were double reference-subtracted and aligned to each other in Octet Data Analysis software v9.0 (FortéBio). The VHH incorporated in VHH3.117-Fc was demonstrated to bind SARS-CoV-2 original (Wuhan) variant RBD with low nanomolar affinity in a 1:1 binding model (FIG. 29A). The VHHs incorporated in VHH3.89-Fc and VHH3.117-Fc bound SARS-CoV-2 Omicron variant RBD-His with subnanomolar affinity in a 1:1 binding model (FIG. 29C,D), whereas the VHHs incorporated in VHH72-S56A_Fc are demonstrated to bind Omicron RBD-His with 10⁻⁷ M affinity (FIG. 29B).

Similarly, the affinity of VHH3.117 and VHH3.89 in a VHH-Fc context for SARS-CoV-2 original (Wuhan, WT) and Omicron variants spike-6P was analysed by BLI. VHH3.117_Fc and VHH3.89_Fc were immobilized on anti-human IgG Fc capture (AHC) biosensors (Sartorius) via the Fc as to present VHH to the surface. Association (420 s) and dissociation (480 s) of 200 nM SARS-CoV-2 BA.1/O micron Spike-6P or WT Spike-6P in kinetics buffer were measured. Between analyses of binding kinetics, biosensors were regenerated by three times 20 s exposure to regeneration buffer (10 mM glycine pH 1.7). Data were double reference-subtracted and aligned to each other in Octet Data Analysis software v9.0 (FortéBio). The VHHs incorporated in VHH3.89-Fc and VHH3.117-Fc bound to Spike-6P (either Omicron or WT) with similar affinity (similar curve shapes) (FIG. 29E,F).

### Example 20. VHH3.117-Fc and VHH3.92-Fc Neutralize VSV Virus Pseudotyped with the SARS-CoV-2 Spike Protein

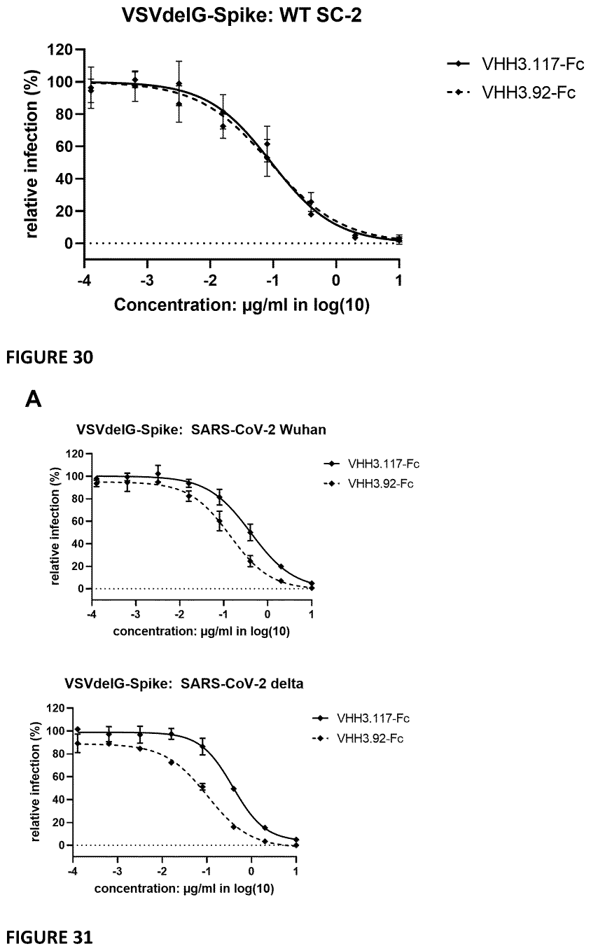

To investigate if Fc fusions of VHH3.117 and its family member VHH3.92 can neutralize SARS-CoV-2 infections, we tested if VHH3.117-Fc and VHH3.92-Fc can control infection of an pseudotyped VSV-delG virus displaying the spike protein of SARS-CoV-2 (VSVdeIG-Spike) on Vero E6 cells. VH3.117-Fc and VHH3.92-Fc neutralized VSVdeIG virus pseudotyped with the SARS-CoV-2 spike protein (FIG. 30).

### Example 21. VHH3.117-Fc can Neutralize SARS-CoV-2 Delta and Gamma Variants

To investigate of Fc fusions of VHH3.117 and its family member VHH3.92 can next to the original SARS-CoV-2 Wuhan variant also neutralize the SARS-CoV-2 delta and gamma variant, we tested if VH3.117-Fc and VHH3.92-Fc can control infection of an VSV-delG virus pseudotyped with the spike protein containing the RBD mutations of the delta or gamma variant.

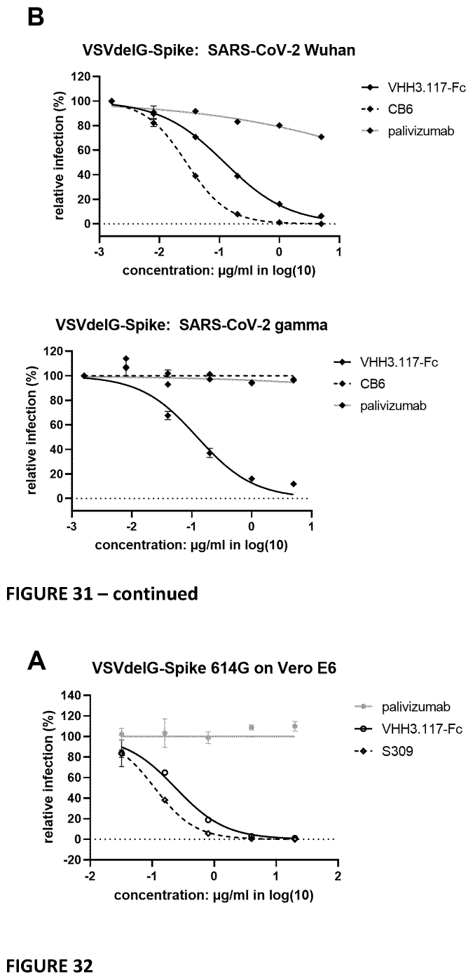

The RBD mutations of the delta variant could not overcome neutralization by VH3.117-Fc and VHH3.92-Fc (FIG. 31A).

In a separate experiment the neutralizing activity of VHH3.117-Fc for pseudotyped VSVdeIG particles displaying the spike protein containing the RBD mutations of the gamma SARS-CoV-2 variant was tested. CB6 a neutralizing antibody targeting the Receptor Binding Motive (RBM) and K417 that is substituted for an T in the gamma variant, was used as control. The VHH3.117-Fc could potently neutralize VSVdeIG virus particles harboring the spike protein of the original Wuhan variant or a spike protein containing the RBD mutations of the gamma variant (FIG. 31B). In contrast to VHH3.117-Fc, CB6 failed to neutralize the VSVdeIG pseudotyped with spike proteins containing the RBD mutations of the gamma variant.

### Example 22. VHH3.117-Fc can Neutralize the SARS-CoV-2 Omicron BA.1 Variant

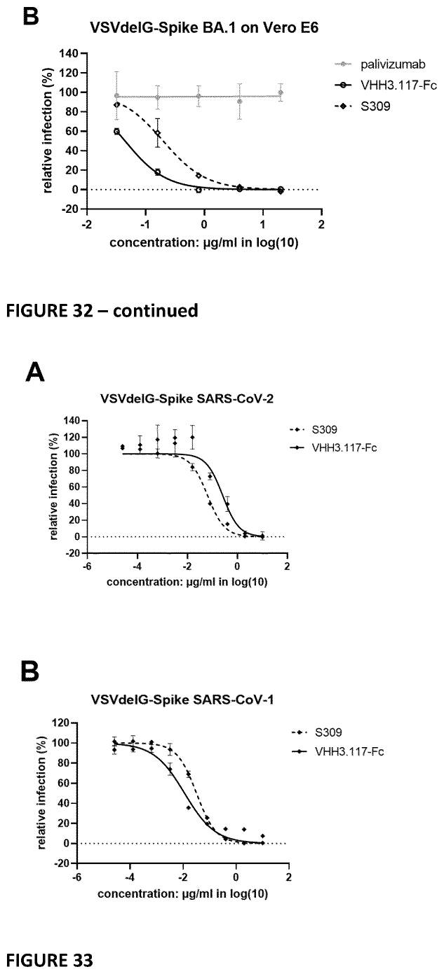

Using ELISA and BLI we demonstrated that VHH3.117-Fc can readily recognize the Spike protein of the SARS-CoV-2 omicron variant despite multiple mutation in the RBD (FIGS. 28B and 29D). To test if VHH3.117-Fc can also neutralize the SARS-CoV-2 omicron variant we performed neutralization assays using the pseudotyped VSVdeIG virus particles expressing the spike protein of the SARS-CoV 614G or the omicron BA.1 variant. As control we used the 5309 monoclonal antibody that was shown to largely retain neutralization activity against the omicron BA.1 variant. VHH3.117-Fc and 5309 neutralized VSVdeIG virus particles pseudotyped with the spike protein of the SARS-CoV 614G or the omicron BA.1 variant (FIG. 32).

### Example 23. VHH3.117-Fc can Neutralize SARS-CoV-1

In contrast to the RBD Receptor Binding Motive (RBM), the VHH3.117 binding site is well conserved between SARS-CoV-1 and SARS-CoV-2. This is illustrated by the ability of VHH3.117-Fc to bind to the RBD of a broad range of sarbecoviruses including SARS-CoV-1 (FIG. 26). To investigate if Fc fusions of VHH3.117 can also neutralize SARS-CoV-1, a neutralization assay was performed using pseudotyped VSVdeIG virus particles decorated with SARS-CoV-1 spike protein. 5309, a monoclonal antibody isolated from a SARS-CoV-1 infected patient that can neutralize both SARS-CoV-1 and SARS-CoV-2 was used as control. FIG. 33 illustrates that 5309 and VHH3.117-Fc potently neutralized both SARS-CoV-2 and SARS-CoV-1 spike protein decorated VSVdeIG virus particles.

### Example 24. VHH3.117-Fc Neutralizes VSVdeIG Virus Particles Pseudotyped with SARS-CoV-2 Spike on Vero E6 Cells that Stably Express Human TMPRSS2

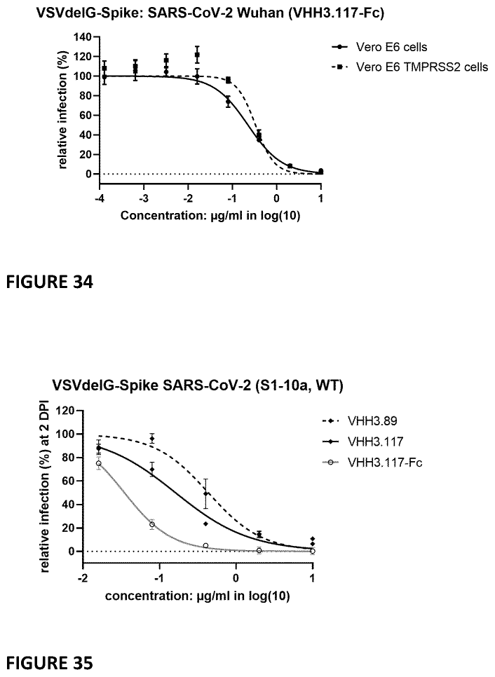

Entry of SARS-CoV viruses can occur in the endosomes after proteolytic activation of the spike protein by cathepsins that cleave the ST site upstream the fusion peptide allowing fusion. Alternatively, SARS-CoV virus can also enter at the cell surface after proteolytic activation of the spike by the transmembrane protease TMPRSS2 (Hoffmann et al. (2020) Cell 181:271-280). Vero E6 cells express undetectable levels of endogenous TMPRSS2, but allow viral entry via the cathepsin-dependent pathway (Bertram et al. (2010) J Virol. 84:10016-10025, JV 2010; Hoffmann et al. 2020). To test if VHH3.117-Fc can also block viral infection via TMPRSS2 a pseudovirus neutralization assay was performed using Vero E6 cells that stably express human TMPRSS2 (NIBIOHN, JCRB1819) (Matsuyama et al. (2020) PNAS 117:7001-7003). FIG. 34 demonstrates that VHH3.117-Fc neutralized pseudotyped VSVdeIG virus particles expressing the SARS-CoV-2 spike protein.

### Example 25. VHH3.117-Fc is Able to Neutralize Replication-Competent VSV Virus Containing the SARS-CoV-2 Spike Protein

Next we investigated if VHH3.89, VHH3.177 and VHH3.117-Fc can neutralize replication-competent VSV virus containing the SARS-CoV-2 Spike protein by making use of the S1-1a WT VSV virus described by Koenig et al. (Koenig et al. (2021) Science 371:eabe6230). FIG. 35 illustrates that VHH3.89, VHH3.117 and VHH3.117-Fc potently neutralized Spike expressing replication-competent VSV virus.

### Example 26. VHH3.117 and VHH3.89-Fc Induce Premature Shedding of the Spike S1 Subunit

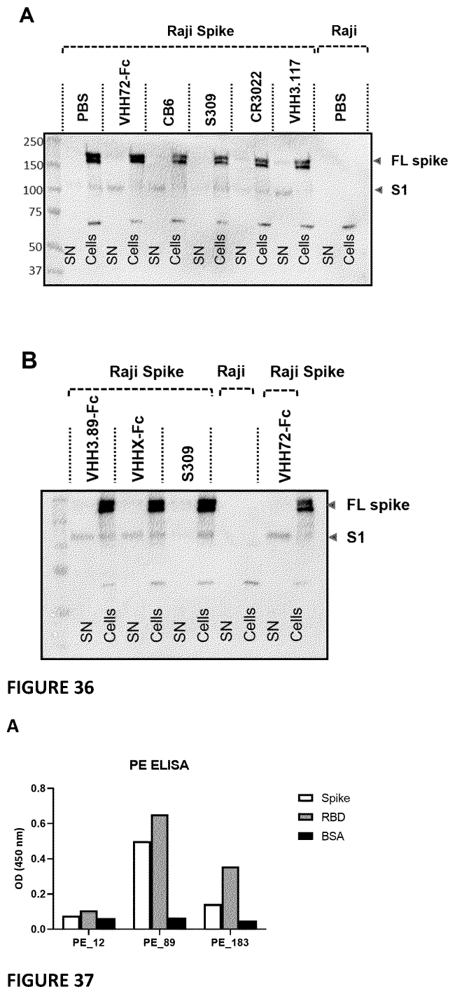

The majority of neutralizing antibodies or nanobodies that target the RBD, neutralize by preventing the binding of the RBD to its receptor ACE2 either by direct binding to the RBM (e.g. CB6) or by sterical hindrance (e.g; VHH72) (Wrapp et all. (2020) Cell 181:1004-1015.e15). Moreover, antibodies that block ACE2 binding are able to induce 51 shedding and as such premature Spike triggering (Wec et al. (2020) Science 369:731-736). We demonstrated that although VHH3.89 and VHH3.117 do neutralize SARS-CoV-2, they cannot block binding of RBD to ACE2 (FIG. 22). As an alternative mechanism of neutralization antibodies might induce 51 shedding and consequently premature spike triggering. To investigate if VHH3.117 and VHH3.89-Fc can induce 51 shedding we incubated cells expressing the SARS-CoV-2 spike protein with these antibodies and detected 51 shedding into the growth medium by Western blotting using a polyclonal 51 specific antiserum. The ACE2 blocking antibodies CB6 and VHH72-Fc were included as positive controls (Schepens et al. (2021) Sci. Transl. Med. 13). The non-neutralizing antibody CR3022 that does not block ACE2 binding and was shown not to induce 51 shedding was included as negative control (Wec et al. (2020)). In addition, we also included the neutralizing antibody 5309 that does not block ACE2 binding (Tortorici et al. (2021) Science 370:950-957). As expected antibodies (CB6 and VHH72-Fc) that can block ACE2 binding to the RBD induced shedding of 51 from the cell surface into the growth medium, as observed by the accumulation of the 51 subunit in the growth medium (SN) and the reduction of what is remained in the cellular fraction as compared to PBS treated cells (FIG. 36A). The two conventional antibodies 5309 and CR3022 that cannot block binding of ACE2 to RBD, did also not induce 51 shedding from spike expressing cells (FIG. 36). In sharp contrast to 5309 and CR3022 and despite not being able to block binding of ACE2 to RBD, VHH3.117 and VHH3.89-Fc did induce 51 shedding (FIG. 36). Without wishing to be bound by any theory, a possible explanation for the 51 shedding induced by these VHHs is that the common binding region of these VHHs is highly occluded within the spike trimer. As such binding of these VHHs might result in the destabilization of the native spike trimer and consequently promote 51 shedding and premature spike triggering.

### Example 27. Identification of the VHH3.89 Family Member VHH3.183 that can Neutralize SARS-CoV-2 Via Binding to the RBD of the SARS-CoV-2 Spike Protein

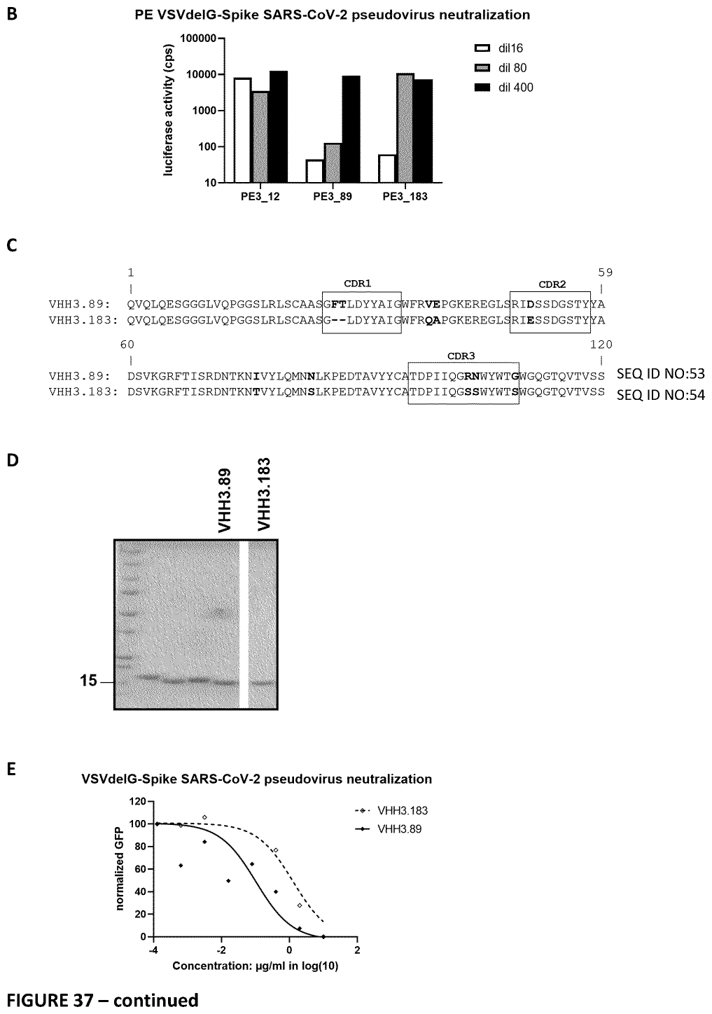

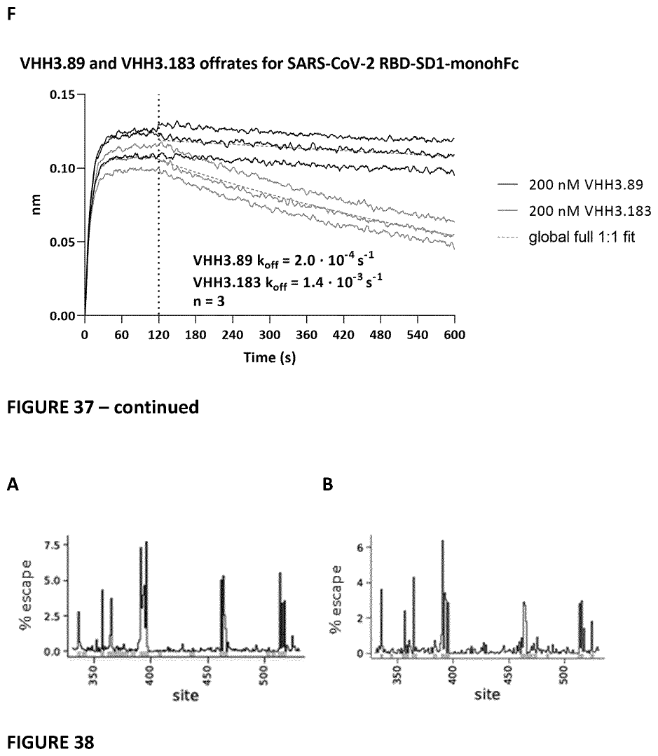

VHH3.183 was isolated in the screen from which also VHH3.89 originates. The VHH present in the crude periplasmic extracts of E. coli cells expressing respectively VHH3.89 (PE_89) and VHH3.183 (PE_183) were able to bind to the SARS-CoV-2 spike and RBD (FIG. 37A) and could neutralize VSVdeIG virus particles pseudotyped with the SARS-CoV-2 spike protein (FIG. 37B). Sequence analysis revealed that VHH3.183 is highly related to VHH3.89, containing a 2 amino acid deletion in CDR1, 1 and 3 amino acid substations in respectively CDR2 and CDR3 and few substitutions in the frame work regions 2 and 3 (FIG. 37C). Alike VHH3.89, VHH3.183 was produced in WK6 E coli cells and purified from periplasmic extracts by Ni-NTA affinity chromatography. After buffer exchange to PBS, the obtained VHHs were quantified and analyzed by SDS-PAGE (FIG. 37D). The neutralizing activity of VHH3.183 was tested by a pseudovirus neutralization assay. Alike VHH3.89, VHH3.183 neutralized VSVdeIG virus particles pseudotyped with the SARS-CoV-2 spike protein (FIG. 37E). Biolayer interferometry demonstrated the affinity of monovalent VHH3.183 for monomeric human Fc-fused SARS-CoV-2_RBD-SD1 immobilized on anti-human IgG Fc capture (AHC) biosensors with a dissociation rate of 1.4.10-3 s-1 (FIG. 37F).

### Example 28. Determination of SARS-CoV-2 RBD Amino Acid Positions that can Lose Binding to VHH3.117 and VHH3.89 when Mutated, by Deep Mutational Scanning

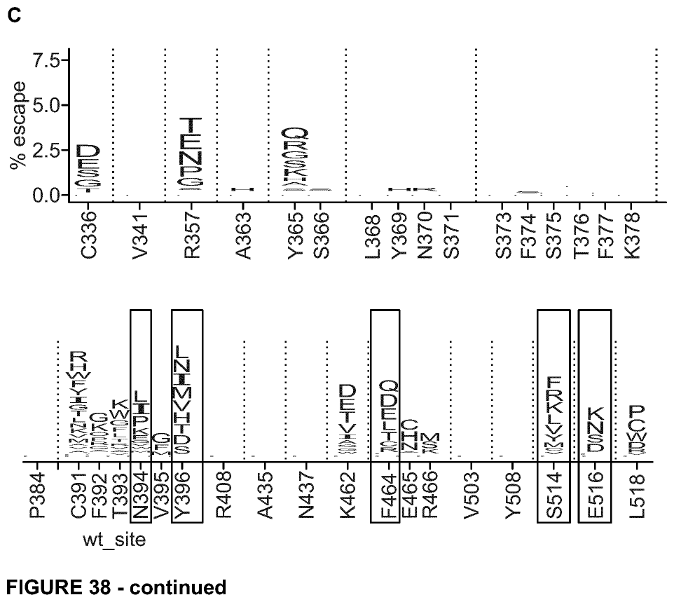

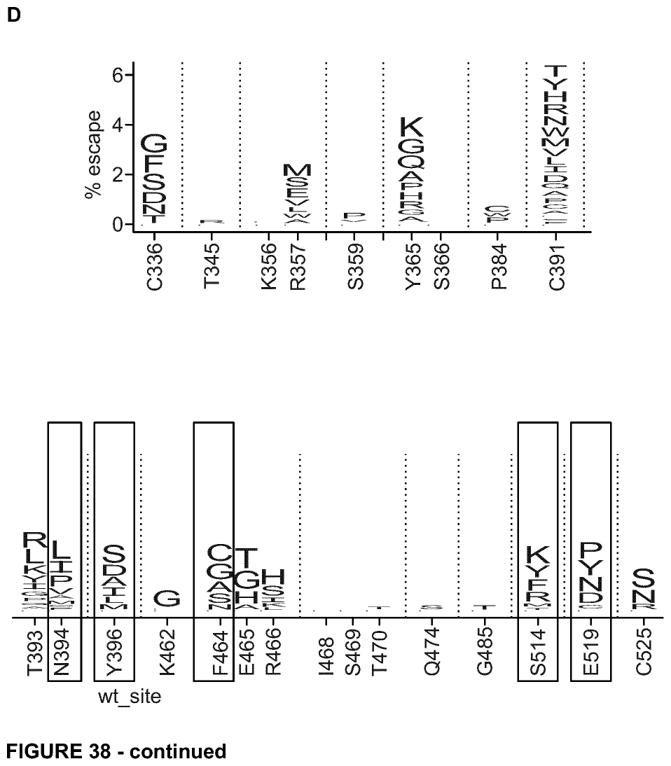

Comparison of the deep mutational scanning signal plotted over the entire length of the RBD shows that the profiles obtained with VHH3.89 and VHH3.117 are highly similar (FIG. 38A-B), demonstrating that these two VHH families are functionally affected in their binding by mutations in a highly similar set of SARS-CoV-2 RBD amino acid positions.

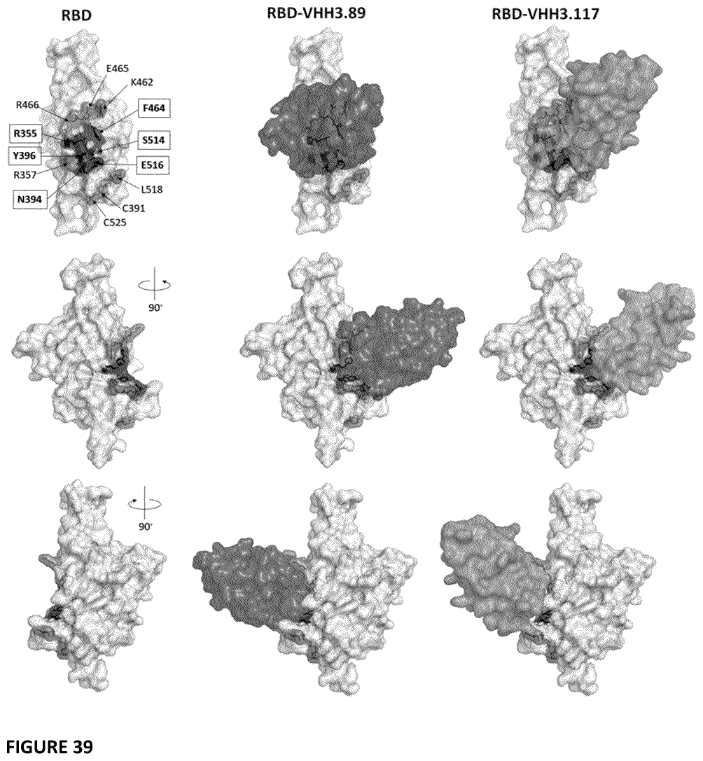

Beyond mutations that affect disulfide bonds that are important for the overall fold integrity of the RBD, the majority of the identified amino acid positions were found to effectively form part of the direct binding contact region of these VHHs with the RBD upon inspection of the corresponding cryoEM-determined structures of the complexes of these VHHs with the SARS-CoV-2 spike protein (FIG. 39), allowing to delineate that the core binding contacts for both VHH3.89 and VHH3.117 comprise the positions that are boxed in FIGS. 38C-D. Remaining positions appear to be either more peripheral contacts or local allosteric modulators of the core contact zone.

### Example 29. Cryo-EM Reconstruction of the SARS-CoV-2 Spike Protein Trimer in Complex with VHH3.89 and VHH3.117

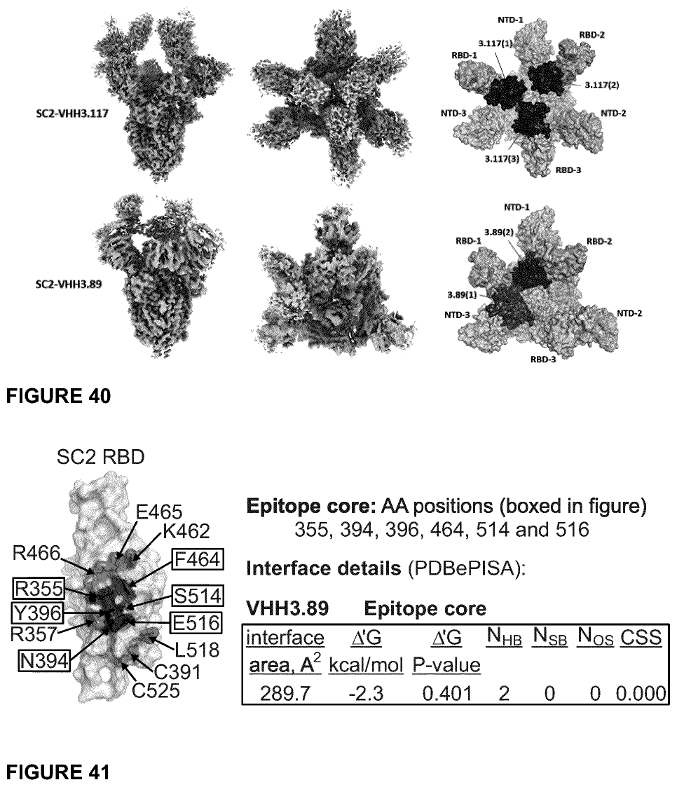

For structure determination of the Spike protein—VHH complexes, VHH3.89 or VHH3.117 were added in 1.3 molar excess to recombinant HexaPro stabilized spike protein (Spike-6P) of the Wuhan SARS-CoV-2 virus. 3 ml of a 0.72 mg/ml SC2—VHH complexes were placed on R2.1 Quantifoil grids prior to snap freezing by plunging the grids into liquid ethane. CryoEM data were collected on a JEOL cryoARM300 electron microscope equipped with Gatan K3 direct electron detector. Single particles were processed using Relion3, resulting in 3D electron potential maps with a nominal resolution of 3.1 Å for the VHH3.117 and VHH3.89 complexes. CryoEM Coulomb potential maps showed unambiguous volumes corresponding to the VHH agents. For the SC2-VHH3.117 complex, all three RBD domains in the SC2 trimer are found in an upright conformation and each have a single copy of VHH3.117 bound (FIG. 40). For the SC2-VHH3.89 complex, all three RBD domains of the SC2 trimer are found in an upright conformation, but with a poor local map density for the RBD of SC2 protomer 3, indicative of a large conformation flexibility in this RBD (FIG. 40). The RBD of SC2 protomers 1 and 2 each have a copy of VHH3.89 bound.

MATERIALS and METHODS

Production of VHHs by Pichia pastoris and Escherichia coli.

Small scale production of VHHs in Pichia pastoris is described in (Wrapp et al. 2020 Cell, supra). For the production of VHH in E. coli, a pMECS vector containing the VHH of interest was transformed into WK6 cells (the non-suppressor E. coli strain) and plated on an LB plate containing Ampicillin. The next day clones were picked and grown overnight in 2 mL LB containing 100 ug/mlampicillin and 1% glucose at 37° C. while shaking at 200 rpm. One ml of this preculture was used to inoculate 25 ml of TB (terrific broth) supplemented with 100 ug/ml ampicillin, 2 mM MgCl₂ and 0.1% glucose and incubated at 37° C. with shaking (200-250 rpm) till an OD₆₀₀ of 0.6-0.9 is reached. VHH production was induced by addition of IPTG to a final concentration of 1 mM. These induced cultures were incubated overnight at 28° C. while shaking at 200 rpm. The produced VHHs were extracted from the periplasm and purified as described in Wrapp et al. In short, the VHHs were purified from the solution using Ni Sepharose beads (GE Healthcare). After elution using 500 mM imidazole the VHH containing flow-through fractions were buffer-exchanged with PBS with a Vivaspin column (5 kDa cutoff, GE Healthcare). The purified VHHs were analyzed by SDS-PAGE and coomassie staining and by intact mass spectrometry.

Enzyme-Linked Immunosorbent Assay.

Wells of microtiter plates (type II, F96 Maxisorp, Nuc) were coated overnight at 4° C. with 100 ng of recombinant SARS-CoV S-2P protein (with foldon), SARS-CoV-1 S-2P protein (with foldon), mouse Fc-tagged SARS-CoV-2 RBD (Sinobiologicals) or BSA. The coated plates were blocked with 5% milk powder in PBS. Dilution series of the VHHs were added to the wells. Binding was detected by incubating the plates sequentially with either: mouse anti-HA (12CA5, Sigma) combined with HRP conjugated sheep anti-mouse IgG antibody (GE healthcare) or HRP-conjugated rabbit anti-camelid VHH antibodies (Genscript). After washing 50 μL of TMB substrate (Tetramethylbenzidine, BD OptETA) was added to the plates and the reaction was stopped by addition of 50 μL of 1 M H₂SO₄. The absorbance at 450 nM was measured with an iMark Microplate Absorbance Reader (Bio Rad). Curve fitting was performed using nonlinear regression (Graphpad 8.0).

For the competition assay in which binding of VHHs to monovalent RBD captured by VHH72-Fc or the human monoclonal antibodies 5309, CB6, CR3022 or palivizumab was tested, ELISA plates were coated with 50 ng of VHH72-Fc or the human monoclonal antibodies in PBS for 16 hours at 4° C. After washing with PBS and then PBS containing 0.1% tween-20, the wells were blocked with PBS containing 5% milk powder for 1 hour at room temperature, 20 ng of monomeric RBD (in house produced RBD-SD1-Avi) was added to the wells and incubated for 1 hour at room temperature. Subsequently, 0.5 ug/ml of the VHHs was added to the wells and incubated for 1 hour at room temperature. After washing 2 times with PBS and 3 times with PBS containing 2% milk and 0.05% tween-20 the bound VHHs were detected using a mouse anti-HIS-tag antibody (Biorad) and an HRP conjugated sheep anti-mouse IgG antibody (GE healthcare).

Biolayer Interferometry

The SARS-CoV-2 RBD binding kinetics of VHH variants were assessed via biolayer interferometry on an Octet RED96 system (FortéBio). To measure the affinity of monovalent VHH variants for RBD, monomeric human Fc-fused SARS-CoV-2_RBD-SD1 (Wrapp et al. 2020, supra) at 15 ug/ml was immobilized on anti-human IgG Fc capture (AHC) biosensors (FortéBio) to a signal of 0.35-0.5 nm. Association (120 s) and dissociation (480 s) of duplicate 200 nM VHHs were measured in kinetics buffer. Between analyses, biosensors were regenerated by three times 20 s exposure to regeneration buffer (10 mM glycine pH 1.7). Data were double reference-subtracted and aligned to each other in Octet Data Analysis software v9.0 (FortéBio). Off-rates (kdis) were fit in a 1:1 model.

Competition amongst VHH variants for SARS-CoV-2 RBD binding was assessed via biolayer interferometry on an Octet RED96 system (FortéBio). Bivalent VHH72-hFc (50 nM) was immobilized on anti-human IgG Fc capture (AHC) biosensors (FortéBio), followed by capture of antigen RBD-SD1_mFc (200 nM) to saturation. Then, competition with 1 uM VHH variants (protein concentrations calculated by a Trinean DropSense machine, Lunatic chip, after subtraction of the turbidity profile extrapolated from the absorbance spectrum at 320-400 nm) was measured for 600 s. Between analyses, biosensors were regenerated by three times 20 s exposure to regeneration buffer (10 mM glycine pH 1.7). Data were double reference-subtracted and aligned to each other in Octet Data Analysis software v9.0 (FortéBio).

Flow Cytometric Analysis of Antibody Binding to Sarbecovirus RBD Displayed on the Surface of Saccharomyces cerevisiae.

A pool of plasmids, based on the pETcon yeast surface display expression vector, that encode the RBDs of a set of SARS-CoV2 homologs was generously provided by Dr. Jesse Bloom (Starr et al. 2020, Cell 182:1295-1310). This pool was transformed to E. coli TOP10 cells by electroporation at the 10 ng scale and plated onto low salt LB agar plates supplemented with carbenicillin. Single clones were selected, grown in liquid low salt LB supplemented with carbenicillin and miniprepped. Selected plasmids were Sanger sequenced with primers covering the entire RBD CDS and the process was repeated until every desired RBD homolog had been picked up as a sequence-verified single clone. Additionally, the CDS of the RBD of SARS-CoV2 was ordered as a yeast codon-optimized gBlock and cloned into the pETcon vector by Gibson assembly. The plasmid was transformed into E. coli, prepped and sequence-verified as described above. DNA of the selected pETcon RBD plasmids was transformed to Saccharomyces cerevisiae strain EBY100 according to the protocol by Gietz & Schiestl (Gietz et al. 2007, Nature Protocols 2:1-8 and 31-41) and plated on yeast drop-out medium (SD agar -trp -ura). Single clones were selected and verified by colony PCR for correct insert length. A single clone of each RBD homolog was selected and grown overnight in 10 ml liquid repressive medium (SRaf -ura -trp) at 28° C. These pre-cultures were then back-diluted to 50 ml liquid inducing medium (SRaf/Gal -ura -trp) at an OD₆₀₀ of 0.67/m1 and grown for 16 hours before harvest. After washing in PBS, the cells were fixed in 1% PFA, washed twice with PBS, blocked with 1% BSA and stained with VHHs at different concentration. Binding of the antibodies was detected using Alexa fluor 633 conjugated anti-human IgG antibodies (Invitrogen). Expression of the surface-displayed myc-tagged RBDs was detected using a FITC conjugated chicken anti-myc antibody (Immunology Consultants Laboratory, Inc.). Following 3 washes with PBS containing 0.5% BSA, the cells were analyzed by flow cytometry using an BD LSRII flow cytometer (BD Biosciences). Binding was calculated as the ratio between the AF647 MFI of the RBD⁺ (FITC⁺) cells over the AF647 MFI of the RBD⁻ (FITC⁻ cells).

RBD Competition Assay on Vero E6 Cells.

SARS-CoV-2 RBD fused to murine IgG Fc (Sino Biological) at a final concentration of 0.4 μg/mL was incubated with 1 ug/ml of monovalent VHH and incubated at room temperature for 20 min followed by an additional 10 min incubation on ice. VeroE6 cells grown at sub-confluency were detached by cell dissociation buffer (Sigma) and trypsin treatment. After washing once with PBS, the cells were blocked with 1% BSA in PBS on ice. All remaining steps were also performed on ice. The mixtures containing RBD and VHHs or VHH-Fc fusions were added to the cells and incubated for 1 h. Subsequently, the cells were washed 3 times with PBS containing 0.5% BSA and stained with an AF647 conjugated donkey anti-mouse IgG antibody (Invitrogen) for 1 h. Following additional 3 washes with PBS containing 0.5% BSA, the cells were analyzed by flow cytometry using an BD LSRII flow cytometer (BD Biosciences).

CoV Pseudovirus Neutralization Assay.

To generate replication-deficient VSV pseudotyped viruses, HEK293T cells, transfected with SARS-CoV-1 S or SARS-CoV-2 S were inoculated with a replication deficient VSV vector containing eGFP and firefly luciferase expression cassettes (Berger and Zimmer 2011, PloS One 6:e25858). After a 1 h incubation at 37° C., the inoculum was removed, cells were washed with PBS and incubated in media supplemented with an anti-VSV G mAb (ATCC) for 16 h. Pseudotyped particles were then harvested and clarified by centrifugation (Wrapp et al. 2020, Cell 181:1004-1015). For the VSV pseudotype neutralization experiments, the pseudoviruses were incubated for 30 min at 37° C. with different dilutions of purified VHH or with GFP-binding protein (GBP: a VHH specific for GFP). The incubated pseudoviruses were subsequently added to subconfluent monolayers of VeroE6 cells. Sixteen h later the cells were washed once with PBS and cell lysates were prepared using passive lysis buffer (Promega). The transduction efficiency was quantified by measuring the GFP fluorescence in cell lysates using a Tecan infinite 200 pro plate reader. As indicated in the legends the GFP fluorescence was normalized using either the GFP fluorescence of non-infected cells and infected cells treated with PBS or the lowest and highest GFP fluorescence value of each dilution series. Alternatively, infection was quantified by measuring the luciferase acitivity using promega luciferase assay system and a GloMax microplate luminometer (Promega). The IC₅₀ was calculated by non-linear regression curve fitting, log(inhibitor) vs. response (four parameters).

AlphaLISA to Test ACE2/RBD Interaction.

Serial dilutions of VHHs (final concentration ranging between 90 nM-0.04 nM) were made in assay buffer (PBS containing 0.5% BSA and 0.05% Tween-20), and mixed with SARS-CoV-2 RBD that was biotinylated through an Avi-tag (AcroBiosystems, Cat nr. SPD-C82E9) (final concentration 1 nM) in white low binding 384-well microtitre plates (F-bottom, Greiner Cat nr 781904). Recombinant human ACE-2-Fc (final concentration 0.2 nM) was added to the mixture. After an incubation of 1 hour at room temperature, donor and acceptor beads were added to a final concentration of 20 ug/mL for each in a final volume of 0.025 mL. RBD was captured on streptavidin coated Alpha Donor beads (Perkin Elmer, Cat nr. 6760002). Human ACE-2-mFc protein (Sino Biological Cat nr. 10108-H05H) was captured on anti-mouse IgG (Fc specific) acceptor beads (Perkin Elmer, Cat nr. AL105C) in an additional incubation of 1 hour at room temperature in the dark. Interaction between beads was assessed after illumination at 680 nm and reading at 615 nm on an Ensight instrument.

Deep Mutational Scanning

Transformation of deep mutational SARS-CoV2 RBD libraries to E. coli. Plasmid preps of two independently generated deep mutational SARS-CoV2 RBD libraries in the pETcon vector were generously provided by Dr. Jesse Bloom (Starr et al. 2020, Cell 182, 1295-1310.e20). Ten ng of these preps were transformed to E. coli TOP10 strain via electroporation, and allowed to recover for one hour in SOC medium at 37° C. The transformation mixture was divided and plated on ten 24.5 cm×24.5 cm large bio-assay dishes containing low salt LB medium supplemented with carbenicillin, at an expected density of 100.000 clones per plate. After growing overnight, all colonies were scraped from the plates and resuspended into 300 ml low salt LB supplemented with carbenicillin. The cultures were grown for 2 hours and a half before pelleting. The cell pellet was washed once with sterile MQ, and plasmid was extracted via the QIAfilter plasmid Giga prep kit (Qiagen) according to the manufacturer's instructions.

Transformation of deep mutational SARS-CoV2 RBD libraries to S. cerevisiae. Ten μg of the resulting plasmid preps were transformed to Saccharomyces cerevisiae strain EBY100, according to the large-scale protocol by Gietz & Schiestl (Gietz et al. 2007, Nature Protocols 2:1-8 and 31-41). Transformants were selected in 100 ml liquid yeast drop-out medium (SD -trp -ura) for 16 hours. Then the cultures were back-diluted into 100 mL fresh SD -trp -ura at 1 OD₆₀₀ for an additional 9 hours passage. Afterwards, the cultures were flash frozen in 1e8 cells aliquots in 15% glycerol and stored at −80° C.

Cloning and transformation of WT RBD of SARS-CoV2 The CDS of the RBD of SARS-CoV2 was ordered as a yeast codon-optimized gBlock and cloned into the pETcon vector by Gibson assembly. The cloning mixture was similarly electroporated into E. coli TOP10 cells, and plasmid was extracted via a Miniprep kit (Promega) according to the manufacturer's instructions. The plasmid was Sanger sequenced with primers covering the entire RBD CDS. Finally, the plasmid was transformed to Saccharomyces cerevisiae strain EBY100, according to the small-scale protocol by Gietz & Schiestl (Gietz et al. 2007, Nature Protocols 2:1-8 and 31-41). Transformants were selected via a yeast colony PCR.

Presorting of deep mutational SARS-CoV2 RBD libraries on ACE2 One aliquot of each library was thawed and grown overnight in 10 ml liquid repressive medium (SRaf -ura -trp) at 28° C. Additionally, the control EBY100 strain containing the pETcon plasmid expressing WT RBD from SARS-CoV2 was inoculated in 10 ml liquid repressive medium and grown overnight at 28° C. These precultures were then back-diluted to 50 ml liquid inducing medium (SRaf/Gal -ura -trp) at an OD600 of 0.67/ml and grown for 16 hours before harvest.

The cells pellets were washed thrice with washing buffer (1×PBS+1 mM EDTA, pH 7.2+1 Complete Inhibitor EDTA-free tablet (Roche) per 50 ml buffer), and stained at an OD₆₀₀ of 8/ml with 9.09 nM hACE2-muFc (Sino Biological) in staining buffer (washing buffer+0.5 mg/ml of Bovine Serum Albumin) for one hour at 4° C. on a rotating wheel. Cells were washed thrice with staining buffer and stained with 1:100 anti-cmyc-FITC (Immunology Consultants Lab), 1:1000 anti-mouse-IgG-AF568 (Molecular Probes) and 1:200 L/D eFluor506 (Thermo Fischer Scientific) for one hour at 4° C. on a rotating wheel. Cells were washed thrice with staining buffer, and filtered over 35 μm cell strainers before sorting on a FACSMelody (BD Biosciences). A selection gate was drawn capturing the ACE2+ cells, such that, after compensation, max. 0.1% of cells of unstained and single stained controls appeared above the background. Approximately 2.5 million ACE2+ cells were collected per library, each in 5 ml polypropylene tubes coated with 2×YPAD+1% BSA.

Sorted cells were recovered in liquid SD -trp -ura medium with 100 U/ml penicillin and 100 μg/ml streptomycin (Thermo Fisher Scientific) for 72 hours at 28° C., and flash frozen at −80° C. in 9 OD₆₀₀ unit aliquots in 15% glycerol.

Nanobody escape mutant sorting on ACE2-sorted deep mutational SARS-CoV2 RBD libraries One ACE2-sorted aliquot of each library was thawed and grown overnight in 10 ml liquid repressive medium (SRaf -ura -trp) at 28° C. Additionally, the control EBY100 strain containing the pETcon plasmid expressing WT RBD from SARS-CoV2 was inoculated in 10 ml liquid repressive medium and grown overnight at 28° C. These precultures were then back-diluted to 50 ml liquid inducing medium (SRaf/Gal --ura -trp) at an OD600 of 0.67/m1 and grown for 16 hours before harvest.

The cells pellets were washed thrice with washing buffer (1×PBS+1 mM EDTA, pH 7.2+1 Complete Inhibitor EDTA-free tablet (Roche) per 50 ml buffer, freshly made and filter sterile) and stained at an OD₆₀₀ of 8/ml with a specific concentration per stained nanobody in staining buffer (washing buffer+0.5 mg/ml of Bovine Serum Albumin) for one hour at 4° C. on a rotating wheel. Specifically, we stained at 400 ng/ml for VHH72h1 S56A, 100 ng/ml for VHH3.117 (epitope map) and 10 ng/ml VHH89 (epitope map). These concentrations were determined in preparatory experiments to result in 50% half-maximal binding to yeast cells displaying the non-mutated RBD. The staining protocol for the monomeric constructs is as follows: Cells were washed thrice with staining buffer and stained with 1:2000 mouse anti-His (Biorad) for 1 h30 at 4° C. on a rotating wheel. Cells were washed thrice with staining buffer and stained with 1:100 anti-cmyc-FITC (Immunology Consultants Lab), 1:1000 anti-mouse-IgG-AF568 (Molecular Probes) and 1:200 L/D eFluor506 (Thermo Fischer Scientific) for one hour at 4° C. on a rotating wheel. After staining, cells were washed thrice with staining buffer, and filtered over 35 μm cell strainers before sorting on a FACSMelody (BD Biosciences). Gating was chosen as such that, after compensation, max. 0.1% of cells of the fully stained WT RBD control appeared in the selection gate. Between 150.000 and 350.000 or between 30.000 and 200.000 (Example 28) escaped cells were collected per library, each in 5 ml polypropylene tubes coated with 2×YPAD+1% BSA.

Sorted cells were recovered in liquid SD -trp -ura medium supplemented with 100 U/ml penicillin and 100 μg/ml streptomycin (Thermo Fisher Scientific) for 16 hours at 28° C.

DNA extraction and Illumina sequencing of nanobody escape sorted deep mutational SARS-CoV2 RBD libraries Plasmids were extracted from sorted cells using the Zymoprep yeast plasmid miniprep II kit (Zymo Research) according to the manufacturer's instructions, but with the exception of a longer (2 hour) incubation with the Zymolyase enzyme, and with the addition of a freeze-thaw cycle in liquid nitrogen after Zymolyase incubation.

A PCR was performed on the extracted plasmids using KAPA HiFi HotStart ReadyMix to add sample indices and remaining Illumina adaptor sequences using NEBNext UDI primers (20 cycles). PCR samples were purified once using Clean NGS magnetic beads (CleanNA), and once using AM Pure magnetic beads (Beckman Coulter). Fragments were eluted in 15 μl 0.1×TE buffer. Size distributions were assessed using the High Sensitivity NGS kit (DNF-474, Advanced Analytical) on a 12-capillary Fragment Analyzer (Advanced Analytical). Hundred bp single-end sequencing was performed on a NovaSeq 6000 by the VIB Nucleomics core (Leuven, Belgium).

Analysis of Sequencing Data and Epitope Calculation Using Mutation Escape Profiles.

Deep sequencing reads were processed as described by Greaney et al. 2021 (Cell Host Microbe 29:44-57) using the code available at https://github.com/jbloomlab/SARS-CoV-2-RBD MAP Crowe antibodies, with adjustments. Briefly, nucleotide barcodes and their corresponding mutations were counted using the dms_variants package (0.8.6). Escape fraction for each barcode was defined as the fraction of reads after enrichment divided by the fraction of reads before enrichment of escape variants. The resulting variants were filtered to remove unreliably low counts and keep variants with sufficient RBD expression and ACE2 binding (based on published data (Starr et al. 2020, Cell 182: 1295-1310). For variants with several mutations, the effects of individual mutations were estimated with global epistasis models, excluding mutations not observed in at least one single mutant variant and two variants overall. The resulting escape measurements correlated well between the duplicate experiments and the average across libraries was thus used for further analysis. To determine the most prominent escape sites for each nanobody, RBD positions were identified where the total site escape was >10× the median across all sites, and was also at least 10% of the maximum total site escape across all positions for a given nanobody.

S1 Shedding Assay

Antibody or VHH was added at a final concentration of 10 μg/ml to 1 million Raji cells expressing either no spike, or SARS-CoV-2 spike. The antibody-cell mixture was incubated for 30 min or 1 h at 37° C. and 5% CO₂. After incubation, cells were pelleted by centrifugation, supernatant was transferred to a fresh tube and the cell pellet was lysed with RIPA lysis buffer (50 mM Tris-HCl pH 8.0, 100 mM NaCl, 1 mM EDTA, 1 mM EGTA, 0.1% SDS, 1% NP-40). 20 μl samples of supernatant and lysate were separated on 8% SDS-PAGE gels, and electroblotted onto nitrocellulose membranes. Membranes were blocked with 4% milk, stained with rabbit anti-SARS-S1 antibody (1/1000, Sino biologics, 40591-T62) followed by anti-rabbit IgG-HRP (1/2000, GE Healthcare, NA934V) and developed using Pierce™ ECL Western Blotting Substrate (Thermofisher Scientific).

VHH-Fc Protein Production in CHO Cells

Cloning of synthetic genes. All genes were ordered synthetically at IDT as gBlocks. Upon arrival, gBlocks were solubilized in ultraclean water at a concentration of 20 ng/μL. gBlocks were A-tailed using the NEBNext-dA-tailing module (NEB), purified using CleanPCR magnetic beads (CleanNA) and inserted in pcDNA3.4-TOPO vector (ThermoFisher). The ORF of positive clones was fully sequenced, and pDNA of selected clones was prepared using the NucleoBond Xtra Midi kit (Machery-Nagel).

CHO transfection and protein purification protocol. VHH-Fc proteins were expressed in ExpiCHO-S™ cells (ThermoFisher Scientific), according to the manufacturer's protocol. Briefly, a 25 mL culture of 6×106 cells per mL, grown at 37° C. and 8% CO₂, was transfected with 20 μg of pcDNA3.3-VHH72-Fc plasmid DNA using ExpiFectamine™ CHO reagent. One day after transfection, 150 μL ExpiCHO™ enhancer and 4 mL ExpiCHO™ feed was added to the cells, and cultures were further incubated at 32° C. and 5% CO₂. Cells were fed a second time day 5 after transfection. Productions were collected as soon as cell viability dropped below 75%. For purification of the VHH-Fc proteins, supernatants were loaded on a 5 mL MAbSelect SuRe column (GE Healthcare). Unbound proteins were washed away with Mcllvaine buffer pH 7.2, and bound proteins were eluted using Mcllvaine buffer pH 3. Immediately after elution, protein-containing fractions were neutralized using 30% (v/v) of a saturated Na 3 PO 4 buffer. Next, these fractions were pooled, and loaded on a HiPrep Desalting column for buffer exchange to PBS pH7.4.

Yeast cell ELISA to test antibody binding to Sarbecovirus RBD displayed on the surface of Saccharomyces cerevisiae. Fixed yeast cells expressing the RBD of various clade 1, 2 and 3 sarbecoviruses were prepared as describe above and coated in ELISA plates in PBS (type II, F96 Maxisorp, Nuc) to obtain about 10-20% confluency. After washing twice with PBS the cells were treated with 3% H₂O₂ for 15 minutes at room temperature to inactivate yeast peroxidases. Subsequently the plates were washed 3 times with PBS and once with PBS containing 0.1% Tween-20. After blocking with 2% BSA for 1 hour, serial dilutions of VHH-Fc proteins or HA-tagged VHHs were prepared in PBS containing 0.5% BSA and 0.05% Tween-20 and added to the cells and allowed to incubate for 90 minutes. After washing 2 times with PBS and 3 times with PBS containing 0.5% BSA and 0.05% Tween-20 the bound VHHs were detected using a mouse anti-HA-tag antibody (12CA5, Sigma) and an HRP conjugated sheep anti-mouse IgG antibody (GE healthcare). Bound VHH-Fc were detected using HRP-conjugated rabbit anti-human IgG serum (Sigma, A8792). After washing 50 μL of TMB substrate (Tetramethylbenzidine, BD OptETA) was added to the plates and the reaction was stopped by addition of 50 μL of 1 M H₂SO₄. The absorbance at 450 nM was measured with an iMark Microplate Absorbance Reader (Bio Rad). Curve fitting was performed using nonlinear regression (Graphpad 8.0).

Generation of Spike Protein Expression Vectors for the Production of VSVdeIG Pseudovirus Particles Expressing Spike Proteins Containing RBD Mutations of SARS-CoV-2 Variants.

pCG1 expression vectors for the SARS-CoV-2 spike proteins containing the RBD mutations of SARS-CoV-2 variants were generated from the pcG1-SARS-2-Sde118 vector by sequentially introducing the specific RBD mutations by QuickChange mutagenisis using appropriate primers, according to the manufacturer's instructions (Aligent). For the pCG1-SARS-2-Sde118 expression vector for the omicron BA.1 variant a codon-optimized spike protein nucleotide sequence containing the BA.1 mutations as defined by the (A67V, 069-70, T951, G142D, 0143-145, N2111, Δ212, ins215EPE, G339D, S371L, S373P, S375F, K417N, N440K, G446S, S477N, T478K, E484A, Q49311, G496S, Q49811, N501Y, Y505H, T547K, D614G, H655Y, N679K, P681H, N764K, D796Y, N856K, Q954H, N969K, L981F) and flanking BamHl and Sall restriction sites was ordered at Geneart (Thermo Fischer Scientific) and cloned in the pCG1 vector as an BamHl/Sall fragment. After sequencing, clones containing the correct spike coding sequence were prepared using the Qiagen plasmide Qiagen kit. Before usage the spike coding sequence of the prepared pCG1 vectors was confirmed by Sanger sequencing.

Mass Spectrometry Analysis of Proteins.

Intact VHH-Fc protein (10 μg) was first reduced with tris(2-carboxyethyl)phosphine (TCEP; 10 mM) for 30 min at 37° C., after which the reduced protein was separated on an Ultimate 3000 HPLC system (Thermo Fisher Scientific, Bremen, Germany) online connected to an LTQ Orbitrap XL mass spectrometer (Thermo Fischer Scientific). Briefly, approximately 8 μg of protein was injected on a Zorbax 3005B-C18 column (5 μm, 300A, 1×250 mm ID×L; Agilent Technologies) and separated using a 30 min gradient from 5% to 80% solvent B at a flow rate of 100 μl/min (solvent A: 0.1% formic acid and 0.05% trifluoroacetic acid in water; solvent B: 0.1% formic acid and 0.05% trifluoroacetic acid in acetonitrile). The column temperature was maintained at 60° C. Eluting proteins were directly sprayed in the mass spectrometer with an ESI source using the following parameters: spray voltage of 4.2 kV, surface-induced dissociation of 30 V, capillary temperature of 325° C., capillary voltage of 35 V and a sheath gas flow rate of 7 (arbitrary units). The mass spectrometer was operated in MS1 mode using the orbitrap analyzer at a resolution of 100,000 (at m/z 400) and a mass range of 600-4000 m/z, in profile mode. The resulting MS spectra were deconvoluted with the BioPharma Finder™ 3.0 software (Thermo Fischer Scientific) using the Xtract deconvolution algorithm (isotopically resolved spectra). The deconvoluted spectra were manually annotated.

Peptide Mapping by Mass Spectrometry.

VHH-Fc protein (15 μg) was diluted with 50 mM triethylammonium bicarbonate (pH 8.5) to a volume of 100 μl. First, protein disulfide bonds were reduced with dithiothreitol (DTT; 5 mM) for 30 min at 55° C. and alkylated with iodoacetamide (IAA; 10 mM) for 15 min at room temperature (in the dark). The protein was then digested with LysC endoproteinase (0.25 μg; NEB) for 4 hours at 37° C., followed by sequencing grade trypsin (0.3 μg; Promega) for 16 hours at 37° C. After digestion, trifluoroacetic acid was added to a final concentration of 1%. Prior to LC-MS analysis, the samples were desalted using the Pierce™ C18 Spin Columns (Thermo Fischer Scientific). First, spin columns were activated with 400 μl 50% acetonitrile (2×) and equilibrated with 0.5% trifluoroacetic acid in 5% acetonitrile (2×), after which samples were slowly added on top of the C18 resin. The flow through of each sample was reapplied on the same spin column for 4 times to maximize peptide binding to the resin. After washing the resin with 200 μl of 0.5% trifluoroacetic acid in 5% acetonitrile (2×), peptides were eluted with 2 times 20 μl 70% acetonitrile. Desalted peptide samples were dried and resuspended in 50 μl 0.1% trifluoroacetic acid in 2% acetonitrile.

For the LC-MS/MS analysis, 5 μl of the desalted peptide samples was injected on an in-house manufactured C18 column (ReprosilPur C18 (Dr. Maisch), 5 μm, 0.25×200 mm ID×L) and separated using a 30 min gradient from 0% to 70% solvent B at a flow rate of 3 μl/min (solvent A: 0.1% formic acid and 0.05% trifluoroacetic acid in water; solvent B: 0.1% formic acid and 0.05% trifluoroacetic acid in 70% acetonitrile). The column temperature was maintained at 40° C. Eluting proteins were directly sprayed in the LTQ Orbitrap XL mass spectrometer with an ESI source using the following parameters: spray voltage of 4.2 kV, capillary temperature of 275° C., capillary voltage of 35 V and a sheath gas flow rate of 5 (arbitrary units). The mass spectrometer was operated in data-dependent mode, automatically switching between MS survey scans and MS/MS fragmentation scans of the 3 most abundant ions in each MS scan. Each MS scan (m/z 250-3000) was followed by up to 3 MS/MS scans (isolation window of 3 Da, CID collision energy of 35%, activation time of 30 ms) that fulfill predefined criteria (minimal signal of 5000 counts, exclusion of unassigned and single charged precursors). Precursor ions were excluded from MS/MS selection for 60 sec after two selections within a 30 sec time frame.

The resulting MS/MS spectra were analyzed with the BioPharma Finder™ 3.0 software (Thermo Fischer Scientific) and mapped onto the appropriate protein sequence. For peptide identification, the following parameters were used: maximum peptide mass of 7000 Da, mass accuracy of 5 ppm and a minimum confidence of 0.80. Cysteine carbamidomethylation was set as a fixed modification. Deamidation of asparagine and glutamine, pyroglutamate formation of N-terminal glutamine, glycation of lysine, and oxidation of methionine and tryptophan were set as variable modifications. The search for glycosylation modifications was enabled (CHO-specific). The maximum number of variable modifications per peptide was set at 3.

Structure determination of SC2-VHH3.89 and SC2-VHH3.117 complexes by cryoEM. Sample preparation and data collection: For structure determination of the Spike protein—VHH complexes, VHH3.89 or VHH3.117 were added in 1.3 fold molar excess to recombinant HexaPro stabilized spike protein (Spike-6P) of the Wuhan SARS-CoV-2 virus. Quantifoil R.2.1 Cu400 holey carbon grids were glow discharged in the ELMO glow discharge system (Corduan Technologies) for 1 min at 11 mA and 0.3 mbar.

The cryo-EM samples were prepared using a CP3 cryoplunger (Gatan). 2 μl of the Spike-6P-VHH complexes at 0.72 mg/ml were applied on a grid and blotted from both sides for 2 s with Whatman No. 2 filter paper at 95% relative ambient humidity, plunge-frozen in liquid ethane at −176° C. and stored in liquid nitrogen prior to data collection.

Cryo-EM images were collected on a JEOL CryoARM 300 microscope at a nominal magnification of 60,000 and the corresponding calibrated pixel size of 0.76 Å, using the Gatan K3 direct electron detector operated in counting mode. For data collection, 3.112 s exposures were dose-fractionated into 60 frames with an electron dose of 1.06 e-Å⁻² per frame. The defocus varied between −0.9 and −2.2 μm. In this way 12915 and 15663 zero-loss micrographs were recorded for the Spike-6P-VHH3.89 and Spike-6P-VHH3.117 complexes, respectively.

EM image processing: The dose-fractionated movies were imported in RELION 4.0 Beta and motion-corrected using RELION's own (CPU-based) implementation of the UCSF motioncor2 program. The Contrast Transfer Function (CTF) parameters were estimated using CTFFIND-4.1.14. References for autopicking were generated by picking a subset of 1000 micrographs using LoG-based auto-picking followed by 2D classification. These references were used for template-based picking of the full datasets, resulting in U.S. Pat. Nos. 1,894,336 and 6,777,098 picked particles for the Spike-6P-VHH3.89 and Spike-6P-VHH3.117 complex, respectively, extracted with a boxsize of 576 pixel, binned to 144 pixel. Three consecutive rounds of 2D-classification were performed to clean the particle stack, resulting in 398264 and 239918 remaining particles in the cleaned particle stack for the Spike-6P-VHH3.89 and Spike-6P-VHH3.117 complex, respectively. These remaining particles were re-extracted, binned to 288 pixel, and six initial 3D models were generated. Particles belonging to the best 3D class for each complex were re-extracted without binning and subjected to three cycles of consecutive 3D auto-refinement, CTF refinement and classification without alignment. For the Spike-6P-VHH3.89 complex, 222258 particles remained after the final round of classification and 3D auto-refinement, followed by Post-processing resulting in a map with a 3.1 Å nominal resolution according to the 0.143 FSC criterion. For the Spike-6P-VHH3.117 complex 183857 particles remained after the final round of classification and 3D auto-refinement, followed by Post-processing resulting in a 3.1 Å resolution map.

1. A sarbecovirus binding agent, wherein the agent binds to the sarbecovirus spike protein Receptor Binding Domain (SPRBD), allows binding of Angiotensin-Converting Enzyme 2 (ACE2) to SPRBD when the sarbecovirus binding agent itself is bound to SPRBD, is at least neutralizing towards SARS-CoV-2 and SARS-CoV-1, and binds:
at least one of the amino acids Thr393 (or alternatively Ser393 in some sarbecoviruses), Asn394 (or alternatively Ser394 in some sarbecoviruses), Val395, or Tyr396 of the SARS-CoV-2 spike protein as defined in SEQ ID NO:30; and
at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses), Arg466, or Arg357 (or alternatively Lys357 in some sarbecoviruses) of the SARS-CoV-2 spike protein as defined in SEQ ID wherein the binding agent comprises an immunoglobulin single variable domain (ISVD) or a functional part thereof, wherein the ISVD or functional part thereof comprises the complementarity determining regions (CDRs) present in any of SEQ ID NOs: 1 to 5 or SEQ ID NO: 53-55, wherein the CDRs are annotated according to Kabat, MacCallum, IMGT, AbM, or Chothia.

2. The sarbecovirus binding agent according to claim 1 which binds to at least amino acids Asn394 (or alternatively Ser394 in some sarbecoviruses) and Tyr396.

3. The sarbecovirus binding agent according to claim 1, which binds to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses), or Arg466 of the SARS-CoV-2 spike protein as defined in SEQ ID NO:30.

4. The sarbecovirus binding agent according to claim 1, which further binds to at least one of the amino acids Ser514, Glu516, or Leu518 of the SARS-CoV-2 spike protein as defined in SEQ ID NO:30.

5. The sarbecovirus binding agent according to claim 4 binds to at least amino acids Ser514 and Glu516.

6. The sarbecovirus binding agent according to claim 1, which further binds to the amino acid Arg355 of the SARS-CoV-2 spike protein as defined in SEQ ID NO:30.

7. A sarbecovirus binding agent wherein the agent binds to the sarbecovirus spike protein Receptor Binding Domain (SPRBD), allows binding of Angiotensin-Converting Enzyme 2 (ACE2) to SPRBD when the sarbecovirus binding agent itself is bound to SPRBD, is at least neutralizing towards SARS-CoV-2 and SARS-CoV-1, and binds to at least one of the amino acids Asn394 (or alternatively Ser394 in some sarbecoviruses), Tyr396, Phe464, Ser514, Glu516, and Arg355 of the SARS-CoV-2 spike protein as defined in SEQ ID NO:30; optionally further binds to amino acid Arg357 (or alternatively Lys357 in some sarbecoviruses) and/or Lys462 (or alternatively Arg462 in some sarbecoviruses) and/or Glu465 (or alternatively Gly465 in some sarbecoviruses) and/or Arg466 and/or Leu518.

8. The sarbecovirus binding agent according to claim 1, which neutralizes a SARS-CoV-2 variant comprising a mutation at position N439, K417, 5477, L452, T478, E484, P384, N501 and/or D614 of the SARS-CoV-2 spike protein as defined in SEQ ID NO:30.

9. The sarbecovirus binding agent according to claim 1, which neutralizes SARS-CoV-2 and/or a SARS-CoV-2 variant and/or SARS-CoV-1 in a pseudotype virus neutralization assay with an IC₅₀ of 10 μg/mL or less.

10. The sarbecovirus binding agent according to claim 1, which induces 51 shedding.

11. The sarbecovirus binding agent according to claim 1, which further allows binding of antibodies VHH72, 5309, or CB6 to SPRBD when the sarbecovirus binding agent itself is bound to SPRBD.

12. (canceled)

13. (canceled)

14. The sarbecovirus binding agent according to claim 1 wherein CDR1 is defined by SEQ ID NO:6, CDR2 defined by SEQ ID NO:7, and CDR3 defined by SEQ ID NO:8, wherein the annotations are according to Kabat.

15. The sarbecovirus binding agent according to claim 14, wherein CDR1 is selected from the group of sequences defined by SEQ ID NO: 9 and SEQ ID NO: 10, wherein CDR2 is selected from the group of sequences defined by SEQ ID NO: 11, SEQ ID NO: 12, SEQ ID NO: 13 and SEQ ID NO: 14, and wherein CDR3 is selected from the group of sequences defined by SEQ ID NO:15 and SEQ ID NO: 16.

16. The sarbecovirus binding agent according to claim 1, further comprising:
a framework region 1 (FR1) defined by SEQ ID NO:17, an FR2 defined by SEQ ID NO:18, an FR3 defined by SEQ ID NO:19, and an FR4 defined by SEQ ID NO:20; or
an FR1 selected from the group of sequences defined by SEQ ID NO: 21, SEQ ID NO: 22 and SEQ ID NO: 23, an FR2 defined by SEQ ID NO:18, an FR3 selected from the group of sequences defined by SEQ ID NO: 24, SEQ ID NO: 25, SEQ ID NO: 26 and SEQ ID NO: 27, and an FR4 selected from the group of sequences defined by SEQ ID NO: 28 and SEQ ID NO: 29; or
FR1, FR2, FR3 and FR4 regions that together have an amino acid sequence that is at least 90% amino acid identical to a combination of an FR1 selected from the group of sequences defined by SEQ ID NO: 21, SEQ ID NO: 22 and SEQ ID NO: 23 23, an FR2 defined by SEQ ID NO:18, an FR3 selected from the group of sequences defined by SEQ ID NO: 24, SEQ ID NO: 25, SEQ ID NO: 26, and SEQ ID NO: 27, and an FR4 selected from the group of sequences defined by SEQ ID NO: 28 and SEQ ID NO: 29.

17. The sarbecovirus binding agent according to claim 1, which comprises an immunoglobulin single variable domain (ISVD) defined by any one of SEQ ID NOs: 1 to 5, or defined by any amino acid sequence that is at least 90% amino acid identical to any of SEQ ID NOs: 1 to 5, wherein non-identical amino acids are located in one or more FRs.

18. The sarbecovirus binding agent according to claim 1 wherein CDR1 is defined by SEQ ID NO:76, CDR2 is defined by SEQ ID NO:77, and CDR3 is defined by SEQ ID NO:78, wherein the annotations are according to Kabat.

19. The sarbecovirus binding agent according to claim 18 wherein CDR1 is selected from the group of sequences defined by SEQ ID NO: 69 and SEQ ID NO: 70, CDR2 is selected from the group of sequences defined by SEQ ID NO: 71 and SEQ ID NO: 82, and CDR3 is selected from the group of sequences defined by SEQ ID NO:73, SEQ ID NO: 74 and SEQ ID NO: 75.

20. The sarbecovirus binding agent according to claim 18 further comprising:
a framework region 1 (FR1) defined by SEQ ID NO:82, an FR2 defined by SEQ ID NO:86, an FR3 defined by SEQ ID NO:90, and an FR4 defined by SEQ ID NO:94; or
an FR1 selected from the group of sequences defined by SEQ ID NO: 79, SEQ ID NO: 80 and SEQ ID NO: 81, an FR2 defined by SEQ ID NO:83, SEQ ID NO: 84 or SEQ ID NO: 85, an FR3 selected from the sequences defined by SEQ ID NO: 87, SEQ ID NO: 88 or SEQ ID NO: 89, and an FR4 selected from the sequences defined by SEQ ID NO: 91, SEQ ID NO: 92 or SEQ ID NO: 93; or
FR1, FR2, FR3 and FR4 regions that together have an amino acid sequence that is at least 90% amino acid identical to a combination of an FR1 selected from the group of sequences defined by SEQ ID NO: 19 to SEQ ID NO: 81, an FR2 defined by SEQ ID NO:83, SEQ ID NO: 84 or SEQ ID NO: 85, an FR3 selected from the group of sequences defined by SEQ ID NO: 87, SEQ ID NO: 88 and SEQ ID NO: 89, and an FR4 selected from the group of sequences defined by SEQ ID NO: 91, SEQ ID NO: 92 and SEQ ID NO: 93.

21. The sarbecovirus binding agent according to claim 18 comprising an immunoglobulin single variable domain (ISVD) defined by any of SEQ ID NOs: 53 to 55, or defined by any amino acid sequence that is at least 90% amino acid identical to any of SEQ ID NOs: 53 to 55, wherein the non-identical amino acids are located in one or more FRs.

22. A multivalent or multispecific sarbecovirus binding agent, wherein one or more of the binding agents according to claim 1 are fused directly or via a linker.

23. An isolated nucleic acid encoding a sarbecovirus binding agent according to claim 1.

24. A recombinant vector comprising the nucleic acid according to claim 23.

25. A pharmaceutical composition comprising a sarbecovirus binding agent according to claim 1.

26. A method of treating a subject comprising administering the sarbecovirus binding agent according to claim 1 as a medicament to the subject.

27. The method of claim 26, wherein the subject is treated for a sarbecovirus infection.

28. The method of claim 26, wherein the subject is treated to induce passive immunization in the subject.

29. The method according to claim 26, wherein the subject either has a sarbecovirus infection or does not have a sarbecovirus infection.

30. A method of diagnosing a sarbecovirus infection comprising using the sarbecovirus binding agent according to claim 1.

31. A method of manufacturing a diagnostic kit comprising obtaining the sarbecovirus binding agent according to claim 1.

32. The sarbecovirus binding agent according to claim 1, wherein the sarbecovirus is SARS-CoV-1 or SARS-CoV-2.

33. The multivalent or multispecific sarbecovirus binding agent according to claim 22, wherein the one or more of the binding agents are fused via an Fc domain.


SEQUENCE LISTING (94 sequences)

SEQ ID NO: 1 - protein, 123 aa, Lama glama
QVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSS

SEQ ID NO: 2 - protein, 123 aa, Lama glama
QVQLQESGGGLVQPGGSLRLSCAASGKAVSISDMGWYRQPPGKQRELVATITKTGNTNYADSAQGRFTISRDNAKSAVYLEMASLKPEDTAVYYCNAWLPYGMGPDYYGMELWGKGTQVTVSS

SEQ ID NO: 3 - protein, 123 aa, Lama glama
QVQLQESGGGLVQPGGSLRLSCAASGKAVSISDMGWYRQPPGKQRELVATITKSGSTNYANSAQGRFTISRDNAKSAVYLEMNSLKPEDTAVYYCNAWLPYGMGPDYYGMELWGEGTQVTVSS

SEQ ID NO: 4 - protein, 123 aa, Lama glama
QVQLQESGGGLVQPGGSLRLSCAASGSAVSINDMGWYRQPPGKQRELVATITKTGSTNYADSVKGRFTISRDNAKNAVYLEMNSLKPEDTATYYCNAWLPYGMGPDYYGMELWGKGTQVTVSS

SEQ ID NO: 5 - protein, 123 aa, Lama glama
QVQLQESGGGSVQAGRSLTLNCAASGKAVSISDMGWYRQPPGKQRELVATITKTGSTNYADSAQGRFTISRDNAKSAVYLEMNSLKPEDTAVYYCNAWLLYGMGPDYYGMELWGEGTQVTVSS

SEQ ID NO: 6 - protein, 5 aa, Artificial Sequence
  CDR1 consensus sequence comprised in immunoglobulin single variable domains VHH3.117, VHH3.42, VHH3.92, VHH3.94 and VHH3.180
  MISC_FEATURE (2)..(2): X (Xaa) at position 2 is S (Ser, serine) or N (Asn, asparagine)
IXDMG

SEQ ID NO: 7 - protein, 16 aa, Artificial Sequence
  CDR2 consensus sequence comprised in immunoglobulin single variable domains VHH3.117, VHH3.42, VHH3.92, VHH3.94 and VHH3.180
  MISC_FEATURE (5)..(5): X (Xaa) at position 5 is T (Thr, threonine) or S (Ser, serine)
  MISC_FEATURE (7)..(7): X (Xaa) at position 7 is S (Ser, serine) or N (Asn, asparagine)
  MISC_FEATURE (12)..(12): X (Xaa) at position 12 is D (Asp, aspartic acid) or N (Asn, asparagine)
  MISC_FEATURE (14)..(14): X (Xaa) at position 14 is A (Ala, alanine) or V (Val, valine)
  MISC_FEATURE (15)..(15): X (Xaa) at position 15 is Q (Gln, glutamine) or K (Lys, lysine)
TITKXGXTNYAXSXXG

SEQ ID NO: 8 - protein, 14 aa, Artificial Sequence
  CDR3 consensus sequence comprised in immunoglobulin single variable domains VHH3.117, VHH3.42, VHH3.92, VHH3.94 and VHH3.180
  MISC_FEATURE (3)..(3): X (Xaa) at position 3 is P (Pro, proline) or L (Leu, leucine)
WLXYGMGPDYYGME

SEQ ID NO: 9 - protein, 5 aa, Artificial Sequence
  CDR1 comprised in immunoglobulin single variable domains VHH3.117, VHH3.92, VHH3.94 and VHH3.180
ISDMG

SEQ ID NO: 10 - protein, 5 aa, Artificial Sequence
  CDR1 comprised in immunoglobulin single variable domain VHH3.42
INDMG

SEQ ID NO: 11 - protein, 16 aa, Artificial Sequence
  CDR2 comprised in immunoglobulin single variable domains VHH3.117 and VHH3.180
TITKTGSTNYADSAQG

SEQ ID NO: 12 - protein, 16 aa, Artificial Sequence
  CDR2 comprised in immunoglobulin single variable domain VHH3.92
TITKTGNTNYADSAQG

SEQ ID NO: 13 - protein, 16 aa, Artificial Sequence
  CDR2 comprised in immunoglobulin single variable domain VHH3.94
TITKSGSTNYANSAQG

SEQ ID NO: 14 - protein, 16 aa, Artificial Sequence
  CDR2 comprised in immunoglobulin single variable domain VHH3.42
TITKTGSTNYADSVKG

SEQ ID NO: 15 - protein, 14 aa, Artificial Sequence
  CDR3 comprised in immunoglobulin single variable domains VHH3.117, VHH3.92, VHH3.94 and VHH3.42
WLPYGMGPDYYGME

SEQ ID NO: 16 - protein, 14 aa, Artificial Sequence
  CDR3 comprised in immunoglobulin single variable domain VHH3.180
WLLYGMGPDYYGME

SEQ ID NO: 17 - protein, 30 aa, Artificial Sequence
  FR1 consensus sequence comprised in immunoglobulin single variable domains VHH3.117, VHH3.42, VHH3.92, VHH3.94 and VHH3.180
  MISC_FEATURE (11)..(11): X(Xaa) at position 11 is L (Leu, leucine) or S (Ser, serine)
  MISC_FEATURE (14)..(14): X(Xaa) at position 14 is P (Pro, proline) or A (Ala, alanine)
  MISC_FEATURE (16)..(16): X(Xaa) at position 16 is G (Gly, glycine) or R (Arg, arginine)
  MISC_FEATURE (19)..(19): X(Xaa) at position 19 is R (Arg, arginine) or T (Thr, threonine)
  MISC_FEATURE (21)..(21): X(Xaa) at position 21 is S (Ser, serine) or N (Asn, asparagine)
  MISC_FEATURE (27)..(27): X(Xaa) at position 27 is K (Lys, lysine) or S (Ser, serine)
QVQLQESGGGXVQXGXSLXLXCAASGXAVS

SEQ ID NO: 18 - protein, 14 aa, Artificial Sequence
  FR2 consensus sequence comprised in immunoglobulin single variable domains VHH3.117, VHH3.42, VHH3.92, VHH3.94 and VHH3.180
WYRQPPGKQRELVA

SEQ ID NO: 19 - protein, 32 aa, Artificial Sequence
  FR3 consensus sequence comprised in immunoglobulin single variable domains VHH3.117, VHH3.42, VHH3.92, VHH3.94 and VHH3.180
  MISC_FEATURE (9)..(9): X(Xaa) at position 9 is T (Thr, threonine) or A (Ala, alanine)
  MISC_FEATURE (11)..(11): X(Xaa) at position 11 is S (Ser, serine) or N (Asn, asparagine)
  MISC_FEATURE (18)..(18): X(Xaa) at position 18 is K (Lys, lysine), A (Ala, alanine) or N (Asn, asparagine)
  MISC_FEATURE (27)..(27): X(Xaa) at position 27 is V (Val, valine) or T (Thr, threonine)
RFTISRDNXKXAVYLEMXSLKPEDTAXYYCNA

SEQ ID NO: 20 - protein, 12 aa, Artificial Sequence
  FR4 consensus sequence comprised in immunoglobulin single variable domains VHH3.117, VHH3.42, VHH3.92, VHH3.94 and VHH3.180
  MISC_FEATURE (4)..(4): X(Xaa) at position 4 is K (Lys, lysine) or E (Glu, glutamine)
LWGXGTQVTVSS

SEQ ID NO: 21 - protein, 30 aa, Artificial Sequence
  FR1 comprised in immunoglobulin single variable domains VHH3.117, VHH3.92 and VHH3.94
QVQLQESGGGLVQPGGSLRLSCAASGKAVS

SEQ ID NO: 22 - protein, 30 aa, Artificial Sequence
  FR1 comprised in immunoglobulin single variable domain VHH3.42
QVQLQESGGGLVQPGGSLRLSCAASGSAVS

SEQ ID NO: 23 - protein, 30 aa, Artificial Sequence
  FR1 comprised in immunoglobulin single variable domain VHH3.180
QVQLQESGGGSVQAGRSLTLNCAASGKAVS

SEQ ID NO: 24 - protein, 32 aa, Artificial Sequence
  FR3 consensus sequence comprised in immunoglobulin single variable domains VHH3.117
RFTISRDNTKSAVYLEMKSLKPEDTAVYYCNA

SEQ ID NO: 25 - protein, 32 aa, Artificial Sequence
  FR3 consensus sequence comprised in immunoglobulin single variable domain VHH3.92
RFTISRDNAKSAVYLEMASLKPEDTAVYYCNA

SEQ ID NO: 26 - protein, 32 aa, Artificial Sequence
  FR3 consensus sequence comprised in immunoglobulin single variable domains VHH3.94 and VHH3.180
RFTISRDNAKSAVYLEMNSLKPEDTAVYYCNA

SEQ ID NO: 27 - protein, 32 aa, Artificial Sequence
  FR3 consensus sequence comprised in immunoglobulin single variable domains VHH3.42
RFTISRDNAKNAVYLEMNSLKPEDTATYYCNA

SEQ ID NO: 28 - protein, 12 aa, Artificial Sequence
  FR4 consensus sequence comprised in immunoglobulin single variable domains VHH3.117, VHH3.42, VHH3.92 and VHH3.94
LWGKGTQVTVSS

SEQ ID NO: 29 - protein, 12 aa, Artificial Sequence
  FR4 consensus sequence comprised in immunoglobulin single variable domain VHH3.180
LWGEGTQVTVSS

SEQ ID NO: 30 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - protein, 1255 aa, Sars-Cov-1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 32 - protein, 254 aa, Severe acute respiratory syndrome coronavirus 2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 33 - protein, 189 aa, Severe acute respiratory syndrome coronavirus 2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELL

SEQ ID NO: 34 - protein, 252 aa, Sars-Cov-1
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSE

SEQ ID NO: 35 - protein, 183 aa, Sars-Cov-1
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFE

SEQ ID NO: 36 - protein, 201 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 37 - protein, 201 aa, GD-Pangolin sarbecovirus RGD
NITNLCPFGEVFNATTFASVYAWNRKRISNCVADYSVLYNSTSFSTFKCYGVSPTKLNDLCFTNVYADSFVVRGDEVRQIAPGQTGRIADYNYKLPDDFTGCVIAWNSNNLDSKVGGNYNYLYRLFRKSNLKPFERDISTEIYQAGSTPCNGVEGFNCYFPLQSYGFHPTNGVGYQPYRVVVLSFELLNAPATVCGPKKST

SEQ ID NO: 38 - protein, 201 aa, RaTG13 sarbecovirus RGD
NITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKST

SEQ ID NO: 39 - protein, 200 aa, WIV1 sarbecovirus RGD
NITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLST

SEQ ID NO: 40 - protein, 200 aa, LYRa11 sarbecovirus RGD
NITNLCPFGEVFNATTFPSVYAWERKRISNCVADYSVLYNSTSFSTFKCYGVSAIKLNDLCFSNVYADSFVVKGDDVRQIAPGQTGVIADYNYKLPDDFMGCVLAWNTRNIDATSSGNFHYKYRSLRHGKLRPFERDISNVPFSPDGKPCTPPAFNCYWPLNDYGFYTTNGIGYQPYRVVVLSFELLNAPATVCGPKLST

SEQ ID NO: 41 - protein, 200 aa, Sars-Cov-1
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLST

SEQ ID NO: 42 - protein, 182 aa, Rp3 sarbecovirus RGD
NITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSHRKTKLKPFERDLSSDENGVRTLSTYDFYPSVPVAYQATRVVVLSFELLNAPATVCGPKLST

SEQ ID NO: 43 - protein, 183 aa, HKU3-1 sarbecovirus RGD
NITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKLST

SEQ ID NO: 44 - protein, 182 aa, ZXC21 sarbecovirus RGD
NITNVCPFHKVFNATRFPSVYAWERTKISDCIADYTVFYNSTSFSTFKCYGVSPSKLIDLCFTSVYADTFLIRFSEVRQVAPGQTGVIADYNYKLPDDFTGCVIAWNTAKQDTGHYFYRSHRSTKLKPFERDLSSDENGVRTLSTYDFNPNVPLEYQATRVVVLSFELLNAPATVCGPKLST

SEQ ID NO: 45 - protein, 182 aa, ZC45 sarbecovirus RGD
NITNVCPFHKVFNATRFPSVYAWERTKISDCIADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSDENGVRTLSTYDFNPNVPLEYQATRVVVLSFELLNAPATVCGPKLST

SEQ ID NO: 46 - protein, 182 aa, Rf1 sarbecovirus RGD
NITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKQDVGSYFYRSHRSSKLKPFERDLSSEENGVRTLSTYDFNQNVPLEYQATRVVVLSFELLNAPATVCGPKLST

SEQ ID NO: 47 - protein, 197 aa, BM48-31 sarbecovirus RGD
NITQLCPFNEVFNITSFPSVYAWERMRITNCVADYSVLYNSSASFSTFQCYGVSPTKLNDLCFSSVYADYFVVKGDDVRQIAPAQTGVIADYNYKLPDDFTGCVIAWNTNSLDSSNEFFYRRFRHGKIKPYGRDLSNVLFNPSGGTCSAEGLNCYKPLASYGFTQSSGIGFQPYRVVVLSFELLNAPATVCGPKQST

SEQ ID NO: 48 - protein, 21 aa, Artificial Sequence
  CDR3 of nanobody n3088 of Wu et al. 2020, Cell Host Microbe 27891-898
ARVREYYDILTGYSDYYGMDV

SEQ ID NO: 49 - protein, 13 aa, Artificial Sequence
  CDR3 of nanobody n3130 of Wu et al. 2020, Cell Host Microbe 27891-898
ATRSPYGDYAFSY

SEQ ID NO: 50 - protein, 10 aa, Artificial Sequence
  CDR3 of nanobody n3086 of Wu et al. 2020, Cell Host Microbe 27891-898
ARDFNWGVDY

SEQ ID NO: 51 - protein, 12 aa, Artificial Sequence
  CDR3 of nanobody n3113 of Wu et al. 2020, Cell Host Microbe 27891-898
VSNWASGSTGDY

SEQ ID NO: 52 - protein, 186 aa, Severe acute respiratory syndrome coronavirus 2
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELL

SEQ ID NO: 53 - protein, 122 aa, Artificial Sequence
  VHH3.89
QVQLQESGGGLVQPGGSLRLSCAASGFTLDYYAIGWFREVPGKEREGLSRIDSSDGSTYYADSVKGRFTISRDNTKNIVYLQMNNLKPEDTAVYYCATDPIIQGRNWYWTGWGQGTQVTVSS

SEQ ID NO: 54 - protein, 120 aa, Artificial Sequence
  VHH3_183
QVQLQESGGGLVQPGGSLRLSCAASGLDYYAIGWFRQAPGKEREGLSRIESSDGSTYYADSVKGRFTISRDNTKNTVYLQMNSLKPEDTAVYYCATDPIIQGSSWYWTSWGQGTQVTVSS

SEQ ID NO: 55 - protein, 122 aa, Artificial Sequence
  VHH3C_80
QVQLQESGGGSVQPGESLRLSCVGSGHTLDDYDVGWFRQAPGKEREVLSRIDSSDGSTYYADSVKGRFTISRDNTKNIVYLQMNMLKPEDTAAYYCATDPIIRGHNWYWTGWSQSTHITVSS

SEQ ID NO: 56 - protein, 122 aa, Artificial Sequence
  Humanized VHH3.89 variant
DVQLVESGGGLVQPGGSLRLSCAASGFTLDYYAIGWFRQAPGKEREGLSRIDSSDGSTYYADSVKGRFTISRDNAKNIVYLQMNSLKPEDTAVYYCATDPIIQGRNWYWTGWGQGTLVTVSS

SEQ ID NO: 57 - protein, 123 aa, Artificial Sequence
  Humanized VHH3.117 variant hum117w
  MISC_FEATURE (103)..(103): Xaa can be any other amino acid, preferably Leu, Ile, Ala, Val
  MISC_FEATURE (110)..(110): Xaa can be any other amino acid, preferably Leu, Ile, Ala, Val
DVQLVESGGGLVQPGGSLRLSCAASGKAVSISDMGWYRQPPGKQRELVATITKTGSTNYADSVKGRFTISRDNTKNTVYLQMNSLRPEDTAVYYCNAWLPYGXGPDYYGXELWGQGTLVTVSS

SEQ ID NO: 58 - protein, 123 aa, Artificial Sequence
  Humanized VHH3.117 variant pIdown
  MISC_FEATURE (103)..(103): Xaa can be any other amino acid, preferably Leu, Ile, Ala, Val
  MISC_FEATURE (110)..(110): Xaa can be any other amino acid, preferably Leu, Ile, Ala, Val
DVQLVESGGGLVQPGGSLRLSCAASGKAVSISDMGWYRQPPGKQRELVATITKTGSTNYADSVQGRFTISRDNTKNTVYLEMNSLRPEDTAVYYCNAWLPYGXGPDYYGXELWGQGTLVTVSS

SEQ ID NO: 59 - protein, 123 aa, Artificial Sequence
  Humanized VHH3.117 variant betw1
  MISC_FEATURE (103)..(103): Xaa can be any other amino acid, preferably Leu, Ile, Ala, Val
  MISC_FEATURE (110)..(110): Xaa can be any other amino acid, preferably Leu, Ile, Ala, Val
DVQLVESGGGLVQPGGSLRLSCAASGKAVSISDMGWYRQPPGKQRELVATITKTGSTNYADSVKGRFTISRDNTKNTVYLEMNSLRPEDTAVYYCNAWLPYGXGPDYYGXELWGQGTLVTVSS

SEQ ID NO: 60 - protein, 123 aa, Artificial Sequence
  Humanized VHH3.117 variant betw2
  MISC_FEATURE (103)..(103): Xaa can be any other amino acid, preferably Leu, Ile, Ala, Val
  MISC_FEATURE (110)..(110): Xaa can be any other amino acid, preferably Leu, Ile, Ala, Val
DVQLVESGGGLVQPGGSLRLSCAASGKAVSISDMGWYRQPPGKQRELVATITKTGSTNYADSVQGRFTISRDNTKNTVYLQMNSLRPEDTAVYYCNAWLPYGXGPDYYGXELWGQGTLVTVSS

SEQ ID NO: 61 - protein, 123 aa, Artificial Sequence
  Humanized VHH3.117 variant humAQ
  MISC_FEATURE (103)..(103): Xaa can be any other amino acid, preferably Leu, Ile, Ala, Val
  MISC_FEATURE (110)..(110): Xaa can be any other amino acid, preferably Leu, Ile, Ala, Val
DVQLVESGGGLVQPGGSLRLSCAASGKAVSISDMGWYRQPPGKQRELVATITKTGSTNYADSAQGRFTISRDNTKNTVYLQMNSLRPEDTAVYYCNAWLPYGXGPDYYGXELWGQGTLVTVSS

SEQ ID NO: 62 - protein, 109 aa, Artificial Sequence
  IGHv3
EVQLVESGGGLVQPGGSLRLSCAASGFTTSSYAMHWVRQAPGKGLEWVSVISSDGSSTYYADSVKGRFTISRDNSKNTLYLQMNSLRAEDTAVYYCARWGQGTLVTVSS

SEQ ID NO: 63 - protein, 359 aa, Artificial Sequence
  VHH3.92-Fc
DVQLQESGGGLVQPGGSLRLSCAASGKAVSISDMGWYRQPPGKQRELVATITKTGNTNYADSAQGRFTISRDNAKSAVYLEMASLKPEDTAVYYCNAWLPYGMGPDYYGMELWGKGTQVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 359 aa, Artificial Sequence
  VHH3.117-Fc
DVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 358 aa, Artificial Sequence
  VHH3.89-Fc
DVQLQESGGGLVQPGGSLRLSCAASGFTLDYYAIGWFREVPGKEREGLSRIDSSDGSTYYADSVKGRFTISRDNTKNIVYLQMNNLKPEDTAVYYCATDPIIQGRNWYWTGWGQGTQVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 361 aa, Artificial Sequence
  VHH72-Fc
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 5 aa, Artificial Sequence
  Camelid polar sequon
KEREG

SEQ ID NO: 68 - protein, 5 aa, Artificial Sequence
  Polar sequence in heavy chain/light chain antibody
KGLEW

SEQ ID NO: 69 - protein, 5 aa, Artificial Sequence
  CDR1 of VHH3.89 and VHH3_183 according to Kabat annotation
YYAIG

SEQ ID NO: 70 - protein, 5 aa, Artificial Sequence
  CDR1 of VHH3C_80 according to Kabat annotation
DYDVG

SEQ ID NO: 71 - protein, 17 aa, Artificial Sequence
  CDR2 of VHH3.89 and VHH3C_80 according to Kabat annotation
RIDSSDGSTYYADSVKG

SEQ ID NO: 72 - protein, 17 aa, Artificial Sequence
  CDR2 of VHH3_183 according to Kabat annotation
RIESSDGSTYYADSVKG

SEQ ID NO: 73 - protein, 12 aa, Artificial Sequence
  CDR3 of VHH3.89 according to Kabat annotation
DPIIQGRNWYWT

SEQ ID NO: 74 - protein, 12 aa, Artificial Sequence
  CDR3 of VHH3_183 according to Kabat annotation
DPIIQGSSWYWT

SEQ ID NO: 75 - protein, 12 aa, Artificial Sequence
  CDR3 of VHH3C_80 according to Kabat annotation
DPIIRGHNWYWT

SEQ ID NO: 76 - protein, 5 aa, Artificial Sequence
  consensus CDR1 of VHH3.89 family
  MISC_FEATURE (1)..(1): Xaa is D (Asp, aspartic acid) or Y (Tyr, tyrosine)
  MISC_FEATURE (3)..(3): Xaa is D (Asp, aspartic acid) or A (Ala, alanine)
  MISC_FEATURE (4)..(4): Xaa is V (Val, valine) or I (Ile, isoleucine)
XYXXG

SEQ ID NO: 77 - protein, 17 aa, Artificial Sequence
  consensus CDR2 of VHH3.89 family
  MISC_FEATURE (3)..(3): Xaa is D (Asp, aspartic acid) or E (Glu, Glutamine)
RIXSSDGSTYYADSVKG

SEQ ID NO: 78 - protein, 12 aa, Artificial Sequence
  consensus CDR3 of VHH3.89 family
  MISC_FEATURE (5)..(5): Xaa is R (Arg, arginine) or Q (Gln, glutamine)
  MISC_FEATURE (7)..(7): Xaa is R (Arg, arginine), S (Ser, serine) or H (His, histidine)
  MISC_FEATURE (8)..(8): Xaa is N (Asn, asparagine) or S (Ser, serine)
DPIIXGXXWYWT

SEQ ID NO: 79 - protein, 30 aa, Artificial Sequence
  FR1 of VHH3.89
QVQLQESGGGLVQPGGSLRLSCAASGFTLD

SEQ ID NO: 80 - protein, 28 aa, Artificial Sequence
  FR1 of VHH3_183
QVQLQESGGGLVQPGGSLRLSCAASGLD

SEQ ID NO: 81 - protein, 30 aa, Artificial Sequence
  FR1 of VHH3C_80
QVQLQESGGGSVQPGESLRLSCVGSGHTLD

SEQ ID NO: 82 - protein, 30 aa, Artificial Sequence
  consensus FR1 of VHH3.89 and VHH3C_80
  MISC_FEATURE (11)..(11): Xaa is S (Ser, serine) or L (Leu, leucine)
  MISC_FEATURE (16)..(16): Xaa is E (Glu, glutamic acid) or G (Gly, glycine)
  MISC_FEATURE (23)..(23): Xaa is A (Ala, alanine) or V (Val, valine)
  MISC_FEATURE (24)..(24): Xaa is G (Gly, glycine) or A (Ala, alanine)
  MISC_FEATURE (27)..(27): Xaa is H (His, histidine) or F (Phe, phenylalanine)
QVQLQESGGGXVQPGXSLRLSCXXSGXTLD

SEQ ID NO: 83 - protein, 14 aa, Artificial Sequence
  FR2 of VHH3.89
WFREVPGKEREGLS

SEQ ID NO: 84 - protein, 14 aa, Artificial Sequence
  FR2 of VHH3_183
WFRQAPGKEREGLS

SEQ ID NO: 85 - protein, 14 aa, Artificial Sequence
  FR2 of VHH3C_80
WFRQAPGKEREVLS

SEQ ID NO: 86 - protein, 14 aa, Artificial Sequence
  consensus FR2 of VHH3.89 family
  MISC_FEATURE (4)..(4): Xaa is Q (Gln, glutamine) or E (Glu, glutamic acid)
  MISC_FEATURE (5)..(5): Xaa is A (Ala, alanine) or V (Val, valine)
  MISC_FEATURE (12)..(12): Xaa is G (Gly, glycine) or V (Val, valine)
WFRXXPGKEREXLS

SEQ ID NO: 87 - protein, 32 aa, Artificial Sequence
  FR3 of VHH3.89
RFTISRDNTKNIVYLQMNNLKPEDTAVYYCAT

SEQ ID NO: 88 - protein, 32 aa, Artificial Sequence
  FR3 of VHH3_183
RFTISRDNTKNTVYLQMNSLKPEDTAVYYCAT

SEQ ID NO: 89 - protein, 32 aa, Artificial Sequence
  FR3 of VHH3C_80
RFTISRDNTKNIVYLQMNMLKPEDTAAYYCAT

SEQ ID NO: 90 - protein, 32 aa, Artificial Sequence
  FR3 consensus of VHH3.89 family
  MISC_FEATURE (12)..(12): Xaa is I (Ile, isoleucine) or T (Thr, threonine)
  MISC_FEATURE (19)..(19): Xaa is M (Met, methionine), N (Asn, asparagine) or S (Ser, serine)
  MISC_FEATURE (27)..(27): Xaa is V (Val, valine) or A (Ala, alanine)
RFTISRDNTKNXVYLQMNXLKPEDTAXYYCAT

SEQ ID NO: 91 - protein, 12 aa, Artificial Sequence
  FR4 of VHH3.89
GWGQGTQVTVSS

SEQ ID NO: 92 - protein, 12 aa, Artificial Sequence
  FR4 of VHH3_183
SWGQGTQVTVSS

SEQ ID NO: 93 - protein, 12 aa, Artificial Sequence
  FR4 of VHH3C_80
GWSQSTHITVSS

SEQ ID NO: 94 - protein, 12 aa, Artificial Sequence
  consensus FR4 of VHH3.89 family
  MISC_FEATURE (1)..(1): Xaa is S (Ser, serine) or G (Gly, glycine)
  MISC_FEATURE (3)..(3): Xaa is G (Gly, glycine) or S (Ser, serine)
  MISC_FEATURE (5)..(5): Xaa is G (Gly, glycine) or S (Ser, serine)
  MISC_FEATURE (7)..(7): Xaa is Q (Gln, glutamine) or H (His, histidine)
  MISC_FEATURE (8)..(8): Xaa is V (Val, valine) or I (Ile, isoleucine)
XWXQXTXXTVSS